(19) United States
(12) Patent Application Publication
(10) Pub. No.: US 2025/0179045 A1
(43) Pub. Date: Jun. 5, 2025

(54) LPAR1 ANTAGONISTS AND USES THEREOF

(71) Applicant: Contineum Therapeutics, Inc., San Diego, CA (US)

(72) Inventors: Jeffrey Roger Roppe, Temecula, CA (US); Austin Chih-Yu Chen, San Diego, CA (US); Yifeng Xiong, San Diego, CA (US); Thomas Schrader, San Diego, CA (US)

(21) Appl. No.: 18/294,513

(22) PCT Filed: Aug. 4, 2022
(86) PCT No.: PCT/US2022/039465

§ 371 Date: Feb. 1, 2024

Related U.S. Application Data
(60) Provisional application No. 63/229,857, filed on Aug. 5, 2021.

(51) Int. Cl. C07D 401/12; A61K 31/337; A61K 31/351; A61K 31/4015; A61K 31/44; A61K 31/4427; A61K 31/443; A61K 31/4433; A61K 31/4439; A61K 31/444; A61K 31/4545; C07C 235/82; C07D 207/277; C07D 213/75; C07D 305/08; C07D 309/08; C07D 405/12; C07D 405/14

(52) U.S. Cl. CPC C07D 401/12; A61K 31/337; A61K 31/351; A61K 31/4015; A61K 31/44; A61K 31/4427; A61K 31/443; A61K 31/4433; A61K 31/4439; A61K 31/444; A61K 31/4545; C07C 235/82; C07D 207/277; C07D 213/75; C07D 305/08; C07D 309/08; C07D 405/12; C07D 405/14; C07C 2601/14

(57) ABSTRACT

Described herein, inter alia, are LPAR1 antagonists and uses thereof.

### CROSS REFERENCE TO RELATED APPLICATIONS

This application claims priority to U.S. Provisional Application No. 63/229,857, filed Aug. 5, 2021, which is hereby incorporated by reference in its entirety and for all purposes.

### BACKGROUND

Lysophosphatidic acid, or LPA, is a family of bioactive phospholipids that are associated with multiple cellular functions. While the family members differ with regards to the length and the degree of saturation of their respective long-chain fatty acid backbone (Fujiwara et al., J. Biol. Chem., 2005, 280, 35038-35050), they are all capped by a glycerol-phosphate group through an ester linkage. LPAs are produced biologically from membrane phospholipids through a multi-step cascade mediated by enzymes that include lysophospholipase D (lysoPLD), autotaxin (ATX), phospholipase A1 (PLA1), phospholipase A2 (PLA2) and acylglycerol kinase (AGK) (Mutoh et al., British J Pharmacol., 2012, 165, 829-844). Once formed, the LPAs can regulate numerous cellular signaling pathways by binding to a class of 7-membrane domain G protein-coupled receptors (GPCRs), collectively known as LPA receptors (LPARs), of which six have been characterized: LPAR1, LPAR2, LPAR3, LPAR4, LPAR5, and LPAR6 (Choi, J. W., Annu. Rev. Pharmacol. Toxicol., 2010, 50, 157-186). The biological responses elicited by the binding of LPAs to LPARs are both wide-ranging and context-dependent (Yung et al., J. Lipid Res. 2014, 55 1192-1214; Yung et al., Neuron 2015, 85, 669-682). These can include induction of cell proliferation, stimulation of cell migration and contraction, promotion of neurite retraction, suppression of apoptosis, initiation of chemotaxis, closure of gap junction, and others (Chun et al., Editors, Lysophospholipid Receptors: Signaling and Biochemistry, 2013, Wiley, ISBN: 978-O-470-56905-4). Furthermore, aberrant upregulation of the LPA pathway has been implicated in multiple diseases, including cancer, inflammatory diseases, infertility, neuropathic pain, psychotic and neurodegenerative disorders, atherosclerosis, as well as fibrosis of the skin, kidney, lung, and liver (Choi, J. W., Annu. Rev. Pharmacol. Toxicol., 2010, 50, 157-186; Noguchi et al., Curr. Opin Pharmacol., 2009, 9 15-23; Yanagida et al., J. Biochem., 2011, 150, 223-232). Consequently, the targeting of LPA receptors has been, and continues to be an area of intense interest for the identification of potential treatments for these disorders. Disclosed herein, inter alia, are solutions to these and other problems in the art.

### BRIEF SUMMARY

In an aspect is provided a compound, or a pharmaceutically acceptable salt or solvate thereof, having the formula:

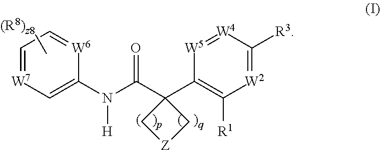

R¹ is unsubstituted C₂-C₅ alkyl.

W² is N or C(R²).

R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

W⁴ is N or C(R⁴).

R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —N^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

W⁵ is N or C(R⁵).

R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —N^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)OR^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² and R³ substituents may optionally be joined to form a substituted or unsubstituted cycloalkyl, substituted or unsubstituted heterocycloalkyl, substituted or unsubstituted aryl, or substituted or unsubstituted heteroaryl.

R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl.

R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl.

W⁶ is N or C(R⁶).

R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —N^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

W⁷ is N, N⁺—O−⁻, or C(R⁷).

R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —NR^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl.

Z is O, C(R¹¹)(R¹²), or C(O)N(R¹³).

R¹¹ is hydrogen, halogen, —CX¹¹₃, —CHX¹¹₂, —CH₂X¹¹, —OCX¹¹₃, —OCH₂X¹¹, —OCHX¹¹₂, —CN, —SOₙ₁₁R^{11D}, —SOᵥ₁₁NR^{11A}R^{11B}, —NR^{11C}NR^{11A}R^{11B}, —ONR^{11A}R^{11B}, —NHC(O)NR^{11C}NR^{11A}R^{11B}, —NHC(O)NR^{11A}R^{11B}, —N(O)ₘ₁₁, —NR^{11A}R^{11B}, —C(O)R^{11C}, —C(O)OR^{11C}, —C(O)NR^{11A}R^{11B}, —OR^{11D}, —SR^{11D}, —NR^{11A} SO₂R^{11D}, —NR^{11A} C(O)R^{11C}, —NR^{11A}C(O)OR^{11C}, —NR^{11A}OR^{11C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹² is hydrogen, halogen, —CX¹²₃, —CHX¹²₂, —CH₂X¹², —OCX¹²₃, —OCH₂X¹², —OCHX¹²₂, —CN, —SOₙ₁₂R^{12D}, —SOᵥ₁₂NR^{12A}R^{12B}, —NR^{12C}NR^{12A}R^{12B}, —ONR^{12A}R^{12B}, —NHC(O)NR^{12C}NR^{12A}R^{12B}, —NHC(O)NR^{12A}R^{12B}, —N(O)ₘ₁₂, —NR^{12A}R^{12B}, —C(O)R^{12C}, —C(O)OR^{12C}, —C(O)NR^{12A}R^{12B}, —OR^{12D}, —SR^{12D}, —NR^{12A}SO₂R^{12D}, —NR^{12A}C(O)R^{12C}, —NR^{12A}C(O)OR^{12C}, —NR^{12A}OR^{12C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹¹ and R¹² substituents may optionally be joined to form an oxo, substituted or unsubstituted cycloalkyl, or substituted or unsubstituted heterocycloalkyl.

R¹³ is hydrogen, halogen, —CX¹³₃, —CHX¹³₂, —CH₂X¹³, —OCX¹³₃, —OCH₂X¹³, —OCHX¹³₂, —SOₙ₁₃R^{13D}, —SOᵥ₁₃NR^{13A}R^{13B}, —C(O)R^{13C}, —C(O)OR^{13C}, —C(O)NR^{13A}R^{13B}, —OR^{13D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}R^{6B}, R^{6C}, R^{6D}, R^{7A}, R^{7B}, R^{7C}, R^{7D}, R^{8A}, R^{8B}, R^{8C}, R^{8D}, R^{11A}, R^{11B}, R^{11C}, R^{11D}, R^{12A}, R^{12B}, R^{12C}R^{12D}, R^{13A}, R^{13B}, R^{13C}, and R^{13D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{12A} and R^{12B} substituents bonded to the same nitrogen atom may optionally be joined to form a substituted or unsubstituted heterocycloalkyl or substituted or unsubstituted heteroaryl; R^{13A} and R^{13B} substituents bonded to the same nitrogen atom may optionally be joined to form a substituted or unsubstituted heterocycloalkyl or substituted or unsubstituted heteroaryl.

X², X³, X⁴, X⁵, X⁶, X⁷, X⁸, X¹¹, X¹², and X¹³ are independently —F, —Cl, —Br, or —I.

The symbols n2, n3, n4, n5, n6, n7, n8, n1l, n12, and n13 are independently an integer from 0 to 4. The symbols m2, m3, m4, m5, m6, m7, m8, m11, m12, v2, v3, v4, v5, v6, v7, v8, v11, v12, and v13 are independently 1 or 2.

The symbol z8 is an integer from 0 to 3.

The symbol p is 1 or 2. The symbol q is 1 or 2.

In an aspect is provided a compound, or a pharmaceutically acceptable salt or solvate thereof, having the formula:

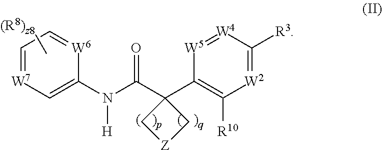

W², R³, W⁴, W⁵, W⁶, W⁷, R⁸, z8, Z, p, and q are as described herein, including in embodiments. At least one of W⁶ or W⁷ is N. If W⁶ is N and W⁷ is C(R⁷), then R¹⁰ is not hydrogen, —F, —Cl, —CH₃, or —OCH₃. If W⁶ is C(R⁶) and W⁷ is N, then R¹⁰ is not hydrogen, —F, —Cl, —CH₃ or —OCH₃. If W⁶ and W⁷ are N and Z is 0 or CH₂, then R¹⁰ is not hydrogen, —F, —Cl, —CH₃ or —OCH₃.

R¹⁰ is hydrogen, halogen, —CX¹⁰₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}, —C(O)R^{10C}, —NR^{10A}AC(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹⁰ and R² substituents may optionally be joined to form a substituted or unsubstituted cycloalkyl, substituted or unsubstituted heterocycloalkyl, substituted or unsubstituted aryl, or substituted or unsubstituted heteroaryl.

R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl.

X¹⁰ is independently —F, —Cl, —Br, or —I.

The symbol n10 is an integer from 0 to 4. The symbols m10 and v10 are independently 1 or 2.

In an aspect is provided a pharmaceutical composition including a compound described herein, or a pharmaceutically acceptable salt or solvate thereof, and a pharmaceutically acceptable excipient.

In an aspect is provided a method of treating a neurodegenerative disorder in a subject in need thereof, the method including administering to the subject in need thereof a therapeutically effective amount of a compound described herein, or a pharmaceutically acceptable salt or solvate thereof.

In an aspect is provided a method of treating an inflammatory disease in a subject in need thereof, the method including administering to the subject in need thereof a therapeutically effective amount of a compound described herein, or a pharmaceutically acceptable salt or solvate thereof.

In an aspect is provided a method of treating a demyelinating disease in a subject in need thereof, the method including administering to the subject in need thereof a therapeutically effective amount of a compound described herein, or a pharmaceutically acceptable salt or solvate thereof.

In an aspect is provided a method of treating fibrotic disease in a subject in need thereof, the method including administering to the subject in need thereof a therapeutically effective amount of a compound described herein, or a pharmaceutically acceptable salt or solvate thereof.

In an aspect is provided a method of treating cancer in a subject in need thereof, the method including administering to the subject in need thereof a therapeutically effective amount of a compound described herein, or a pharmaceutically acceptable salt or solvate thereof.

In an aspect is provided a method of treating an LPAR1-associated disease in a subject in need thereof, the method including administering to the subject in need thereof a therapeutically effective amount of a compound described herein, or a pharmaceutically acceptable salt or solvate thereof.

In an aspect is provided a method of modulating LPAR1 activity in a subject, the method including administering to the subject a compound described herein, or a pharmaceutically acceptable salt or solvate thereof.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

Bridged rings are two or more rings that share three or more atoms, separating the two bridgehead atoms by a bridge containing at least one atom. Individual rings in bridged rings may be substituted or unsubstituted and may have different substituents from other individual rings within a set of bridged rings. Possible substituents for individual rings within bridged rings are the possible substituents for the same ring when not part of bridged rings (e.g., substituents for cycloalkyl or heterocycloalkyl rings). Bridged rings may be substituted or unsubstituted cycloalkyl, substituted or unsubstituted cycloalkylene, substituted or unsubstituted heterocycloalkyl or substituted or unsubstituted heterocycloalkylene and individual rings within a bridged ring group may be any of the immediately previous list, including having all rings of one type (e.g., all rings being substituted heterocycloalkylene wherein each ring may be the same or different substituted heterocycloalkylene). When referring to a bridged ring system, heterocyclic bridged rings means bridged rings wherein at least one ring is a heterocyclic ring and wherein each ring may be a different ring. When referring to a bridged ring system, substituted bridged rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

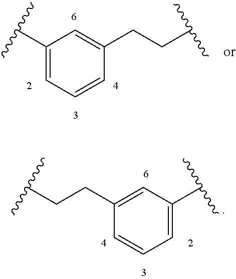 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′C(O)NR″R″′, —NR″C(O)₂R′, —NRC(NR′R″R″′)═NR″″, —NRC(NR′R″)═NR″′, —S(O)R′, —S(O)₂R′, —S(O)₂NR′R″, —NRSO₂R′, —NR′NR″R″′, —ONR′R″, —NR′C(O)NR″NR″′ R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R″′, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R″′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —NR′NR″R″′, —ONR′R″, —NR′C(O)NR″NR″′ R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R″′, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R″′)_{d}—, where s and d are independently integers of from 0 to 3, and X is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1} L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1} respectively. In turn, each first substituent group (e.g., R^{1.1}, R^{2.1}, R^{3.1}, R^{4.1}, R_{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

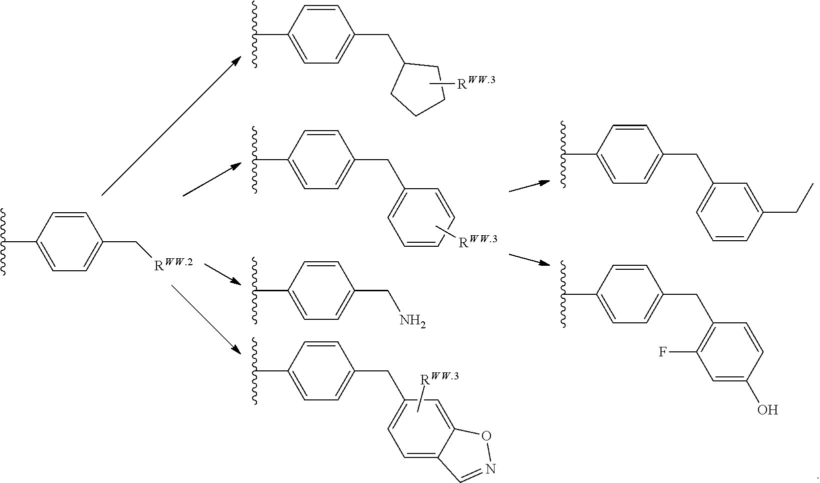

R^{WW.1} is independently oxo, halogen, —CX^{WW1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100A.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}_substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)— or (S)— or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)— and (S)—, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS), tert-butyldimethylsilyl (TBS)). In embodiments the protecting group is an amine protecting group. Non-limiting examples of amine protecting groups include carbobenzyloxy (Cbz), tert-butyloxycarbonyl (BOC), 9-Fluorenylmethyloxycarbonyl (FMOC), acetyl, benzoyl, benzyl, carbamate, p-methoxybenzyl ether (PMB), and tosyl (Ts).

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is not prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “lysophosphatidic acid receptor 1 antagonist” or “LPAR1 antagonist” refers to any exogenously administered compound or agent that is capable of partially or completely inhibiting, or reversing, the effect of an agonist (e.g., lysophosphatidic acid) on the LPAR1 receptor. The term is inclusive of compounds or agents characterized or described as antagonists, partial antagonists, and negative allosteric modulators.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a neurodegenerative disease. In embodiments, the disease is an inflammatory disease. In embodiments, the disease is post-hemorrhagic encephalitis. In embodiments, the disease is a demyelinating disease. In embodiments, the disease is multiple sclerosis. In embodiments, the disease is a fibrotic disease. In embodiments, the disease is pulmonary fibrosis. In embodiments, the disease is idiopathic pulmonary fibrosis. In embodiments, the disease is a cancer. In embodiments, the disease is glioblastoma.

As used herein, the term “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

As used herein, the term “inflammatory disease” refers to a disease or condition characterized by aberrant inflammation (e.g., an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, scleroderma, and atopic dermatitis.

As used herein, the term “demyelinating disease” refers to any disease or condition characterized by damage to the protective covering (e.g., myelin sheath) that surrounds nerve fibers (e.g., in the brain, optic nerves, or spinal cord). In embodiments, the demyelinating disease is a demyelinating disease of the central nervous system. In embodiments, the demyelinating disease is multiple sclerosis. In embodiments, the demyelinating disease is a demyelinating disease of the peripheral nervous system.

As used herein, the terms “fibrotic disease” and “fibrosis” refer to any disease or condition characterized by the formation of excess fibrous connective tissue. The formation of excess fibrous connective tissue may be in response to a reparative or reactive process. Fibrotic diseases include but are not limited to pulmonary fibrosis (e.g., idiopathic pulmonary fibrosis (IPF)), liver fibrosis (e.g., nonalcoholic steatohepatitis (NASH)), myelofibrosis, skin fibrosis (e.g., scleroderma), ocular fibrosis, mediastinal fibrosis, cardiac fibrosis, kidney fibrosis, stromal fibrosis, epidural fibrosis, epithelial fibrosis, or idiopathic fibrosis.

As used herein, the term “cardiovascular disorder” or “cardiovascular disease” is used in accordance with its plain ordinary meaning. In embodiments, cardiovascular diseases that may be treated with a compound, pharmaceutical composition, or method described herein include, but are not limited to, stroke, heart failure, hypertension, hypertensive heart disease, myocardial infarction, angina pectoris, tachycardia, cardiomyopathy, rheumatic heart disease, cardiomyopathy, heart arrhythmia, congenital heart disease, valvular heart disease, carditis, aortic aneurysms, peripheral artery disease, thromboembolic disease, and venous thrombosis.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “LPAR1-associated disease” refers to any disease or condition caused by aberrant activity or signaling of LPAR1. In embodiments, the LPAR1-associated disease is a neurodegenerative disease. In embodiments, the LPAR1-associated disease is an inflammatory disease. In embodiments, the LPAR1-associated disease is post-hemorrhagic encephalitis. In embodiments, the LPAR1-associated disease is a demyelinating disease. In embodiments, the LPAR1-associated disease is multiple sclerosis. In embodiments, the LPAR1-associated disease is a fibrotic disease. In embodiments, the LPAR1-associated disease is pulmonary fibrosis. In embodiments, the LPAR1-associated disease is idiopathic pulmonary fibrosis. In embodiments, the LPAR1-associated disease is a cancer. In embodiments, the LPAR1-associated disease is glioblastoma.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P ³³P, ⁴⁵Ti, ⁴⁷Sc, ^{5.2}Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P ³³P, ⁴⁵Ti, ⁴⁷Sc, ^{5.2}Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In ¹²³I, ¹²⁴I, ¹²⁵I, ¹³¹I, ¹⁴²Pr, ¹⁴³Pr, ¹⁴⁹Pm ¹⁵³Sm, ¹⁵⁴⁻¹⁵⁸¹Gd ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴r ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., multiple sclerosis, fibrotic disease, encephalitis, or cancer) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., multiple sclerosis, fibrotic disease, encephalitis, or cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “lysophosphatidic acid receptor” or “LPAR” refers to one or more of the family of G protein-coupled receptors for lysophosphatidic acid (LPA). In embodiments, LPAR includes LPAR1, LPAR2, LPAR3, LPAR4, LPAR5, and LPAR6.

The term “lysophosphatidic acid receptor 1” or “LPAR1” refers to a G protein-coupled receptor (including homologs, isoforms, and functional fragments thereof) that binds the lipid signaling molecule lysophosphatidic acid (LPA). The term includes any recombinant or naturally-occurring form of LPAR1 variants thereof that maintain LPAR1 activity (e.g., within at least 30%, 40%, 50%, 60%, 70%, 80%, 90%, 95%, or 100% activity compared to wildtype LPAR1). In embodiments, the LPAR1 protein encoded by the LPAR1 gene has the amino acid sequence set forth in or corresponding to Entrez 1902, UniProt Q92633, RefSeq (protein) NP_001392.2 or RefSeq (protein) NP 476500.1. In embodiments, the LPAR1 gene has the nucleic acid sequence set forth in RefSeq (mRNA) NM_001401.3 or RefSeq (mRNA) NM_057159.2. In embodiments, the amino acid sequence or nucleic acid sequence is the sequence known at the time of filing of the present application.

The term “selective” or “selectivity” or the like in reference to a compound or agent refers to the compound's or agent's ability to cause an increase or decrease in activity of a particular molecular target (e.g., protein, enzyme, etc.) preferentially over one or more different molecular targets (e.g., a compound having selectivity toward lysophosphatidic acid receptor 1 (LPAR1) would preferentially inhibit LPAR1 over other lysophosphatidic acid receptors). In embodiments, an “lysophosphatidic acid receptor 1 selective compound” or “LPAR1-selective compound” refers to a compound (e.g., compound described herein) having selectivity towards lysophosphatidic acid receptor 1 (LPAR1). In embodiments, the compound (e.g., compound described herein) is about 5-fold, 10-fold, 20-fold, 30-fold, 40-fold, 50-fold, or about 100-fold more selective for lysophosphatidic acid receptor 1 (LPAR1) over one or more of LPAR2, LPAR3, LPAR4, LPAR5, or LPAR6. In embodiments, the compound (e.g., compound described herein) is at least 5-fold, 10-fold, 20-fold, 30-fold, 40-fold, 50-fold, or at least 100-fold more selective for lysophosphatidic acid receptor 1 (LPAR1) over one or more of LPAR2, LPAR3, LPAR4, LPAR5, or LPAR6.

### II. Compounds

In an aspect is provided a compound, or a pharmaceutically acceptable salt or solvate thereof, having the formula:

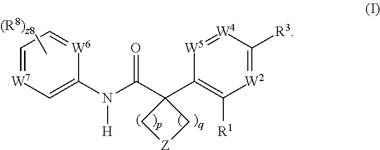

R¹ is substituted or unsubstituted alkyl (e.g., C₁-C₈, C₁-C₆, C₁-C₄, or C₁-C₂).
W² is N or C(R²).
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

W⁴ is N or C(R⁴).

R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —N^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}—NR^{4A}C(O)R^{4C}, —NR^{4A}C(O)OR^{4C}, —NR^{4A}OR^{4C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

W⁵ is N or C(R⁵).

R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —N^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R² and R³ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³ and R⁴ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁴ and R⁵ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

W⁶ is N or C(R⁶).

R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

W⁷ is N, N⁺—O−⁻, or C(R⁷).

R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —N^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁸ is independently halogen, —CX⁸₃, —CHX⁸₂, —CH₂X⁸, —OCX⁸₃, —OCH₂X⁸, —OCHX⁸₂, —CN, —SOng₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two R⁸ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

Z is O, C(R¹¹)(R¹²), or C(O)N(R¹³).

R¹¹ is hydrogen, halogen, —CX¹¹₃, —CHX¹¹₂, —CH₂X¹¹, —OCX¹¹₃, —OCH₂X¹¹, —OCHX¹¹₂, —CN, —SOₙ₁₁R^{11D}, —SOᵥ₁₁NR^{11A}R^{11B}, —NR^{11C}NR^{11A}R^{11B}, —ONR^{11A}R^{11B}, —NHC(O)NR^{11C}NR^{11A}R^{11B}, —NHC(O)NR^{11A}R^{11B}, —N(O)ₘ₁₁, —NR¹AR^{11B}, —C(O)R^{11C}, —C(O)OR^{11C}, —C(O)NR^{11A}R^{11B}, —OR^{11D}, —SR^{11D}, —NR^{11A} SO₂R^{11D}, —NR^{11A} C(O)R^{11C}, —NR^{11A} C(O)OR^{11C}, —NR^{11A} OR^{11C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹² is hydrogen, halogen, —CX¹²₃, —CHX¹²₂, —CH₂X¹², —OCX¹²₃, —OCH₂X¹², —OCHX¹²₂, —CN, —SOₙ₁₂R^{12D}, —SOᵥ₁₂NR^{12A}R^{12B}, —NR^{12C}NR^{12A}R^{12B}, —ONR^{12A}R^{12B}, —NHC(O)NR^{12C}NR^{12A}R^{12B}, —NHC(O)NR^{12A}R^{12B}, —N(O)ₘ₁₂, —NR^{12A}R^{12B}, —C(O)R^{12C}, —C(O)OR^{12C}, —C(O)NR^{12A}R^{12B}, —OR^{12D}, —SR^{12D}, —NR^{12A}SO₂R^{12D}, —NR^{12A}C(O)R^{12C}, —NR^{12A}C(O)OR^{12C}, —NR^{12A} OR^{12C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹¹ and R¹² substituents may optionally be joined to form an oxo, substituted or unsubstituted cycloalkyl (e.g., C₃-C₈, C₃-C₆, C₄-C₆, or C₅-C₆), or substituted or unsubstituted heterocycloalkyl (e.g., 3 to 8 membered, 3 to 6 membered, 4 to 6 membered, 4 to 5 membered, or 5 to 6 membered).

R¹³ is hydrogen, halogen, —CX¹³₃, —CHX¹³₂, —CH₂X¹³, —OCX¹³₃, —OCH₂X¹³, —OCHX¹³₂, —SOₙ₁₃R^{13D}, —SOᵥ₁₃NR^{13A}R^{13B}, —C(O)R^{13C}, —C(O)OR^{13C}, —C(O)NR^{13A}R^{13B}, —OR^{13D}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}R^{6B}, R^{6C}, R^{6D}, R^{7A}, R^{7B}, R^{7C}, R^{7D}, R^{8A}, R^{8B}, Rsc, R^{8D}, R^{11A}, R^{11B}, R^{11C}, R^{11D}, R^{12A}, R^{12B}, R^{12C}, R^{12D}, R^{13A}, R^{13B}, R^{13C}, and R^{13D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{5A} and R^{5B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{6A} and R^{6B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{7A} and R^{7B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{8A} and R^{8B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{11A} and R^{11B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{12A} and R^{12B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{13B} and R^{13B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X², X³, X⁴, X⁵, X⁶, X⁷, X⁸, X¹¹, X¹², and X¹³ are independently —F, —Cl, —Br, or —I.

The symbols n2, n3, n4, n5, n6, n7, n8, n1l, n12, and n13 are independently an integer from 0 to 4.

The symbols m2, m3, m4, m5, m6, m7, m8, m11, m12, v2, v3, v4, v5, v6, v7, v8, v11, v12, and v13 are independently 1 or 2.

The symbol z8 is an integer from 0 to 3.

The symbol p is 1 or 2.

The symbol q is 1 or 2.

In embodiments, the compound has the formula:

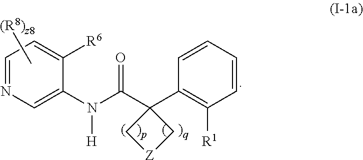

R¹, R⁶, R⁸, z8, Z, p, and q are as described herein, including in embodiments.

In embodiments, the compound has the formula:

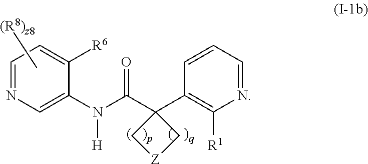

R¹, R⁶, R⁸, z8, Z, p, and q are as described herein, including in embodiments.

In embodiments, the compound has the formula:

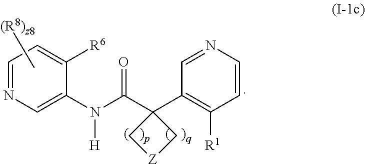

R¹, R⁶, R⁸, z8, Z, p, and q are as described herein, including in embodiments.

In embodiments, the compound has the formula:

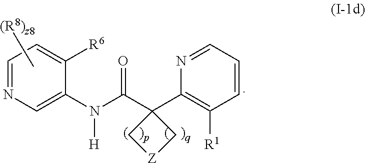

R¹, R⁶, R⁸, z8, Z, p, and q are as described herein, including in embodiments.

In embodiments, the compound has the formula:

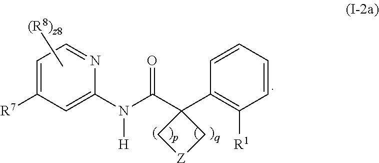

R¹, R⁷, R⁸, z8, Z, p, and q are as described herein, including in embodiments.

In embodiments, the compound has the formula:

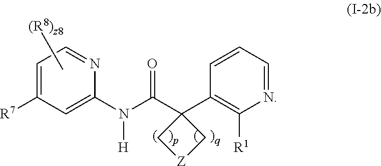

R¹, R⁷, R⁸, z8, Z, p, and q are as described herein, including in embodiments.

In embodiments, the compound has the formula:

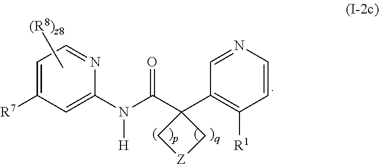

R¹, R⁷, R⁸, z8, Z, p, and q are as described herein, including in embodiments.

In embodiments, the compound has the formula:

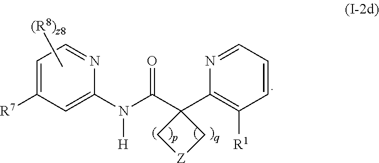

R¹, R⁷, R⁸, z8, Z, p, and q are as described herein, including in embodiments.

In embodiments, the compound has the formula:

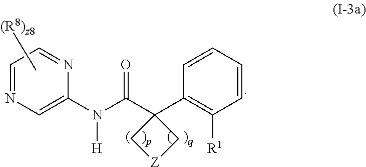

R¹, R⁸, z8, Z, p, and q are as described herein, including in embodiments.

In embodiments, the compound has the formula:

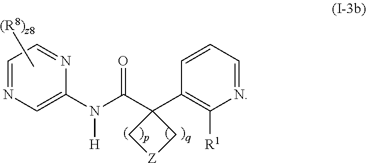

R¹, R⁸, z8, Z, p, and q are as described herein, including in embodiments.

In embodiments, the compound has the formula:

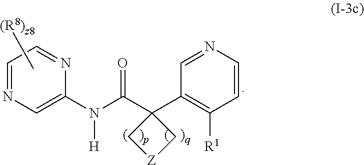

R¹, R⁸, z8, Z, p, and q are as described herein, including in embodiments.

In embodiments, the compound has the formula:

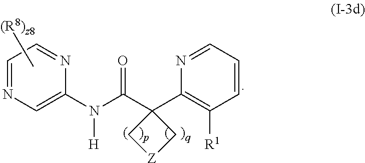

R¹, R⁸, z8, Z, p, and q are as described herein, including in embodiments.

In embodiments, the compound has the formula:

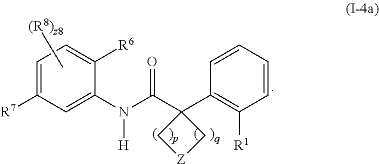

R¹, R⁶, R⁷, R⁸, z8, Z, p, and q are as described herein, including in embodiments.

In embodiments, the compound has the formula:

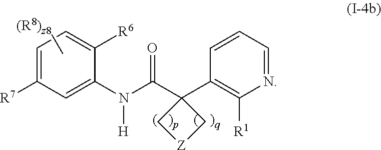

R¹, R⁶, R⁷, R⁸, z8, Z, p, and q are as described herein, including in embodiments.

In embodiments, the compound has the formula:

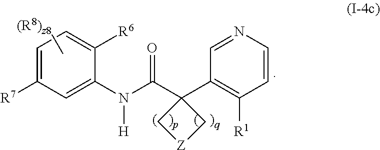

R¹, R⁶, R⁷, R⁸, z8, Z, p, and q are as described herein, including in embodiments.

In embodiments, the compound has the formula:

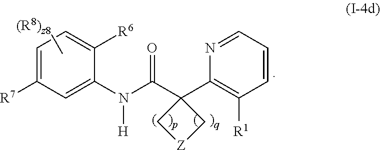

R¹, R⁶, R⁷, R⁸, z8, Z, p, and q are as described herein, including in embodiments.

In embodiments, a substituted R¹ (e.g., substituted alkyl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is unsubstituted alkyl. In embodiments, R¹ is unsubstituted C₂-C₅ alkyl. In embodiments, R¹ is unsubstituted C₂ alkyl. In embodiments, R¹ is unsubstituted ethyl. In embodiments, R¹ is unsubstituted C₃ alkyl. In embodiments, R¹ is unsubstituted propyl. In embodiments, R¹ is unsubstituted n-propyl. In embodiments, R¹ is unsubstituted isopropyl (i.e., isopropyl). In embodiments, R¹ is unsubstituted C₄ alkyl. In embodiments, R¹ is unsubstituted butyl. In embodiments, R¹ is unsubstituted n-butyl. In embodiments, R¹ is unsubstituted isobutyl. In embodiments, R¹ is unsubstituted tert-butyl. In embodiments, R¹ is unsubstituted C₅ alkyl. In embodiments, R¹ is unsubstituted pentyl. In embodiments, R¹ is unsubstituted n-pentyl. In embodiments, R¹ is unsubstituted tert-pentyl. In embodiments, R¹ is unsubstituted neopentyl. In embodiments, R¹ is unsubstituted isopentyl. In embodiments, R¹ is unsubstituted sec-pentyl. In embodiments, R¹ is unsubstituted 3-pentyl. In embodiments, R¹ is unsubstituted sec-isopentyl. In embodiments, R¹ is unsubstituted 2-methylbutyl.

In an aspect is provided a compound, or a pharmaceutically acceptable salt or solvate thereof, having the formula:

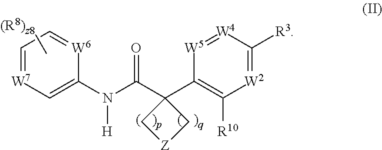

W², R³, W⁴, W⁵, W⁶, W⁷, R⁸, z8, Z, p, and q are as described herein, including in embodiments. At least one of W⁶ or W⁷ is N. If W⁶ is N and W⁷ is C(R⁷), then R¹⁰ is not hydrogen, —F, —Cl, —CH₃, or —OCH₃. If W⁶ is C(R⁶) and W⁷ is N, then R¹⁰ is not hydrogen, —F, —Cl, —CH₃ or —OCH₃. If W⁶ and W⁷ are N and Z is 0 or CH₂, then R¹⁰ is not hydrogen, —F, —Cl, —CH₃ or —OCH₃.

R¹⁰ is hydrogen, halogen, —CX¹⁰₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹⁰ and R² substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{10A} and R^{10B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X¹⁰ is independently —F, —Cl, —Br, or —I.

The symbol n10 is an integer from 0 to 4.

The symbols m10 and v10 are independently 1 or 2.

In embodiments, the compound has the formula:

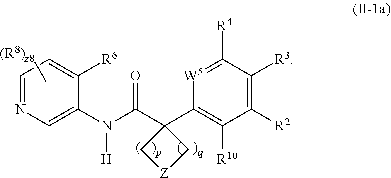

R², R³, R⁴, W⁵, R⁶, R⁸, z8, R¹⁰, Z, p, and q are as described herein, including in embodiments.

In embodiments, the compound has the formula:

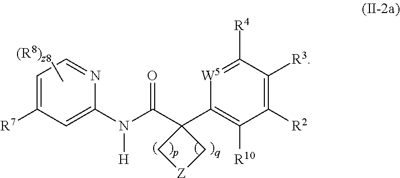

R², R³, R⁴, W⁵, R⁷, R⁸, z8, R¹⁰, Z, p, and q are as described herein, including in embodiments.

In embodiments, the compound has the formula:

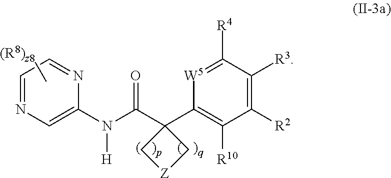

R², R³, R⁴, W⁵, R⁸, z8, R¹⁰, Z, p, and q are as described herein, including in embodiments.

In embodiments, the compound has the formula:

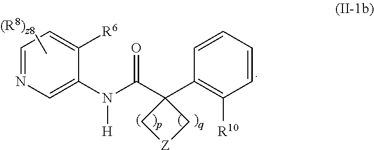

R⁶, R⁸, z8, R¹⁰, Z, p, and q are as described herein, including in embodiments.

In embodiments, the compound has the formula:

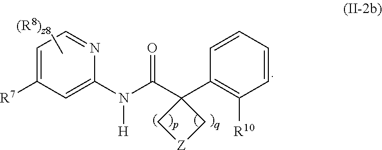

R⁶, R⁸, z8, R¹⁰, Z, p, and q are as described herein, including in embodiments.

In embodiments, the compound has the formula:

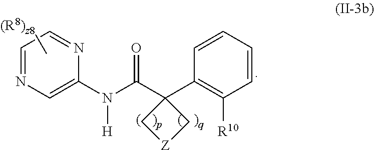

R⁶, R⁸, z8, R¹⁰, Z, p, and q are as described herein, including in embodiments.

In embodiments, a substituted R¹⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, a substituted R^{10A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10A} is substituted with a plurality of groups selected from substituent groups, size-limited substituent groups, and lower substituent groups; each substituent group, size-limited substituent group, and/or lower substituent group may optionally be different. In embodiments, when R^{10A} is substituted, it is substituted with at least one substituent group. In embodiments, when R^{10A} is substituted, it is substituted with at least one size-limited substituent group. In embodiments, when R^{10A} is substituted, it is substituted with at least one lower substituent group.

In embodiments, a substituted R^{10B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10B} is substituted with a plurality of groups selected from substituent groups, size-limited substituent groups, and lower substituent groups; each substituent group, size-limited substituent group, and/or lower substituent group may optionally be different. In embodiments, when R^{10B} is substituted, it is substituted with at least one substituent group. In embodiments, when R^{10B} is substituted, it is substituted with at least one size-limited substituent group. In embodiments, when R^{10B} is substituted, it is substituted with at least one lower substituent group.

In embodiments, a substituted ring formed when R^{10A} and R^{10B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{10A} and R^{10B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{10A} and R^{10B} substituents bonded to the same nitrogen atom are joined is substituted, it is substituted with at least one substituent group. In embodiments, when the substituted ring formed when R^{10A} and R^{10B} substituents bonded to the same nitrogen atom are joined is substituted, it is substituted with at least one size-limited substituent group. In embodiments, when the substituted ring formed when R^{10A} and R^{10B} substituents bonded to the same nitrogen atom are joined is substituted, it is substituted with at least one lower substituent group.

In embodiments, a substituted R^{10C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10C} is substituted with a plurality of groups selected from substituent groups, size-limited substituent groups, and lower substituent groups; each substituent group, size-limited substituent group, and/or lower substituent group may optionally be different. In embodiments, when R^{10C} is substituted, it is substituted with at least one substituent group. In embodiments, when R^{10C} is substituted, it is substituted with at least one size-limited substituent group. In embodiments, when R^{10C} is substituted, it is substituted with at least one lower substituent group.

In embodiments, a substituted R^{10D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10D} is substituted with a plurality of groups selected from substituent groups, size-limited substituent groups, and lower substituent groups; each substituent group, size-limited substituent group, and/or lower substituent group may optionally be different. In embodiments, when R^{10D} is substituted, it is substituted with at least one substituent group. In embodiments, when R^{10D} is substituted, it is substituted with at least one size-limited substituent group. In embodiments, when R^{10D} is substituted, it is substituted with at least one lower substituent group.

In embodiments, R¹⁰ is hydrogen, halogen, —CX¹⁰₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NHC(O)NR^{10A}R^{10B}, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁰ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁰ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹⁰ is hydrogen, —CHF₂, substituted or unsubstituted C₁-C₆ alkyl, or substituted or unsubstituted 2 to 6 membered heteroalkyl. In embodiments, R¹⁰ is hydrogen or unsubstituted C₁-C₆ alkyl. In embodiments, R¹⁰ is hydrogen. In embodiments, R¹⁰ is unsubstituted C₁ alkyl. In embodiments, R¹⁰ is unsubstituted methyl. In embodiments, R¹⁰ is unsubstituted C₂ alkyl. In embodiments, R¹⁰ is unsubstituted ethyl. In embodiments, R¹⁰ is unsubstituted C₃ alkyl. In embodiments, R¹⁰ is unsubstituted propyl. In embodiments, R¹⁰ is unsubstituted n-propyl. In embodiments, R¹⁰ is unsubstituted isopropyl (i.e., isopropyl). In embodiments, R¹⁰ is unsubstituted C₄ alkyl. In embodiments, R¹⁰ is unsubstituted butyl. In embodiments, R¹⁰ is unsubstituted n-butyl. In embodiments, R¹⁰ is unsubstituted isobutyl. In embodiments, R¹⁰ is unsubstituted tert-butyl. In embodiments, R¹⁰ is unsubstituted C₅ alkyl. In embodiments, R¹⁰ is unsubstituted pentyl. In embodiments, R¹⁰ is unsubstituted n-pentyl. In embodiments, R¹⁰ is unsubstituted tert-pentyl. In embodiments, R¹⁰ is unsubstituted neopentyl. In embodiments, R¹⁰ is unsubstituted isopentyl. In embodiments, R¹⁰ is unsubstituted sec-pentyl. In embodiments, R¹⁰ is unsubstituted 3-pentyl. In embodiments, R¹⁰ is unsubstituted sec-isopentyl. In embodiments, R¹⁰ is unsubstituted 2-methylbutyl. In embodiments, R¹⁰ is unsubstituted C₆ alkyl. In embodiments, R¹⁰ is unsubstituted hexyl.

In embodiments, W² is N. In embodiments, W² is C(R²). In embodiments, W² is CH.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NHC(O)NR^{2A}R^{2B}, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is hydrogen or unsubstituted C₁-C₆ alkyl. In embodiments, R² is hydrogen. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl. In embodiments, R² is unsubstituted butyl. In embodiments, R² is unsubstituted n-butyl. In embodiments, R² is unsubstituted isobutyl. In embodiments, R² is unsubstituted tert-butyl. In embodiments, R² is unsubstituted pentyl. In embodiments, R² is unsubstituted hexyl.

In embodiments, a substituted ring formed when R¹⁰ and R² substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when R¹⁰ and R² substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R¹⁰ and R² substituents are joined is substituted, it is substituted with at least one substituent group. In embodiments, when the substituted ring formed when R¹⁰ and R² substituents are joined is substituted, it is substituted with at least one size-limited substituent group. In embodiments, when the substituted ring formed when R¹⁰ and R² substituents are joined is substituted, it is substituted with at least one lower substituent group.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted ring formed when R^{3A} and R^{3B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3A} and R^{3B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3A} and R^{3B} substituents bonded to the same nitrogen atom are joined is substituted, it is substituted with at least one substituent group. In embodiments, when the substituted ring formed when R^{3A} and R^{3B} substituents bonded to the same nitrogen atom are joined is substituted, it is substituted with at least one size-limited substituent group. In embodiments, when the substituted ring formed when R^{3A} and R^{3B} substituents bonded to the same nitrogen atom are joined is substituted, it is substituted with at least one lower substituent group.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, a substituted R^{3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3D} is substituted with a plurality of groups selected from substituent groups, size-limited substituent groups, and lower substituent groups; each substituent group, size-limited substituent group, and/or lower substituent group may optionally be different. In embodiments, when R^{3D} is substituted, it is substituted with at least one substituent group. In embodiments, when R^{3D} is substituted, it is substituted with at least one size-limited substituent group. In embodiments, when R^{3D} is substituted, it is substituted with at least one lower substituent group.

In embodiments, R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NHC(O)NR^{3A}R^{3B}, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is hydrogen or unsubstituted C₁-C₆ alkyl. In embodiments, R³ is hydrogen. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted isobutyl. In embodiments, R³ is unsubstituted tert-butyl. In embodiments, R³ is unsubstituted pentyl. In embodiments, R³ is unsubstituted hexyl.

In embodiments, a substituted ring formed when R² and R³ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when R² and R³ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R² and R³ substituents are joined is substituted, it is substituted with at least one substituent group. In embodiments, when the substituted ring formed when R² and R³ substituents are joined is substituted, it is substituted with at least one size-limited substituent group. In embodiments, when the substituted ring formed when R² and R³ substituents are joined is substituted, it is substituted with at least one lower substituent group.

In embodiments, W⁴ is N. In embodiments, W⁴ is C(R⁴). In embodiments, W⁴ is CH.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted ring formed when R^{4A} and R^{4B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{4A} and R^{4B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4A} and R^{4B} substituents bonded to the same nitrogen atom are joined is substituted, it is substituted with at least one substituent group. In embodiments, when the substituted ring formed when R^{4A} and R^{4B} substituents bonded to the same nitrogen atom are joined is substituted, it is substituted with at least one size-limited substituent group. In embodiments, when the substituted ring formed when R^{4A} and R^{4B} substituents bonded to the same nitrogen atom are joined is substituted, it is substituted with at least one lower substituent group.

In embodiments, a substituted R^{4C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, a substituted R^{4D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D} is substituted, it is substituted with at least one size-limited substituent group. In embodiments, when R^{4D} is substituted, it is substituted with at least one lower substituent group.

In embodiments, R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NHC(O)NR^{4A}R^{4B}, —N^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is hydrogen or unsubstituted C₁-C₆ alkyl. In embodiments, R⁴ is hydrogen. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl. In embodiments, R⁴ is unsubstituted pentyl. In embodiments, R⁴ is unsubstituted hexyl.

In embodiments, a substituted ring formed when R³ and R⁴ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when R³ and R⁴ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R³ and R⁴ substituents are joined is substituted, it is substituted with at least one substituent group. In embodiments, when the substituted ring formed when R³ and R⁴ substituents are joined is substituted, it is substituted with at least one size-limited substituent group. In embodiments, when the substituted ring formed when R³ and R⁴ substituents are joined is substituted, it is substituted with at least one lower substituent group.

In embodiments, W⁵ is N. In embodiments, W⁵ is C(R⁵). In embodiments, W⁵ is CH.

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, a substituted R^{5B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5B} is substituted with a plurality of groups selected from substituent groups, size-limited substituent groups, and lower substituent groups; each substituent group, size-limited substituent group, and/or lower substituent group may optionally be different. In embodiments, when R^{5B} is substituted, it is substituted with at least one substituent group. In embodiments, when R^{5B} is substituted, it is substituted with at least one size-limited substituent group. In embodiments, when R^{5B} is substituted, it is substituted with at least one lower substituent group.

In embodiments, a substituted ring formed when R^{5A} and R^{5B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{5A} and R^{5.1} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{5A} and R^{5B} substituents bonded to the same nitrogen atom are joined is substituted, it is substituted with at least one substituent group. In embodiments, when the substituted ring formed when R^{5A} and R^{5B} substituents bonded to the same nitrogen atom are joined is substituted, it is substituted with at least one size-limited substituent group. In embodiments, when the substituted ring formed when R^{5A} and R^{5B} substituents bonded to the same nitrogen atom are joined is substituted, it is substituted with at least one lower substituent group.

In embodiments, a substituted R^{5C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5C} is substituted with a plurality of groups selected from substituent groups, size-limited substituent groups, and lower substituent groups; each substituent group, size-limited substituent group, and/or lower substituent group may optionally be different. In embodiments, when R^{5C} is substituted, it is substituted with at least one substituent group. In embodiments, when R^{5C} is substituted, it is substituted with at least one size-limited substituent group. In embodiments, when R^{5C} is substituted, it is substituted with at least one lower substituent group.

In embodiments, a substituted R^{5D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5D} is substituted with a plurality of groups selected from substituent groups, size-limited substituent groups, and lower substituent groups; each substituent group, size-limited substituent group, and/or lower substituent group may optionally be different. In embodiments, when R^{5D} is substituted, it is substituted with at least one substituent group. In embodiments, when R^{5D} is substituted, it is substituted with at least one size-limited substituent group. In embodiments, when R^{5D} is substituted, it is substituted with at least one lower substituent group.

In embodiments, R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NHC(O)NR^{5A}R^{5B}, —NR^{5A}R^{5B}, —C(O)R^{5C}, —C(O)OR^{5C}, —C(O)NR^{5A}R^{5B}, —OR^{5D}, —SR^{5D}, —NR^{5A}SO_{2R}^{5D}, —NR^{5A}C(O)R^{5C}, —NR^{5A}C(O)OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is hydrogen or unsubstituted C₁-C₆ alkyl. In embodiments, R⁵ is hydrogen. In embodiments, R⁵ is unsubstituted methyl. In embodiments, R⁵ is unsubstituted ethyl. In embodiments, R⁵ is unsubstituted propyl. In embodiments, R⁵ is unsubstituted n-propyl. In embodiments, R⁵ is unsubstituted isopropyl. In embodiments, R⁵ is unsubstituted butyl. In embodiments, R⁵ is unsubstituted n-butyl. In embodiments, R⁵ is unsubstituted isobutyl. In embodiments, R⁵ is unsubstituted tert-butyl. In embodiments, R⁵ is unsubstituted pentyl. In embodiments, R⁵ is unsubstituted hexyl.

In embodiments, a substituted ring formed when R⁴ and R⁵ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when R⁴ and R⁵ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R⁴ and R⁵ substituents are joined is substituted, it is substituted with at least one substituent group. In embodiments, when the substituted ring formed when R⁴ and R⁵ substituents are joined is substituted, it is substituted with at least one size-limited substituent group. In embodiments, when the substituted ring formed when R⁴ and R⁵ substituents are joined is substituted, it is substituted with at least one lower substituent group.

In embodiments, W⁶ is N. In embodiments, W⁶ is C(R⁶). In embodiments, W⁶ is CH.

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, a substituted R^{6A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6A} is substituted with a plurality of groups selected from substituent groups, size-limited substituent groups, and lower substituent groups; each substituent group, size-limited substituent group, and/or lower substituent group may optionally be different. In embodiments, when R^{6A} is substituted, it is substituted with at least one substituent group. In embodiments, when R^{6A} is substituted, it is substituted with at least one size-limited substituent group. In embodiments, when R^{6A} is substituted, it is substituted with at least one lower substituent group.

In embodiments, a substituted R^{6B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6B} is substituted with a plurality of groups selected from substituent groups, size-limited substituent groups, and lower substituent groups; each substituent group, size-limited substituent group, and/or lower substituent group may optionally be different. In embodiments, when R^{6B} is substituted, it is substituted with at least one substituent group. In embodiments, when R^{6B} is substituted, it is substituted with at least one size-limited substituent group. In embodiments, when R^{6B} is substituted, it is substituted with at least one lower substituent group.

In embodiments, a substituted ring formed when R^{6A} and R^{6B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{6A} and R^{6B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{6A} and R^{6B}, substituents bonded to the same nitrogen atom are joined is substituted, it is substituted with at least one substituent group. In embodiments, when the substituted ring formed when R^{6A} and R^{6B} substituents bonded to the same nitrogen atom are joined is substituted, it is substituted with at least one size-limited substituent group. In embodiments, when the substituted ring formed when R^{6A} and R^{6B} substituents bonded to the same nitrogen atom are joined is substituted, it is substituted with at least one lower substituent group.

In embodiments, a substituted R^{6C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6C} is substituted with a plurality of groups selected from substituent groups, size-limited substituent groups, and lower substituent groups; each substituent group, size-limited substituent group, and/or lower substituent group may optionally be different. In embodiments, when R^{6C} is substituted, it is substituted with at least one substituent group. In embodiments, when R^{6C} is substituted, it is substituted with at least one size-limited substituent group. In embodiments, when R^{6C} is substituted, it is substituted with at least one lower substituent group.

In embodiments, a substituted R^{6D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6D} is substituted with a plurality of groups selected from substituent groups, size-limited substituent groups, and lower substituent groups; each substituent group, size-limited substituent group, and/or lower substituent group may optionally be different. In embodiments, when R^{6D} is substituted, it is substituted with at least one substituent group. In embodiments, when R^{6D} is substituted, it is substituted with at least one size-limited substituent group. In embodiments, when R^{6D} is substituted, it is substituted with at least one lower substituent group.

In embodiments, R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NHC(O)NR^{6A}R^{6B}, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is hydrogen or unsubstituted C₁-C₆ alkyl. In embodiments, R⁶ is hydrogen. In embodiments, R⁶ is unsubstituted methyl. In embodiments, R⁶ is unsubstituted ethyl. In embodiments, R⁶ is unsubstituted propyl. In embodiments, R⁶ is unsubstituted n-propyl. In embodiments, R⁶ is unsubstituted isopropyl. In embodiments, R⁶ is unsubstituted butyl. In embodiments, R⁶ is unsubstituted n-butyl. In embodiments, R⁶ is unsubstituted isobutyl. In embodiments, R⁶ is unsubstituted tert-butyl. In embodiments, R⁶ is unsubstituted pentyl. In embodiments, R⁶ is unsubstituted hexyl. In embodiments, R⁶ is —F. In embodiments, R⁶ is —Cl. In embodiments, R⁶ is —Br. In embodiments, R⁶ is —CHF₂. In embodiments, R⁶ is —CHF₂. In embodiments, R⁶ is —OCHF₂. In embodiments, R⁶ is unsubstituted 2 to 6 membered heteroalkyl. In embodiments, R⁶ is unsubstituted methoxy. In embodiments, R⁶ is unsubstituted ethoxy. In embodiments, R⁶ is unsubstituted propoxy. In embodiments, R⁶ is unsubstituted n-propoxy. In embodiments, R⁶ is unsubstituted isopropoxy. In embodiments, R⁶ is unsubstituted butoxy. In embodiments, R⁶ is

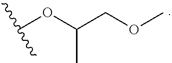 [SMILES: *OC(C)COC]

In embodiments, R⁶ is

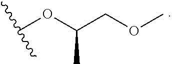 [SMILES: *O[C@H](C)COC]

In embodiments, R⁶ is

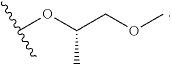 [SMILES: *O[C@@H](C)COC]

In embodiments, R⁶ is —O-(unsubstituted cyclopropyl). In embodiments, R⁶ is —O-(unsubstituted cyclobutyl). In embodiments, R⁶ is —O-(unsubstituted cyclopentyl). In embodiments, R⁶ is —O-(unsubstituted cyclohexyl). In embodiments, R⁶ is -(unsubstituted C₁-C₄ alkylene)-OTBS. In embodiments, R⁶ is —CH₂OTBS. In embodiments, R⁶ is -(unsubstituted C₁-C₄ alkylene)-OH. In embodiments, R⁶ is —CH₂OH.

In embodiments, W⁷ is N. In embodiments, W⁷ is N⁺—O⁻. In embodiments, W⁷ is C(R⁷). In embodiments, W⁷ is CH.

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, a substituted R^{7A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7A} is substituted with a plurality of groups selected from substituent groups, size-limited substituent groups, and lower substituent groups; each substituent group, size-limited substituent group, and/or lower substituent group may optionally be different. In embodiments, when R^{7A} is substituted, it is substituted with at least one substituent group. In embodiments, when R^{7A} is substituted, it is substituted with at least one size-limited substituent group. In embodiments, when R^{7A} is substituted, it is substituted with at least one lower substituent group.

In embodiments, a substituted R^{7B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7B} is substituted with a plurality of groups selected from substituent groups, size-limited substituent groups, and lower substituent groups; each substituent group, size-limited substituent group, and/or lower substituent group may optionally be different. In embodiments, when R^{7B} is substituted, it is substituted with at least one substituent group. In embodiments, when R^{7B} is substituted, it is substituted with at least one size-limited substituent group. In embodiments, when R^{7B} is substituted, it is substituted with at least one lower substituent group.

In embodiments, a substituted ring formed when R^{7A} and R^{7B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{7A} and R^{7B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{7A} and R^{7B} substituents bonded to the same nitrogen atom are joined is substituted, it is substituted with at least one substituent group. In embodiments, when the substituted ring formed when R^{7A} and R^{7B} substituents bonded to the same nitrogen atom are joined is substituted, it is substituted with at least one size-limited substituent group. In embodiments, when the substituted ring formed when R^{7A} and R^{7B} substituents bonded to the same nitrogen atom are joined is substituted, it is substituted with at least one lower substituent group.

In embodiments, a substituted R^{7C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7C} is substituted with a plurality of groups selected from substituent groups, size-limited substituent groups, and lower substituent groups; each substituent group, size-limited substituent group, and/or lower substituent group may optionally be different. In embodiments, when R^{7C} is substituted, it is substituted with at least one substituent group. In embodiments, when R^{7C} is substituted, it is substituted with at least one size-limited substituent group. In embodiments, when R^{7C} is substituted, it is substituted with at least one lower substituent group.

In embodiments, a substituted R^{7D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7D} is substituted with a plurality of groups selected from substituent groups, size-limited substituent groups, and lower substituent groups; each substituent group, size-limited substituent group, and/or lower substituent group may optionally be different. In embodiments, when R^{7D} is substituted, it is substituted with at least one substituent group. In embodiments, when R^{7D} is substituted, it is substituted with at least one size-limited substituent group. In embodiments, when R^{7D} is substituted, it is substituted with at least one lower substituent group.

In embodiments, R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NHC(O)NR^{7A}R^{7B}, —NR^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁷ is hydrogen or unsubstituted C₁-C₆ alkyl. In embodiments, R⁷ is hydrogen. In embodiments, R⁷ is unsubstituted methyl. In embodiments, R⁷ is unsubstituted ethyl. In embodiments, R⁷ is unsubstituted propyl. In embodiments, R⁷ is unsubstituted n-propyl. In embodiments, R⁷ is unsubstituted isopropyl. In embodiments, R⁷ is unsubstituted butyl. In embodiments, R⁷ is unsubstituted n-butyl. In embodiments, R⁷ is unsubstituted isobutyl. In embodiments, R⁷ is unsubstituted tert-butyl. In embodiments, R⁷ is unsubstituted pentyl. In embodiments, R⁷ is unsubstituted hexyl. In embodiments, R⁷ is —F. In embodiments, R⁷ is —Cl. In embodiments, R⁷ is —Br. In embodiments, R⁷ is —CHF₂. In embodiments, R⁷ is —CHF₂. In embodiments, R⁷ is —OCHF₂. In embodiments, R⁷ is unsubstituted 2 to 6 membered heteroalkyl. In embodiments, R⁷ is unsubstituted methoxy. In embodiments, R⁷ is unsubstituted ethoxy. In embodiments, R⁷ is unsubstituted propoxy. In embodiments, R⁷ is unsubstituted n-propoxy. In embodiments, R⁷ is unsubstituted isopropoxy. In embodiments, R⁷ is unsubstituted butoxy. In embodiments, R⁷ is

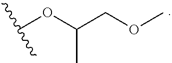 [SMILES: *OC(C)COC]

In embodiments, R⁷ is

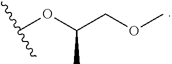 [SMILES: *O[C@H](C)COC]

In embodiments, R⁷ is

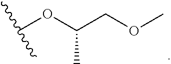 [SMILES: *O[C@@H](C)COC]

In embodiments, R⁷ is —O-(unsubstituted cyclopropyl). In embodiments, R⁷ is —O-(unsubstituted cyclobutyl). In embodiments, R⁷ is —O-(unsubstituted cyclopentyl). In embodiments, R⁷ is —O-(unsubstituted cyclohexyl). In embodiments, R⁷ is -(unsubstituted C₁-C₄ alkylene)-OTBS. In embodiments, R⁷ is —CH₂OTBS. In embodiments, R⁷ is -(unsubstituted C₁-C₄ alkylene)-OH. In embodiments, R⁷ is —CH₂OH.

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, a substituted R^{8A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8A} is substituted with a plurality of groups selected from substituent groups, size-limited substituent groups, and lower substituent groups; each substituent group, size-limited substituent group, and/or lower substituent group may optionally be different. In embodiments, when R^{8A} is substituted, it is substituted with at least one substituent group. In embodiments, when R^{8A} is substituted, it is substituted with at least one size-limited substituent group. In embodiments, when R^{8A} is substituted, it is substituted with at least one lower substituent group.

In embodiments, a substituted R^{8B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8B} is substituted with a plurality of groups selected from substituent groups, size-limited substituent groups, and lower substituent groups; each substituent group, size-limited substituent group, and/or lower substituent group may optionally be different. In embodiments, when R^{8B} is substituted, it is substituted with at least one substituent group. In embodiments, when R^{8B} is substituted, it is substituted with at least one size-limited substituent group. In embodiments, when R^{8B} is substituted, it is substituted with at least one lower substituent group.

In embodiments, a substituted ring formed when R^{8A} and R^{8B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{8A} and R^{8B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{8A} and R^{8B}, substituents bonded to the same nitrogen atom are joined is substituted, it is substituted with at least one substituent group. In embodiments, when the substituted ring formed when R^{8A} and R^{8B} substituents bonded to the same nitrogen atom are joined is substituted, it is substituted with at least one size-limited substituent group. In embodiments, when the substituted ring formed when R^{8A} and R^{8B} substituents bonded to the same nitrogen atom are joined is substituted, it is substituted with at least one lower substituent group.

In embodiments, a substituted R^{8C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8C} is substituted with a plurality of groups selected from substituent groups, size-limited substituent groups, and lower substituent groups; each substituent group, size-limited substituent group, and/or lower substituent group may optionally be different. In embodiments, when R^{8C} is substituted, it is substituted with at least one substituent group. In embodiments, when R^{8C} is substituted, it is substituted with at least one size-limited substituent group. In embodiments, when R^{8C} is substituted, it is substituted with at least one lower substituent group.

In embodiments, a substituted R^{8D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8D} is substituted with a plurality of groups selected from substituent groups, size-limited substituent groups, and lower substituent groups; each substituent group, size-limited substituent group, and/or lower substituent group may optionally be different. In embodiments, when R^{8D} is substituted, it is substituted with at least one substituent group. In embodiments, when R^{8D} is substituted, it is substituted with at least one size-limited substituent group. In embodiments, when R^{8D} is substituted, it is substituted with at least one lower substituent group.

In embodiments, R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NHC(O)NR^{8A}R^{8B}, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl.

In embodiments, R⁸ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁸ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁸ is independently halogen, —CF₃, —CHF₂, —OCHF₂, —OR^{8D}, unsubstituted C₁-C₆ alkyl, or unsubstituted 2 to 8 membered heteroalkyl.

In embodiments, R^{8D} is independently unsubstituted C₁-C₆ alkyl or unsubstituted C₃-C₈ cycloalkyl. In embodiments, R^{8D} is independently unsubstituted methyl. In embodiments, R^{8D} is independently unsubstituted ethyl. In embodiments, R^{8D} is independently unsubstituted propyl. In embodiments, R^{8D} is independently unsubstituted n-propyl. In embodiments, R^{8D} is independently unsubstituted isopropyl. In embodiments, R^{8D} is independently unsubstituted butyl. In embodiments, R^{8D} is independently unsubstituted n-butyl. In embodiments, R^{8D} is independently unsubstituted isobutyl. In embodiments, R^{8D} is independently unsubstituted tert-butyl. In embodiments, R^{8D} is independently unsubstituted pentyl. In embodiments, R^{8D} is independently unsubstituted hexyl. In embodiments, R^{8D} is independently unsubstituted cyclopropyl. In embodiments, R^{8D} is independently unsubstituted cyclobutyl. In embodiments, R^{8D} is independently unsubstituted cyclopentyl. In embodiments, R^{8D} is independently unsubstituted cyclohexyl.

In embodiments, R^{8D} is independently —F, —Cl, —Br, —CHF₂, —OCHF₂, —OCH₃, —OCH(CH₃)₂, —OCH(CH₃)CH₂OCH₃, —O-(unsubstituted cyclopropyl), or unsubstituted methyl. In embodiments, R^{8D} is independently —F, —Cl, —Br, —CH₂F, —CHF₂, —OCHF₂, —OCH₃, —OCH(CH₃)₂, —OCH(CH₃)CH₂OCH₃, —O-(unsubstituted cyclopropyl), or substituted or unsubstituted methyl.

In embodiments, R⁸ is independently —F. In embodiments, R⁸ is independently —Cl. In embodiments, R⁸ is independently —Br. In embodiments, R⁸ is independently —CH₂F. In embodiments, R⁸ is independently —CHF₂. In embodiments, R⁸ is independently —OCHF₂. In embodiments, R⁸ is independently unsubstituted C₁-C₆ alkyl. In embodiments, R⁸ is independently unsubstituted methyl. In embodiments, R⁸ is independently unsubstituted ethyl. In embodiments, R⁸ is independently unsubstituted propyl. In embodiments, R⁸ is independently unsubstituted n-propyl. In embodiments, R⁸ is independently unsubstituted isopropyl. In embodiments, R⁸ is independently unsubstituted butyl. In embodiments, R⁸ is independently unsubstituted n-butyl. In embodiments, R⁸ is independently unsubstituted isobutyl. In embodiments, R⁸ is independently unsubstituted tert-butyl. In embodiments, R⁸ is independently unsubstituted pentyl. In embodiments, R⁸ is independently unsubstituted hexyl. In embodiments, R⁸ is independently unsubstituted 2 to 6 membered heteroalkyl. In embodiments, R⁸ is independently unsubstituted methoxy. In embodiments, R⁸ is independently unsubstituted ethoxy. In embodiments, R⁸ is independently unsubstituted propoxy. In embodiments, R⁸ is independently unsubstituted n-propoxy. In embodiments, R⁸ is independently unsubstituted isopropoxy. In embodiments, R⁸ is independently unsubstituted butoxy. In embodiments, R⁸ is independently

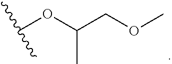 [SMILES: *OC(C)COC]

In embodiments, R⁸ is independently

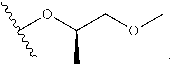 [SMILES: *O[C@H](C)COC]

In embodiments, R⁸ is independently

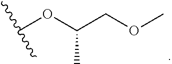 [SMILES: *O[C@@H](C)COC]

In embodiments, R⁸ is independently —O-(unsubstituted cyclopropyl). In embodiments, R⁸ is independently —O-(unsubstituted cyclobutyl). In embodiments, R⁸ is independently —O-(unsubstituted cyclopentyl). In embodiments, R⁸ is independently —O-(unsubstituted cyclohexyl). In embodiments, R⁸ is independently-(unsubstituted C₁-C₄ alkylene)-OTBS. In embodiments, R⁸ is independently —CH₂OTBS. In embodiments, R⁸ is independently -(unsubstituted C₁-C₄ alkylene)-OH. In embodiments, R⁸ is independently —CH₂OH.

In embodiments, z8 is 0. In embodiments, z8 is 1. In embodiments, z8 is 2. In embodiments, z8 is 3.

In embodiments,

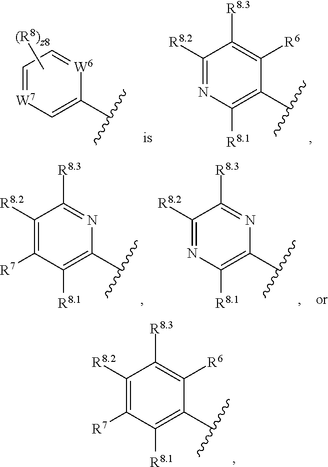

wherein R⁶ and R⁷ are as described herein, including in embodiments. R^{8.1}, R^{8.2}, and R^{8.3} are independently hydrogen or any value of R⁸ as described herein, including in embodiments.

In embodiments,

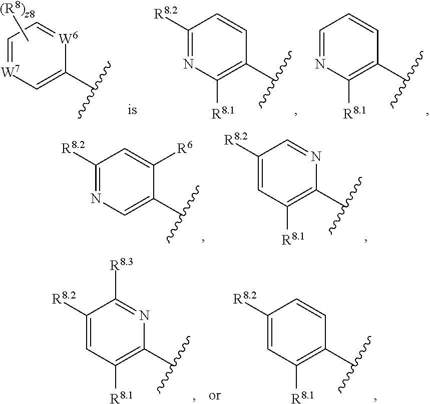

wherein R⁶ is as described herein, including in embodiments. R^{8.1}, R^{8.2}, and R^{8.3} are independently hydrogen or any value of R⁸ as described herein, including in embodiments.

In embodiments,

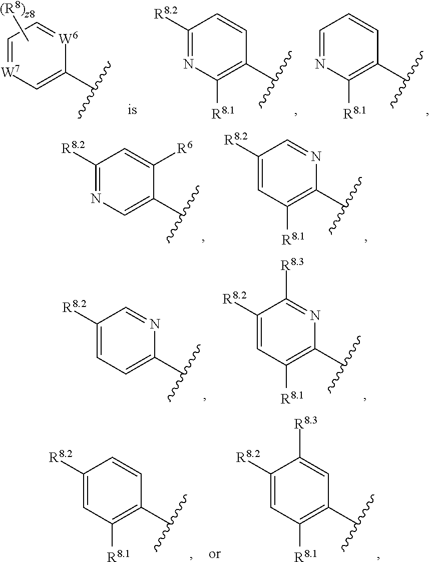

wherein R⁶ is as described herein, including in embodiments. R^{8.1}, R^{8.2}, and R^{8.3} are independently hydrogen or any value of R⁸ as described herein, including in embodiments.

In embodiments, a substituted R^{8.1} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8.1} is substituted with a plurality of groups selected from substituent groups, size-limited substituent groups, and lower substituent groups; each substituent group, size-limited substituent group, and/or lower substituent group may optionally be different. In embodiments, when R^{8.1} is substituted, it is substituted with at least one substituent group. In embodiments, when R^{8.1} is substituted, it is substituted with at least one size-limited substituent group. In embodiments, when R^{8.1} is substituted, it is substituted with at least one lower substituent group.

In embodiments, a substituted R^{8.2} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8.2} is substituted with a plurality of groups selected from substituent groups, size-limited substituent groups, and lower substituent groups; each substituent group, size-limited substituent group, and/or lower substituent group may optionally be different. In embodiments, when R^{8.2} is substituted, it is substituted with at least one substituent group. In embodiments, when R^{8.2} is substituted, it is substituted with at least one size-limited substituent group. In embodiments, when R^{8.2} is substituted, it is substituted with at least one lower substituent group.

In embodiments, a substituted R^{8.3} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8.3} is substituted with a plurality of groups selected from substituent groups, size-limited substituent groups, and lower substituent groups; each substituent group, size-limited substituent group, and/or lower substituent group may optionally be different. In embodiments, when R^{8.3} is substituted, it is substituted with at least one substituent group. In embodiments, when R^{8.3} is substituted, it is substituted with at least one size-limited substituent group. In embodiments, when R^{8.3} is substituted, it is substituted with at least one lower substituent group.

In embodiments, a substituted ring formed when R^{8.2} and R^{8.3} substituents are joined (e.g., substituted cycloalkyl and/or substituted heterocycloalkyl) is substituted with at least one substituent group, size-limited substituent group, or lower substituent group; wherein if the substituted ring formed when R^{8.2} and R^{8.3} substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{8.2} and R^{8.3} substituents are joined is substituted, it is substituted with at least one substituent group. In embodiments, when the substituted ring formed when R^{8.2} and R^{8.3} substituents are joined is substituted, it is substituted with at least one size-limited substituent group. In embodiments, when the substituted ring formed when R^{8.2} and R^{8.3} substituents are joined is substituted, it is substituted with at least one lower substituent group.

In embodiments, R^{0.1}, R^{8.2}, and R^{8.3} are independently hydrogen, halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{8.2} and R^{8.3} substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R^{0.1}, R^{8.2}, and R^{8.3} are independently hydrogen, halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NHC(O)NR^{8A}R^{8B}, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{8.2} and R^{8.3} substituents may optionally be joined to form a substituted or unsubstituted cycloalkyl, substituted or unsubstituted heterocycloalkyl, substituted or unsubstituted aryl, or substituted or unsubstituted heteroaryl.

In embodiments, R^{8.1}, R^{8.2}, and R^{8.3} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂J, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{8.1}, R², and R^{8.3} are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂J, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{8.1} is —F. In embodiments, R^{8.1} is —Cl. In embodiments, R^{8.1} is —Br. In embodiments, R^{8.1} is —CH₂F. In embodiments, R^{8.1} is —CHF₂. In embodiments, R^{8.1} is —OCHF₂. In embodiments, R^{8.1} is unsubstituted C₁-C₆ alkyl. In embodiments, R^{8.1} is unsubstituted methyl. In embodiments, R^{8.1} is unsubstituted ethyl. In embodiments, R^{8.1} is unsubstituted propyl. In embodiments, R^{8.1} is unsubstituted n-propyl. In embodiments, R^{8.1} is unsubstituted isopropyl. In embodiments, R^{8.1} is unsubstituted butyl. In embodiments, R^{8.1} is unsubstituted n-butyl. In embodiments, R^{8.1} is unsubstituted isobutyl. In embodiments, R^{8.1} is unsubstituted tert-butyl. In embodiments, R^{8.1} is unsubstituted pentyl. In embodiments, R^{8.1} is unsubstituted hexyl. In embodiments, R^{8.1} is unsubstituted 2 to 6 membered heteroalkyl. In embodiments, R^{8.1} is unsubstituted methoxy. In embodiments, R^{8.1} is unsubstituted ethoxy. In embodiments, R^{8.1} is unsubstituted propoxy. In embodiments, R^{8.1} is unsubstituted n-propoxy. In embodiments, R^{8.1} is unsubstituted isopropoxy. In embodiments, R^{8.1} is unsubstituted butoxy. In embodiments, R^{8.1} is

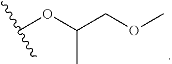 [SMILES: *OC(C)COC]

In embodiments, R^{8.1} is

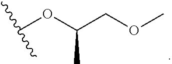 [SMILES: *O[C@H](C)COC]

In embodiments, R^{8.1} is

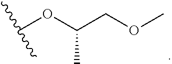 [SMILES: *O[C@@H](C)COC]

In embodiments, R^{8.1} is —O-(unsubstituted cyclopropyl). In embodiments, R^{8.1} is —O-(unsubstituted cyclobutyl). In embodiments, R^{8.1} is —O-(unsubstituted cyclopentyl). In embodiments, R^{8.1} is —O-(unsubstituted cyclohexyl). In embodiments, R^{8.1} is -(unsubstituted C₁-C₄ alkylene)-OTBS. In embodiments, R^{8.1} is —CH₂OTBS. In embodiments, R^{8.1} is -(unsubstituted C₁-C₄ alkylene)-OH. In embodiments, R^{8.1} is —CH₂OH.

In embodiments, R^{8.2} is —F. In embodiments, R^{8.2} is —Cl. In embodiments, R^{8.2} is —Br. In embodiments, R^{8.2} is —CH₂F. In embodiments, R^{8.2} is —CHF₂. In embodiments, R^{8.2} is —OCHF₂. In embodiments, R^{8.2} is unsubstituted C₁-C₆ alkyl. In embodiments, R^{8.2} is unsubstituted methyl. In embodiments, R^{8.2} is unsubstituted ethyl. In embodiments, R^{8.2} is unsubstituted propyl. In embodiments, R^{8.2} is unsubstituted n-propyl. In embodiments, R^{8.2} is unsubstituted isopropyl. In embodiments, R^{8.2} is unsubstituted butyl. In embodiments, R^{8.2} is unsubstituted n-butyl. In embodiments, R^{8.2} is unsubstituted isobutyl. In embodiments, R^{8.2} is unsubstituted tert-butyl. In embodiments, R^{8.2} is unsubstituted pentyl. In embodiments, R^{8.2} is unsubstituted hexyl. In embodiments, R^{8.2} is unsubstituted 2 to 6 membered heteroalkyl. In embodiments, R^{8.2} is unsubstituted methoxy. In embodiments, R^{8.2} is unsubstituted ethoxy. In embodiments, R^{8.2} is unsubstituted propoxy. In embodiments, R^{8.2} is unsubstituted n-propoxy. In embodiments, R^{8.2} is unsubstituted isopropoxy. In embodiments, R^{8.2} is unsubstituted butoxy. In embodiments, R^{8.2} is

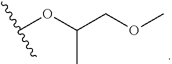 [SMILES: *OC(C)COC]

In embodiments R^{8.2} is

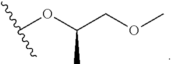 [SMILES: *O[C@H](C)COC]

In embodiments, R^{8.2} is

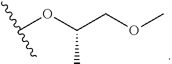 [SMILES: *O[C@@H](C)COC]

In embodiments, R^{8.2} is —O-(unsubstituted cyclopropyl). In embodiments, R^{8.2} is —O-(unsubstituted cyclobutyl). In embodiments, R^{8.2} is —O-(unsubstituted cyclopentyl). In embodiments, R^{8.2} is —O-(unsubstituted cyclohexyl). In embodiments, R^{8.2} is -(unsubstituted C₁-C₄ alkylene)-OTBS. In embodiments, R^{8.2} is —CH₂OTBS. In embodiments, R^{8.2} is -(unsubstituted C₁-C₄ alkylene)-OH. In embodiments, R^{8.2} is —CH₂OH.

In embodiments, R^{8.3} is —F. In embodiments, R^{8.3} is —Cl. In embodiments, R^{8.3} is —Br. In embodiments, R^{8.3} is —CH₂F. In embodiments, R^{8.3} is —CHF₂. In embodiments, R^{8.3} is —OCHF₂. In embodiments, R^{8.3} is unsubstituted C₁-C₆ alkyl. In embodiments, R^{8.3} is unsubstituted methyl. In embodiments, R^{8.3} is unsubstituted ethyl. In embodiments, R^{8.3} is unsubstituted propyl. In embodiments, R^{8.3} is unsubstituted n-propyl. In embodiments, R^{8.3} is unsubstituted isopropyl. In embodiments, R^{8.3} is unsubstituted butyl. In embodiments, R^{8.3} is unsubstituted n-butyl. In embodiments, R^{8.3} is unsubstituted isobutyl. In embodiments, R^{8.3} is unsubstituted tert-butyl. In embodiments, R^{8.3} is unsubstituted pentyl. In embodiments, R^{8.3} is unsubstituted hexyl. In embodiments, R^{8.3} is unsubstituted 2 to 6 membered heteroalkyl. In embodiments, R^{8.3} is unsubstituted methoxy. In embodiments, R^{8.3} is unsubstituted ethoxy. In embodiments, R^{8.3} is unsubstituted propoxy. In embodiments, R^{8.3} is unsubstituted n-propoxy. In embodiments, R^{8.3} is unsubstituted isopropoxy. In embodiments, R^{8.3} is unsubstituted butoxy. In embodiments, R^{8.3} is

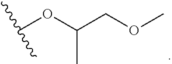 [SMILES: *OC(C)COC]

In embodiments, R^{8.3} is

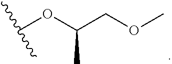 [SMILES: *O[C@H](C)COC]

In embodiments, R^{8.3} is

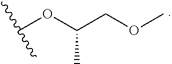 [SMILES: *O[C@@H](C)COC]

In embodiments, R^{8.3} is —O-(unsubstituted cyclopropyl). In embodiments, R^{8.3} is —O-(unsubstituted cyclobutyl). In embodiments, R^{8.3} is —O-(unsubstituted cyclopentyl). In embodiments, R^{8.3} is —O-(unsubstituted cyclohexyl). In embodiments, R^{8.3} is -(unsubstituted C₁-C₄ alkylene)-OTBS. In embodiments, R^{8.3} is —CH₂OTBS. In embodiments, R^{8.3} is -(unsubstituted C₁-C₄ alkylene)-OH. In embodiments, R^{8.3} is —CH₂OH.

In embodiments,

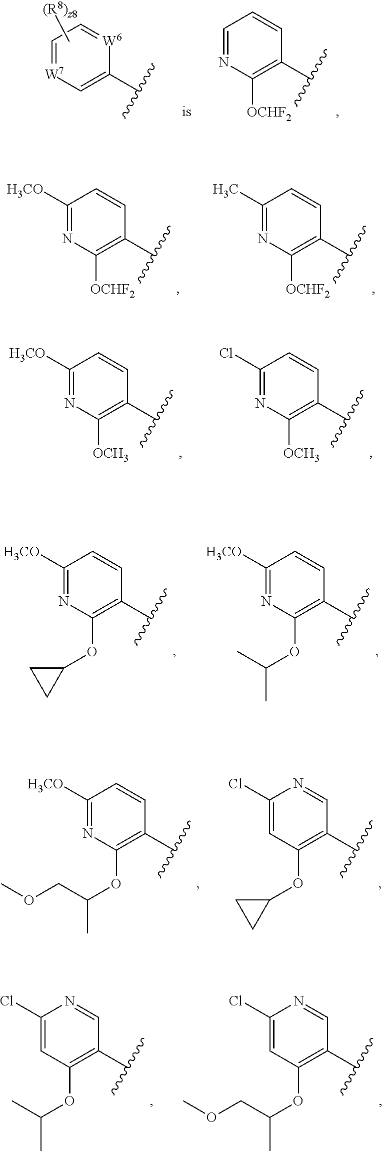

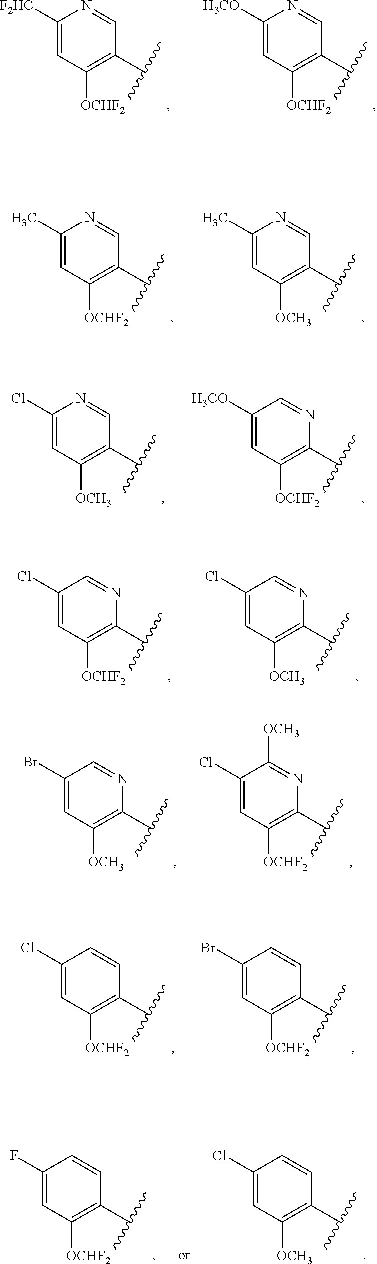

In embodiments,

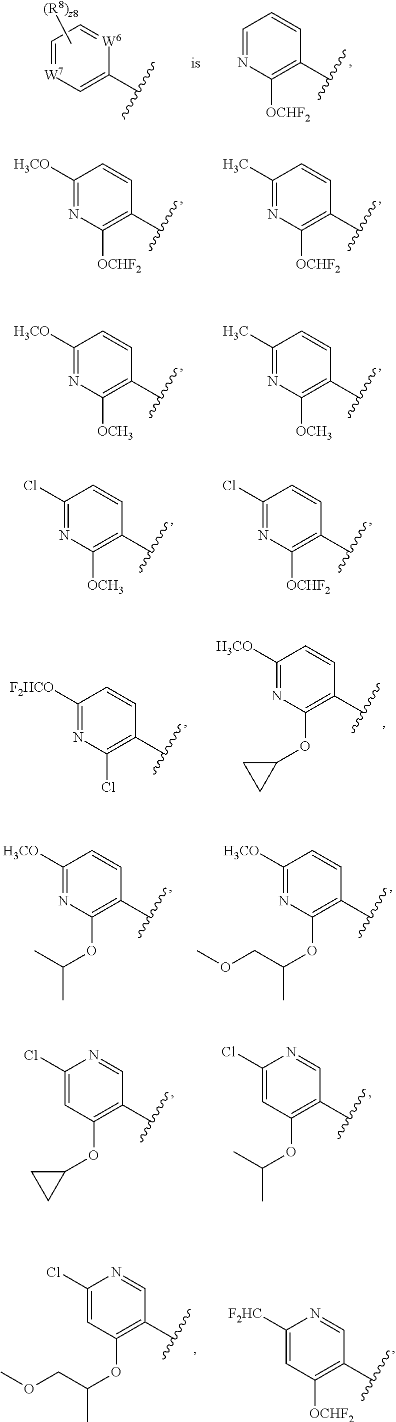

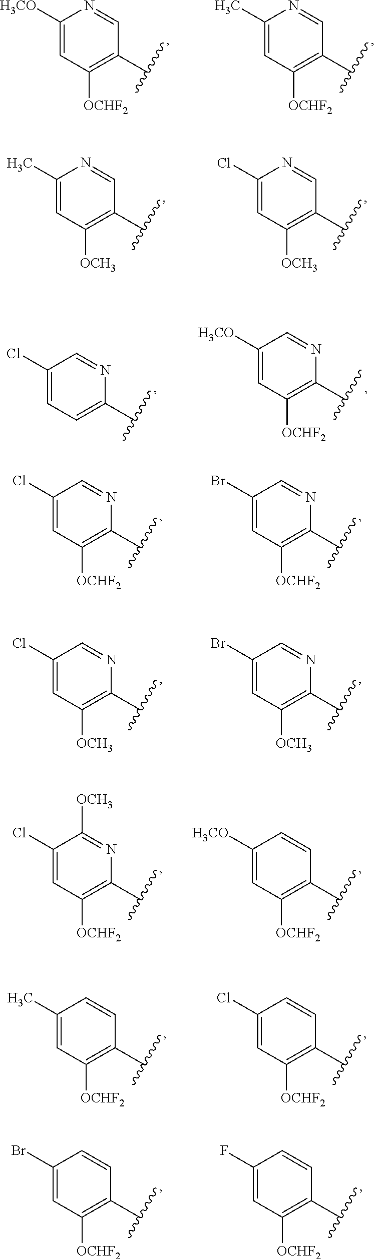

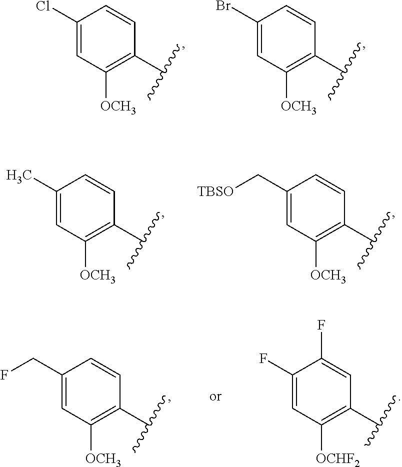

In embodiments,

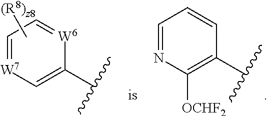

In embodiments,

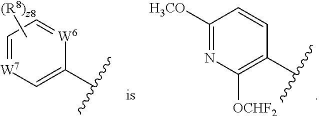

In embodiments,

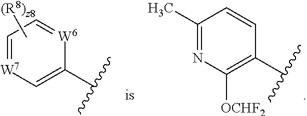

In embodiments,

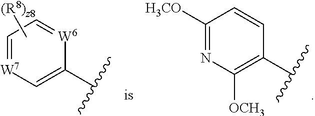

In embodiments,

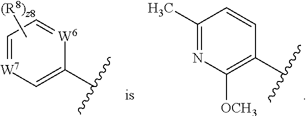

In embodiments,

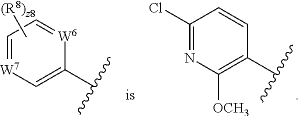

In embodiments,

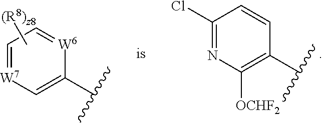

In embodiments,

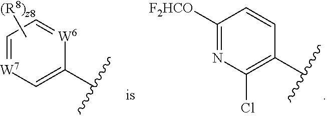

In embodiments,

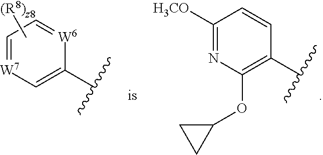

In embodiments,

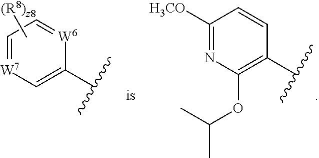

In embodiments,

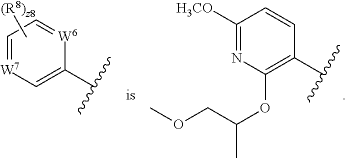

In embodiments,

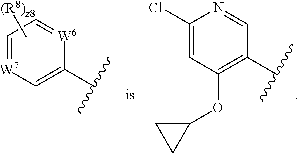

In embodiments,

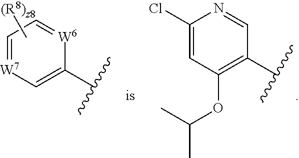

In embodiments,

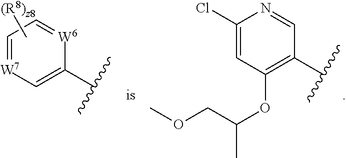

In embodiments,

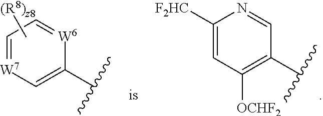

In embodiments,

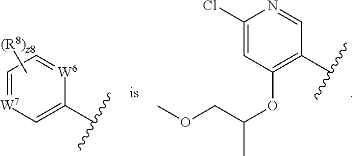

In embodiments,

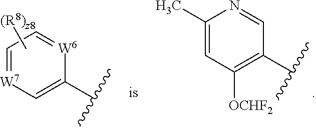

In embodiments,

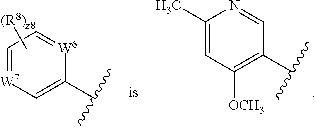

In embodiments,

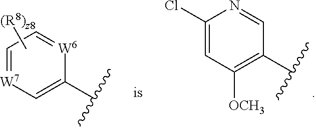

In embodiments,

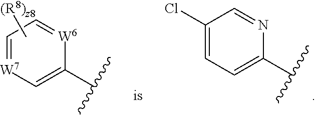

In embodiments,

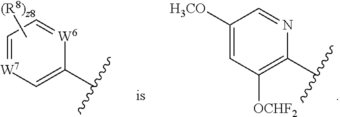

In embodiments,

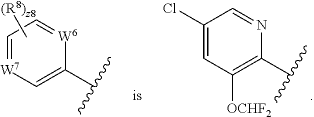

In embodiments,

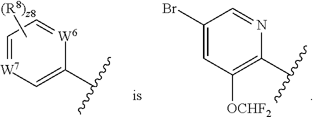

In embodiments,

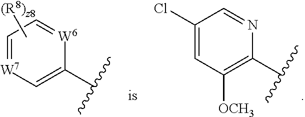

In embodiments,

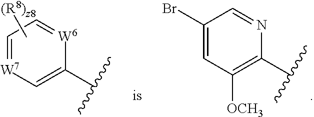

In embodiments,

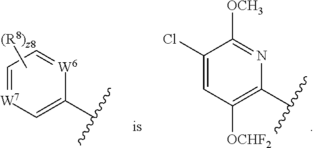

In embodiments,

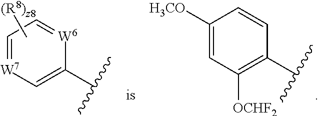

In embodiments,

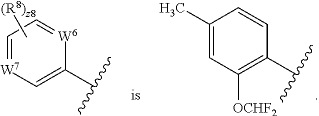

In embodiments,

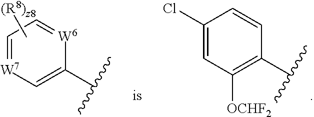

In embodiments,

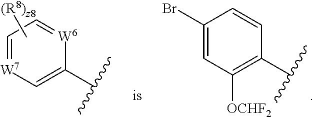

In embodiments,

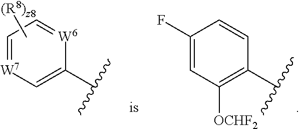

In embodiments,

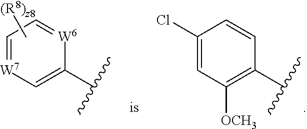

In embodiments,

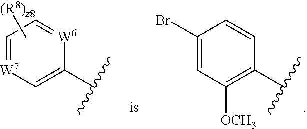

In embodiments,

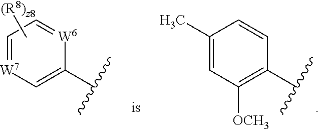

In embodiments,

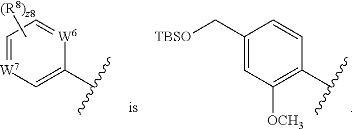

In embodiments,

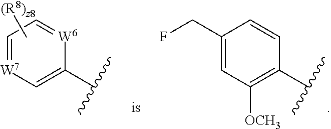

In embodiments,

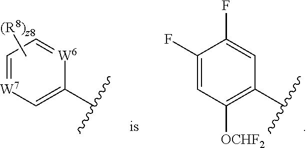

In embodiments,

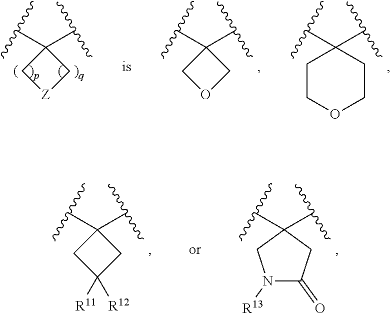

wherein R¹¹, R¹², and R¹³ are as described herein, including in embodiments. In embodiments,

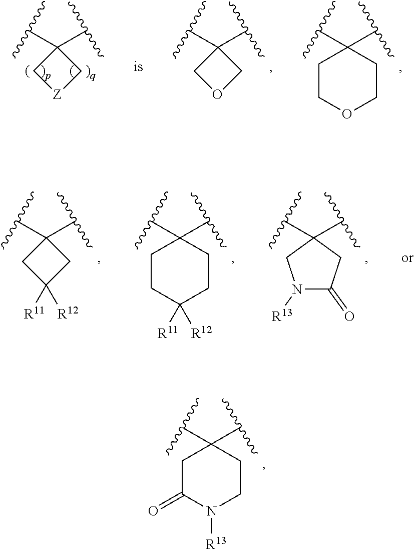

wherein R¹¹, R¹², and R¹³ are as described herein, including in embodiments. In embodiments,

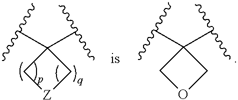

In embodiments,

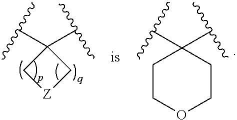

In embodiments,

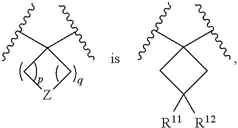

wherein R¹¹ and R¹² are as described herein, including in embodiments. In embodiments,

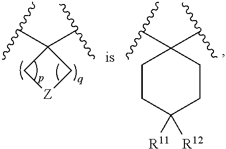

wherein R¹¹ and R¹² are as described herein, including in embodiments. In embodiments

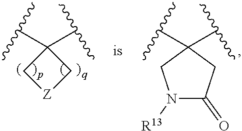

wherein R¹³ is as described herein, including in embodiments. In embodiments,

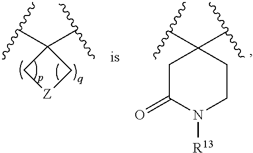

wherein R¹³ is as described herein, including in embodiments.

In embodiments, a substituted R¹¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹¹ is substituted with a plurality of groups selected from substituent groups, size-limited substituent groups, and lower substituent groups; each substituent group, size-limited substituent group, and/or lower substituent group may optionally be different. In embodiments, when R¹¹ is substituted, it is substituted with at least one substituent group. In embodiments, when R¹¹ is substituted, it is substituted with at least one size-limited substituent group. In embodiments, when R¹¹ is substituted, it is substituted with at least one lower substituent group.

In embodiments, a substituted R^{11A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11A} is substituted with a plurality of groups selected from substituent groups, size-limited substituent groups, and lower substituent groups; each substituent group, size-limited substituent group, and/or lower substituent group may optionally be different. In embodiments, when R^{11A} is substituted, it is substituted with at least one substituent group. In embodiments, when R^{11A} is substituted, it is substituted with at least one size-limited substituent group. In embodiments, when R^{11A} is substituted, it is substituted with at least one lower substituent group.

In embodiments, a substituted R^{11B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11B} is substituted with a plurality of groups selected from substituent groups, size-limited substituent groups, and lower substituent groups; each substituent group, size-limited substituent group, and/or lower substituent group may optionally be different. In embodiments, when R^{11B} is substituted, it is substituted with at least one substituent group. In embodiments, when R^{11B} is substituted, it is substituted with at least one size-limited substituent group. In embodiments, when R^{11B} is substituted, it is substituted with at least one lower substituent group.

In embodiments, a substituted ring formed when R^{11A} and R^{11B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{11A} and R^{11B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{11A} and R^{11B} substituents bonded to the same nitrogen atom are joined is substituted, it is substituted with at least one substituent group. In embodiments, when the substituted ring formed when R^{11A} and R^{11B} substituents bonded to the same nitrogen atom are joined is substituted, it is substituted with at least one size-limited substituent group. In embodiments, when the substituted ring formed when R^{11A} and R^{11B} substituents bonded to the same nitrogen atom are joined is substituted, it is substituted with at least one lower substituent group.

In embodiments, a substituted R^{11C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11C} is substituted with a plurality of groups selected from substituent groups, size-limited substituent groups, and lower substituent groups; each substituent group, size-limited substituent group, and/or lower substituent group may optionally be different. In embodiments, when R^{11C} is substituted, it is substituted with at least one substituent group. In embodiments, when R^{11C} is substituted, it is substituted with at least one size-limited substituent group. In embodiments, when R^{11C} is substituted, it is substituted with at least one lower substituent group.

In embodiments, a substituted R^{11D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11D} is substituted with a plurality of groups selected from substituent groups, size-limited substituent groups, and lower substituent groups; each substituent group, size-limited substituent group, and/or lower substituent group may optionally be different. In embodiments, when R^{11D} is substituted, it is substituted with at least one substituent group. In embodiments, when R^{11D} is substituted, it is substituted with at least one size-limited substituent group. In embodiments, when R^{11D} is substituted, it is substituted with at least one lower substituent group.

In embodiments, R¹¹ is hydrogen, halogen, —CX¹¹₃, —CHX¹¹₂, —CH₂X¹¹, —OCX¹¹₃, —OCH₂X¹¹, —OCHX¹¹₂, —CN, —SOₙ₁₁R^{11D}, —SOᵥ₁₁NR^{11A}R^{11B}, —NHC(O)NR^{11A}R^{11B}, —NR^{11A}R^{11B}, —C(O)R^{11C}, —C(O)OR^{11C}, —C(O)NR^{11A}R^{11B}, —OR^{11D}, —SR^{11D}, —NR^{11A}SO₂R^{11D}, —NR^{11A} C(O)R^{11C}, —NR^{11A} C(O)OR^{11C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹¹ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹¹ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹¹ is hydrogen, halogen, —SOₙ₁₁R^{11D}, —SOᵥ₁₁NR^{11A}R^{11B}, —NR^{11A}R^{11B}, —C(O)OR^{11C}, —C(O)NR^{11A}R^{11B}, —OR^{11D}, —NR^{11A}SO₂R^{11D}, —NR^{11A} C(O)R^{11C} substituted or unsubstituted C₁-C₆ alkyl, substituted or unsubstituted 2 to 6 membered heteroalkyl, substituted or unsubstituted 3 to 8 membered heterocycloalkyl, or substituted or unsubstituted 5 to 6 membered heteroaryl. In embodiments, R^{11A} and R^{11B} are independently hydrogen, unsubstituted C₁-C₆ alkyl, or unsubstituted C₃-C₈ cycloalkyl. In embodiments, R^{11C} is hydrogen or unsubstituted C₁-C₆ alkyl. In embodiments, R^{11D} is hydrogen or unsubstituted C₁-C₆ alkyl.

In embodiments, R^{11A} is hydrogen. In embodiments, R^{11A} is unsubstituted C₁-C₆ alkyl. In embodiments, R^{11A} is unsubstituted C₃-C₈ cycloalkyl. In embodiments, R^{11A} is unsubstituted methyl. In embodiments, R^{11A} is unsubstituted ethyl. In embodiments, R^{11A} is unsubstituted propyl. In embodiments, R^{11A} is unsubstituted n-propyl. In embodiments, R^{11A} is unsubstituted isopropyl. In embodiments, R^{11A} is unsubstituted butyl. In embodiments, R^{11A} is unsubstituted n-butyl. In embodiments, R^{11A} is unsubstituted isobutyl. In embodiments, R^{11A} is unsubstituted tert-butyl. In embodiments, R^{11A} is unsubstituted pentyl. In embodiments, R^{11A} is unsubstituted hexyl. In embodiments, R^{11A} is unsubstituted cyclopropyl. In embodiments, R^{11A} is unsubstituted cyclobutyl. In embodiments, R^{11A} is unsubstituted cyclopentyl. In embodiments, R^{11A} is unsubstituted cyclohexyl.

In embodiments, R^{11B} is hydrogen. In embodiments, R^{11B} is unsubstituted C₁-C₆ alkyl. In embodiments, R^{11B} is unsubstituted C₃-C₈ cycloalkyl. In embodiments, R^{11B} is unsubstituted methyl. In embodiments, R^{11B} is unsubstituted ethyl. In embodiments, R^{11B} is unsubstituted propyl. In embodiments, R^{11B} is unsubstituted n-propyl. In embodiments, R^{11B} is unsubstituted isopropyl. In embodiments, R^{11B} is unsubstituted butyl. In embodiments, R^{11B} is unsubstituted n-butyl. In embodiments, R^{11B} is unsubstituted isobutyl. In embodiments, R^{11B} is unsubstituted tert-butyl. In embodiments, R^{11B} is unsubstituted pentyl. In embodiments, R^{11B} is unsubstituted hexyl. In embodiments, R^{11B} is unsubstituted cyclopropyl. In embodiments, R^{11B} is unsubstituted cyclobutyl. In embodiments, R^{11B} is unsubstituted cyclopentyl. In embodiments, R^{11B} is unsubstituted cyclohexyl.

In embodiments, R^{11C} is hydrogen. In embodiments, R^{11C} is unsubstituted C₁-C₆ alkyl. In embodiments, R^{11C} is unsubstituted methyl. In embodiments, R^{11C} is unsubstituted ethyl. In embodiments, R^{11C} is unsubstituted propyl. In embodiments, R^{11C} is unsubstituted n-propyl. In embodiments, R^{11C} is unsubstituted isopropyl. In embodiments, R^{11C} is unsubstituted butyl. In embodiments, R^{11C} is unsubstituted n-butyl. In embodiments, R^{11C} is unsubstituted isobutyl. In embodiments, R^{11C} is unsubstituted tert-butyl. In embodiments, R^{11C} is unsubstituted pentyl. In embodiments, R^{11C} is unsubstituted hexyl.

In embodiments, R^{11D} is hydrogen. In embodiments, R^{11D} is unsubstituted C₁-C₆ alkyl. In embodiments, R^{11D} is unsubstituted methyl. In embodiments, R^{11D} is unsubstituted ethyl. In embodiments, R^{11D} is unsubstituted propyl. In embodiments, R^{11D} is unsubstituted n-propyl. In embodiments, R^{11D} is unsubstituted isopropyl. In embodiments, R^{11D} is unsubstituted butyl. In embodiments, R^{11D} is unsubstituted n-butyl. In embodiments, R^{11D} is unsubstituted isobutyl. In embodiments, R^{11D} is unsubstituted tert-butyl. In embodiments, R^{11D} is unsubstituted pentyl. In embodiments, R^{11D} is unsubstituted hexyl.

In embodiments, R¹¹ is hydrogen, —F, —OH, —CH₃, —CH₂CH₃, —NHCH₃, —N(CH₃)₂, —C(O)OH, —C(O)N(CH₃)₂, —S(O)₂CH₃, —S(O)₂NH₂, —S(O)₂NH(CH₃)₂,

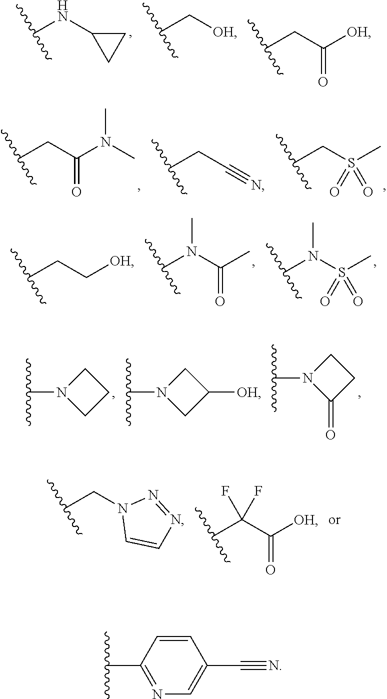

In embodiments, R¹¹ is hydrogen, —F, —OH, —CN, —CH₃, —CH₂CH₃, —NHCH₃, —N(CH₃)₂, —C(O)OH, —C(O)NH₂, —C(O)N(CH₃)₂, —S(O)₂CH₃, —S(O)₂CH₃, —S(O)₂NH₂(CH₃), —S(O)₂N(CH₃)₂, —N₃,

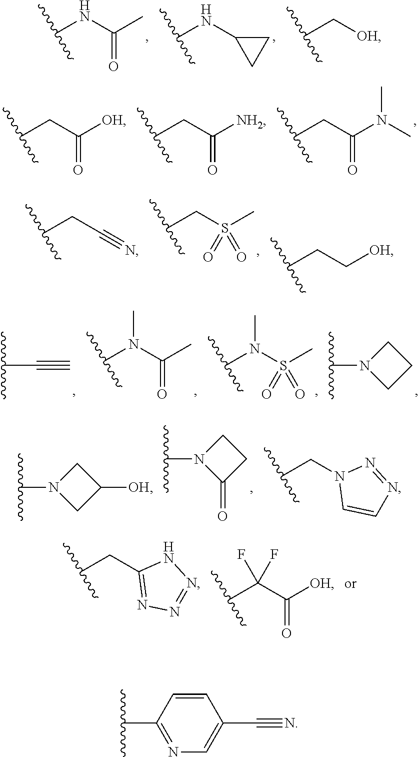

In embodiments, R¹¹ is hydrogen. In embodiments, R¹¹ is halogen. In embodiments, R¹¹ is —F. In embodiments, R¹¹ is —OH. In embodiments, R¹¹ is —CN. In embodiments, R¹¹ is —NHCH₃. In embodiments, R¹¹ is —N(CH₃)₂. In embodiments, R¹¹ is —C(O)OH. In embodiments, R¹¹ is —C(O)NH₂. In embodiments, R¹¹ is —C(O)N(CH₃)₂. In embodiments, R¹¹ is —S(O)₂CH₃. In embodiments, R¹¹ is —S(O)₂NH₂. In embodiments, R¹¹ is —S(O)₂NH(CH₃). In embodiments, R¹¹ is —S(O)₂N(CH₃)₂. In embodiments, R¹¹ is —N₃. In embodiments, R¹¹ is unsubstituted C₁-C₆ alkyl. In embodiments, R¹¹ is unsubstituted methyl. In embodiments, R¹¹ is -CD₃. In embodiments, R¹¹ is unsubstituted ethyl. In embodiments, R¹¹ is unsubstituted propyl. In embodiments, R¹¹ is unsubstituted n-propyl. In embodiments, R¹¹ is unsubstituted isopropyl. In embodiments, R¹¹ is unsubstituted butyl. In embodiments, R¹¹ is unsubstituted n-butyl. In embodiments, R¹¹ is unsubstituted isobutyl. In embodiments, R¹¹ is unsubstituted tert-butyl. In embodiments, R¹¹ is unsubstituted pentyl. In embodiments, R¹¹ is unsubstituted hexyl. In embodiments, R¹¹ is unsubstituted C₂-C₆ alkynyl. In embodiments, R¹¹ is unsubstituted ethynyl. In embodiments, R¹¹ is unsubstituted propynyl. In embodiments, R¹¹ is unsubstituted butynyl. In embodiments, R¹¹ is unsubstituted pentynyl. In embodiments, R¹¹ is unsubstituted hexynyl. In embodiments, R¹¹ is substituted or unsubstituted 2 to 6 membered heteroalkyl. In embodiments, R¹¹ is unsubstituted methoxy. In embodiments, R¹¹ is unsubstituted ethoxy. In embodiments, R¹¹ is unsubstituted propoxy. In embodiments, R¹¹ is unsubstituted n-propoxy. In embodiments, R¹¹ is unsubstituted isopropoxy. In embodiments, R¹¹ is unsubstituted butoxy. In embodiments, R¹¹ is substituted or unsubstituted 3 to 8 membered heterocycloalkyl. In embodiments, R¹¹ is substituted or unsubstituted azetidinyl. In embodiments, R¹¹ is substituted or unsubstituted 5 to 6 membered heteroaryl. In embodiments, R¹¹ is substituted or unsubstituted triazolyl. In embodiments, R¹¹ is substituted or unsubstituted pyridyl. In embodiments, R¹¹ is

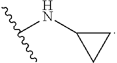 [SMILES: *NC1CC1]

In embodiments, R¹¹ is

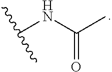 [SMILES: *NC(C)=O]

In embodiments, R¹¹ is

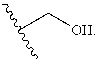 [SMILES: *CO]

In embodiments, R¹¹ is

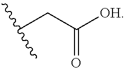 [SMILES: *CC(=O)O]

In embodiments, R¹¹ is

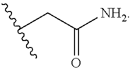 [SMILES: *CC(N)=O]

In embodiments, R¹¹ is

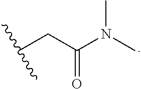 [SMILES: *CC(=O)N(C)C]

In embodiments, R¹¹ is

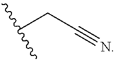 [SMILES: *CC#N]

In embodiments, R¹¹ is

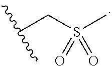 [SMILES: *CS(C)(=O)=O]

In embodiments, R¹¹ is

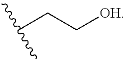 [SMILES: *CCO]

In embodiments, R¹¹ is

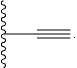 [SMILES: *C#C]

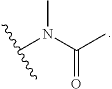 [SMILES: *N(C)C(C)=O]

In embodiments, R¹¹ is

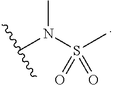 [SMILES: *N(C)S(C)(=O)=O]

In embodiments, R¹¹ is

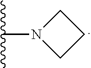 [SMILES: *N1CCC1]

In embodiments, R¹¹ is

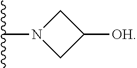 [SMILES: *N1CC(O)C1]

In embodiments, R¹¹ is

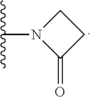 [SMILES: *N1CCC1=O]

In embodiments, R¹¹ is

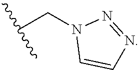 [SMILES: *Cn1ccnn1]

In embodiments, R¹¹ is

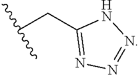 [SMILES: *Cc1nnn[nH]1]

In embodiments, R¹¹ is

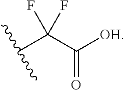 [SMILES: *C(F)(F)C(=O)O]

In embodiments, R¹¹ is

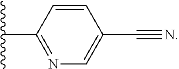 [SMILES: *c1ccc(C#N)cn1]

In embodiments, a substituted R¹² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹² is substituted with a plurality of groups selected from substituent groups, size-limited substituent groups, and lower substituent groups; each substituent group, size-limited substituent group, and/or lower substituent group may optionally be different. In embodiments, when R¹² is substituted, it is substituted with at least one substituent group. In embodiments, when R¹² is substituted, it is substituted with at least one size-limited substituent group. In embodiments, when R¹² is substituted, it is substituted with at least one lower substituent group.

In embodiments, a substituted R^{12A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12A} is substituted with a plurality of groups selected from substituent groups, size-limited substituent groups, and lower substituent groups; each substituent group, size-limited substituent group, and/or lower substituent group may optionally be different. In embodiments, when R^{12A} is substituted, it is substituted with at least one substituent group. In embodiments, when R^{12A} is substituted, it is substituted with at least one size-limited substituent group. In embodiments, when R^{12A} is substituted, it is substituted with at least one lower substituent group.

In embodiments, a substituted R^{12B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12B} is substituted with a plurality of groups selected from substituent groups, size-limited substituent groups, and lower substituent groups; each substituent group, size-limited substituent group, and/or lower substituent group may optionally be different. In embodiments, when R^{12B} is substituted, it is substituted with at least one substituent group. In embodiments, when R^{12B} is substituted, it is substituted with at least one size-limited substituent group. In embodiments, when R^{12B} is substituted, it is substituted with at least one lower substituent group.

In embodiments, a substituted ring formed when R^{12A} and R^{12B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{12A} and R^{12B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{12A} and R^{12B} substituents bonded to the same nitrogen atom are joined is substituted, it is substituted with at least one substituent group. In embodiments, when the substituted ring formed when R^{12A} and R^{12B} substituents bonded to the same nitrogen atom are joined is substituted, it is substituted with at least one size-limited substituent group. In embodiments, when the substituted ring formed when R^{12A} and R^{12B} substituents bonded to the same nitrogen atom are joined is substituted, it is substituted with at least one lower substituent group.

In embodiments, a substituted R^{12C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12C} is substituted with a plurality of groups selected from substituent groups, size-limited substituent groups, and lower substituent groups; each substituent group, size-limited substituent group, and/or lower substituent group may optionally be different. In embodiments, when R^{12C} is substituted, it is substituted with at least one substituent group. In embodiments, when R^{12C} is substituted, it is substituted with at least one size-limited substituent group. In embodiments, when R^{12C} is substituted, it is substituted with at least one lower substituent group.

In embodiments, a substituted R^{12D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12D} is substituted with a plurality of groups selected from substituent groups, size-limited substituent groups, and lower substituent groups; each substituent group, size-limited substituent group, and/or lower substituent group may optionally be different. In embodiments, when R^{12D} is substituted, it is substituted with at least one substituent group. In embodiments, when R^{12D} is substituted, it is substituted with at least one size-limited substituent group. In embodiments, when R^{12D} is substituted, it is substituted with at least one lower substituent group.

In embodiments, R¹² is hydrogen, halogen, —CX¹²₃, —CHX¹²₂, —CH₂X¹², —OCX¹²₃, —OCH₂X¹², —OCHX¹²₂, —CN, —SOₙ₁₂R^{12D}, —SOᵥ₁₂NR^{12A}R^{12B}, —NHC(O)NR^{12A}R^{12B}, —NR^{12A}R^{12B}, —C(O)R^{12C}, —C(O)OR^{12C}, —C(O)NR^{12A}R^{12B}, —OR^{12D}, —SR^{12D}, —NR^{12A}SO₂R^{12D}, —NR^{12A}C(O)R^{12C}, —NR^{12A}C(O)OR^{12C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹² is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHC(O)NH₂, —NH₂, —C(O)H, —C(O)OH, —CONH₂, —OH, —SH, —NHSO₂H, —NHC(O)H, —NHC(O)OH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹² is hydrogen, halogen, —SOₙ₁₂R^{12D}, —SOᵥ₁₂NR^{12A}R^{12B}, —NR^{12A}R^{12B}, —C(O)OR^{12C}, —C(O)NR^{12A}R^{12B}, —OR^{12D}, —NR^{12A}SO₂R^{12D}, —NR^{12A}C(O)R^{12C} substituted or unsubstituted C₁-C₆ alkyl, substituted or unsubstituted 2 to 6 membered heteroalkyl, substituted or unsubstituted 3 to 8 membered heterocycloalkyl, or substituted or unsubstituted 5 to 6 membered heteroaryl. In embodiments, R^{12A} and R^{12B} are independently hydrogen, unsubstituted C₁-C₆ alkyl, or unsubstituted C₃-C₈ cycloalkyl. In embodiments, R^{12C} is hydrogen or unsubstituted C₁-C₆ alkyl. In embodiments, R^{12D} is hydrogen or unsubstituted C₁-C₆ alkyl.

In embodiments, R^{12A} is hydrogen. In embodiments, R^{12A} is unsubstituted C₁-C₆ alkyl. In embodiments, R^{12A} is unsubstituted C₃-C₈ cycloalkyl. In embodiments, R^{12A} is unsubstituted methyl. In embodiments, R^{12A} is unsubstituted ethyl. In embodiments, R^{12A} is unsubstituted propyl. In embodiments, R^{12A} is unsubstituted n-propyl. In embodiments, R^{12A} is unsubstituted isopropyl. In embodiments, R^{12A} is unsubstituted butyl. In embodiments, R^{12A} is unsubstituted n-butyl. In embodiments, R^{12A} is unsubstituted isobutyl. In embodiments, R^{12A} is unsubstituted tert-butyl. In embodiments, R^{12A} is unsubstituted pentyl. In embodiments, R^{12A} is unsubstituted hexyl. In embodiments, R^{12A} is unsubstituted cyclopropyl. In embodiments, R^{12A} is unsubstituted cyclobutyl. In embodiments, R^{12A} is unsubstituted cyclopentyl. In embodiments, R^{12A} is unsubstituted cyclohexyl.

In embodiments, R^{12B} is hydrogen. In embodiments, R^{12B} is unsubstituted C₁-C₆ alkyl. In embodiments, R^{12B} is unsubstituted C₃-C₈ cycloalkyl. In embodiments, R^{12B} is unsubstituted methyl. In embodiments, R^{12B} is unsubstituted ethyl. In embodiments, R^{12B} is unsubstituted propyl. In embodiments, R^{12B} is unsubstituted n-propyl. In embodiments, R^{12B} is unsubstituted isopropyl. In embodiments, R^{12B} is unsubstituted butyl. In embodiments, R^{12B} is unsubstituted n-butyl. In embodiments, R^{12B} is unsubstituted isobutyl. In embodiments, R^{12B} is unsubstituted tert-butyl. In embodiments, R^{12B} is unsubstituted pentyl. In embodiments, R^{12B} is unsubstituted hexyl. In embodiments, R^{12B} is unsubstituted cyclopropyl. In embodiments, R^{12B} is unsubstituted cyclobutyl. In embodiments, R^{12B} is unsubstituted cyclopentyl. In embodiments, R^{12B} is unsubstituted cyclohexyl.

In embodiments, R^{12C} is hydrogen. In embodiments, R^{12C} is unsubstituted C₁-C₆ alkyl. In embodiments, R^{12C} is unsubstituted methyl. In embodiments, R^{12C} is unsubstituted ethyl. In embodiments, R^{12C} is unsubstituted propyl. In embodiments, R^{12C} is unsubstituted n-propyl. In embodiments, R^{12C} is unsubstituted isopropyl. In embodiments, R^{12C} is unsubstituted butyl. In embodiments, R^{12C} is unsubstituted n-butyl. In embodiments, R^{12C} is unsubstituted isobutyl. In embodiments, R^{12C} is unsubstituted tert-butyl. In embodiments, R^{12C} is unsubstituted pentyl. In embodiments, R^{12C} is unsubstituted hexyl.

In embodiments, R^{12D} is hydrogen. In embodiments, R^{12D} is unsubstituted C₁-C₆ alkyl. In embodiments, R^{12D} is unsubstituted methyl. In embodiments, R^{12D} is unsubstituted ethyl. In embodiments, R^{12D} is unsubstituted propyl. In embodiments, R^{12D} is unsubstituted n-propyl. In embodiments, R^{12D} is unsubstituted isopropyl. In embodiments, R^{12D} is unsubstituted butyl. In embodiments, R^{12D} is unsubstituted n-butyl. In embodiments, R^{12D} is unsubstituted isobutyl. In embodiments, R^{12D} is unsubstituted tert-butyl. In embodiments, R^{12D} is unsubstituted pentyl. In embodiments, R^{12D} is unsubstituted hexyl.

In embodiments, R¹² is hydrogen, —F, —OH, —CH₃, —CH₂CH₃, —NHCH₃, —N(CH₃)₂, —C(O)OH, —C(O)N(CH₃)₂, —S(O)₂CH₃, —S(O)₂NH₂, —S(O)₂NH(CH₃)₂,

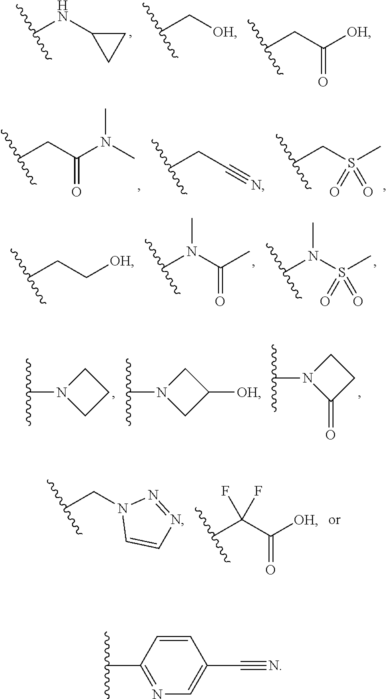

In embodiments, R¹² is hydrogen, —F, —OH, —CN, —CH₃, —CH₂CH₃, —NHCH₃, —N(CH₃)₂, —C(O)OH, —C(O)NH₂, —C(O)N(CH₃)₂, —S(O)₂CH₃, —S(O)₂NH₂, —S(O)₂NH(CH₃), —S(O)₂N(CH₃)₂, —N₃,

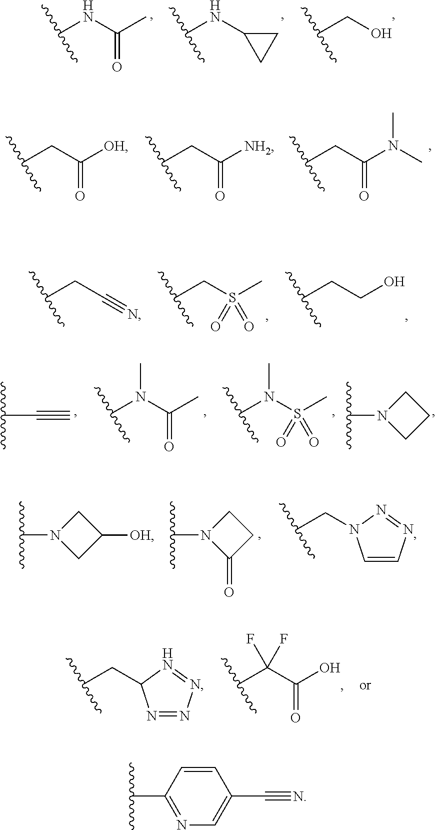

In embodiments, R¹² is hydrogen. In embodiments, R¹² is halogen. In embodiments, R¹² is —F. In embodiments, R¹² is —OH. In embodiments, R¹² is —CN. In embodiments, R¹² is —NHCH₃. In embodiments, R¹² is —N(CH₃)₂. In embodiments, R¹² is —C(O)OH. In embodiments, R¹² is —C(O)NH₂. In embodiments, R¹² is —C(O)N(CH₃)₂. In embodiments, R¹² is —S(O)₂CH₃. In embodiments, R¹² is —S(O)₂NH₂. In embodiments, R¹² is —S(O)₂NH(CH₃). In embodiments, R¹² is —S(O)₂N(CH₃)₂. In embodiments, R¹² is —N₃. In embodiments, R¹² is unsubstituted C₁-C₆ alkyl. In embodiments, R¹² is unsubstituted methyl. In embodiments, R¹² is -CD₃. In embodiments, R¹² is unsubstituted ethyl. In embodiments, R¹² is unsubstituted propyl. In embodiments, R¹² is unsubstituted n-propyl. In embodiments, R¹² is unsubstituted isopropyl. In embodiments, R¹² is unsubstituted butyl. In embodiments, R¹² is unsubstituted n-butyl. In embodiments, R¹² is unsubstituted isobutyl. In embodiments, R¹² is unsubstituted tert-butyl. In embodiments, R¹² is unsubstituted pentyl. In embodiments, R¹² is unsubstituted hexyl. In embodiments, R¹² is unsubstituted C₂-C₆ alkynyl. In embodiments, R¹² is unsubstituted ethynyl. In embodiments, R¹² is unsubstituted propynyl. In embodiments, R¹² is unsubstituted butynyl. In embodiments, R¹² is unsubstituted pentynyl. In embodiments, R¹² is unsubstituted hexynyl. In embodiments, R¹² is substituted or unsubstituted 2 to 6 membered heteroalkyl. In embodiments, R¹² is unsubstituted methoxy. In embodiments, R¹² is unsubstituted ethoxy. In embodiments, R¹² is unsubstituted propoxy. In embodiments, R¹² is unsubstituted n-propoxy. In embodiments, R¹² is unsubstituted isopropoxy. In embodiments, R¹² is unsubstituted butoxy. In embodiments, R¹² is substituted or unsubstituted 3 to 8 membered heterocycloalkyl. In embodiments, R¹² is substituted or unsubstituted azetidinyl. In embodiments, R¹² is substituted or unsubstituted 5 to 6 membered heteroaryl. In embodiments, R¹² is substituted or unsubstituted triazolyl. In embodiments, R¹² is substituted or unsubstituted pyridyl. In embodiments, R¹² is

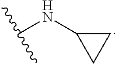 [SMILES: *NC1CC1]

In embodiments, R¹² is

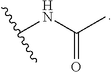 [SMILES: *NC(C)=O]

In embodiments, R¹² is

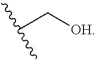 [SMILES: *CO]

In embodiments, R¹² is

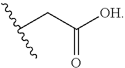 [SMILES: *CC(=O)O]

In embodiments, R¹² is

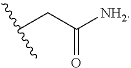 [SMILES: *CC(N)=O]

In embodiments, R¹² is

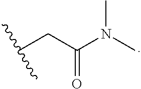 [SMILES: *CC(=O)N(C)C]

In embodiments, R¹² is

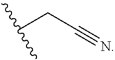 [SMILES: *CC#N]

In embodiments, R¹² is

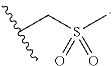 [SMILES: *CS(C)(=O)=O]

In embodiments, R¹² is

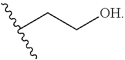 [SMILES: *CCO]

In embodiments, R¹² is

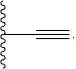 [SMILES: *C#C]

In embodiments, R¹² is

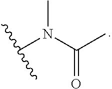 [SMILES: *N(C)C(C)=O]

In embodiments, R¹² is

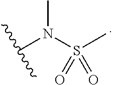 [SMILES: *N(C)S(C)(=O)=O]

In embodiments, R¹² is

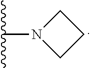 [SMILES: *N1CCC1]

In embodiments, R¹² is

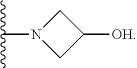 [SMILES: *N1CC(O)C1]

In embodiments, R¹² is

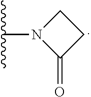 [SMILES: *N1CCC1=O]

In embodiments, R¹² is

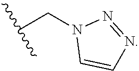 [SMILES: *Cn1ccnn1]

In embodiments, R¹² is

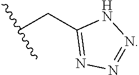 [SMILES: *Cc1nnn[nH]1]

In embodiments, R¹² is

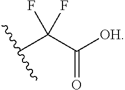 [SMILES: *C(F)(F)C(=O)O]

In embodiments, R¹² is

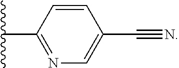 [SMILES: *c1ccc(C#N)cn1]

In embodiments, a substituted ring formed when R¹¹ and R¹² substituents are joined (e.g., substituted cycloalkyl and/or substituted heterocycloalkyl) is substituted with at least one substituent group, size-limited substituent group, or lower substituent group; wherein if the substituted ring formed when R¹¹ and R¹² substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R¹¹ and R¹² substituents are joined is substituted, it is substituted with at least one substituent group. In embodiments, when the substituted ring formed when R¹¹ and R¹² substituents are joined is substituted, it is substituted with at least one size-limited substituent group. In embodiments, when the substituted ring formed when R¹¹ and R¹² substituents are joined is substituted, it is substituted with at least one lower substituent group.

In embodiments, R¹¹ and R¹² substituents are joined to form a substituted or unsubstituted C₃-C₈ cycloalkyl or substituted or unsubstituted 3 to 8 membered heterocycloalkyl. In embodiments, R¹¹ and R¹² substituents are joined to form a substituted or unsubstituted C₃-C₈ cycloalkyl. In embodiments, R¹¹ and R¹² substituents are joined to form a substituted or unsubstituted 3 to 8 membered heterocycloalkyl. In embodiments, R¹¹ and R¹² substituents are joined to form a substituted azetidinyl. In embodiments, R¹¹ and R¹² substituents are joined to form an unsubstituted dioxolanyl. In embodiments, R¹¹ and R¹² substituents are joined to form

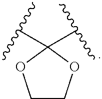 [SMILES: *C1(*)OCCO1]

In embodiments, R¹¹ and R¹² substituents are joined to form

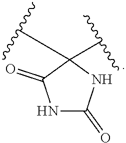 [SMILES: *C1(*)NC(=O)NC1=O]

In embodiments, R¹¹ and R¹² substituents are joined to form an oxo.

In embodiments,

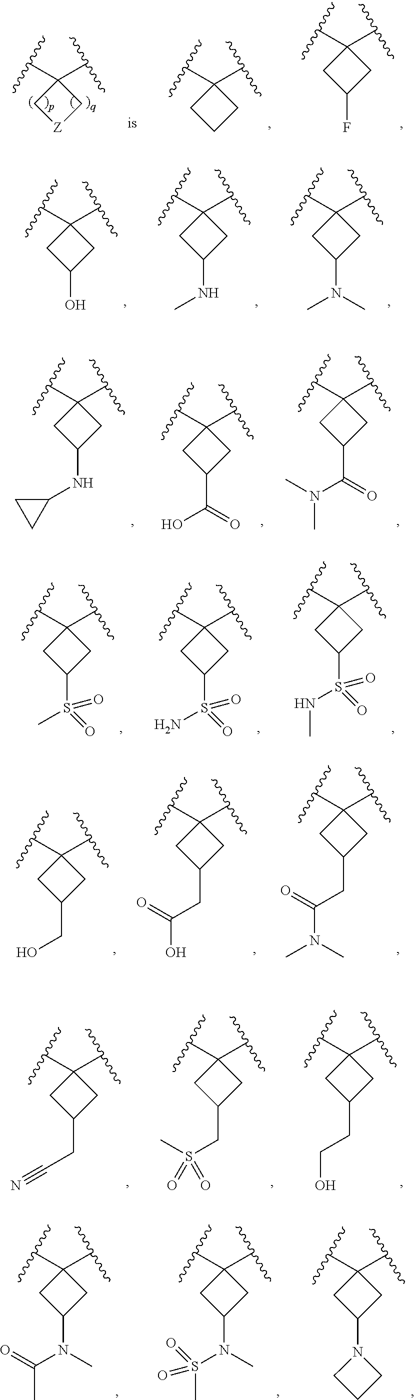

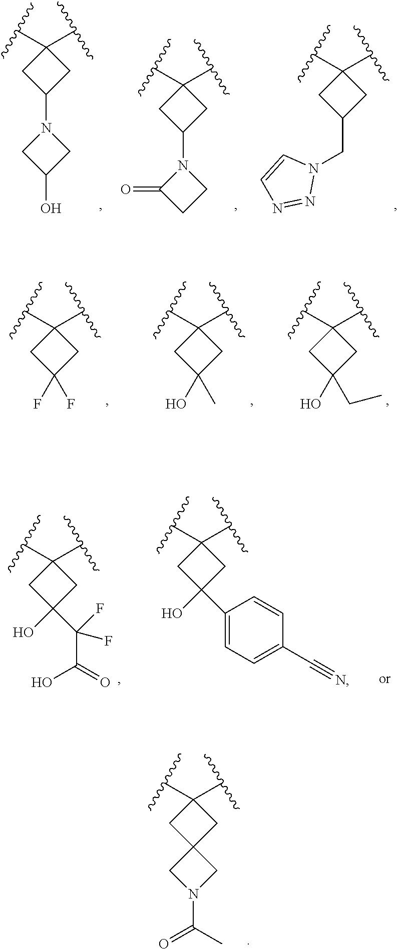

In embodiments,

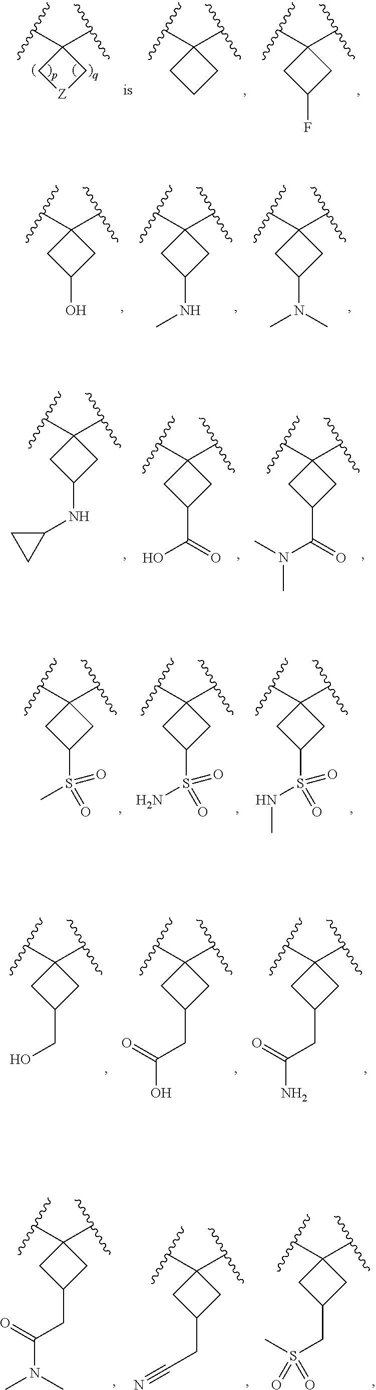

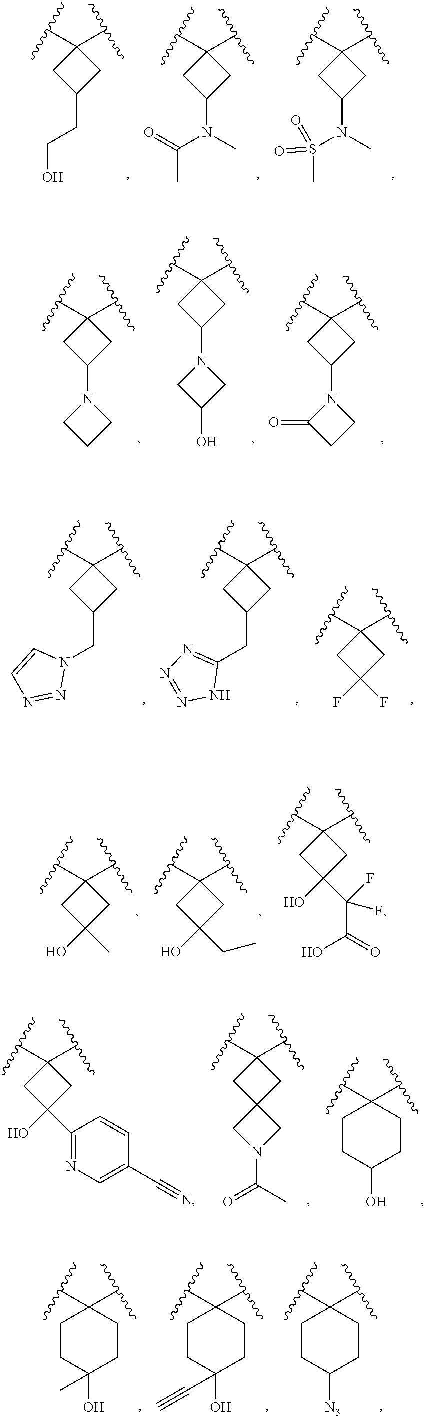

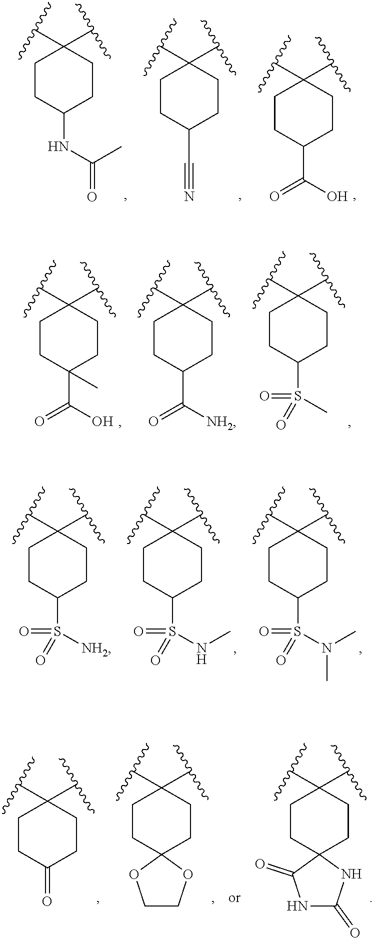

In embodiments,

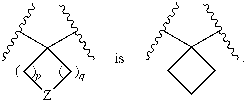

In embodiments,

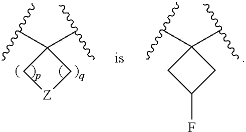

In embodiments,

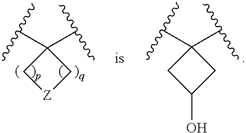

In embodiments,

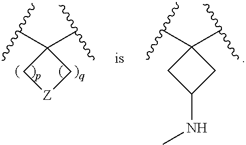

In embodiments,

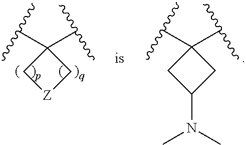

In embodiments,

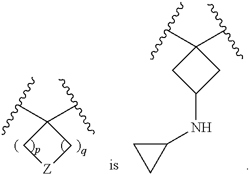

In embodiments,

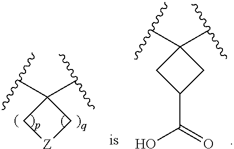

In embodiments,

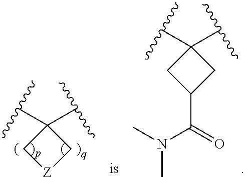

In embodiments,

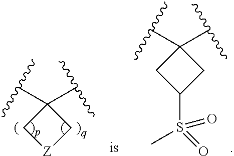

In embodiments,

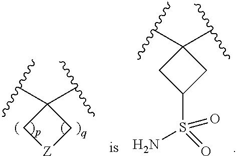

In embodiments,

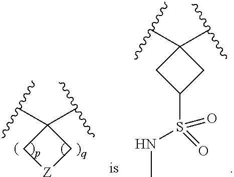

In embodiments,

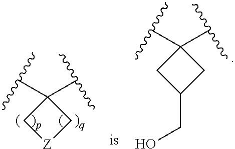

In embodiments,

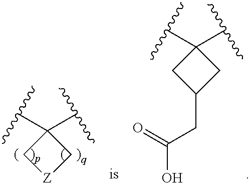

In embodiments,

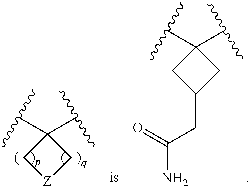

In embodiments,

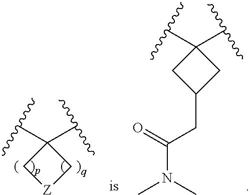

In embodiments,

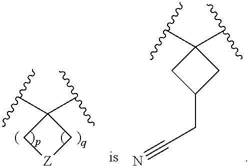

In embodiments,

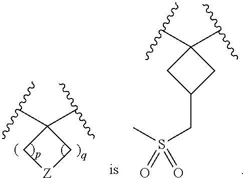

In embodiments,

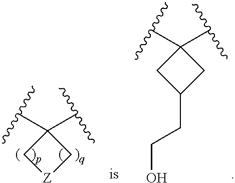

In embodiments,

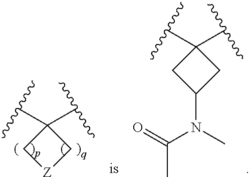

In embodiments,

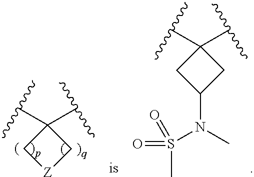

In embodiments,

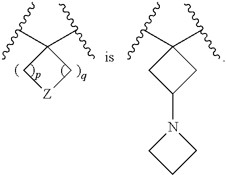

In embodiments,

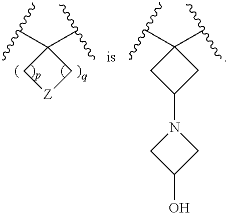

In embodiments,

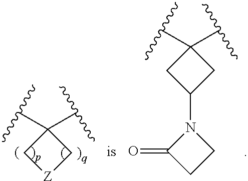

In embodiments,

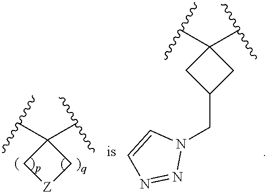

In embodiments,

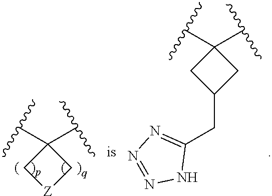

In embodiments,

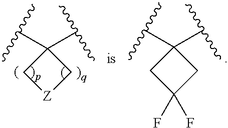

In embodiments,

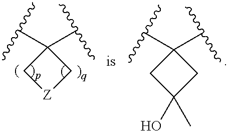

In embodiments,

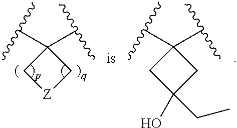

In embodiments,

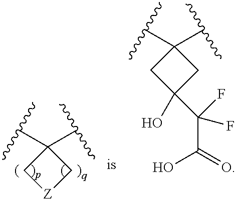

In embodiments,

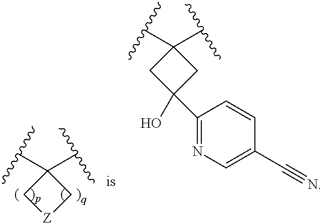

In embodiments,

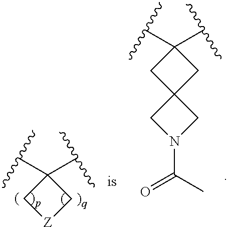

In embodiments,

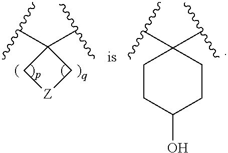

In embodiments,

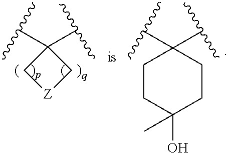

In embodiments,

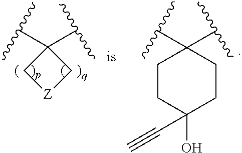

In embodiments,

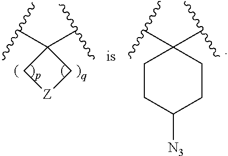

In embodiments,

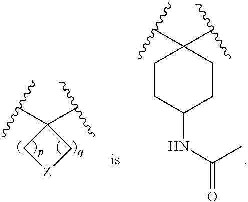

In embodiments,

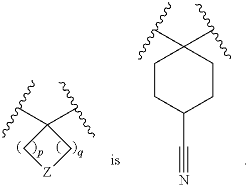

In embodiments,

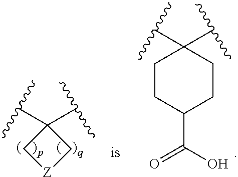

In embodiments,

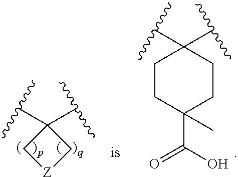

In embodiments,

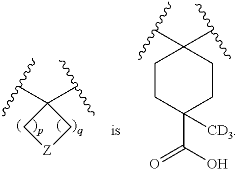

In embodiments,

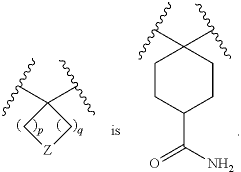

In embodiments,

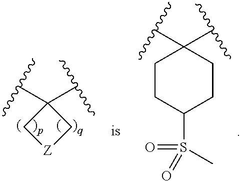

In embodiments,

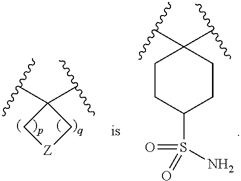

In embodiments,

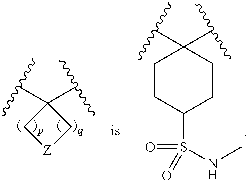

In embodiments,

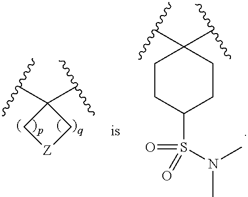

In embodiments,

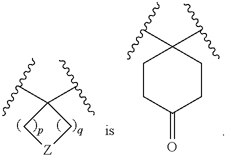

In embodiments,

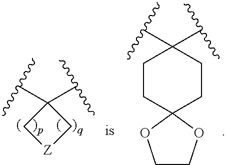

In embodiments,

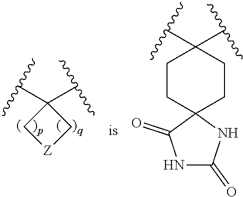

In embodiments, a substituted R¹³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹³ is substituted with a plurality of groups selected from substituent groups, size-limited substituent groups, and lower substituent groups; each substituent group, size-limited substituent group, and/or lower substituent group may optionally be different. In embodiments, when R¹³ is substituted, it is substituted with at least one substituent group. In embodiments, when R¹³ is substituted, it is substituted with at least one size-limited substituent group. In embodiments, when R¹³ is substituted, it is substituted with at least one lower substituent group.

In embodiments, a substituted R^{13B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{13B} is substituted with a plurality of groups selected from substituent groups, size-limited substituent groups, and lower substituent groups; each substituent group, size-limited substituent group, and/or lower substituent group may optionally be different. In embodiments, when R^{13B} is substituted, it is substituted with at least one substituent group. In embodiments, when R^{13B} is substituted, it is substituted with at least one size-limited substituent group. In embodiments, when R^{13B} is substituted, it is substituted with at least one lower substituent group.

In embodiments, a substituted R^{13B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{13B} is substituted with a plurality of groups selected from substituent groups, size-limited substituent groups, and lower substituent groups; each substituent group, size-limited substituent group, and/or lower substituent group may optionally be different. In embodiments, when R^{13B} is substituted, it is substituted with at least one substituent group. In embodiments, when R^{13B} is substituted, it is substituted with at least one size-limited substituent group. In embodiments, when R^{13B} is substituted, it is substituted with at least one lower substituent group.

In embodiments, a substituted ring formed when R^{13B} and R^{13B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{13A} and R^{13B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{13B} and R^{13B} substituents bonded to the same nitrogen atom are joined is substituted, it is substituted with at least one substituent group. In embodiments, when the substituted ring formed when R^{13B} and R^{13B} substituents bonded to the same nitrogen atom are joined is substituted, it is substituted with at least one size-limited substituent group. In embodiments, when the substituted ring formed when R^{13B} and R^{13B} substituents bonded to the same nitrogen atom are joined is substituted, it is substituted with at least one lower substituent group.

In embodiments, a substituted R^{13C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{13C} is substituted with a plurality of groups selected from substituent groups, size-limited substituent groups, and lower substituent groups; each substituent group, size-limited substituent group, and/or lower substituent group may optionally be different. In embodiments, when R^{13C} is substituted, it is substituted with at least one substituent group. In embodiments, when R^{13C} is substituted, it is substituted with at least one size-limited substituent group. In embodiments, when R^{13C} is substituted, it is substituted with at least one lower substituent group.

In embodiments, a substituted R^{13D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{13D} is substituted with a plurality of groups selected from substituent groups, size-limited substituent groups, and lower substituent groups; each substituent group, size-limited substituent group, and/or lower substituent group may optionally be different. In embodiments, when R^{13D} is substituted, it is substituted with at least one substituent group. In embodiments, when R^{13D} is substituted, it is substituted with at least one size-limited substituent group. In embodiments, when R^{13D} is substituted, it is substituted with at least one lower substituent group.

In embodiments, R¹³ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —C(O)H, —C(O)OH, —CONH₂, —OH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹³ is hydrogen, unsubstituted C₁-C₆ alkyl, or unsubstituted 2 to 6 membered heteroalkyl. In embodiments, R¹³ is hydrogen. In embodiments, R¹³ is unsubstituted C₁-C₆ alkyl. In embodiments, R¹³ is unsubstituted methyl. In embodiments, R¹³ is unsubstituted ethyl. In embodiments, R¹³ is unsubstituted propyl. In embodiments, R¹³ is unsubstituted n-propyl. In embodiments, R¹³ is unsubstituted isopropyl. In embodiments, R¹³ is unsubstituted butyl. In embodiments, R¹³ is unsubstituted n-butyl. In embodiments, R¹³ is unsubstituted isobutyl. In embodiments, R¹³ is unsubstituted tert-butyl. In embodiments, R¹³ is unsubstituted pentyl. In embodiments, R¹³ is unsubstituted hexyl. In embodiments, R¹³ is unsubstituted 2 to 6 membered heteroalkyl. In embodiments, R¹³ is —CH₂OCH₂CH₃.

In embodiments,

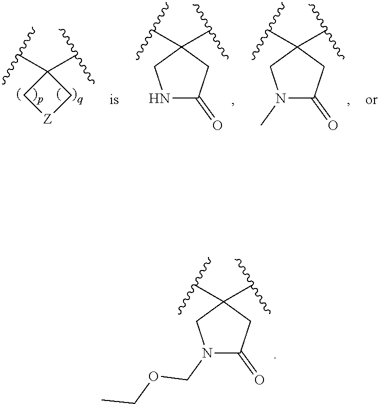

In embodiments,

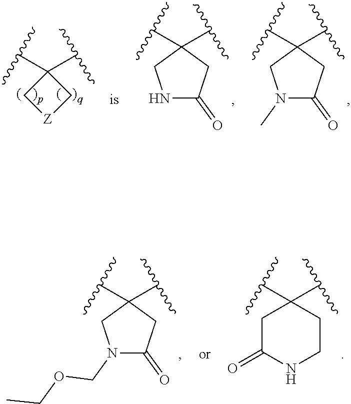

In embodiments,

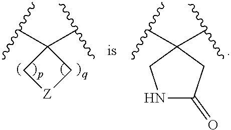

In embodiments,

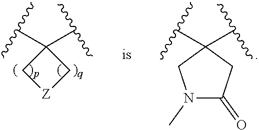

In embodiments,

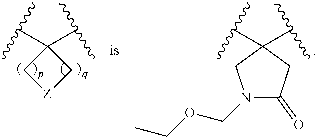

In embodiments,

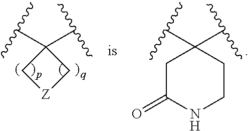

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW2} and R^{WW.3} correspond to R¹, R^{1.1}, R^{1.2}, and R^{1.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.1} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{2B}, R^{2B.1}, R^{2B.2}, and R^{2B.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A.1}, R^{2A.2}, and R^{2A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2}, and R^{2A.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{2D}, R^{2D.1}, R^{2D.2}, and R^{2D.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R³, R^{3.1}, R^{3.2}, and R^{3.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{3B}, R^{3B.1}, R^{3B.2}, and R^{3B.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A.1}, R^{3A.2}, and R^{3A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3A.1}, R^{3A.2}, and R^{3A.3}, respectively.

In embodiments, when R^{3A} and R^{3B} substituents bonded to the same nitrogen atom are optionally joined to form a moiety that is substituted (e.g., a substituted heterocycloalkyl or substituted heteroaryl), the moiety is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B.1}, R^{3B.2}, and R^{3B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3B.1}, R^{3B.2}, and R^{3B.3}, respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{3C}, R^{3C.1}, R^{3C.2}, and R^{3C.3}, respectively.

In embodiments, when R^{3D} is substituted, R^{3D} is substituted with one or more first substituent groups denoted by R^{3D.1} as explained in the definitions section above in the description of “first substituent group(s)”. In embodiments, when an R^{3D.1} substituent group is substituted, the R^{3D.1} substituent group is substituted with one or more second substituent groups denoted by R^{3D.2} as explained in the definitions section above in the description of “first substituent group(s)”. In embodiments, when an R^{3D.2} substituent group is substituted, the R^{3D.2} substituent group is substituted with one or more third substituent groups denoted by R^{3D.3} as explained in the definitions section above in the description of “first substituent group(s)”. In the above embodiments, R^{3D}, R^{3D.1}, R^{3D.2}, and R^{3D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{3D}, R^{3D.1}, R^{3D.2}, and R^{3D.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R⁴, R^{4.1}, R^{4.2}, and R^{4.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW2} and R^{WW.3} correspond to R^{4B}, R^{4B.1}, R^{4B.2}, and R^{4B.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A.1}, R^{4A.2}, and R^{4A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4A.1}, R^{4A.2}, and R^{4A.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B.1}, R^{4B.2}, and R^{4B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4B.1}, R^{4B.2}, and R^{4B.3}, respectively.

In embodiments, when R^{4C} is substituted, R^{4C} is substituted with one or more first substituent groups denoted by R^{4C.1} as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{4C}, R^{4C.1}, R^{4C.2}, and R^{4C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{4C}, R^{4C.1}, R^{4C.2}, and R^{4C.3}, respectively.

In embodiments, when R^{4D} is substituted, R^{4D} is substituted with one or more first substituent groups denoted by R^{4D.1} as explained in the definitions section above in the description of “first substituent group(s)”. In embodiments, when an R^{4D.1} substituent group is substituted, the R^{4D.1} substituent group is substituted with one or more second substituent groups denoted by R^{4D.2} as explained in the definitions section above in the description of “first substituent group(s)”. In embodiments, when an R^{4D.2} substituent group is substituted, the R^{4D.2} substituent group is substituted with one or more third substituent groups denoted by R^{4D.3} as explained in the definitions section above in the description of “first substituent group(s)”. In the above embodiments, R^{4D}, R^{4D.1}, R^{4D.2}, and R^{4D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{4D}, R^{4D.1}, R^{4D.2}, and R^{4D.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R⁵, R^{5.1}, R^{5.2}, and R^{5.3}, respectively.

In embodiments, when R^{5A} is substituted, R^{5A} is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A}, R^{5A.1}, R^{5A.2}, and R^{5A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{5A}, R^{5A.1}, R^{5A.2}, and R^{5A.3}, respectively.

In embodiments, when R^{5B} is substituted, R^{5B} is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B}, R^{5B.1}, R^{5B.2}, and R^{5B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{5B}, R^{5B.1}, R^{5B.2}, and R^{5B.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A.1}, R^{5A.2}, and R^{5A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5A.1}, R^{5A.2}, and R^{5A.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B.1}, R^{5B.2}, and R^{5B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5B.1}, R^{5B.2}, and R^{5B.3}, respectively.

In embodiments, when R^{5C} is substituted, R^{5C} is substituted with one or more first substituent groups denoted by R^{5C.1} as explained in the definitions section above in the description of “first substituent group(s)”. In embodiments, when an R^{5C.1} substituent group is substituted, the R^{5C.1} substituent group is substituted with one or more second substituent groups denoted by R^{5C.2} as explained in the definitions section above in the description of “first substituent group(s)”. In embodiments, when an R^{5C.2} substituent group is substituted, the R^{5C.2} substituent group is substituted with one or more third substituent groups denoted by R^{5C.3} as explained in the definitions section above in the description of “first substituent group(s)”. In the above embodiments, R^{5C}, R^{5C.1}, R^{5C.2}, and R^{5C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{5C}, R^{5C.1}, R^{5C.2}, and R^{5C.3}, respectively.

In embodiments, when R^{5D} is substituted, R^{5D} is substituted with one or more first substituent groups denoted by R^{5D.1} as explained in the definitions section above in the description of “first substituent group(s)”. In embodiments, when an R^{5D.1} substituent group is substituted, the R^{5D.1} substituent group is substituted with one or more second substituent groups denoted by R^{5D.2} as explained in the definitions section above in the description of “first substituent group(s)”. In embodiments, when an R^{5D.2} substituent group is substituted, the R^{5D.2} substituent group is substituted with one or more third substituent groups denoted by R^{5D.3} as explained in the definitions section above in the description of “first substituent group(s)”. In the above embodiments, R^{5D}, R^{5D.1}, R^{5D.2}, and R^{5D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{5D}, R^{5D.1}, R^{5D.2}, and R^{5D.3}, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R⁶, R^{6.1}, R^{6.2}, and R^{6.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R⁶, R^{6.1}, R^{6.2}, and R^{6.3}, respectively.

In embodiments, when R^{6A} is substituted, R^{6A} is substituted with one or more first substituent groups denoted by R^{6A.1} as explained in the definitions section above in the description of “first substituent group(s)”. In embodiments, when an R^{6A.1} substituent group is substituted, the R^{6A.1} substituent group is substituted with one or more second substituent groups denoted by R^{6A.2} as explained in the definitions section above in the description of “first substituent group(s)”. In embodiments, when an R^{6A.2} substituent group is substituted, the R^{6A.2} substituent group is substituted with one or more third substituent groups denoted by R^{6A.3} as explained in the definitions section above in the description of “first substituent group(s)”. In the above embodiments, R^{6A}, R^{6A.1}, R^{6A.2}, and R^{6A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{6A}, R^{6A.1}, R^{6A.2}, and R^{6A.3}, respectively.

In embodiments, when R^{6B} is substituted, R^{6B} is substituted with one or more first substituent groups denoted by R^{6B.1} as explained in the definitions section above in the description of “first substituent group(s)”. In embodiments, when an R^{6B.1} substituent group is substituted, the R^{6B.1} substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B}, R^{6B.1}, R^{6B.2}, and R^{6B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{6B}, R^{6B.1}, R^{6B.2}, and R^{6B.3}, respectively.

In embodiments, when R^{6A} and R^{6B} substituents bonded to the same nitrogen atom are optionally joined to form a moiety that is substituted (e.g., a substituted heterocycloalkyl or substituted heteroaryl), the moiety is substituted with one or more first substituent groups denoted by R^{6A.1} as explained in the definitions section above in the description of “first substituent group(s)”. In embodiments, when an R^{6A.1} substituent group is substituted, the R^{6A.1} substituent group is substituted with one or more second substituent groups denoted by R^{6A.2} as explained in the definitions section above in the description of “first substituent group(s)”. In embodiments, when an R^{6A.2} substituent group is substituted, the R^{6A.2} substituent group is substituted with one or more third substituent groups denoted by R^{6A.3} as explained in the definitions section above in the description of “first substituent group(s)”. In the above embodiments, R^{6A.1}, R^{6A.2}, and R^{6A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{6A.1}, R^{6A.2}, and R^{6A.3}, respectively.

In embodiments, when R^{6A} and R^{6B} substituents bonded to the same nitrogen atom are optionally joined to form a moiety that is substituted (e.g., a substituted heterocycloalkyl or substituted heteroaryl), the moiety is substituted with one or more first substituent groups denoted by R^{6B.1} as explained in the definitions section above in the description of “first substituent group(s)”. In embodiments, when an R^{6B.1} substituent group is substituted, the R^{6B.1} substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B.1}, R^{6B.2}, and R^{6B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{6B.1}, R^{6B.2}, and R^{6B.3}, respectively.

In embodiments, when R^{6C} is substituted, R^{6C} is substituted with one or more first substituent groups denoted by R^{6C.1} as explained in the definitions section above in the description of “first substituent group(s)”. In embodiments, when an R^{6C.1} substituent group is substituted, the R^{6C.1} substituent group is substituted with one or more second substituent groups denoted by R^{6C.2} as explained in the definitions section above in the description of “first substituent group(s)”. In embodiments, when an R^{6C.2} substituent group is substituted, the R^{6C.2} substituent group is substituted with one or more third substituent groups denoted by R^{6C.3} as explained in the definitions section above in the description of “first substituent group(s)”. In the above embodiments, R^{6C}, R^{6C.1}, R^{6C.2}, and R^{6C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{6C}, R^{6C.1}, R^{6C.2}, and R^{6C.3}, respectively.

In embodiments, when R^{6D} is substituted, R^{6D} is substituted with one or more first substituent groups denoted by R^{6D.1} as explained in the definitions section above in the description of “first substituent group(s)”. In embodiments, when an R^{6D.1} substituent group is substituted, the R^{6D.1} substituent group is substituted with one or more second substituent groups denoted by R^{6D.2} as explained in the definitions section above in the description of “first substituent group(s)”. In embodiments, when an R^{6D.2} substituent group is substituted, the R^{6D.2} substituent group is substituted with one or more third substituent groups denoted by R^{6D.3} as explained in the definitions section above in the description of “first substituent group(s)”. In the above embodiments, R^{6D}, R^{6D.1}, R^{6D.2}, and R^{6D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{6D}, R^{6D.1}, R^{6D.2}, and R^{6D.3}, respectively.

In embodiments, when R⁷ is substituted, R⁷ is substituted with one or more first substituent groups denoted by R^{7.1} as explained in the definitions section above in the description of “first substituent group(s)”. In embodiments, when an R^{7.1} substituent group is substituted, the R^{7.1} substituent group is substituted with one or more second substituent groups denoted by R^{7.2} as explained in the definitions section above in the description of “first substituent group(s)”. In embodiments, when an R^{7.2} substituent group is substituted, the R^{7.2} substituent group is substituted with one or more third substituent groups denoted by R^{7.3} as explained in the definitions section above in the description of “first substituent group(s)”. In the above embodiments, R⁷, R^{7.1}, R^{7.2}, and R^{7.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R⁷, R^{7.1}, R^{7.2}, and R^{7.3}, respectively.

In embodiments, when R^{7A} is substituted, R^{7A} is substituted with one or more first substituent groups denoted by R^{7A.1} as explained in the definitions section above in the description of “first substituent group(s)”. In embodiments, when an R^{7A.1} substituent group is substituted, the R^{7A.1} substituent group is substituted with one or more second substituent groups denoted by R^{7A.2} as explained in the definitions section above in the description of “first substituent group(s)”. In embodiments, when an R^{7A.2} substituent group is substituted, the R^{7A.2} substituent group is substituted with one or more third substituent groups denoted by R^{7A.3} as explained in the definitions section above in the description of “first substituent group(s)”. In the above embodiments, R^{7A}, R^{7A.1}, R^{7A.2}, and R^{7A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{7A}, R^{7A.1}, R^{7A.2}, and R^{7A.3}, respectively.

In embodiments, when R^{7B} is substituted, R^{7B} is substituted with one or more first substituent groups denoted by R^{7B.1} as explained in the definitions section above in the description of “first substituent group(s)”. In embodiments, when an R^{7B.1} substituent group is substituted, the R^{7B.1} substituent group is substituted with one or more second substituent groups denoted by R^{7B.2} as explained in the definitions section above in the description of “first substituent group(s)”. In embodiments, when an R^{7B.2} substituent group is substituted, the R^{7B.2} substituent group is substituted with one or more third substituent groups denoted by R^{7B.3} as explained in the definitions section above in the description of “first substituent group(s)”. In the above embodiments, R^{7B}, R^{7B.1}, R^{7B.2}, and R^{7B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{7B}, R^{7B.1}, R^{7B.2}, and R^{7B.3}, respectively.

In embodiments, when R^{7A} and R^{7B} substituents bonded to the same nitrogen atom are optionally joined to form a moiety that is substituted (e.g., a substituted heterocycloalkyl or substituted heteroaryl), the moiety is substituted with one or more first substituent groups denoted by R^{7A.1} as explained in the definitions section above in the description of “first substituent group(s)”. In embodiments, when an R^{7A.1} substituent group is substituted, the R^{7A.1} substituent group is substituted with one or more second substituent groups denoted by R^{7A.2} as explained in the definitions section above in the description of “first substituent group(s)”. In embodiments, when an R^{7A.2} substituent group is substituted, the R^{7A.2} substituent group is substituted with one or more third substituent groups denoted by R^{7A.3} as explained in the definitions section above in the description of “first substituent group(s)”. In the above embodiments, R^{7A.1}, R^{7A.2}, and R^{7A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{7A.1}, R^{7A.2}, and R^{7A.3}, respectively.

In embodiments, when R^{7A} and R^{7B} substituents bonded to the same nitrogen atom are optionally joined to form a moiety that is substituted (e.g., a substituted heterocycloalkyl or substituted heteroaryl), the moiety is substituted with one or more first substituent groups denoted by R^{7B.1} as explained in the definitions section above in the description of “first substituent group(s)”. In embodiments, when an R^{7B.1} substituent group is substituted, the R^{7B.1} substituent group is substituted with one or more second substituent groups denoted by R^{7B.2} as explained in the definitions section above in the description of “first substituent group(s)”. In embodiments, when an R^{7B.2} substituent group is substituted, the R^{7B.2} substituent group is substituted with one or more third substituent groups denoted by R^{7B.3} as explained in the definitions section above in the description of “first substituent group(s)”. In the above embodiments, R^{7B.1}, R^{7B.2}, and R^{7B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{7B.1}, R^{7B.2}, and R^{7B.3}, respectively.

In embodiments, when R^{7C} is substituted, R^{7C} is substituted with one or more first substituent groups denoted by R^{7C.1} as explained in the definitions section above in the description of “first substituent group(s)”. In embodiments, when an R^{7C.1} substituent group is substituted, the R^{7C.1} substituent group is substituted with one or more second substituent groups denoted by R^{7C.2} as explained in the definitions section above in the description of “first substituent group(s)”. In embodiments, when an R^{7C.2} substituent group is substituted, the R^{7C.2} substituent group is substituted with one or more third substituent groups denoted by R^{7C.3} as explained in the definitions section above in the description of “first substituent group(s)”. In the above embodiments, R^{7C}, R^{7C.1}, R^{7C.2}, and R^{7C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{7C}, R^{7C.1}, R^{7C.2}, and R^{7C.3}, respectively.

In embodiments, when R^{7D} is substituted, R^{7D} is substituted with one or more first substituent groups denoted by R^{7D.1} as explained in the definitions section above in the description of “first substituent group(s)”. In embodiments, when an R^{7D.1} substituent group is substituted, the R^{7D.1} substituent group is substituted with one or more second substituent groups denoted by R^{7D.2} as explained in the definitions section above in the description of “first substituent group(s)”. In embodiments, when an R^{7D.2} substituent group is substituted, the R^{7D.2} substituent group is substituted with one or more third substituent groups denoted by R^{7D.3} as explained in the definitions section above in the description of “first substituent group(s)”. In the above embodiments, R^{7D}, R^{7D.1}, R^{7D.2}, and R^{7D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{7D}, R^{7D.1}, R^{7D.2}, and R^{7D.3}, respectively.

In embodiments, when R⁸ is substituted, R⁸ is substituted with one or more first substituent groups denoted by R^{8.1} as explained in the definitions section above in the description of “first substituent group(s)”. In embodiments, when an R^{8.1} substituent group is substituted, the R^{8.1} substituent group is substituted with one or more second substituent groups denoted by R^{8.2} as explained in the definitions section above in the description of “first substituent group(s)”. In embodiments, when an R^{8.2} substituent group is substituted, the R^{8.2} substituent group is substituted with one or more third substituent groups denoted by R^{8.3} as explained in the definitions section above in the description of “first substituent group(s)”. In the above embodiments, R⁸, R^{8.1}, R^{8.2}, and R^{8.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R⁸, R^{8.1}, R^{8.2}, and R^{8.3}, respectively.

In embodiments, when R^{8A} is substituted, R^{8A} is substituted with one or more first substituent groups denoted by R^{8A.1} as explained in the definitions section above in the description of “first substituent group(s)”. In embodiments, when an R^{8A.1} substituent group is substituted, the R^{8A.1} substituent group is substituted with one or more second substituent groups denoted by R^{8A.2} as explained in the definitions section above in the description of “first substituent group(s)”. In embodiments, when an R^{8A.2} substituent group is substituted, the R^{8A.2} substituent group is substituted with one or more third substituent groups denoted by R^{8A.3} as explained in the definitions section above in the description of “first substituent group(s)”. In the above embodiments, R^{8A}, R^{8A.1}, R^{8A.2}, and R^{8A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{8A}, R^{8A.1}, R^{8A.2}, and R^{8A.3}, respectively.

In embodiments, when R^{8B} is substituted, R^{8B} is substituted with one or more first substituent groups denoted by R^{8B.1} as explained in the definitions section above in the description of “first substituent group(s)”. In embodiments, when an R^{8B.1} substituent group is substituted, the R^{8B.1} substituent group is substituted with one or more second substituent groups denoted by R^{8B.2} as explained in the definitions section above in the description of “first substituent group(s)”. In embodiments, when an R^{8B.2} substituent group is substituted, the R^{8B.2} substituent group is substituted with one or more third substituent groups denoted by R^{8B.3} as explained in the definitions section above in the description of “first substituent group(s)”. In the above embodiments, R^{8B}, R^{8B.1}, R^{8B.2}, and R^{8B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{8B}, R^{8B.1}, R^{8B.2}, and R^{8B.3}, respectively.

In embodiments, when R^{8A} and R^{8B} substituents bonded to the same nitrogen atom are optionally joined to form a moiety that is substituted (e.g., a substituted heterocycloalkyl or substituted heteroaryl), the moiety is substituted with one or more first substituent groups denoted by R^{8A.1} as explained in the definitions section above in the description of “first substituent group(s)”. In embodiments, when an R^{8A.1} substituent group is substituted, the R^{8A.1} substituent group is substituted with one or more second substituent groups denoted by R^{8A.2} as explained in the definitions section above in the description of “first substituent group(s)”. In embodiments, when an R^{8A.2} substituent group is substituted, the R^{8A.2} substituent group is substituted with one or more third substituent groups denoted by R^{8A.3} as explained in the definitions section above in the description of “first substituent group(s)”. In the above embodiments, R^{8A.1}, R^{8A.2}, and R^{8A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{8A.1}, R^{8A.2}, and R^{8A.3}, respectively.

In embodiments, when R^{8A} and R^{8B} substituents bonded to the same nitrogen atom are optionally joined to form a moiety that is substituted (e.g., a substituted heterocycloalkyl or substituted heteroaryl), the moiety is substituted with one or more first substituent groups denoted by R^{8B.1} as explained in the definitions section above in the description of “first substituent group(s)”. In embodiments, when an R^{8B.1} substituent group is substituted, the R^{8B.1} substituent group is substituted with one or more second substituent groups denoted by R^{8B.2} as explained in the definitions section above in the description of “first substituent group(s)”. In embodiments, when an R^{8B.2} substituent group is substituted, the R^{8B.2} substituent group is substituted with one or more third substituent groups denoted by R^{8B.3} as explained in the definitions section above in the description of “first substituent group(s)”. In the above embodiments, R^{8B.1}, R^{8B.2}, and R^{8B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{8B.1}, R^{8B.2}, and R^{8B.3}, respectively.

In embodiments, when R^{8C} is substituted, R^{8C} is substituted with one or more first substituent groups denoted by R^{8C.1} as explained in the definitions section above in the description of “first substituent group(s)”. In embodiments, when an R^{8C.1} substituent group is substituted, the R^{8C.1} substituent group is substituted with one or more second substituent groups denoted by R^{8C.2} as explained in the definitions section above in the description of “first substituent group(s)”. In embodiments, when an R^{8C.2} substituent group is substituted, the R^{8C.2} substituent group is substituted with one or more third substituent groups denoted by R^{8C.3} as explained in the definitions section above in the description of “first substituent group(s)”. In the above embodiments, R^{8C}, R^{8C.1}, R^{8C.2}, and R^{8C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{8C}, R^{8C.1}, R^{8C.2}, and R^{8C.3}, respectively.

In embodiments, when R^{8D} is substituted, R^{8D} is substituted with one or more first substituent groups denoted by R^{8D.1} as explained in the definitions section above in the description of “first substituent group(s)”. In embodiments, when an R^{8D.1} substituent group is substituted, the R^{8D.1} substituent group is substituted with one or more second substituent groups denoted by R^{8D.2} as explained in the definitions section above in the description of “first substituent group(s)”. In embodiments, when an R^{8D.2} substituent group is substituted, the R^{8D.2} substituent group is substituted with one or more third substituent groups denoted by R^{8D.3} as explained in the definitions section above in the description of “first substituent group(s)”. In the above embodiments, R^{8D}, R^{8D.1}, R^{8D.2}, and R^{8D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{8D}, R^{8D.1}, R^{8D.2}, and R^{8D.3}, respectively.

In embodiments, when R^{8.1} is substituted, R^{8.1} is substituted with one or more first substituent groups denoted by R^{8.1.1} as explained in the definitions section above in the description of “first substituent group(s)”. In embodiments, when an R^{8.1.1} substituent group is substituted, the R^{8.1.1} substituent group is substituted with one or more second substituent groups denoted by R^{8.1.2} as explained in the definitions section above in the description of “first substituent group(s)”. In embodiments, when an R^{8.1.2} substituent group is substituted, the R^{8.1.2} substituent group is substituted with one or more third substituent groups denoted by R^{8.1.3} as explained in the definitions section above in the description of “first substituent group(s)”. In the above embodiments, R^{8.1}, R^{8.1.1}, R^{8.1.2}, and R^{8.1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R¹, R^{8.1.1}, R^{8.1.2}, and R^{8.1.3}, respectively.

In embodiments, when R^{8.2} is substituted, R^{8.2} is substituted with one or more first substituent groups denoted by R^{8.2.1} as explained in the definitions section above in the description of “first substituent group(s)”. In embodiments, when an R^{8.2.1} substituent group is substituted, the R^{8.2.1} substituent group is substituted with one or more second substituent groups denoted by R^{8.2.2} as explained in the definitions section above in the description of “first substituent group(s)”. In embodiments, when an R^{8.2.2} substituent group is substituted, the R^{8.2.2} substituent group is substituted with one or more third substituent groups denoted by R^{8.2.3} as explained in the definitions section above in the description of “first substituent group(s)”. In the above embodiments, R^{8.2}, R^{8.2.1}, R^{8.2.2}, and R^{8.2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{8.2}, R^{8.2.1}, R^{8.2.2}, and R^{8.2.3}, respectively.

In embodiments, when R^{8.3} is substituted, R^{8.3} is substituted with one or more first substituent groups denoted by R^{8.3.1} as explained in the definitions section above in the description of “first substituent group(s)”. In embodiments, when an R^{8.3.1} substituent group is substituted, the R^{8.3.1} substituent group is substituted with one or more second substituent groups denoted by R^{8.3.2} as explained in the definitions section above in the description of “first substituent group(s)”. In embodiments, when an R^{8.3.2} substituent group is substituted, the R^{8.3.2} substituent group is substituted with one or more third substituent groups denoted by R^{8.3.3} as explained in the definitions section above in the description of “first substituent group(s)”. In the above embodiments, R^{8.3}, R^{8.3.1}, R^{8.3.2}, and R^{8.3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{8.3}, R^{8.3.1}, R^{8.3.2}, and R^{8.3.3}, respectively.

In embodiments, when R^{8.2} and R^{8.3} substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{8.2.1} as explained in the definitions section above in the description of “first substituent group(s)”. In embodiments, when an R^{8.2.1} substituent group is substituted, the R^{8.2.1} substituent group is substituted with one or more second substituent groups denoted by R^{8.2.2} as explained in the definitions section above in the description of “first substituent group(s)”. In embodiments, when an R^{8.2.2} substituent group is substituted, the R^{8.2.2} substituent group is substituted with one or more third substituent groups denoted by R^{8.2.3} as explained in the definitions section above in the description of “first substituent group(s)”. In the above embodiments, R^{8.2.1}, R^{8.2.2}, and R^{8.2.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{8.2.1}, R^{8.2.2}, and R^{8.2.3}, respectively.

In embodiments, when R^{8.2} and R^{8.3} substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{8.3.1} as explained in the definitions section above in the description of “first substituent group(s)”. In embodiments, when an R^{8.3.1} substituent group is substituted, the R^{8.3.1} substituent group is substituted with one or more second substituent groups denoted by R^{8.3.2} as explained in the definitions section above in the description of “first substituent group(s)”. In embodiments, when an R^{8.3.2} substituent group is substituted, the R^{8.3.2} substituent group is substituted with one or more third substituent groups denoted by R^{8.3.3} as explained in the definitions section above in the description of “first substituent group(s)”. In the above embodiments, R^{8.3.1}, R^{8.3.2}, and R^{8.3.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{8.3.1}, R^{8.3.2}, and R^{8.3.3}, respectively.

In embodiments, when R¹⁰ is substituted, R¹⁰ is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R¹⁰, R^{10.1}, R^{10.2}, and R^{10.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R¹⁰, R^{10.1}, R^{10.2}, and R^{10.3}, respectively.

In embodiments, when R^{10A} is substituted, R^{10A} is substituted with one or more first substituent groups denoted by R^{10A.1} as explained in the definitions section above in the description of “first substituent group(s)”. In embodiments, when an R^{10A.1} substituent group is substituted, the R^{10A.1} substituent group is substituted with one or more second substituent groups denoted by R^{10A.2} as explained in the definitions section above in the description of “first substituent group(s)”. In embodiments, when an R^{10A.2} substituent group is substituted, the R^{10A.2} substituent group is substituted with one or more third substituent groups denoted by R^{10A.3} as explained in the definitions section above in the description of “first substituent group(s)”. In the above embodiments, R^{10A}, R^{10A.1}, R^{10A.2}, and R^{10A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10A}, R^{10A.1}, R^{10A.2}, and R^{10A.3} respectively.

In embodiments, when R^{10B} is substituted, R^{10B} is substituted with one or more first substituent groups denoted by R^{10B} as explained in the definitions section above in the description of “first substituent group(s)”. In embodiments, when an R^{10B.1} substituent group is substituted, the R^{10B.1} substituent group is substituted with one or more second substituent groups denoted by R^{10B.2} as explained in the definitions section above in the description of “first substituent group(s)”. In embodiments, when an R^{10B.2} substituent group is substituted, the R^{10B.2} substituent group is substituted with one or more third substituent groups denoted by R^{10B.3} as explained in the definitions section above in the description of “first substituent group(s)”. In the above embodiments, R^{10B}, R^{10B.1}, R^{10B.2}, and R^{10B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10B}, R^{10B.1}, R^{10B.2}, and R^{10B.3}, respectively.

In embodiments, when R^{10A} and R^{10B} substituents bonded to the same nitrogen atom are optionally joined to form a moiety that is substituted (e.g., a substituted heterocycloalkyl or substituted heteroaryl), the moiety is substituted with one or more first substituent groups denoted by R^{10A.1} as explained in the definitions section above in the description of “first substituent group(s)”. In embodiments, when an R^{10A.1} substituent group is substituted, the R^{10A.1} substituent group is substituted with one or more second substituent groups denoted by R^{10A.2} as explained in the definitions section above in the description of “first substituent group(s)”. In embodiments, when an R^{10A.2} substituent group is substituted, the R^{10A.2} substituent group is substituted with one or more third substituent groups denoted by R^{10A.3} as explained in the definitions section above in the description of “first substituent group(s)”. In the above embodiments, R^{10A.1}, R^{10A.2}, and R^{10A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{10A.1}, R^{10A.2}, and R^{10A.3}, respectively.

In embodiments, when R^{10A} and R^{10B} substituents bonded to the same nitrogen atom are optionally joined to form a moiety that is substituted (e.g., a substituted heterocycloalkyl or substituted heteroaryl), the moiety is substituted with one or more first substituent groups denoted by R^{10B.1} as explained in the definitions section above in the description of “first substituent group(s)”. In embodiments, when an R^{10B.1} substituent group is substituted, the R^{10B.1} substituent group is substituted with one or more second substituent groups denoted by R^{10B.2} as explained in the definitions section above in the description of “first substituent group(s)”. In embodiments, when an R^{10B.2} substituent group is substituted, the R^{10B.2} substituent group is substituted with one or more third substituent groups denoted by R^{10B.3} as explained in the definitions section above in the description of “first substituent group(s)”. In the above embodiments, R^{10B.1}, R^{10B.2}, and R^{10B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{10B.1}, R^{10B.2}, and R^{10B.3}, respectively.

In embodiments, when R^{10C} is substituted, R^{10C} is substituted with one or more first substituent groups denoted by R^{10C.1} as explained in the definitions section above in the description of “first substituent group(s)”. In embodiments, when an R^{10C.1} substituent group is substituted, the R^{10C.1} substituent group is substituted with one or more second substituent groups denoted by R^{10C.2} as explained in the definitions section above in the description of “first substituent group(s)”. In embodiments, when an R^{10C.2} substituent group is substituted, the R^{10C.2} substituent group is substituted with one or more third substituent groups denoted by R^{10C.3} as explained in the definitions section above in the description of “first substituent group(s)”. In the above embodiments, R^{10C}, R^{10C.1}, R^{10C.2}, and R^{10C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10C}, R^{10C.1}, R^{10C.2}, and R^{10C.3} respectively.

In embodiments, when R^{10D} is substituted, R^{10D} is substituted with one or more first substituent groups denoted by R^{10D} as explained in the definitions section above in the description of “first substituent group(s)”. In embodiments, when an R^{10D.1} substituent group is substituted, the R^{10D.1} substituent group is substituted with one or more second substituent groups denoted by R^{10D.2} as explained in the definitions section above in the description of “first substituent group(s)”. In embodiments, when an R^{10D.2} substituent group is substituted, the R^{10D.2} substituent group is substituted with one or more third substituent groups denoted by R^{10D.3} as explained in the definitions section above in the description of “first substituent group(s)”. In the above embodiments, R^{10D}, R^{10D.1}, R^{10D.2}, and R^{10D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10D}, R^{10D.1}, R^{10D.2}, and R^{10D.3} respectively.

In embodiments, when R¹¹ is substituted, R¹¹ is substituted with one or more first substituent groups denoted by R^{11.1} as explained in the definitions section above in the description of “first substituent group(s)”. In embodiments, when an R^{11.1} substituent group is substituted, the R^{11.1} substituent group is substituted with one or more second substituent groups denoted by R^{11.2} as explained in the definitions section above in the description of “first substituent group(s)”. In embodiments, when an R^{11.2} substituent group is substituted, the R^{11.2} substituent group is substituted with one or more third substituent groups denoted by R^{11.3} as explained in the definitions section above in the description of “first substituent group(s)”. In the above embodiments, R¹¹, R^{11.1}, R^{11.2}, and R^{11.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R¹¹, R^{11.1}, R^{11.2}, and R^{11.3}, respectively.

In embodiments, when R^{11A} is substituted, R^{11A} is substituted with one or more first substituent groups denoted by R^{11A.1} as explained in the definitions section above in the description of “first substituent group(s)”. In embodiments, when an R^{11A.1} substituent group is substituted, the R^{11A.1} substituent group is substituted with one or more second substituent groups denoted by R^{11A.2} as explained in the definitions section above in the description of “first substituent group(s)”. In embodiments, when an R^{11A.2} substituent group is substituted, the R^{11A.2} substituent group is substituted with one or more third substituent groups denoted by R^{11A.3} as explained in the definitions section above in the description of “first substituent group(s)”. In the above embodiments, R^{11A}, R^{11A.1}, R^{11A.2}, and R^{11A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11A}, R^{11A.1}, R^{11A.2}, and R^{11A.3} respectively.

In embodiments, when R^{11B} is substituted, R^{11B} is substituted with one or more first substituent groups denoted by R^{11B.1} as explained in the definitions section above in the description of “first substituent group(s)”. In embodiments, when an R^{11B.1} substituent group is substituted, the R^{11B.1} substituent group is substituted with one or more second substituent groups denoted by R^{11B.2} as explained in the definitions section above in the description of “first substituent group(s)”. In embodiments, when an R^{11B.2} substituent group is substituted, the R^{11B.2} substituent group is substituted with one or more third substituent groups denoted by R^{11B.3} as explained in the definitions section above in the description of “first substituent group(s)”. In the above embodiments, R^{11B}, R^{11B.1}, R^{11B.2}, and R^{11B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11B}, R^{11B.1}, R^{11B.2}, and R^{11B.3} respectively.

In embodiments, when R^{11A} and R^{11B} substituents bonded to the same nitrogen atom are optionally joined to form a moiety that is substituted (e.g., a substituted heterocycloalkyl or substituted heteroaryl), the moiety is substituted with one or more first substituent groups denoted by R^{11A.1} as explained in the definitions section above in the description of “first substituent group(s)”. In embodiments, when an R^{11A.1} substituent group is substituted, the R^{11A.1} substituent group is substituted with one or more second substituent groups denoted by R^{11A.2} as explained in the definitions section above in the description of “first substituent group(s)”. In embodiments, when an R^{11A.2} substituent group is substituted, the R^{11A.2} substituent group is substituted with one or more third substituent groups denoted by R^{11A.3} as explained in the definitions section above in the description of “first substituent group(s)”. In the above embodiments, R^{11A.1}, R^{11A.2}, and R^{11A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{11A.1}, R^{11A.2}, and R^{11A.3}, respectively.

In embodiments, when R^{11A} and R^{11B} substituents bonded to the same nitrogen atom are optionally joined to form a moiety that is substituted (e.g., a substituted heterocycloalkyl or substituted heteroaryl), the moiety is substituted with one or more first substituent groups denoted by R^{11B.1} as explained in the definitions section above in the description of “first substituent group(s)”. In embodiments, when an R^{11B.1} substituent group is substituted, the R^{11B.1} substituent group is substituted with one or more second substituent groups denoted by R^{11B.2} as explained in the definitions section above in the description of “first substituent group(s)”. In embodiments, when an R^{11B.2} substituent group is substituted, the R^{11B.2} substituent group is substituted with one or more third substituent groups denoted by R^{11B.3} as explained in the definitions section above in the description of “first substituent group(s)”. In the above embodiments, R^{11B.1}, R^{11B.2}, and R^{11B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{11B.1}, R^{11B.2}, and R^{11B.3}, respectively.

In embodiments, when R^{11C} is substituted, R^{11C} is substituted with one or more first substituent groups denoted by R^{11C.1} as explained in the definitions section above in the description of “first substituent group(s)”. In embodiments, when an R^{11C.1} substituent group is substituted, the R^{11C.1} substituent group is substituted with one or more second substituent groups denoted by R^{11C.2} as explained in the definitions section above in the description of “first substituent group(s)”. In embodiments, when an R^{11C.2} substituent group is substituted, the R^{11C.2} substituent group is substituted with one or more third substituent groups denoted by R^{11C.3} as explained in the definitions section above in the description of “first substituent group(s)”. In the above embodiments, R^{11C}, R^{11C.1}, R^{11C.2}, and R^{11C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11C}, R^{11C.1}, R^{11C.2}, and R^{11C.3} respectively.

In embodiments, when R^{11D} is substituted, R^{11D} is substituted with one or more first substituent groups denoted by R^{11D.1} as explained in the definitions section above in the description of “first substituent group(s)”. In embodiments, when an R^{11D.1} substituent group is substituted, the R^{11D.1} substituent group is substituted with one or more second substituent groups denoted by R^{11D.2} as explained in the definitions section above in the description of “first substituent group(s)”. In embodiments, when an R^{11D.2} substituent group is substituted, the R^{11D.2} substituent group is substituted with one or more third substituent groups denoted by R^{11D.3} as explained in the definitions section above in the description of “first substituent group(s)”. In the above embodiments, R^{11D}, R^{11D.1}, R^{11D.2}, and R^{11D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11D}, R^{11D.1}, R^{11D.2}, and R^{11D.3} respectively.

In embodiments, when R¹² is substituted, R¹² is substituted with one or more first substituent groups denoted by R^{12.1} as explained in the definitions section above in the description of “first substituent group(s)”. In embodiments, when an R^{12.1} substituent group is substituted, the R^{12.1} substituent group is substituted with one or more second substituent groups denoted by R^{12.2} as explained in the definitions section above in the description of “first substituent group(s)”. In embodiments, when an R^{12.2} substituent group is substituted, the R^{12.2} substituent group is substituted with one or more third substituent groups denoted by R^{12.3} as explained in the definitions section above in the description of “first substituent group(s)”. In the above embodiments, R¹², R^{12.1}, R^{12.2}, and R^{12.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R¹², R^{12.1}, R^{12.2}, and R^{12.3}, respectively.

In embodiments, when R^{12A} is substituted, R^{12A} is substituted with one or more first substituent groups denoted by R^{12A.1} as explained in the definitions section above in the description of “first substituent group(s)”. In embodiments, when an R^{12A.1} substituent group is substituted, the R^{12A.1} substituent group is substituted with one or more second substituent groups denoted by R^{12A.2} as explained in the definitions section above in the description of “first substituent group(s)”. In embodiments, when an R^{12A.2} substituent group is substituted, the R^{12A.2} substituent group is substituted with one or more third substituent groups denoted by R^{12A.3} as explained in the definitions section above in the description of “first substituent group(s)”. In the above embodiments, R^{12A}, R^{12A.1}, R^{12A.2}, and R^{12A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12A}, R^{12A.1}, R^{12A.2}, and R^{12A.3} respectively.

In embodiments, when R^{12B} is substituted, R^{12B} is substituted with one or more first substituent groups denoted by R^{12B.1} as explained in the definitions section above in the description of “first substituent group(s)”. In embodiments, when an R^{12B.1} substituent group is substituted, the R^{12B.1} substituent group is substituted with one or more second substituent groups denoted by R^{12B.2} as explained in the definitions section above in the description of “first substituent group(s)”. In embodiments, when an R^{12B.2} substituent group is substituted, the R^{12B.2} substituent group is substituted with one or more third substituent groups denoted by R^{12B.3} as explained in the definitions section above in the description of “first substituent group(s)”. In the above embodiments, R^{12B}, R^{12B.1}, R^{12B.2}, and R^{12B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12B}, R^{12B.1}, R^{12B.2}, and R^{12B.3}, respectively.

In embodiments, when R^{12A} and R^{12B} substituents bonded to the same nitrogen atom are optionally joined to form a moiety that is substituted (e.g., a substituted heterocycloalkyl or substituted heteroaryl), the moiety is substituted with one or more first substituent groups denoted by R^{12A.1} as explained in the definitions section above in the description of “first substituent group(s)”. In embodiments, when an R^{12A.1} substituent group is substituted, the R^{12A.1} substituent group is substituted with one or more second substituent groups denoted by R^{12A.2} as explained in the definitions section above in the description of “first substituent group(s)”. In embodiments, when an R^{12A.2} substituent group is substituted, the R^{12A.2} substituent group is substituted with one or more third substituent groups denoted by R^{12A.3} as explained in the definitions section above in the description of “first substituent group(s)”. In the above embodiments, R^{12A.1}, R^{12A.2}, and R^{12A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{12A.1}, R^{12A.2}, and R^{12A.3}, respectively.

In embodiments, when R^{12A} and R^{12B} substituents bonded to the same nitrogen atom are optionally joined to form a moiety that is substituted (e.g., a substituted heterocycloalkyl or substituted heteroaryl), the moiety is substituted with one or more first substituent groups denoted by R^{12B.1} as explained in the definitions section above in the description of “first substituent group(s)”. In embodiments, when an R^{12B.1} substituent group is substituted, the R^{12B.1} substituent group is substituted with one or more second substituent groups denoted by R^{12B.2} as explained in the definitions section above in the description of “first substituent group(s)”. In embodiments, when an R^{12B.2} substituent group is substituted, the R^{12B.2} substituent group is substituted with one or more third substituent groups denoted by R^{12B.3} as explained in the definitions section above in the description of “first substituent group(s)”. In the above embodiments, R^{12B.1}, R^{12B.2}, and R^{12B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{12B.1}, R^{12B.2}, and R^{12B.3}, respectively.

In embodiments, when R^{12C} is substituted, R^{12C} is substituted with one or more first substituent groups denoted by R^{12C.1} as explained in the definitions section above in the description of “first substituent group(s)”. In embodiments, when an R^{12C.1} substituent group is substituted, the R^{12C.1} substituent group is substituted with one or more second substituent groups denoted by R^{12C.2} as explained in the definitions section above in the description of “first substituent group(s)”. In embodiments, when an R^{12C.2} substituent group is substituted, the R^{12C.2} substituent group is substituted with one or more third substituent groups denoted by R^{12C.3} as explained in the definitions section above in the description of “first substituent group(s)”. In the above embodiments, R^{12C}, R^{12C.1}, R^{12C.2}, and R^{12C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12C}, R^{12C.1}, R^{12C.2}, and R^{12C.3} respectively.

In embodiments, when R^{12D} is substituted, R^{12D} is substituted with one or more first substituent groups denoted by R^{12D.1} as explained in the definitions section above in the description of “first substituent group(s)”. In embodiments, when an R^{12D.1} substituent group is substituted, the R^{12D.1} substituent group is substituted with one or more second substituent groups denoted by R^{12D.2} as explained in the definitions section above in the description of “first substituent group(s)”. In embodiments, when an R^{12D.2} substituent group is substituted, the R^{12D.2} substituent group is substituted with one or more third substituent groups denoted by R^{12D.3} as explained in the definitions section above in the description of “first substituent group(s)”. In the above embodiments, R^{12D}, R^{12D.1}, R^{12D.2}, and R^{12D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12D}, R^{12D.1}, R^{12D.2}, and R^{12D.3} respectively.

In embodiments, when R¹³ is substituted, R¹³ is substituted with one or more first substituent groups denoted by R^{13.1} as explained in the definitions section above in the description of “first substituent group(s)”. In embodiments, when an R^{13.1} substituent group is substituted, the R^{13.1} substituent group is substituted with one or more second substituent groups denoted by R^{13.2} as explained in the definitions section above in the description of “first substituent group(s)”. In embodiments, when an R^{13.2} substituent group is substituted, the R^{13.2} substituent group is substituted with one or more third substituent groups denoted by R^{13.3} as explained in the definitions section above in the description of “first substituent group(s)”. In the above embodiments, R¹³, R^{13.1}, R^{13.2}, and R^{13.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW2} and R^{WW.3} correspond to R¹³, R^{13.1}, R^{13.2}, and R^{13.3}, respectively.

In embodiments, when R^{13B} is substituted, R^{13B} is substituted with one or more first substituent groups denoted by R^{13A.1} as explained in the definitions section above in the description of “first substituent group(s)”. In embodiments, when an R^{13A.1} substituent group is substituted, the R^{13A.1} substituent group is substituted with one or more second substituent groups denoted by R^{13A.2} as explained in the definitions section above in the description of “first substituent group(s)”. In embodiments, when an R^{13A.2} substituent group is substituted, the R^{13A.2} substituent group is substituted with one or more third substituent groups denoted by R^{13A.3} as explained in the definitions section above in the description of “first substituent group(s)”. In the above embodiments, R^{13A}, R^{13A.1}, R^{13A.2}, and R^{13A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{13A}, R^{13A.1}, R^{13A.2}, and R^{13A.3} respectively.

In embodiments, when R^{13B} is substituted, R^{13B} is substituted with one or more first substituent groups denoted by R^{13B.1} as explained in the definitions section above in the description of “first substituent group(s)”. In embodiments, when an R^{13B.1} substituent group is substituted, the R^{13B.1} substituent group is substituted with one or more second substituent groups denoted by R^{13B.2} as explained in the definitions section above in the description of “first substituent group(s)”. In embodiments, when an R^{13B.2} substituent group is substituted, the R^{13B.2} substituent group is substituted with one or more third substituent groups denoted by R^{13B.3} as explained in the definitions section above in the description of “first substituent group(s)”. In the above embodiments, R^{13B}, R^{13B.1}, R^{13B.2}, and R^{13B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{13B}, R^{13B.1}, R^{13B.2}, and R^{13B.3} respectively.

In embodiments, when R^{13B} and R^{13B} substituents bonded to the same nitrogen atom are optionally joined to form a moiety that is substituted (e.g., a substituted heterocycloalkyl or substituted heteroaryl), the moiety is substituted with one or more first substituent groups denoted by R^{13A.1} as explained in the definitions section above in the description of “first substituent group(s)”. In embodiments, when an R^{13A.1} substituent group is substituted, the R^{13A.1} substituent group is substituted with one or more second substituent groups denoted by R^{13A.2} as explained in the definitions section above in the description of “first substituent group(s)”. In embodiments, when an R^{13A.2} substituent group is substituted, the R^{13A.2} substituent group is substituted with one or more third substituent groups denoted by R^{13A.3} as explained in the definitions section above in the description of “first substituent group(s)”. In the above embodiments, R^{13A.1}, R^{13A.2}, and R^{13A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{13A.1}, R^{13A.2}, and R^{13A.3}, respectively.

In embodiments, when R^{13B} and R^{13B} substituents bonded to the same nitrogen atom are optionally joined to form a moiety that is substituted (e.g., a substituted heterocycloalkyl or substituted heteroaryl), the moiety is substituted with one or more first substituent groups denoted by R^{13B.1} as explained in the definitions section above in the description of “first substituent group(s)”. In embodiments, when an R^{13B.1} substituent group is substituted, the R^{13B.1} substituent group is substituted with one or more second substituent groups denoted by R^{13B.2} as explained in the definitions section above in the description of “first substituent group(s)”. In embodiments, when an R^{13B.2} substituent group is substituted, the R^{13B.2} substituent group is substituted with one or more third substituent groups denoted by R^{13B.3} as explained in the definitions section above in the description of “first substituent group(s)”. In the above embodiments, R^{13B.1}, R^{13B.2}, and R^{13B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{13B.1}, R^{13B.2}, and R^{13B.3}, respectively.

In embodiments, when R^{13C} is substituted, R^{13C} is substituted with one or more first substituent groups denoted by R^{13C.1} as explained in the definitions section above in the description of “first substituent group(s)”. In embodiments, when an R^{13C.1} substituent group is substituted, the R^{13C.1} substituent group is substituted with one or more second substituent groups denoted by R^{13C.2} as explained in the definitions section above in the description of “first substituent group(s)”. In embodiments, when an R^{13C.2} substituent group is substituted, the R^{13C.2} substituent group is substituted with one or more third substituent groups denoted by R^{13C.3} as explained in the definitions section above in the description of “first substituent group(s)”. In the above embodiments, R^{13C}, R^{13C.1}, R^{13C.2}, and R^{13C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{13C}R^{13C.1}, R^{13C.2}, and R^{13C.3} respectively.

In embodiments, when R^{13D} is substituted, R^{13D} is substituted with one or more first substituent groups denoted by R^{13D.1} as explained in the definitions section above in the description of “first substituent group(s)”. In embodiments, when an R^{13D.1} substituent group is substituted, the R^{13D.1} substituent group is substituted with one or more second substituent groups denoted by R^{13D.2} as explained in the definitions section above in the description of “first substituent group(s)”. In embodiments, when an R^{13D.2} substituent group is substituted, the R^{13D.2} substituent group is substituted with one or more third substituent groups denoted by R^{13D.3} as explained in the definitions section above in the description of “first substituent group(s)”. In the above embodiments, R^{13D}, R^{13D.1}, R^{13D.2}, and R^{13D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{13D}, R^{13D.1}, R^{13D.2}, and R^{13D.3}, respectively.

In embodiments, when R² and R³ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.1} substituent group is substituted, the R^{2.2} substituent group is substituted with one or more third substituent groups denoted by R^{2.3} as explained in the definitions section above in the description of “first substituent group(s)”. In the above embodiments, R^{2.1}, R^{2.2}, and R^{2.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2.1}, R^{2.2}, and R^{2.3}, respectively.

In embodiments, when R² and R³ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R^{3.1}, R^{3.2}, and R^{3.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{3.1}, R^{3.2}, and R^{3.3}, respectively.

In embodiments, when R³ and R⁴ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R^{3.1}, R^{3.2}, and R^{3.3} have values corresponding to the values of R^{WW.1}, R^{WW.2} and R^{WW.3}, respectively, as explained in the definitions section above in the description of “first substituent group(s)”, wherein R^{WW.1}, R^{WW.2}, and R^{WW.3} correspond to R^{3.1}, R^{3.2}, and R^{3.3}, respectively.

In embodiments, when R³ and R⁴ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R^{4.1}, R^{4.2}, and R^{4.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4.1}, R^{4.2}, and R^{4.3}, respectively.

In embodiments, when R⁴ and R⁵ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R^{4.1}, R^{4.2}, and R^{4.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4.1}, R^{4.2}, and R^{4.3}, respectively.

In embodiments, when R⁴ and R⁵ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R^{5.1}, R^{5.2}, and R^{5.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5.1}, R^{5.2}, and R^{5.3}., respectively.

In embodiments, when R¹⁰ and R² substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R¹⁰, R^{10.2}, and R^{10.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R¹⁰, R^{10.2}, and R^{10.3}, respectively.

In embodiments, when R¹⁰ and R² substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.1} substituent group is substituted, the R^{2.2} substituent group is substituted with one or more third substituent groups denoted by R^{2.3} as explained in the definitions section above in the description of “first substituent group(s)”. In the above embodiments, R^{2.1}, R^{2.2}, and R^{2.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2.1}, R^{2.2}, and R^{2.3}, respectively.

In embodiments, when R¹¹ and R¹² substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{11.1} as explained in the definitions section above in the description of “first substituent group(s)”. In embodiments, when an R^{11.1} substituent group is substituted, the R^{11.1} substituent group is substituted with one or more second substituent groups denoted by R^{11.2} as explained in the definitions section above in the description of “first substituent group(s)”. In embodiments, when an R^{11.2} substituent group is substituted, the R^{11.2} substituent group is substituted with one or more third substituent groups denoted by R^{11.3} as explained in the definitions section above in the description of “first substituent group(s)”. In the above embodiments, R^{11.1}, R^{11.2}, and R^{11.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{11.1}, R^{11.2}, and R^{11.3}, respectively.

In embodiments, when R¹¹ and R¹² substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{12.1} as explained in the definitions section above in the description of “first substituent group(s)”. In embodiments, when an R^{12.1} substituent group is substituted, the R^{12.1} substituent group is substituted with one or more second substituent groups denoted by R^{12.2} as explained in the definitions section above in the description of “first substituent group(s)”. In embodiments, when an R^{12.2} substituent group is substituted, the R^{12.2} substituent group is substituted with one or more third substituent groups denoted by R^{12.3} as explained in the definitions section above in the description of “first substituent group(s)”. In the above embodiments, R^{12.1}, R^{12.2}, and R^{12.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{12.1}, R^{12.2}, and R^{12.3}, respectively.

In embodiments, the compound is

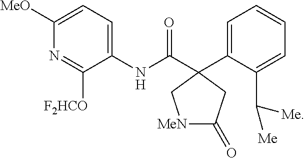 [SMILES: COc1ccc(NC(=O)C2(c3ccccc3C(C)C)CC(=O)N(C)C2)c(OC(F)F)n1]

In embodiments, the compound is

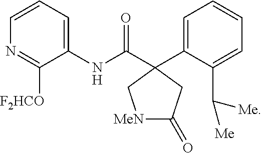 [SMILES: CC(C)c1ccccc1C1(C(=O)Nc2cccnc2OC(F)F)CC(=O)N(C)C1]

In embodiments, the compound is

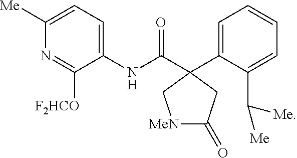 [SMILES: Cc1ccc(NC(=O)C2(c3ccccc3C(C)C)CC(=O)N(C)C2)c(OC(F)F)n1]

In embodiments, the compound is

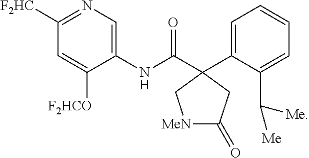 [SMILES: CC(C)c1ccccc1C1(C(=O)Nc2cnc(C(F)F)cc2OC(F)F)CC(=O)N(C)C1]

In embodiments, the compound is

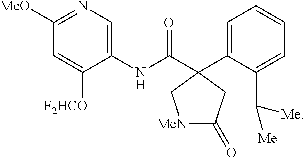 [SMILES: COc1cc(OC(F)F)c(NC(=O)C2(c3ccccc3C(C)C)CC(=O)N(C)C2)cn1]

In embodiments, the compound is

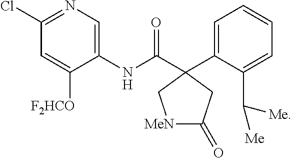 [SMILES: CC(C)c1ccccc1C1(C(=O)Nc2cnc(Cl)cc2OC(F)F)CC(=O)N(C)C1]

In embodiments, the compound is

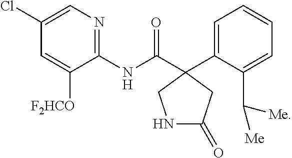 [SMILES: CC(C)c1ccccc1C1(C(=O)Nc2ncc(Cl)cc2OC(F)F)CNC(=O)C1]

In embodiments, the compound is

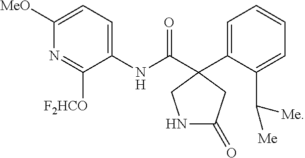 [SMILES: COc1ccc(NC(=O)C2(c3ccccc3C(C)C)CNC(=O)C2)c(OC(F)F)n1]

In embodiments, the compound is

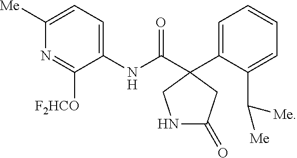 [SMILES: Cc1ccc(NC(=O)C2(c3ccccc3C(C)C)CNC(=O)C2)c(OC(F)F)n1]

In embodiments, the compound is

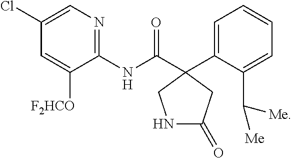 [SMILES: CC(C)c1ccccc1C1(C(=O)Nc2ncc(Cl)cc2OC(F)F)CNC(=O)C1]

In embodiments, the compound is

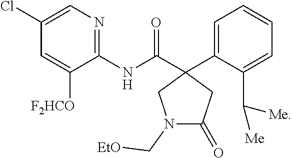 [SMILES: CCOCN1CC(C(=O)Nc2ncc(Cl)cc2OC(F)F)(c2ccccc2C(C)C)CC1=O]

In embodiments, the compound is

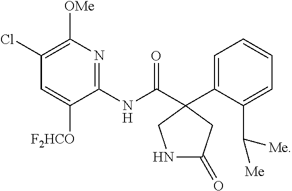 [SMILES: COc1nc(NC(=O)C2(c3ccccc3C(C)C)CNC(=O)C2)c(OC(F)F)cc1Cl]

In embodiments, the compound is

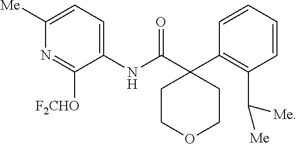

In embodiments, the compound is

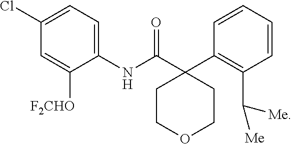

In embodiments, the compound is

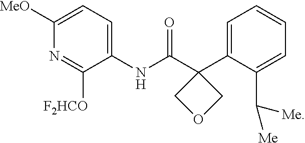 [SMILES: COc1ccc(NC(=O)C2(c3ccccc3C(C)C)COC2)c(OC(F)F)n1]

In embodiments, the compound is

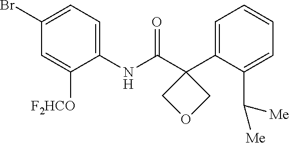 [SMILES: CC(C)c1ccccc1C1(C(=O)Nc2ccc(Br)cc2OC(F)F)COC1]

In embodiments, the compound is

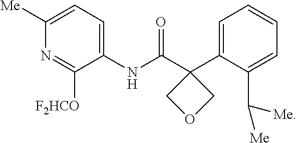 [SMILES: Cc1ccc(NC(=O)C2(c3ccccc3C(C)C)COC2)c(OC(F)F)n1]

In embodiments, the compound is

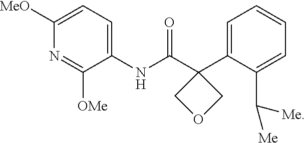 [SMILES: COc1ccc(NC(=O)C2(c3ccccc3C(C)C)COC2)c(OC)n1]

In embodiments, the compound is

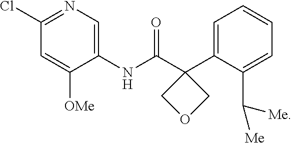 [SMILES: COc1cc(Cl)ncc1NC(=O)C1(c2ccccc2C(C)C)COC1]

In embodiments, the compound is

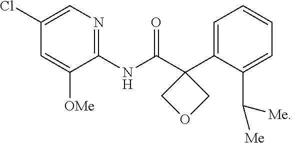 [SMILES: COc1cc(Cl)cnc1NC(=O)C1(c2ccccc2C(C)C)COC1]

In embodiments, the compound is

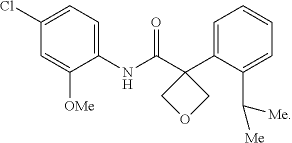 [SMILES: COc1cc(Cl)ccc1NC(=O)C1(c2ccccc2C(C)C)COC1]

In embodiments, the compound is

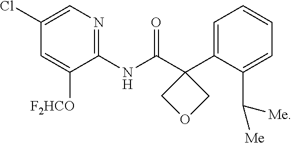 [SMILES: CC(C)c1ccccc1C1(C(=O)Nc2ncc(Cl)cc2OC(F)F)COC1]

In embodiments, the compound is

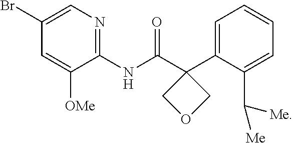 [SMILES: COc1cc(Br)cnc1NC(=O)C1(c2ccccc2C(C)C)COC1]

In embodiments, the compound is

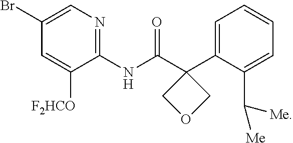 [SMILES: CC(C)c1ccccc1C1(C(=O)Nc2ncc(Br)cc2OC(F)F)COC1]

In embodiments, the compound is

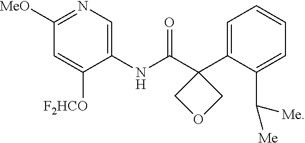 [SMILES: COc1cc(OC(F)F)c(NC(=O)C2(c3ccccc3C(C)C)COC2)cn1]

In embodiments, the compound is

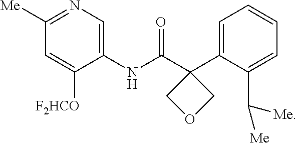 [SMILES: Cc1cc(OC(F)F)c(NC(=O)C2(c3ccccc3C(C)C)COC2)cn1]

In embodiments, the compound is

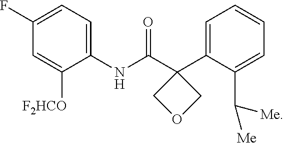 [SMILES: CC(C)c1ccccc1C1(C(=O)Nc2ccc(F)cc2OC(F)F)COC1]

In embodiments, the compound is

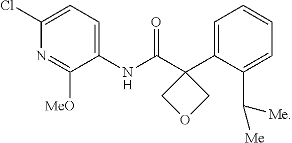 [SMILES: COc1nc(Cl)ccc1NC(=O)C1(c2ccccc2C(C)C)COC1]

In embodiments, the compound is

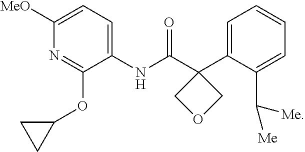 [SMILES: COc1ccc(NC(=O)C2(c3ccccc3C(C)C)COC2)c(OC2CC2)n1]

In embodiments, the compound is

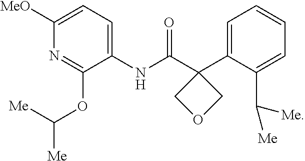 [SMILES: COc1ccc(NC(=O)C2(c3ccccc3C(C)C)COC2)c(OC(C)C)n1]

In embodiments, the compound is

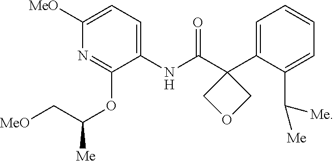 [SMILES: COC[C@H](C)Oc1nc(OC)ccc1NC(=O)C1(c2ccccc2C(C)C)COC1]

In embodiments, the compound is

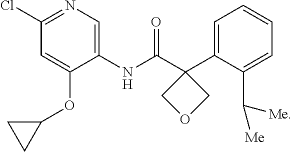 [SMILES: CC(C)c1ccccc1C1(C(=O)Nc2cnc(Cl)cc2OC2CC2)COC1]

In embodiments, the compound is

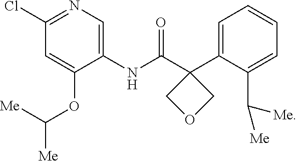 [SMILES: CC(C)Oc1cc(Cl)ncc1NC(=O)C1(c2ccccc2C(C)C)COC1]

In embodiments, the compound is

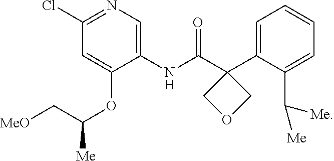 [SMILES: COC[C@H](C)Oc1cc(Cl)ncc1NC(=O)C1(c2ccccc2C(C)C)COC1]

In embodiments, the compound is

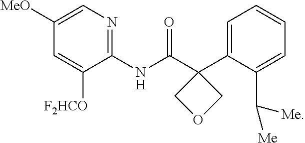 [SMILES: COc1cnc(NC(=O)C2(c3ccccc3C(C)C)COC2)c(OC(F)F)c1]

In embodiments, the compound is

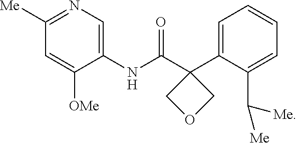 [SMILES: COc1cc(C)ncc1NC(=O)C1(c2ccccc2C(C)C)COC1]

In embodiments, the compound is

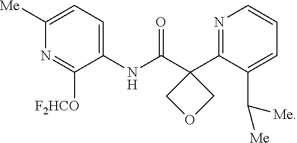 [SMILES: Cc1ccc(NC(=O)C2(c3ncccc3C(C)C)COC2)c(OC(F)F)n1]

In embodiments, the compound is

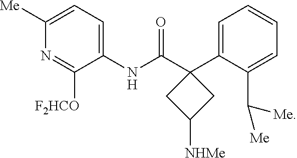 [SMILES: CNC1CC(C(=O)Nc2ccc(C)nc2OC(F)F)(c2ccccc2C(C)C)C1]

In embodiments, the compound is

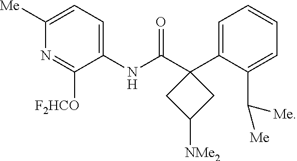 [SMILES: Cc1ccc(NC(=O)C2(c3ccccc3C(C)C)CC(N(C)C)C2)c(OC(F)F)n1]

In embodiments, the compound is

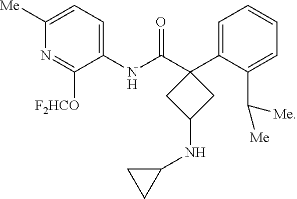 [SMILES: Cc1ccc(NC(=O)C2(c3ccccc3C(C)C)CC(NC3CC3)C2)c(OC(F)F)n1]

In embodiments, the compound is

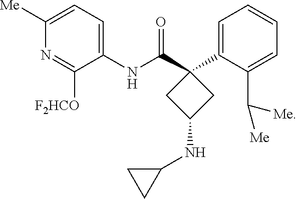 [SMILES: Cc1ccc(NC(=O)[C@]2(c3ccccc3C(C)C)C[C@@H](NC3CC3)C2)c(OC(F)F)n1]

In embodiments, the compound is

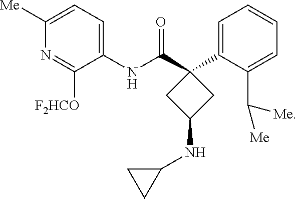 [SMILES: Cc1ccc(NC(=O)[C@]2(c3ccccc3C(C)C)C[C@H](NC3CC3)C2)c(OC(F)F)n1]

In embodiments, the compound is

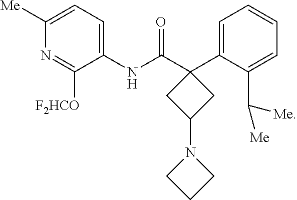 [SMILES: Cc1ccc(NC(=O)C2(c3ccccc3C(C)C)CC(N3CCC3)C2)c(OC(F)F)n1]

In embodiments, the compound is

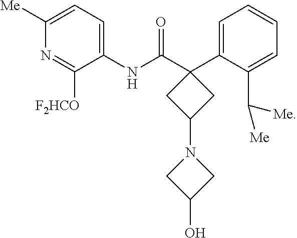 [SMILES: Cc1ccc(NC(=O)C2(c3ccccc3C(C)C)CC(N3CC(O)C3)C2)c(OC(F)F)n1]

In embodiments, the compound is

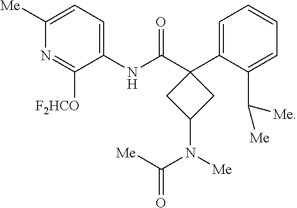 [SMILES: CC(=O)N(C)C1CC(C(=O)Nc2ccc(C)nc2OC(F)F)(c2ccccc2C(C)C)C1]

In embodiments, the compound is

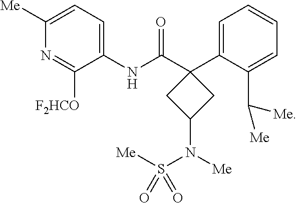 [SMILES: Cc1ccc(NC(=O)C2(c3ccccc3C(C)C)CC(N(C)S(C)(=O)=O)C2)c(OC(F)F)n1]

In embodiments, the compound is

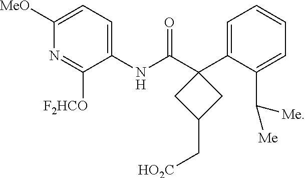 [SMILES: COc1ccc(NC(=O)C2(c3ccccc3C(C)C)CC(CC(=O)O)C2)c(OC(F)F)n1]

In embodiments, the compound is

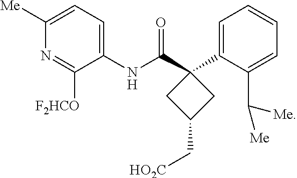 [SMILES: Cc1ccc(NC(=O)[C@]2(c3ccccc3C(C)C)C[C@@H](CC(=O)O)C2)c(OC(F)F)n1]

In embodiments, the compound is

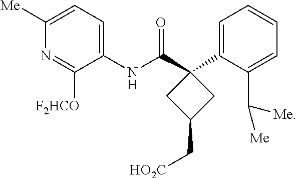 [SMILES: Cc1ccc(NC(=O)[C@]2(c3ccccc3C(C)C)C[C@H](CC(=O)O)C2)c(OC(F)F)n1]

In embodiments, the compound is

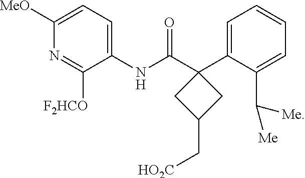 [SMILES: COc1ccc(NC(=O)C2(c3ccccc3C(C)C)CC(CC(=O)O)C2)c(OC(F)F)n1]

In embodiments, the compound is

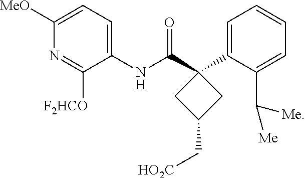 [SMILES: COc1ccc(NC(=O)[C@]2(c3ccccc3C(C)C)C[C@@H](CC(=O)O)C2)c(OC(F)F)n1]

In embodiments, the compound is

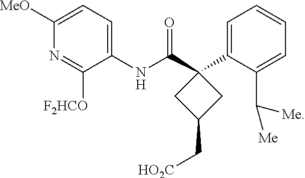 [SMILES: COc1ccc(NC(=O)[C@]2(c3ccccc3C(C)C)C[C@H](CC(=O)O)C2)c(OC(F)F)n1]

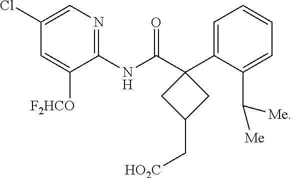 [SMILES: CC(C)c1ccccc1C1(C(=O)Nc2ncc(Cl)cc2OC(F)F)CC(CC(=O)O)C1]

In embodiments, the compound is

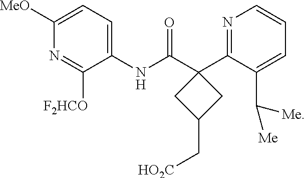 [SMILES: COc1ccc(NC(=O)C2(c3ncccc3C(C)C)CC(CC(=O)O)C2)c(OC(F)F)n1]

In embodiments, the compound is

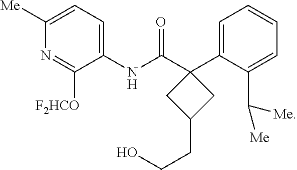 [SMILES: Cc1ccc(NC(=O)C2(c3ccccc3C(C)C)CC(CCO)C2)c(OC(F)F)n1]

In embodiments, the compound is

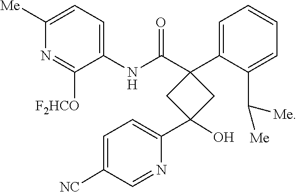 [SMILES: Cc1ccc(NC(=O)C2(c3ccccc3C(C)C)CC(O)(c3ccc(C#N)cn3)C2)c(OC(F)F)n1]

In embodiments, the compound is

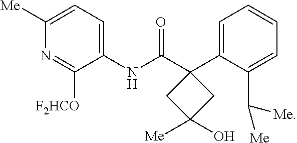 [SMILES: Cc1ccc(NC(=O)C2(c3ccccc3C(C)C)CC(C)(O)C2)c(OC(F)F)n1]

In embodiments, the compound is

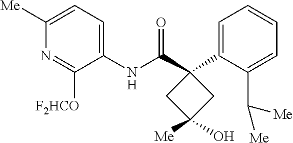 [SMILES: Cc1ccc(NC(=O)[C@]2(c3ccccc3C(C)C)C[C@@](C)(O)C2)c(OC(F)F)n1]

In embodiments, the compound is

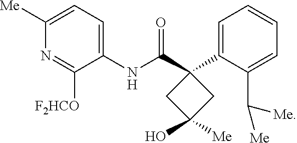 [SMILES: Cc1ccc(NC(=O)[C@]2(c3ccccc3C(C)C)C[C@](C)(O)C2)c(OC(F)F)n1]

In embodiments, the compound is

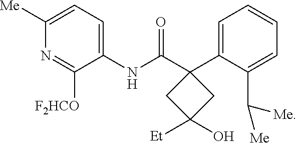 [SMILES: CCC1(O)CC(C(=O)Nc2ccc(C)nc2OC(F)F)(c2ccccc2C(C)C)C1]

In embodiments, the compound is

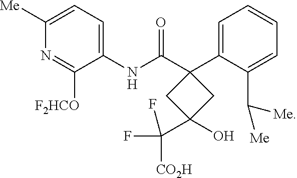 [SMILES: Cc1ccc(NC(=O)C2(c3ccccc3C(C)C)CC(O)(C(F)(F)C(=O)O)C2)c(OC(F)F)n1]

In embodiments, the compound is

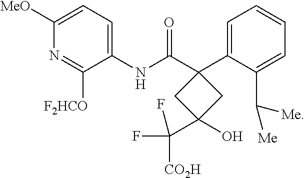 [SMILES: COc1ccc(NC(=O)C2(c3ccccc3C(C)C)CC(O)(C(F)(F)C(=O)O)C2)c(OC(F)F)n1]

In embodiments, the compound is

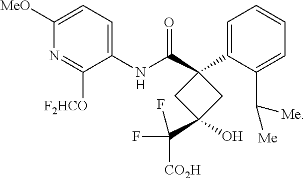 [SMILES: COc1ccc(NC(=O)[C@]2(c3ccccc3C(C)C)C[C@](O)(C(F)(F)C(=O)O)C2)c(OC(F)F)n1]

In embodiments, the compound is

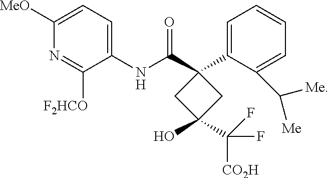 [SMILES: COc1ccc(NC(=O)[C@]2(c3ccccc3C(C)C)C[C@@](O)(C(F)(F)C(=O)O)C2)c(OC(F)F)n1]

In embodiments, the compound is

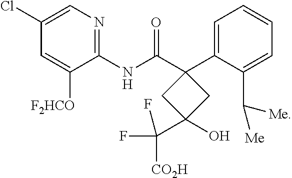 [SMILES: CC(C)c1ccccc1C1(C(=O)Nc2ncc(Cl)cc2OC(F)F)CC(O)(C(F)(F)C(=O)O)C1]

In embodiments, the compound is

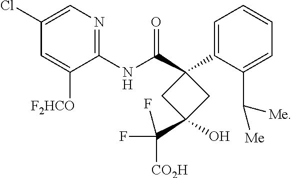 [SMILES: CC(C)c1ccccc1[C@]1(C(=O)Nc2ncc(Cl)cc2OC(F)F)C[C@@](O)(C(F)(F)C(=O)O)C1]

In embodiments, the compound is

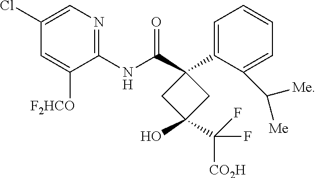 [SMILES: CC(C)c1ccccc1[C@]1(C(=O)Nc2ncc(Cl)cc2OC(F)F)C[C@](O)(C(F)(F)C(=O)O)C1]

In embodiments, the compound is

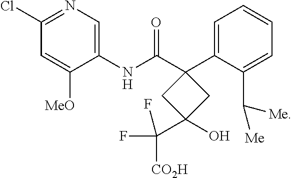 [SMILES: COc1cc(Cl)ncc1NC(=O)C1(c2ccccc2C(C)C)CC(O)(C(F)(F)C(=O)O)C1]

In embodiments, the compound is

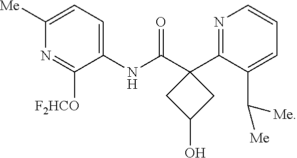 [SMILES: Cc1ccc(NC(=O)C2(c3ncccc3C(C)C)CC(O)C2)c(OC(F)F)n1]

In embodiments, the compound is

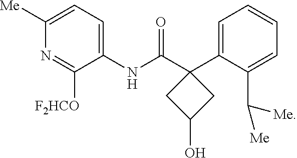 [SMILES: Cc1ccc(NC(=O)C2(c3ccccc3C(C)C)CC(O)C2)c(OC(F)F)n1]

In embodiments, the compound is

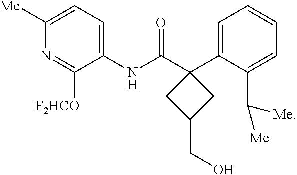 [SMILES: Cc1ccc(NC(=O)C2(c3ccccc3C(C)C)CC(CO)C2)c(OC(F)F)n1]

In embodiments, the compound is

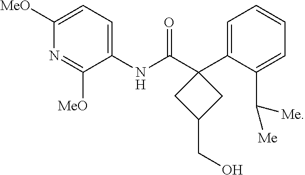 [SMILES: COc1ccc(NC(=O)C2(c3ccccc3C(C)C)CC(CO)C2)c(OC)n1]

In embodiments, the compound is

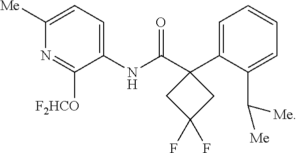 [SMILES: Cc1ccc(NC(=O)C2(c3ccccc3C(C)C)CC(F)(F)C2)c(OC(F)F)n1]

In embodiments, the compound is

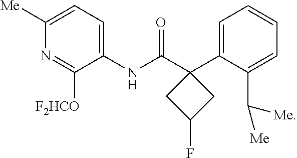 [SMILES: Cc1ccc(NC(=O)C2(c3ccccc3C(C)C)CC(F)C2)c(OC(F)F)n1]

In embodiments, the compound is

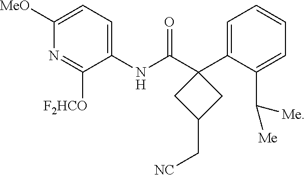 [SMILES: COc1ccc(NC(=O)C2(c3ccccc3C(C)C)CC(CC#N)C2)c(OC(F)F)n1]

In embodiments, the compound is

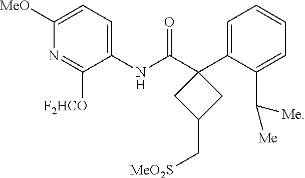 [SMILES: COc1ccc(NC(=O)C2(c3ccccc3C(C)C)CC(CS(C)(=O)=O)C2)c(OC(F)F)n1]

In embodiments, the compound is

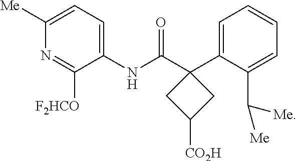 [SMILES: Cc1ccc(NC(=O)C2(c3ccccc3C(C)C)CC(C(=O)O)C2)c(OC(F)F)n1]

In embodiments, the compound is

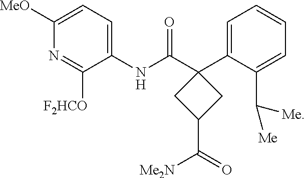 [SMILES: COc1ccc(NC(=O)C2(c3ccccc3C(C)C)CC(C(=O)N(C)C)C2)c(OC(F)F)n1]

In embodiments, the

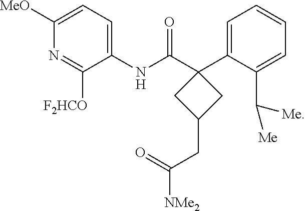 [SMILES: COc1ccc(NC(=O)C2(c3ccccc3C(C)C)CC(CC(=O)N(C)C)C2)c(OC(F)F)n1]

In embodiments, the compound is

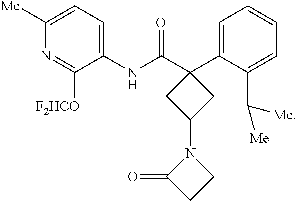 [SMILES: Cc1ccc(NC(=O)C2(c3ccccc3C(C)C)CC(N3CCC3=O)C2)c(OC(F)F)n1]

In embodiments, the compound is

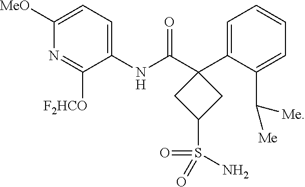 [SMILES: COc1ccc(NC(=O)C2(c3ccccc3C(C)C)CC(S(N)(=O)=O)C2)c(OC(F)F)n1]

In embodiments, the compound is

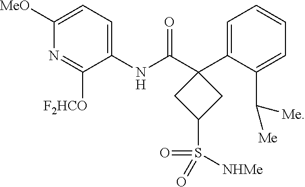 [SMILES: CNS(=O)(=O)C1CC(C(=O)Nc2ccc(OC)nc2OC(F)F)(c2ccccc2C(C)C)C1]

In embodiments, the compound is

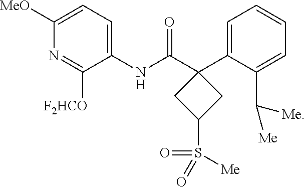 [SMILES: COc1ccc(NC(=O)C2(c3ccccc3C(C)C)CC(S(C)(=O)=O)C2)c(OC(F)F)n1]

In embodiments, the compound is

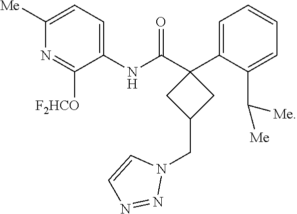 [SMILES: Cc1ccc(NC(=O)C2(c3ccccc3C(C)C)CC(Cn3ccnn3)C2)c(OC(F)F)n1]

In embodiments, the compound is

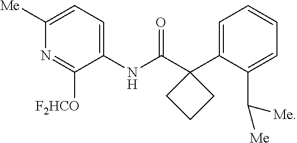 [SMILES: Cc1ccc(NC(=O)C2(c3ccccc3C(C)C)CCC2)c(OC(F)F)n1]

In embodiments, the compound is

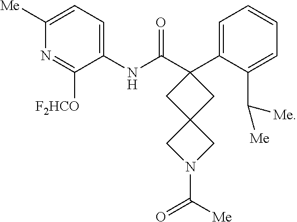 [SMILES: CC(=O)N1CC2(C1)CC(C(=O)Nc1ccc(C)nc1OC(F)F)(c1ccccc1C(C)C)C2]

In embodiments, the compound is

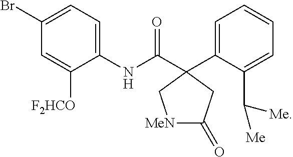 [SMILES: CC(C)c1ccccc1C1(C(=O)Nc2ccc(Br)cc2OC(F)F)CC(=O)N(C)C1]

In embodiments, the compound is

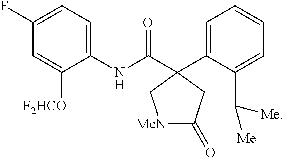 [SMILES: CC(C)c1ccccc1C1(C(=O)Nc2ccc(F)cc2OC(F)F)CC(=O)N(C)C1]

In embodiments, the compound is

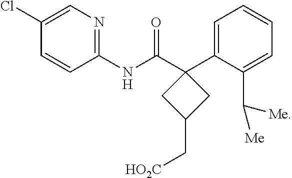 [SMILES: CC(C)c1ccccc1C1(C(=O)Nc2ccc(Cl)cn2)CC(CC(=O)O)C1]

In embodiments, the compound is

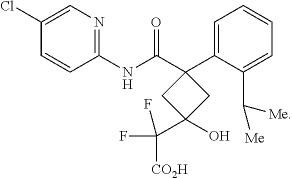 [SMILES: CC(C)c1ccccc1C1(C(=O)Nc2ccc(Cl)cn2)CC(O)(C(F)(F)C(=O)O)C1]

In embodiments, the compound is

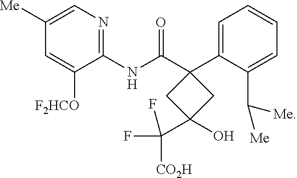 [SMILES: Cc1cnc(NC(=O)C2(c3ccccc3C(C)C)CC(O)(C(F)(F)C(=O)O)C2)c(OC(F)F)c1]

In embodiments, the compound is

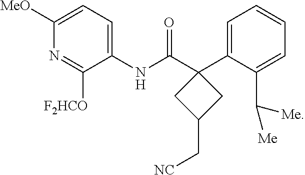 [SMILES: COc1ccc(NC(=O)C2(c3ccccc3C(C)C)CC(CC#N)C2)c(OC(F)F)n1]

In embodiments, the compound is

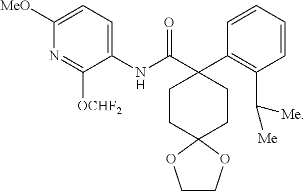 [SMILES: COc1ccc(NC(=O)C2(c3ccccc3C(C)C)CCC3(CC2)OCCO3)c(OC(F)F)n1]

In embodiments, the compound is

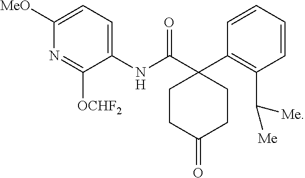 [SMILES: COc1ccc(NC(=O)C2(c3ccccc3C(C)C)CCC(=O)CC2)c(OC(F)F)n1]

In embodiments, the compound is

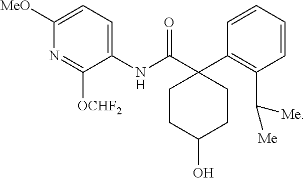 [SMILES: COc1ccc(NC(=O)C2(c3ccccc3C(C)C)CCC(O)CC2)c(OC(F)F)n1]

In embodiments, the compound is

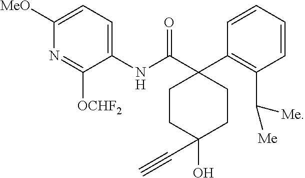 [SMILES: C#CC1(O)CCC(C(=O)Nc2ccc(OC)nc2OC(F)F)(c2ccccc2C(C)C)CC1]

In embodiments, the compound is

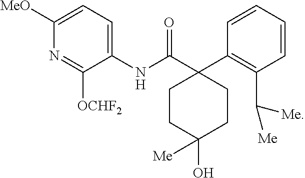 [SMILES: COc1ccc(NC(=O)C2(c3ccccc3C(C)C)CCC(C)(O)CC2)c(OC(F)F)n1]

In embodiments, the compound is

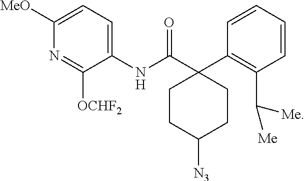 [SMILES: COc1ccc(NC(=O)C2(c3ccccc3C(C)C)CCC(N=[N+]=[N-])CC2)c(OC(F)F)n1]

In embodiments, the compound is

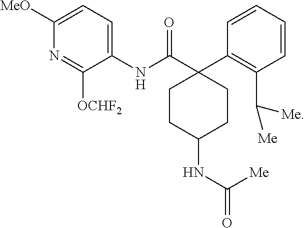 [SMILES: COc1ccc(NC(=O)C2(c3ccccc3C(C)C)CCC(NC(C)=O)CC2)c(OC(F)F)n1]

In embodiments, the compound is

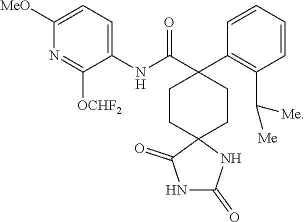 [SMILES: COc1ccc(NC(=O)C2(c3ccccc3C(C)C)CCC3(CC2)NC(=O)NC3=O)c(OC(F)F)n1]

In embodiments, the compound is

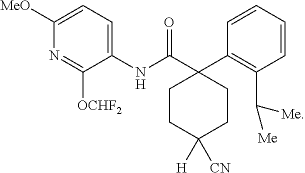 [SMILES: COc1ccc(NC(=O)C2(c3ccccc3C(C)C)CCC(C#N)CC2)c(OC(F)F)n1]

In embodiments, the compound is

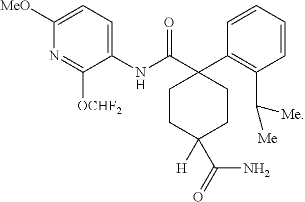 [SMILES: COc1ccc(NC(=O)C2(c3ccccc3C(C)C)CCC(C(N)=O)CC2)c(OC(F)F)n1]

In embodiments, the compound is

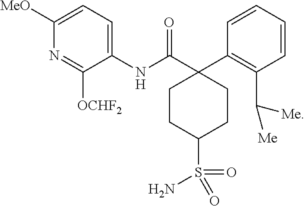 [SMILES: COc1ccc(NC(=O)C2(c3ccccc3C(C)C)CCC(S(N)(=O)=O)CC2)c(OC(F)F)n1]

In embodiments, the compound is

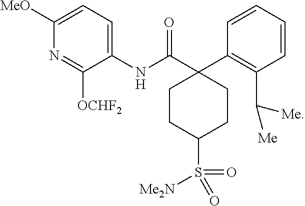 [SMILES: COc1ccc(NC(=O)C2(c3ccccc3C(C)C)CCC(S(=O)(=O)N(C)C)CC2)c(OC(F)F)n1]

In embodiments, the compound is

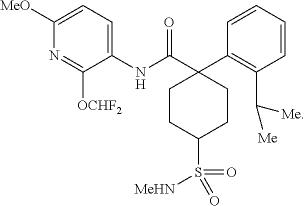 [SMILES: CNS(=O)(=O)C1CCC(C(=O)Nc2ccc(OC)nc2OC(F)F)(c2ccccc2C(C)C)CC1]

In embodiments, the compound is

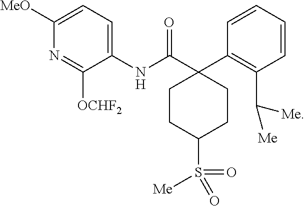 [SMILES: COc1ccc(NC(=O)C2(c3ccccc3C(C)C)CCC(S(C)(=O)=O)CC2)c(OC(F)F)n1]

In embodiments, the compound is

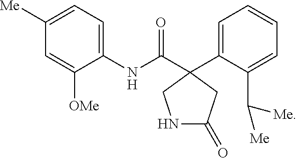 [SMILES: COc1cc(C)ccc1NC(=O)C1(c2ccccc2C(C)C)CNC(=O)C1]

In embodiments, the compound is

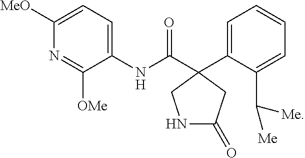 [SMILES: COc1ccc(NC(=O)C2(c3ccccc3C(C)C)CNC(=O)C2)c(OC)n1]

In embodiments, the compound is

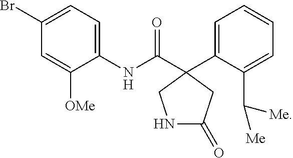 [SMILES: COc1cc(Br)ccc1NC(=O)C1(c2ccccc2C(C)C)CNC(=O)C1]

In embodiments, the compound is

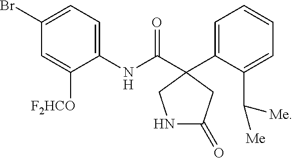 [SMILES: CC(C)c1ccccc1C1(C(=O)Nc2ccc(Br)cc2OC(F)F)CNC(=O)C1]

In embodiments, the compound is

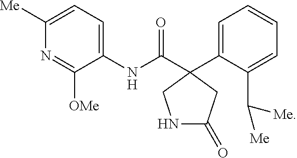 [SMILES: COc1nc(C)ccc1NC(=O)C1(c2ccccc2C(C)C)CNC(=O)C1]

In embodiments, the compound is

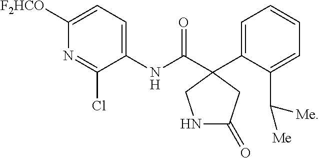 [SMILES: CC(C)c1ccccc1C1(C(=O)Nc2ccc(OC(F)F)nc2Cl)CNC(=O)C1]

In embodiments, the compound is

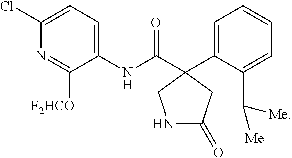 [SMILES: CC(C)c1ccccc1C1(C(=O)Nc2ccc(Cl)nc2OC(F)F)CNC(=O)C1]

In embodiments, the compound is

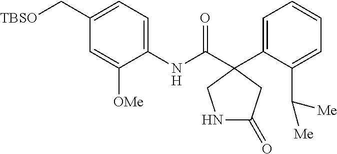 [SMILES: COc1cc(CO[Si](C)(C)C(C)(C)C)ccc1NC(=O)C1(c2ccccc2C(C)C)CNC(=O)C1]

In embodiments, the compound is

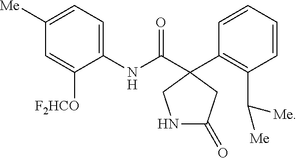 [SMILES: Cc1ccc(NC(=O)C2(c3ccccc3C(C)C)CNC(=O)C2)c(OC(F)F)c1]

In embodiments, the compound is

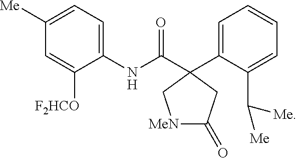 [SMILES: Cc1ccc(NC(=O)C2(c3ccccc3C(C)C)CC(=O)N(C)C2)c(OC(F)F)c1]

In embodiments, the compound is

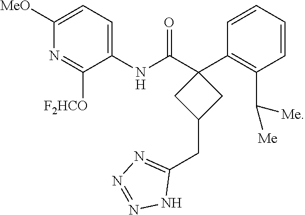 [SMILES: COc1ccc(NC(=O)C2(c3ccccc3C(C)C)CC(Cc3nnn[nH]3)C2)c(OC(F)F)n1]

In embodiments, the compound is

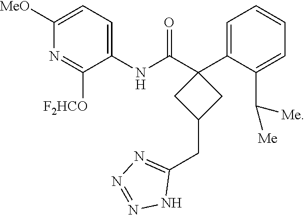 [SMILES: COc1ccc(NC(=O)C2(c3ccccc3C(C)C)CC(Cc3nnn[nH]3)C2)c(OC(F)F)n1]

In embodiments, the compound is

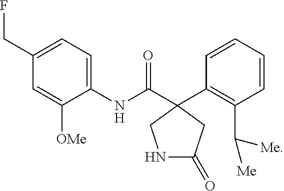 [SMILES: COc1cc(CF)ccc1NC(=O)C1(c2ccccc2C(C)C)CNC(=O)C1]

In embodiments, the compound is

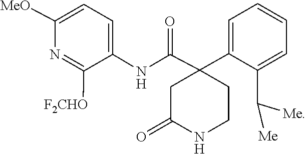

In embodiments, the compound is

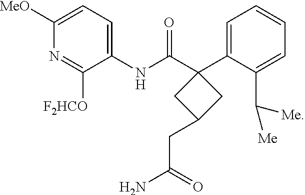 [SMILES: COc1ccc(NC(=O)C2(c3ccccc3C(C)C)CC(CC(N)=O)C2)c(OC(F)F)n1]

In embodiments, the compound is

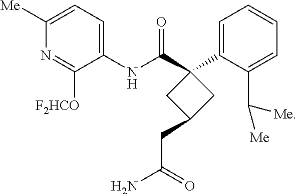 [SMILES: Cc1ccc(NC(=O)[C@]2(c3ccccc3C(C)C)C[C@H](CC(N)=O)C2)c(OC(F)F)n1]

In embodiments, the compound is

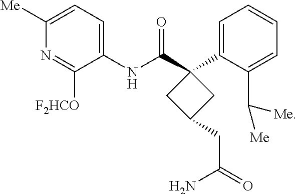 [SMILES: Cc1ccc(NC(=O)[C@]2(c3ccccc3C(C)C)C[C@@H](CC(N)=O)C2)c(OC(F)F)n1]

In embodiments, the compound is

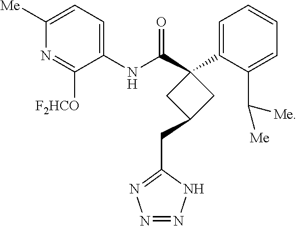 [SMILES: Cc1ccc(NC(=O)[C@]2(c3ccccc3C(C)C)C[C@H](Cc3nnn[nH]3)C2)c(OC(F)F)n1]

In embodiments, the compound is

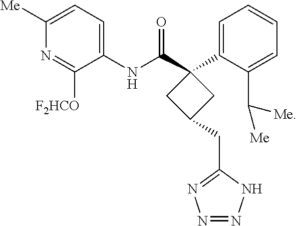 [SMILES: Cc1ccc(NC(=O)[C@]2(c3ccccc3C(C)C)C[C@@H](Cc3nnn[nH]3)C2)c(OC(F)F)n1]

In embodiments, the compound is

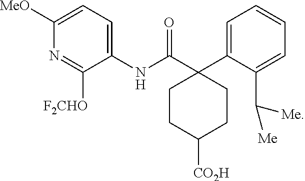

In embodiments, the compound is

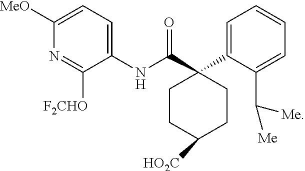

In embodiments, the compound is

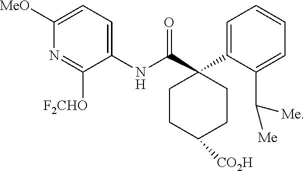

In embodiments, the compound is

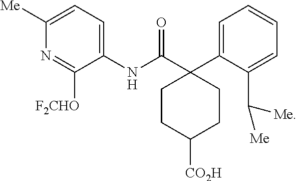

In embodiments, the compound is

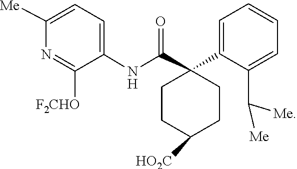

In embodiments, the compound is

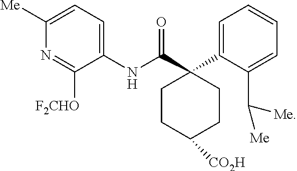

In embodiments, the compound is

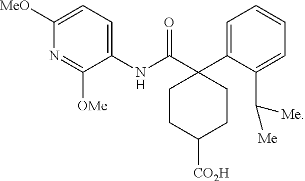 [SMILES: COc1ccc(NC(=O)C2(c3ccccc3C(C)C)CCC(C(=O)O)CC2)c(OC)n1]

In embodiments, the compound is

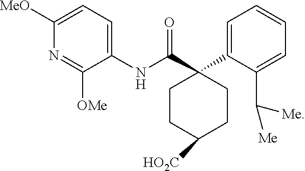 [SMILES: COc1ccc(NC(=O)[C@]2(c3ccccc3C(C)C)CC[C@H](C(=O)O)CC2)c(OC)n1]

In embodiments, the compound is

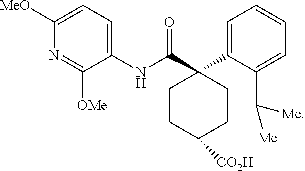

In embodiments, the compound is

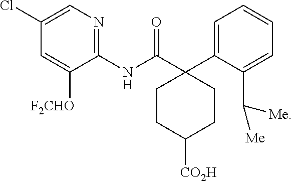

In embodiments, the compound is

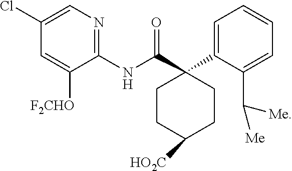

In embodiments, the compound is

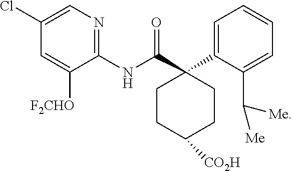

In embodiments, the compound is

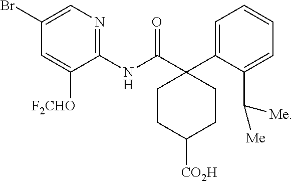

In embodiments, the compound is

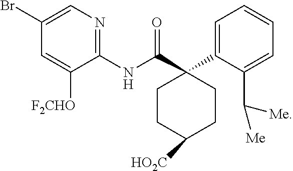

In embodiments, the compound is

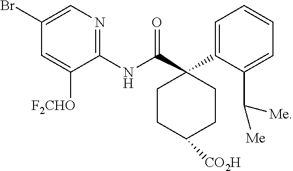

In embodiments, the compound is

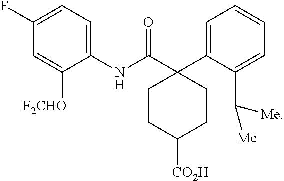

In embodiments, the compound is

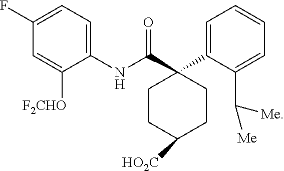

In embodiments, the compound is

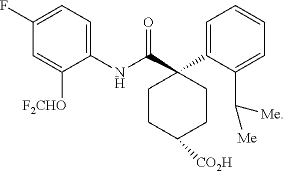

In embodiments, the compound is

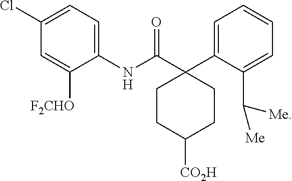

In embodiments, the compound is

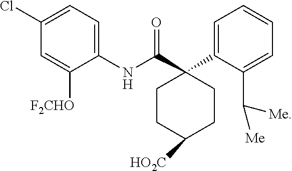

In embodiments, the compound is

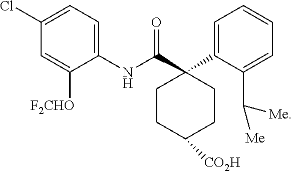

In embodiments, the compound is

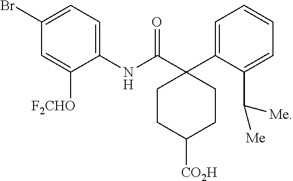

In embodiments, the compound is

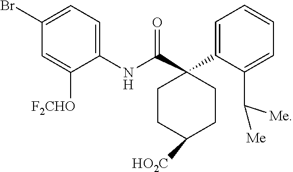

In embodiments, the compound is

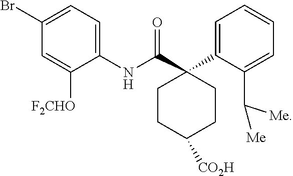

In embodiments, the compound is

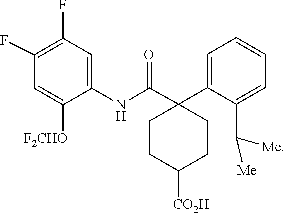

In embodiments, the compound is

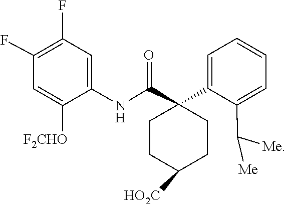

In embodiments, the compound is

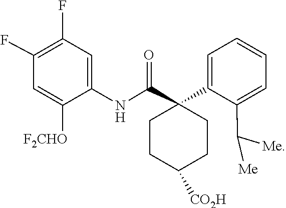

In embodiments, the compound is

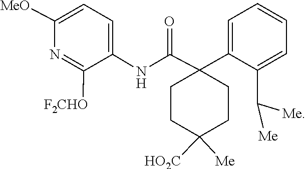

In embodiments, the compound is

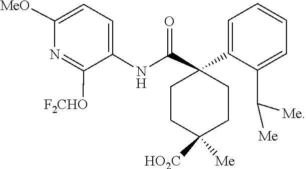

In embodiments, the compound is

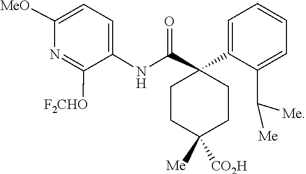

In embodiments, the compound is

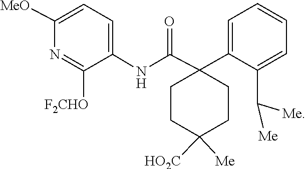

In embodiments, the compound is

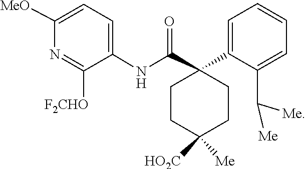

In embodiments, the compound is

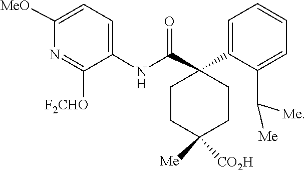

In embodiments, the compound is

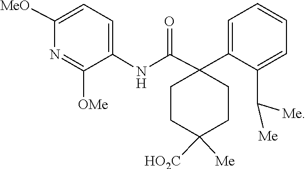 [SMILES: COc1ccc(NC(=O)C2(c3ccccc3C(C)C)CCC(C)(C(=O)O)CC2)c(OC)n1]

In embodiments, the compound is

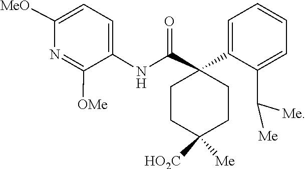 [SMILES: COc1ccc(NC(=O)[C@]2(c3ccccc3C(C)C)CC[C@](C)(C(=O)O)CC2)c(OC)n1]

In embodiments, the compound is

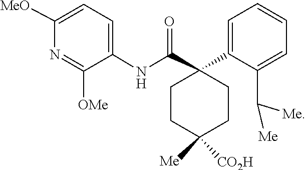 [SMILES: COc1ccc(NC(=O)[C@]2(c3ccccc3C(C)C)CC[C@@](C)(C(=O)O)CC2)c(OC)n1]

In embodiments, the compound is

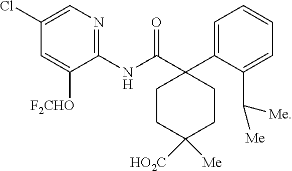

In embodiments, the compound is

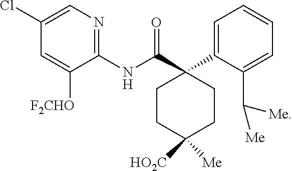

In embodiments, the compound is

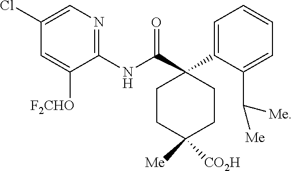

In embodiments, the compound is

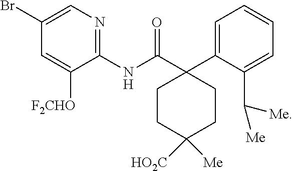

In embodiments, the compound is

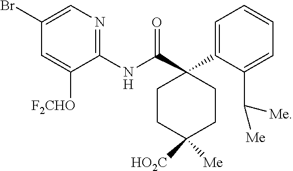

In embodiments, the compound is

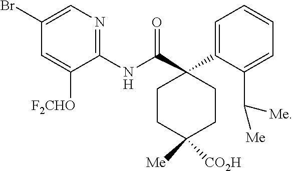

In embodiments, the compound is

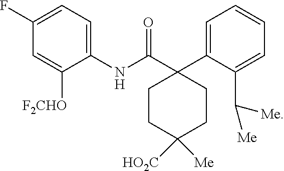

In embodiments, the compound is

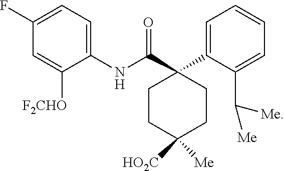

In embodiments, the compound is

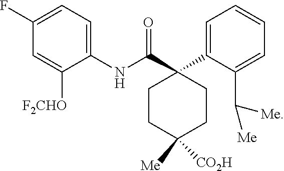

In embodiments, the compound is

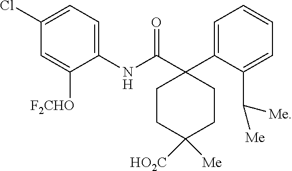

In embodiments, the compound is

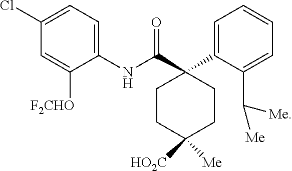

In embodiments, the compound is

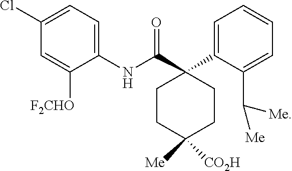

In embodiments, the compound is

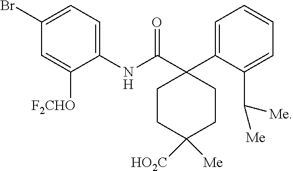

In embodiments, the compound is

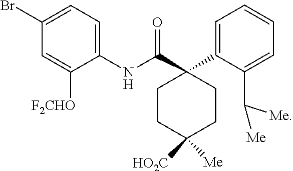

In embodiments, the compound is

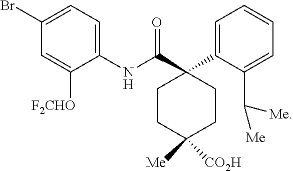

In embodiments, the compound is

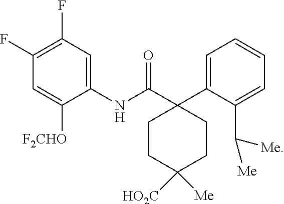

In embodiments, the compound is

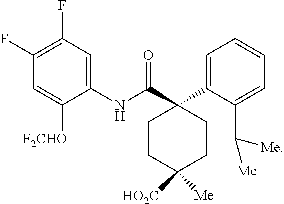

In embodiments, the compound is

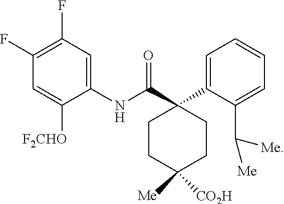

In embodiments, the compound is

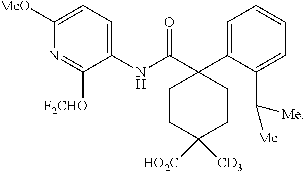

In embodiments, the compound is

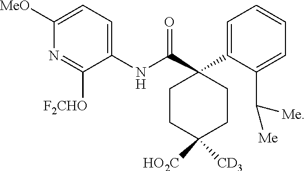

In embodiments, the compound is

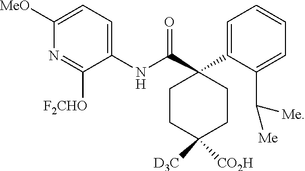

In embodiments, the compound is

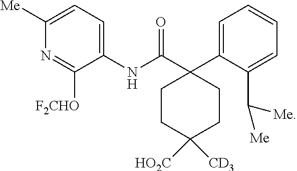

In embodiments, the compound is

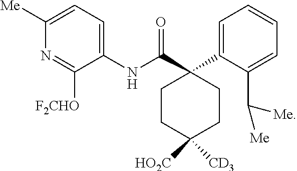

In embodiments, the compound is

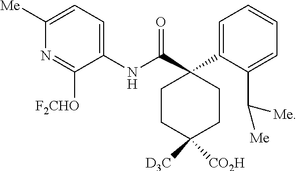

In embodiments, the compound is

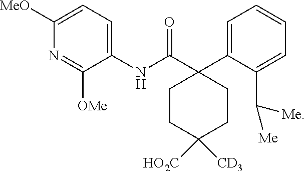 [SMILES: [2H]C([2H])([2H])C1(C(=O)O)CCC(C(=O)Nc2ccc(OC)nc2OC)(c2ccccc2C(C)C)CC1]

In embodiments, the compound is

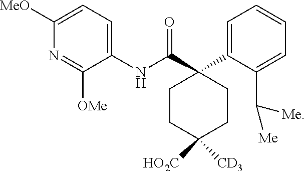

In embodiments, the compound is

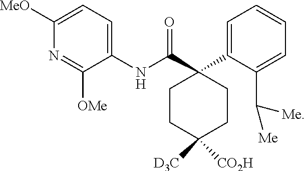

In embodiments, the compound is

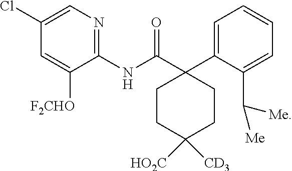

In embodiments, the compound is

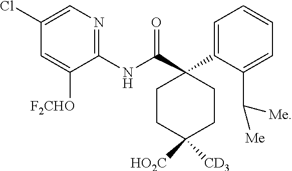

In embodiments, the compound is

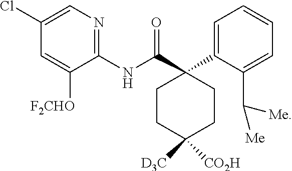

In embodiments, the compound is

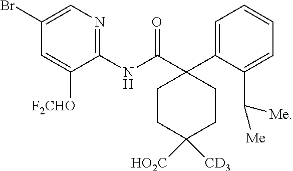

In embodiments, the compound is

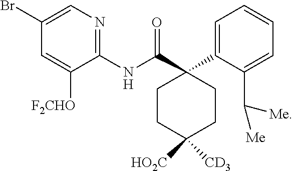

In embodiments, the compound is

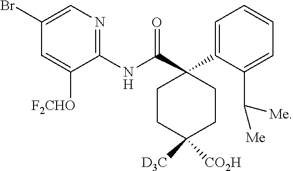

In embodiments, the compound is

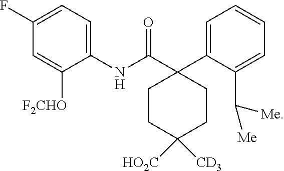

In embodiments, the compound is

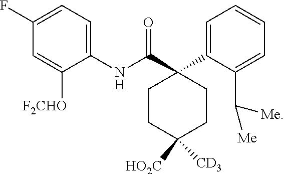

In embodiments, the compound is

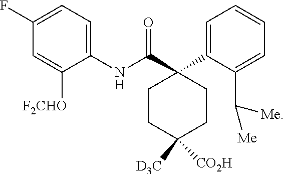

In embodiments, the compound is

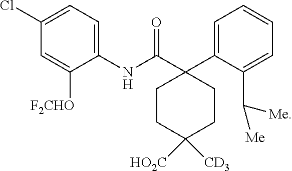

In embodiments, the compound is

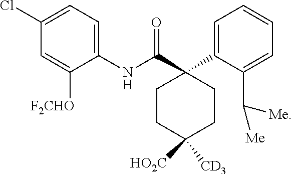

In embodiments, the compound is

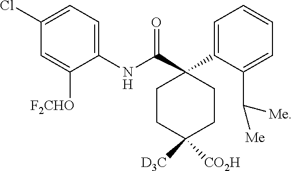

In embodiments, the compound is

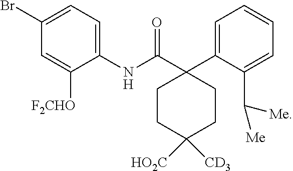

In embodiments, the compound is

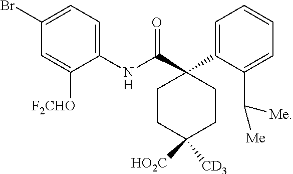

In embodiments, the compound is

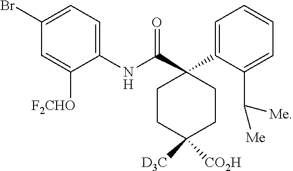

In embodiments, the compound is

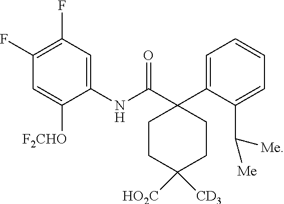

In embodiments, the compound is

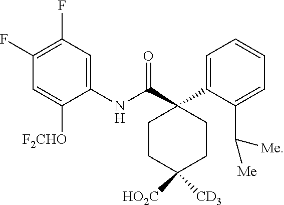

In embodiments, the compound is

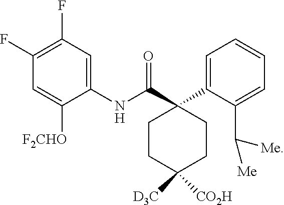

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

In embodiments, R¹⁰ is not hydrogen, halogen, —CH₃, or —OCH₃. In embodiments, R¹⁰ is not hydrogen. In embodiments, R¹⁰ is not halogen. In embodiments, R¹⁰ is not —F. In embodiments, R¹⁰ is not —Cl. In embodiments, R¹⁰ is not —CH₃. In embodiments, R¹⁰ is not substituted or unsubstituted heteroalkyl. In embodiments, R¹⁰ is not substituted or unsubstituted 2 to 6 membered heteroalkyl. In embodiments, R¹⁰ is not —OCH₃.

In embodiments,

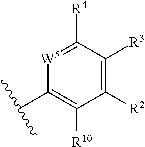

is not

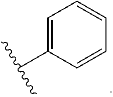 [SMILES: *c1ccccc1]

In embodiments,

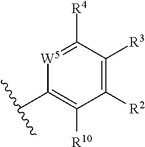

is not

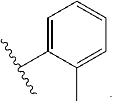 [SMILES: *c1ccccc1C]

In embodiments,

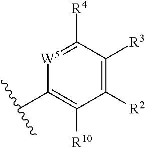

is not

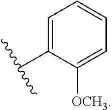 [SMILES: *c1ccccc1OC]

In embodiments,

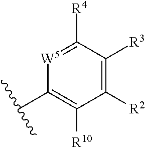

is not

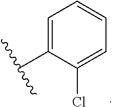 [SMILES: *c1ccccc1Cl]

In embodiments,

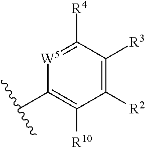

is not

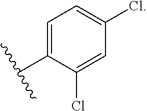 [SMILES: *c1ccc(Cl)cc1Cl]

In embodiments,

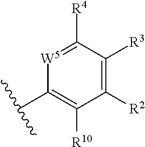

is not

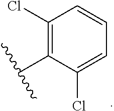 [SMILES: *c1c(Cl)cccc1Cl]

In embodiments,

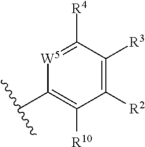

is not

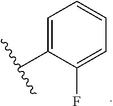 [SMILES: *c1ccccc1F]

In embodiments,

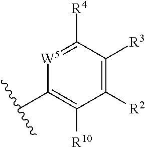

is not

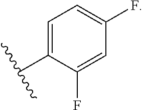 [SMILES: *c1ccc(F)cc1F]

In embodiments

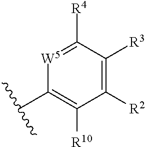

is not

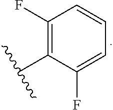 [SMILES: *c1c(F)cccc1F]

### III. Pharmaceutical Compositions

In an aspect is provided a pharmaceutical composition including a compound described herein, or a pharmaceutically acceptable salt or solvate thereof, and a pharmaceutically acceptable excipient.

In embodiments, the compound is a compound of formula (I), (I-1a), (I-1b), (I-1c), (I-1d), (I-2a), (I-2b), (I-2c), (I-2d), (I-3a), (I-3b), (I-3c), (I-3d), (I-4a), (I-4b), (I-4c), (I-4d), (II), (II-1a), (II-1b), (II-2a), (II-2b), (II-3a), or (II-3b). In embodiments, the compound is a compound of formula (I). In embodiments, the compound is a compound of formula (I-1a). In embodiments, the compound is a compound of formula (I-1b). In embodiments, the compound is a compound of formula (I-1c). In embodiments, the compound is a compound of formula (I-1d). In embodiments, the compound is a compound of formula (I-2a). In embodiments, the compound is a compound of formula (I-2b). In embodiments, the compound is a compound of formula (I-2c). In embodiments, the compound is a compound of formula (I-2d). In embodiments, the compound is a compound of formula (I-3a). In embodiments, the compound is a compound of formula (I-3b). In embodiments, the compound is a compound of formula (I-3c). In embodiments, the compound is a compound of formula (I-3d). In embodiments, the compound is a compound of formula (I-4a). In embodiments, the compound is a compound of formula (I-4b). In embodiments, the compound is a compound of formula (I-4c). In embodiments, the compound is a compound of formula (I-4d). In embodiments, the compound is a compound of formula (II). In embodiments, the compound is a compound of formula (II-1a). In embodiments, the compound is a compound of formula (II-1b). In embodiments, the compound is a compound of formula (II-2a). In embodiments, the compound is a compound of formula (II-2b). In embodiments, the compound is a compound of formula (II-3a). In embodiments, the compound is a compound of formula (II-3b).

In embodiments, the pharmaceutical composition includes an effective amount of the compound. In embodiments, the pharmaceutical composition includes a therapeutically effective amount of the compound.

### IV. Methods of Use

In an aspect is provided a method of treating a neurodegenerative disorder in a subject in need thereof, the method including administering to the subject in need thereof a therapeutically effective amount of a compound described herein, or a pharmaceutically acceptable salt or solvate thereof.

In an aspect is provided a method of treating an inflammatory disease in a subject in need thereof, the method including administering to the subject in need thereof a therapeutically effective amount of a compound described herein, or a pharmaceutically acceptable salt or solvate thereof.

In embodiments, the inflammatory disease is encephalitis. In embodiments, the inflammatory disease is post-hemorrhagic encephalitis. In embodiments, the inflammatory disease is ocular inflammation. In embodiments, the inflammatory disease is conjunctivitis. In embodiments, the inflammatory disease is allergic conjunctivitis. In embodiments, the inflammatory disease is vernal keratoconjunctivitis. In embodiments, the inflammatory disease is papillary conjunctivitis. In embodiments, the inflammatory disease is Sjogren's syndrome. In embodiments, the inflammatory disease is inflammatory disease with dry eyes.

In an aspect is provided a method of treating a demyelinating disease in a subject in need thereof, the method including administering to the subject in need thereof a therapeutically effective amount of a compound described herein, or a pharmaceutically acceptable salt or solvate thereof.

In embodiments, the demyelinating disease is a demyelinating disease of the central nervous system. In embodiments, the demyelinating disease is multiple sclerosis. In embodiments, the demyelinating disease is a demyelinating disease of the peripheral nervous system.

In an aspect is provided a method of treating fibrotic disease in a subject in need thereof, the method including administering to the subject in need thereof a therapeutically effective amount of a compound described herein, or a pharmaceutically acceptable salt or solvate thereof.

In embodiments, the fibrotic disease is pulmonary fibrosis. In embodiments, the fibrotic disease is skin fibrosis. In embodiments, the fibrotic disease is liver fibrosis. In embodiments, the fibrotic disease is ocular fibrosis. In embodiments, the fibrotic disease is idiopathic pulmonary fibrosis. In embodiments, the fibrotic disease is scleroderma. In embodiments, the fibrotic disease is nonalcoholic steatohepatitis. In embodiments, the fibrotic disease is ocular fibrosis. In embodiments, the fibrotic disease is hypertrophic scarring or keloids (e.g., burn induced or surgical, sarcoidosis, scleroderma, spinal cord injury/fibrosis, myelofibrosis, vascular restenosis, atherosclerosis, arteriosclerosis, Wegener's granulomatosis, mixed connective tissue disease, or Peyronie's disease). In embodiments, the fibrotic disease is iatrogenic pulmonary fibrosis. In embodiments, the fibrotic disease is radiation-induced fibrosis. In embodiments, the fibrotic disease is silicosis-induced pulmonary fibrosis. In embodiments, the fibrotic disease is asbestos-induced pulmonary fibrosis. In embodiments, the fibrotic disease is pleural fibrosis. In embodiments, the fibrotic disease is pulmonary fibrosis associated with SARS-CoV-2 infection and/or COVID-19. In embodiments, the fibrotic disease is pulmonary fibrosis secondary to systemic inflammatory disease. In embodiments, the fibrotic disease is pulmonary fibrosis secondary to sarcoidosis. In embodiments, the fibrotic disease is gut fibrosis. In embodiments, the fibrotic disease is head and neck fibrosis. In embodiments, the fibrotic disease is cirrhosis. In embodiments, the fibrotic disease is alcohol-induced liver fibrosis. In embodiments, the fibrotic disease is endometriosis. In embodiments, the fibrotic disease is spinal cord fibrosis. In embodiments, the fibrotic disease is myelofibrosis. In embodiments, the fibrotic disease is cardiac fibrosis. In embodiments, the fibrotic disease is perivascular fibrosis. In embodiments, the fibrotic disease is Peyronie's disease. In embodiments, the fibrotic disease is abdominal or bowel adhesions. In embodiments, the fibrotic disease is bladder fibrosis. In embodiments, the fibrotic disease is fibrosis of the nasal passages. In embodiments, the fibrotic disease is fibrosis mediated by fibroblasts. In embodiments, the fibrotic disease is renal fibrosis associated with chronic kidney disease (CKD).

In an aspect is provided a method of treating cancer in a subject in need thereof, the method including administering to the subject in need thereof a therapeutically effective amount of a compound described herein, or a pharmaceutically acceptable salt or solvate thereof.

In embodiments, the cancer is brain cancer. In embodiments, the cancer is glioblastoma. In embodiments, the cancer is a solid tumor (e.g., of the bladder, bowel, brain, breast, endometrium, heart, kidney, lung, lymphatic tissue (e.g., lymphoma), ovary, pancreas or other endocrine organ (e.g., thyroid), prostate, skin (e.g., melanoma or basal cell cancer)) or hematological tumors (e.g., leukemia) at any stage of the disease with or without metastases. In embodiments, the cancer is acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (e.g., osteosarcoma or malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-cell lymphoma, embryonal tumors, endometrial cancer, ependymoblastoma, ependymoma, esophageal cancer, Ewing sarcoma family of tumors, eye cancer, retinoblastoma, gallbladder cancer, gastrointestinal carcinoid tumor, gastrointestinal stromal tumor (GIST), germ cell tumor, glioma, hairy cell leukemia, head and neck cancer, hepatocellular cancer, Hodgkin lymphoma, hypopharyngeal cancer, intraocular melanoma, islet cell tumors (e.g., endocrine pancreas), Kaposi's sarcoma, kidney cancer, Langerhans cell histiocytosis, laryngeal cancer, leukemia, liver cancer, lymphoma, medulloblastoma, medulloepithelioma, melanoma, mesothelioma, mouth cancer, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell cancer, retinoblastoma, rhabdomyosarcoma, salivary gland cancer, sarcoma, Sezary syndrome, skin cancer, small cell lung cancer, small intestine cancer, soft tissue sarcoma, squamous cell carcinoma, stomach (e.g., gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, or Wilms' tumor.

In an aspect is provided a method of treating an LPAR1-associated disease in a subject in need thereof, the method including administering to the subject in need thereof a therapeutically effective amount of a compound described herein, or a pharmaceutically acceptable salt or solvate thereof.

In embodiments, the LPAR1-associated disease is a neurodegenerative disease. In embodiments, the LPAR1-associated disease is an inflammatory disease. In embodiments, the LPAR1-associated disease is post-hemorrhagic encephalitis. In embodiments, the LPAR1-associated disease is a demyelinating disease. In embodiments, the LPAR1-associated disease is multiple sclerosis. In embodiments, the LPAR1-associated disease is a fibrotic disease. In embodiments, the LPAR1-associated disease is pulmonary fibrosis. In embodiments, the LPAR1-associated disease is idiopathic pulmonary fibrosis. In embodiments, the LPAR1-associated disease is cancer (e.g., brain cancer, ovarian cancer, colon cancer, prostate cancer, breast cancer, melanoma, head and neck cancer, bowel cancer, colorectal cancer, or thyroid cancer). In embodiments, the LPAR1-associated disease is pain (e.g., neuropathic pain, acute pain, or chronic pain).

In embodiments, the LPAR1-associated disease is a respiratory or allergic disorder. In embodiments, the respiratory or allergic disorder is asthma, peribronchiolar fibrosis, obliterative bronchiolitis, or chronic obstructive pulmonary disease (COPD). In embodiments, the COPD is chronic bronchitis or emphysema, pulmonary hypertension, interstitial lung fibrosis and/or airway inflammation, or cystic fibrosis. In embodiments, the respiratory disease is adult respiratory distress syndrome or allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, and hypoxia.

In embodiments, the LPAR1-associated disease is a nervous system disorder. In embodiments, the nervous system disorder is Alzheimer's Disease, cerebral edema, cerebral ischemia, stroke, multiple sclerosis, neuropathies, Parkinson's Disease, a nervous condition found after blunt or surgical trauma (including post-surgical cognitive dysfunction and spinal cord or bram stem injury), degenerative disk disease, or sciatica.

In embodiments, the LPAR1-associated disease is a cardiovascular disorder. In embodiments, the cardiovascular disorder is arrhythmia (e.g., atrial or ventricular); atherosclerosis and its sequelae; angina; cardiac rhythm disturbances; myocardial ischemia; myocardial infarction; cardiac or vascular aneurysm; vasculitis; stroke; peripheral obstructive arteriopathy of a limb, an organ, or a tissue; reperfusion injury following ischemia of the brain, heart or other organ or tissue; endotoxic, surgical, or traumatic shock; hypertension; valvular heart disease; heart failure; abnormal blood pressure; shock; vasoconstriction (including that associated with migraines); vascular abnormality, or a cardiovascular insufficiency limited to a single organ or tissue.

In embodiments, the LPAR1-associated disease is lung fibrosis, kidney fibrosis, liver fibrosis, scarring, asthma, rhinitis, chronic obstructive pulmonary disease (COPD), pulmonary hypertension, interstitial lung fibrosis, arthritis, allergy, psoriasis, inflammatory bowel disease, adult respiratory distress syndrome, myocardial infarction, aneurysm, stroke, cancer, pain, proliferative disorders, or inflammatory conditions.

In embodiments, the LPAR1-associated disease is a liver disease. In embodiments, the liver disease is hepatitis C, liver cancer, familial combined hyperlipidemia, non-alcoholic fatty liver disease (NAFLD), progressive familial intrahepatic cholestasis, primary biliary cirrhosis (PBC), or primary sclerosing cholangitis (PSC). In embodiments, the liver disease is primary sclerosing cholangitis (PSC). In embodiments, the liver disease includes portal hypertension. In embodiments, liver cancer includes hepatocellular carcinoma (HCC), cholangiocarcinoma, angiosarcoma, or hemangiosarcoma. In embodiments, NAFLD includes steatosis. In embodiments, NAFLD includes NASH. In embodiments, NAFLD or NASH includes liver fibrosis. In embodiments, NAFLD or NASH includes liver cirrhosis. In embodiments, NAFLD or NASH includes compensated liver cirrhosis. In embodiments, NAFLD or NASH includes decompensated liver fibrosis. In embodiments, NAFLD includes hepatocellular carcinoma (HCC). In embodiments, the liver disease is NASH.

In an aspect is provided a method of modulating LPAR1 activity in a subject, the method including administering to the subject a compound described herein, or a pharmaceutically acceptable salt or solvate thereof.

### V. Embodiments

Embodiment P1. A compound, or a pharmaceutically acceptable salt or solvate thereof, having the formula:

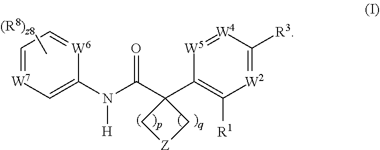

wherein
R¹ is unsubstituted C₂-C₅ alkyl;
W² is N or C(R²
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —N^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁴ is N or C(R⁴);
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —N^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁵ is N or C(R⁵);
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted cycloalkyl, substituted or unsubstituted heterocycloalkyl, substituted or unsubstituted aryl, or substituted or unsubstituted heteroaryl;
R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl;
W⁶ is N or C(R⁶)
R⁶ is hydrogen, halogen, —CX⁶³, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —NR^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}—NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁷ is N, N⁺—O⁻, or C(R⁷);
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —N₇^{A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl;
Z is O, C(R¹¹)(R¹²), or C(O)N(R¹³)
R¹¹ is hydrogen, halogen, —CX¹¹₃, —CHX¹¹₂, —CH₂X¹¹, —OCX¹¹₃, —OCH₂X¹¹, —OCHX¹¹₂, —CN, —SOₙ₁₁R^{11D}, —SO^{v11}NR^{11A}R^{11B}, —NR^{11C}NR^{11A}R^{11B}, —ONR^{11A}R^{11B}, —NHC(O)NR^{11C}NR^{11A}R^{11B}, —NHC(O)NR^{11A}R^{11B}, —N(O)ₘ₁₁, —NR^{11A}R^{11B}, —C(O)R^{11C}, —C(O)OR^{11C}, —C(O)NR^{11A}R^{11B}, —OR^{11D}, —SR^{11D}, —NR^{11A}SO₂R^{11D}, —NR^{11A} C(O)R^{11C}, —NR^{11A} C(O)OR^{11C}, —NR^{11A} OR^{11C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —CN, —SOₙ₁₂R^{12D}, —SOᵥ₁₂NR^{12A}R^{12B}, —NR^{12C}NR^{12A}R^{12B} ONR^{12A}R^{12B}, —NHC(O)NR^{12C}NR^{12A}R^{12B}, —NHC(O)NR^{12A}R^{12B}, —N(O)ₘ₁₂, —NR^{12A}R^{12B}, —C(O)R^{12C}, —C(O)OR^{12C}, —C(O)NR^{12A}R^{12B}, —OR^{12D}, —SR^{12D}, —NR^{12A}SO₂R^{12D}, —NR^{12A}C(O)R^{12C}, —NR^{12A}C(O)OR^{12C}, —NR^{12A} OR^{12C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹¹ and R¹² substituents may optionally be joined to form an oxo, substituted or unsubstituted cycloalkyl, or substituted or unsubstituted heterocycloalkyl;
R¹³ is hydrogen, halogen, —CX¹³₃, —CHX¹³₂, —CH₂X¹³, —OCX¹³₃, —OCH₂X¹³, —OCHX¹³₂, —SOₙ₁₃R^{13D}, —SOᵥ₁₃NR^{13A}R^{13B}, —C(O)R^{13C}, —C(O)OR^{13C}, —C(O)NR^{13A}R^{13B}, —OR^{13D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, R^{8D}, R^{11A}, R^{11B}, R^{11C}, R^{11D}, R^{12A}, R^{12B}, R^{12C}, R^{12D}R^{13A}, R^{13B}, R^{13C}, and R^{13D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{12A} and R^{12B} substituents bonded to the same nitrogen atom may optionally be joined to form a substituted or unsubstituted heterocycloalkyl or substituted or unsubstituted heteroaryl; R^{13A} and R^{13B} substituents bonded to the same nitrogen atom may optionally be joined to form a substituted or unsubstituted heterocycloalkyl or substituted or unsubstituted heteroaryl;
X², X³, X⁴, X⁵, X⁶, X⁷, X⁸, X¹¹, X¹², and X¹³ are independently —F, —Cl, —Br, or —I;
n2, n3, n4, n5, n6, n7, n8, n11, n12, and n13 are independently an integer from 0 to 4;
m2, m3, m4, m5, m6, m7, m8,m11, m12, v2, v3, v4, v5, v6, v7, v8, v11, v12, and v13 are independently 1 or 2;
z8 is an integer from 0 to 3;
p is 1 or 2; and
q is 1 or 2.

Embodiment P2. The compound of embodiment P1, having the formula:

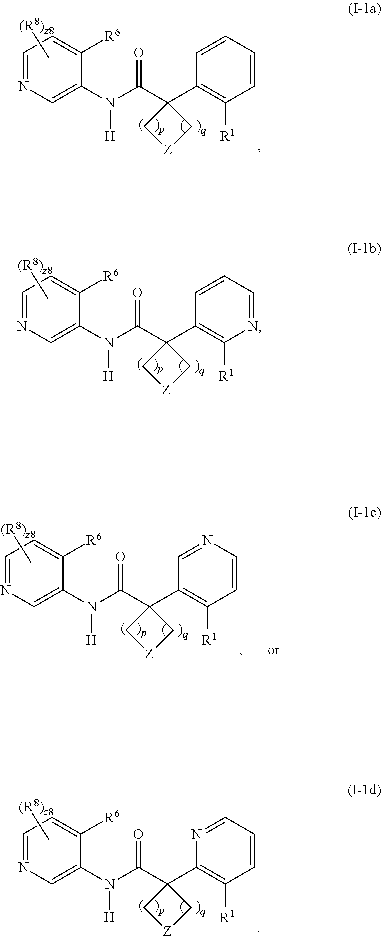

Embodiment P3. The compound of embodiment P1, having the formula:

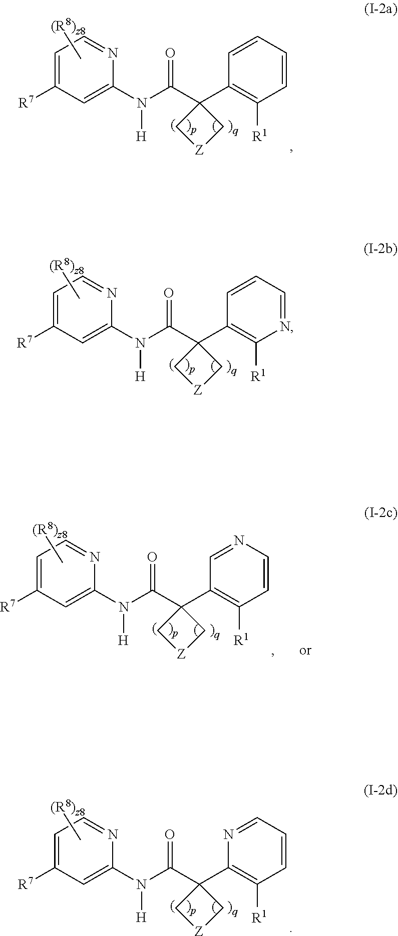

Embodiment P4. The compound of embodiment P1, having the formula:

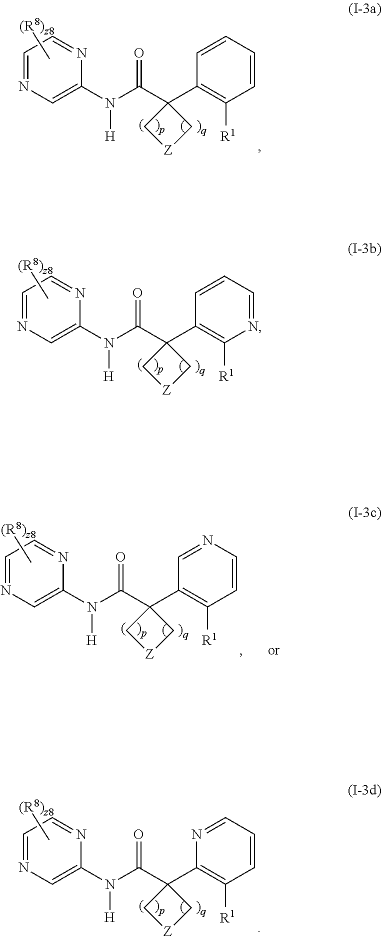

Embodiment P5. The compound of embodiment P1, having the formula:

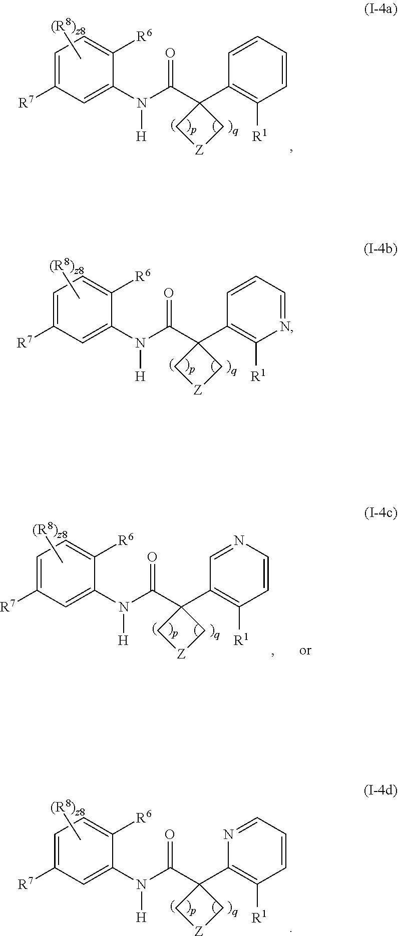

Embodiment P6. The compound of one of embodiments P1 to P5, wherein R¹ is unsubstituted C₃ alkyl.

Embodiment P7. The compound of one of embodiments P1 to P5, wherein R¹ is isopropyl.

Embodiment P8. A compound, or a pharmaceutically acceptable salt or solvate thereof, having the formula:

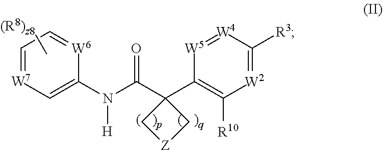

wherein
W² is N or C(R²)
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —N^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁴ is N or C(R⁴);
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —N^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁵ is N or C(R⁵);
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —N^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted cycloalkyl, substituted or unsubstituted heterocycloalkyl, substituted or unsubstituted aryl, or substituted or unsubstituted heteroaryl;
R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl;
W⁶ is N or C(R⁶)
R⁶ is hydrogen, halogen, —CX⁶₃, —CHX⁶₂, —CH₂X⁶, —OCX⁶₃, —OCH₂X⁶, —OCHX⁶₂, —CN, —SOₙ₅R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NHC(O)NR^{6A}R^{6B}, —N(O)ₘ₆, —N^{6A}R^{6B}, —C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁷ is N, N⁺—O⁻, or C(R⁷);
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —NR^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{98A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl;
R¹⁰ is hydrogen, halogen, —CX¹⁰₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R² substituents may optionally be joined to form a substituted or unsubstituted cycloalkyl, substituted or unsubstituted heterocycloalkyl, substituted or unsubstituted aryl, or substituted or unsubstituted heteroaryl;
Z is O, C(R¹¹)(R¹²), or C(O)N(R¹³)
R¹¹ is hydrogen, halogen, —CX¹¹₃, —CHX¹¹₂, —CH₂X¹¹, —OCX¹¹₃, —OCH₂X¹¹, —OCHX¹¹₂, —CN, —SOₙ₁₁R_{11D}, —SOᵥ₁₁NR^{11A}R^{11B}, —NR^{11C}NR^{11A}R^{11B}, —ONR^{11A}R^{11B}, —NHC(O)NR^{11C}NR^{11A}R^{11B}, —NHC(O)NR^{11A}R^{11B}, —N(O)ₘ₁₁, —NR^{11A}R^{11B}, —C(O)R^{11C}, —C(O)OR^{11C}, —C(O)NR^{11A}R^{11B}, —OR^{11D}, —SR^{11D}, —NR^{11A} SO₂R^{11D}, —NR^{11A} C(O)R^{11C}, —NR^{11A} C(O)OR^{11C}, —NR^{11A} OR^{11C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —CN, —SOₙ₁₂R^{12D}, —SOᵥ₁₂NR^{12A}R^{12B}, —NR^{12C}NR^{12A}R^{12B} ONR^{12A}R^{12B}, —NHC(O)NR^{12C}NR^{12A}R^{12B}, —NHC(O)NR^{12A}R^{12B}, —N(O)ₘ₁₂, —NR^{12A}R^{12B}, —C(O)R^{12C}, —C(O)OR^{12C}, —C(O)NR^{12A}R^{12B}, —OR^{12D}, —SR^{12D}, —NR^{12A}SO₂R^{12D}, —NR^{12A}C(O)R^{12C}, —NR^{12A}C(O)OR^{12C}, —NR^{12A} OR^{12C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹¹ and R¹² substituents may optionally be joined to form an oxo, substituted or unsubstituted cycloalkyl, or substituted or unsubstituted heterocycloalkyl;
R¹³ is hydrogen, halogen, —CX¹³₃, —CHX¹³₂, —CH₂X¹³, —OCX¹³₃, —OCH₂X¹³, —OCHX¹³₂, —SOₙ₁₃R^{13D}, —SOᵥ₁₃NR^{13A}R^{13B}, —C(O)R^{13C}, —C(O)OR^{13C}, —C(O)NR^{13A}R^{13B}, —OR^{13D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, R^{8D}, R^{10A}, R^{10B}, R^{10C}, R^{10D}, R^{11A}, R^{11B}, R^{11C}, R^{11D}R^{12A}, R^{12B}, R^{12C}, R^{12D}, R^{13A}, R^{13B}, R^{13C}, and R^{13D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{12A} and R^{12B} substituents bonded to the same nitrogen atom may optionally be joined to form a substituted or unsubstituted heterocycloalkyl or substituted or unsubstituted heteroaryl; R^{13A} and R^{13B} substituents bonded to the same nitrogen atom may optionally be joined to form a substituted or unsubstituted heterocycloalkyl or substituted or unsubstituted heteroaryl;
X², X³, X⁴, X⁵, X⁶, X⁷, X⁸, X¹⁰, X, X², and X¹³ are independently —F, —Cl, —Br, or —I;
n2, n3, n4, n5, n6, n7, n8, n10, n11, n12, and n13 are independently an integer from 0 to 4;
m2, m3, m4, m5, m6, m7, m8, m10, m11, m12, v2, v3, v4, v5, v6, v7, v8, v10, v11, v12, and v13 are independently 1 or 2;
z8 is an integer from 0 to 3;
p is 1 or 2; and
q is 1 or 2;
wherein at least one of W⁶ or W⁷ is N;
wherein if W⁶ is N and W⁷ is C(R⁷), then R¹⁰ is not hydrogen, —F, —Cl, —CH₃, or —OCH₃;
wherein if W⁶ is C(R⁶) and W⁷ is N, then R¹⁰ is not hydrogen, —F, —Cl, —CH₃ or —OCH₃; and
wherein if W⁶ and W⁷ are N and Z is O or CH₂, then R¹⁰ is not hydrogen, —F, —Cl, —CH₃ or —OCH₃.

Embodiment P9. The compound of embodiment P8, having the formula:

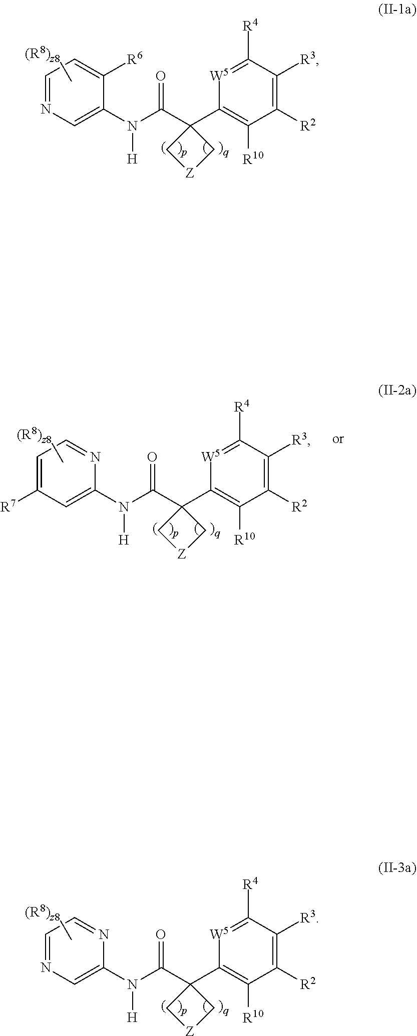

Embodiment P10. The compound of embodiment P8, having the formula:

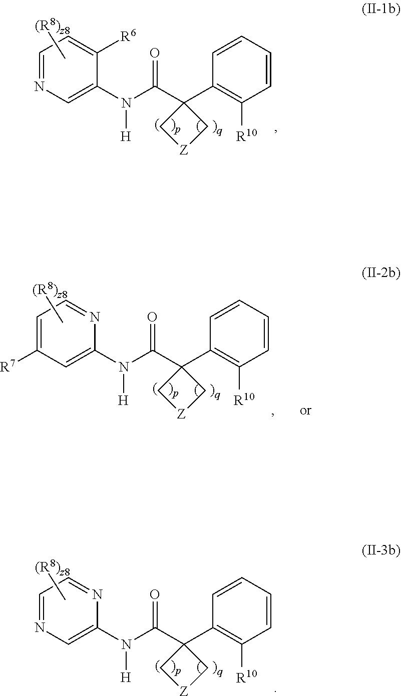

Embodiment P11. The compound of one of embodiments P8 to P10, wherein R¹⁰ is hydrogen or unsubstituted C₁-C₆ alkyl.

Embodiment P12. The compound of one of embodiments P8 to P10, wherein R¹⁰ is isopropyl.

Embodiment P13. The compound of one of embodiments P1 to P12, wherein R⁸ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P14. The compound of one of embodiments P1 to P12, wherein R⁸ is independently halogen, —CF₃, —CHF₂, —OCHF₂, —OR^{8D}, unsubstituted C₁-C₆ alkyl, or unsubstituted 2 to 8 membered heteroalkyl.

Embodiment P15. The compound of embodiment P14, wherein R^{8D} is independently unsubstituted C₁-C₆ alkyl or unsubstituted C₃-C₈ cycloalkyl.

Embodiment P16. The compound of one of embodiments P1 to P12, wherein R⁸ is independently —F, —Cl, —Br, —CHF₂, —OCHF₂, —OCH₃, —OCH(CH₃)₂, —OCH(CH₃)CH₂OCH₃, —O-(unsubstituted cyclopropyl), or unsubstituted methyl.

Embodiment P17. The compound of one of embodiments P1 to P16, wherein

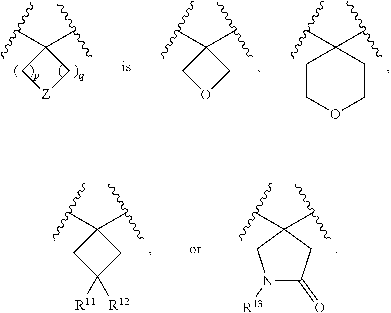

Embodiment P18. The compound of one of embodiments P1 to P17, wherein

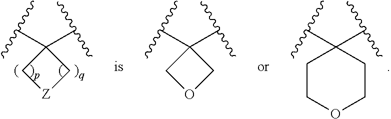

Embodiment P19. The compound of embodiment P17, wherein R¹¹ is hydrogen, halogen, —SOₙ₁₁R^{11D}, —SOᵥ₁₁NR^{11A}R^{11B}, —NR^{11A}R^{11B}, —C(O)OR^{11C}, —C(O)NR^{11A}R^{11B}, —OR^{11D}, —NR^{11A} SO₂R^{11D}, —NR^{11A} C(O)R^{11C}, substituted or unsubstituted C₁-C₆ alkyl, substituted or unsubstituted 2 to 6 membered heteroalkyl, substituted or unsubstituted 3 to 8 membered heterocycloalkyl, or substituted or unsubstituted 5 to 6 membered heteroaryl.

Embodiment P20. The compound of embodiment P19, wherein R^{11A} and R^{11B} are independently hydrogen, unsubstituted C₁-C₆ alkyl, or unsubstituted C₃-C₈ cycloalkyl.

Embodiment P21. The compound of embodiment P19, wherein R^{11C} is hydrogen or unsubstituted C₁-C₆ alkyl.

Embodiment P22. The compound of embodiment P19, wherein R^{11D} is hydrogen or unsubstituted C₁-C₆ alkyl.

Embodiment P23. The compound of embodiment P17, wherein R¹¹ is hydrogen, —F, —OH, —CH₃, —CH₂CH₃, —NHCH₃, —N(CH₃)₂, —C(O)OH, —C(O)N(CH₃)₂, —S(O)₂CH₃, —S(O)₂NH₂, —S(O)₂NH(CH₃)₂,

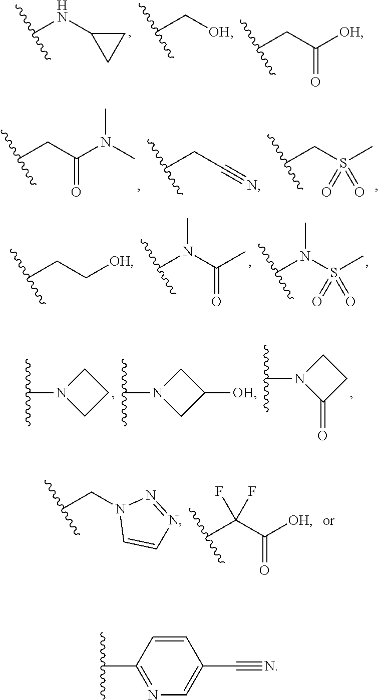

Embodiment P24. The compound of embodiment P17, wherein R¹² is hydrogen, halogen, —SOₙ₁₂R^{12D}, —SOᵥ₁₂NR^{12A}R^{12B}, —NR^{12A}R^{12B}, —C(O)OR^{12C}, —C(O)NR^{12A}R^{12B}, —OR^{12D}, —NR^{12A} SO₂R^{12D}, —NR^{12A}C(O)R^{12C}, substituted or unsubstituted C₁-C₆ alkyl, substituted or unsubstituted 2 to 6 membered heteroalkyl, substituted or unsubstituted 3 to 8 membered heterocycloalkyl, or substituted or unsubstituted 5 to 6 membered heteroaryl.

Embodiment P25. The compound of embodiment P24, wherein R^{12A} and R^{12B} are independently hydrogen, unsubstituted C₁-C₆ alkyl, or unsubstituted C₃-C₈ cycloalkyl.

Embodiment P26. The compound of embodiment P24, wherein R^{12C} is hydrogen or unsubstituted C₁-C₆ alkyl.

Embodiment P27. The compound of embodiment P24, wherein R^{12D} is hydrogen or unsubstituted C₁-C₆ alkyl.

Embodiment P28. The compound of embodiment P17, wherein R¹² is hydrogen, —F, —OH, —CH₃, —CH₂CH₃, —NHCH₃, —N(CH₃)₂, —C(O)OH, —C(O)N(CH₃)₂, —S(O)₂CH₃, —S(O)₂NH₂, —S(O)₂NH(CH₃)₂

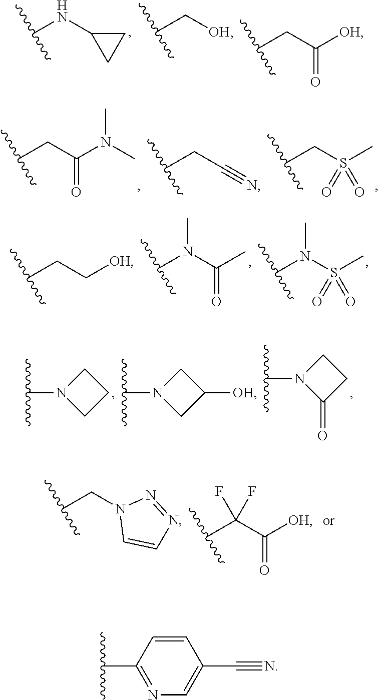

Embodiment P29. The compound of embodiment P17, wherein R¹¹ and R¹² substituents are joined to form a substituted or unsubstituted C₃-C₈ cycloalkyl or substituted or unsubstituted 3 to 8 membered heterocycloalkyl.

Embodiment P30. The compound of one of embodiments P1 to P17, wherein

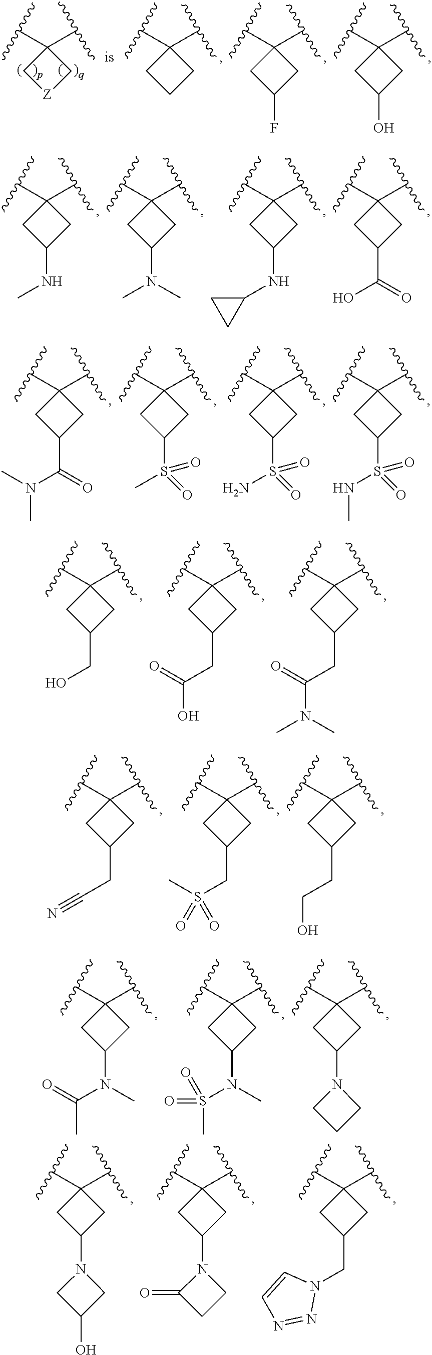

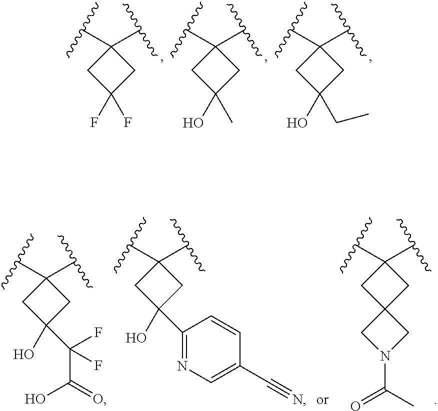

Embodiment P31. The compound of embodiment P17, wherein R¹³ is hydrogen, unsubstituted C₁-C₆ alkyl, or unsubstituted 2 to 6 membered heteroalkyl.

Embodiment P32. The compound of one of embodiments P1 to P17, wherein

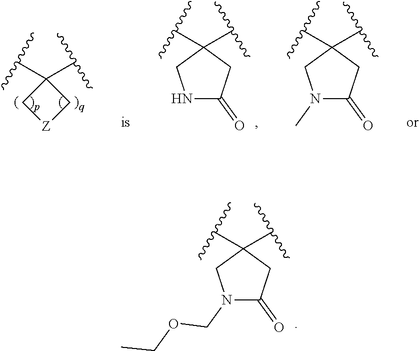

Embodiment P33. A compound, or a pharmaceutically acceptable salt or solvate thereof, having the formula:

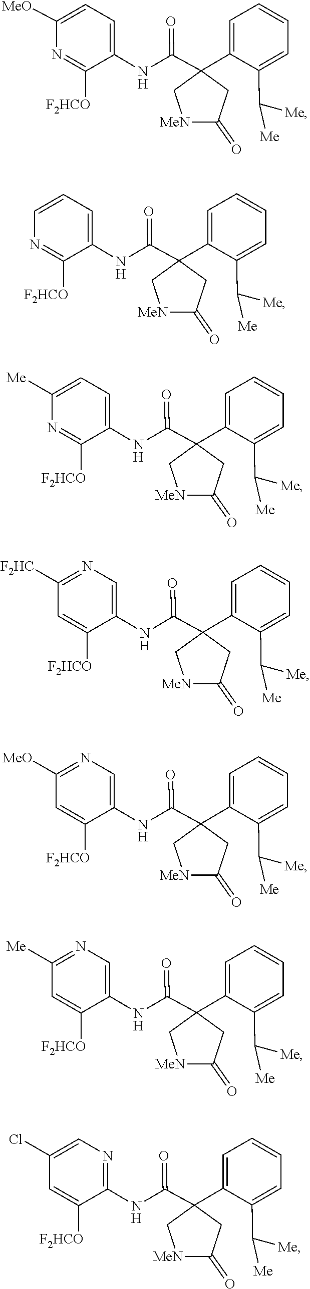

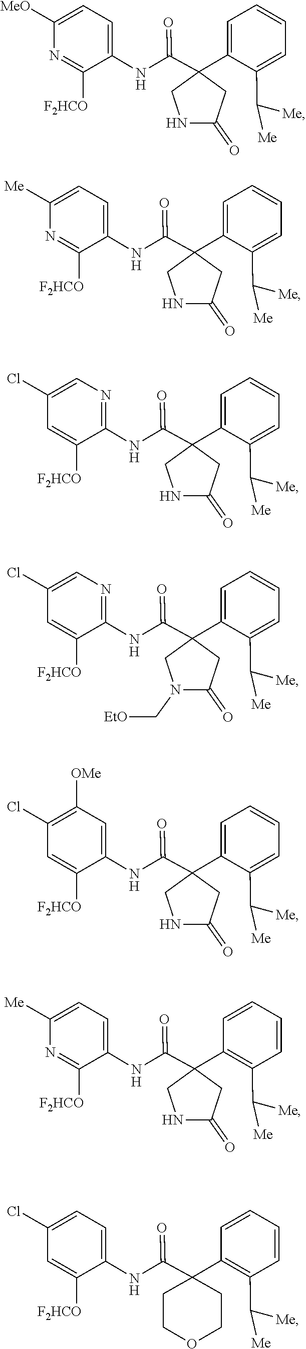

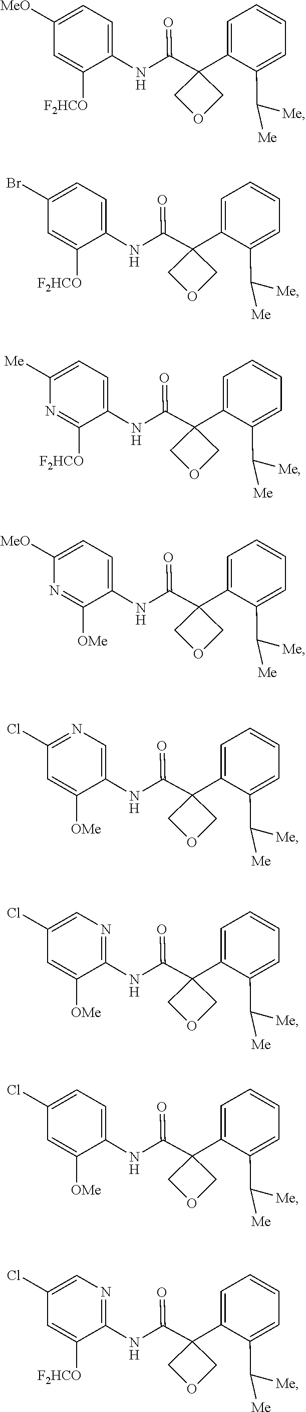

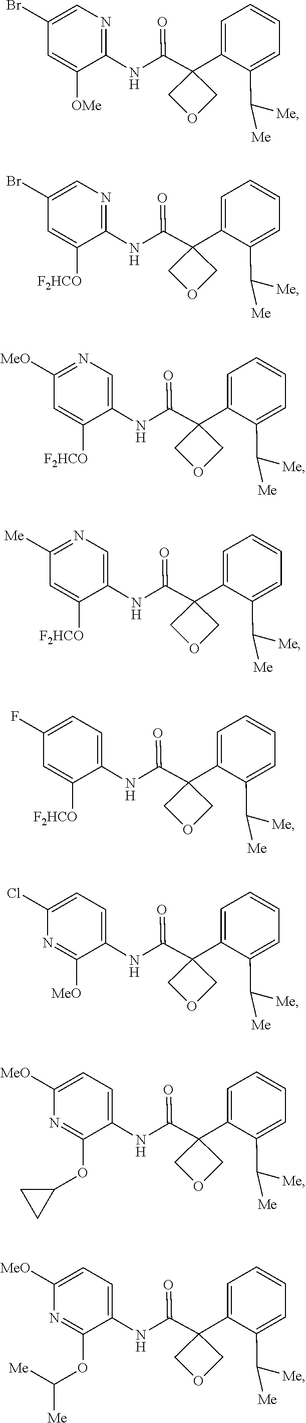

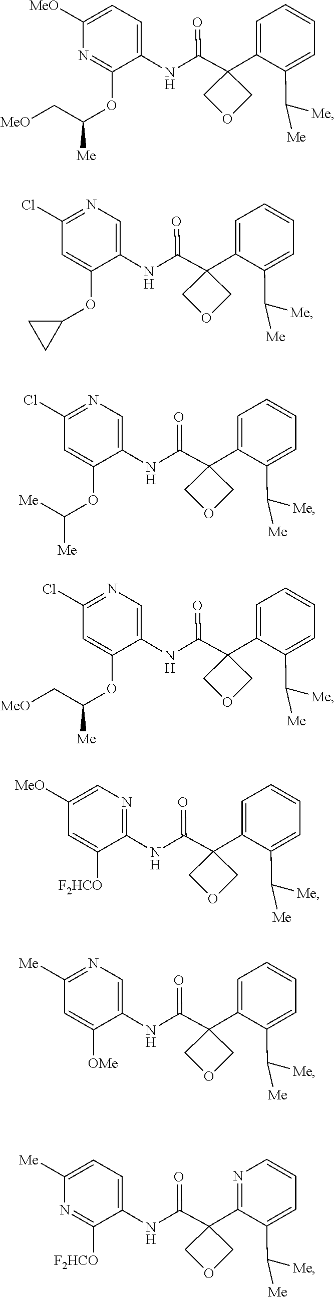

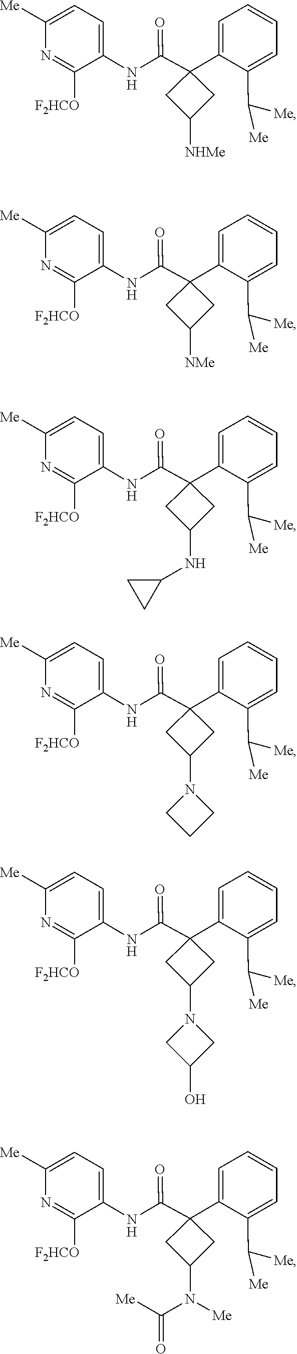

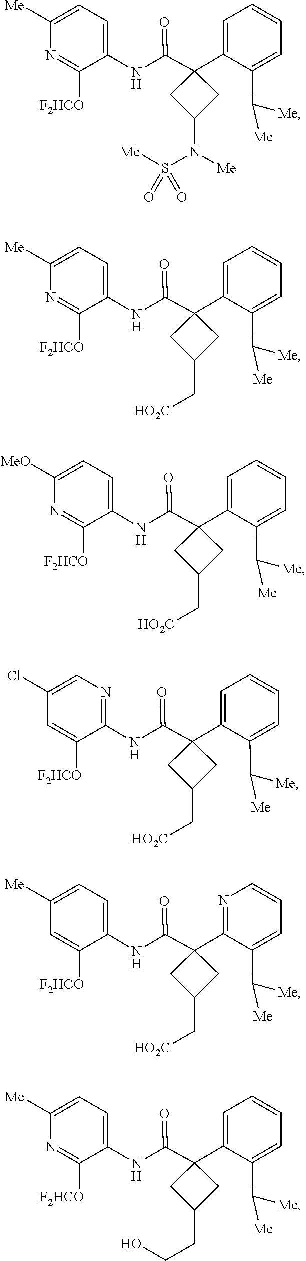 [SMILES: Cc1ccc(NC(=O)C2(c3ccccc3C(C)C)CC(N(C)S(C)(=O)=O)C2)c(OC(F)F)n1 ; Cc1ccc(NC(=O)C2(c3ccccc3C(C)C)CC(CC(=O)O)C2)c(OC(F)F)n1 ; COc1ccc(NC(=O)C2(c3ccccc3C(C)C)CC(CC(=O)O)C2)c(OC(F)F)n1 ; CC(C)c1ccccc1C1(C(=O)Nc2ncc(Cl)cc2OC(F)F)CC(CC(=O)O)C1 ; Cc1ccc(NC(=O)C2(c3ncccc3C(C)C)CC(CC(=O)O)C2)c(OC(F)F)c1 ; Cc1ccc(NC(=O)C2(c3ccccc3C(C)C)CC(CCO)C2)c(OC(F)F)n1]

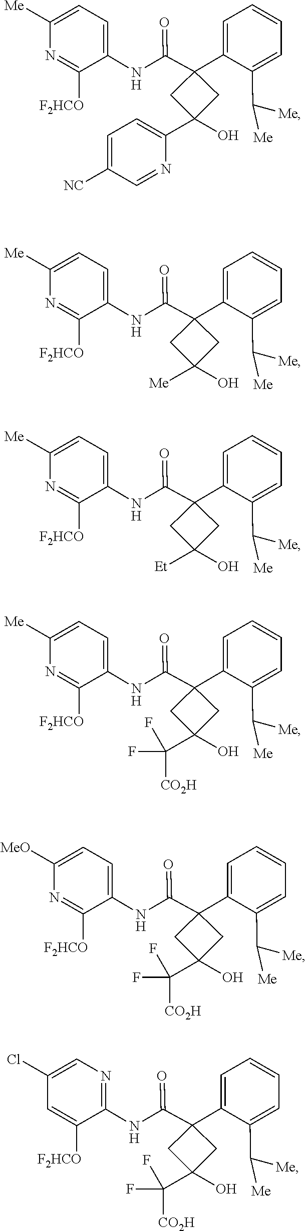 [SMILES: Cc1ccc(NC(=O)C2(c3ccccc3C(C)C)CC(O)(c3ccc(C#N)cn3)C2)c(OC(F)F)n1 ; Cc1ccc(NC(=O)C2(c3ccccc3C(C)C)CC(C)(O)C2)c(OC(F)F)n1 ; CCC1(O)CC(C(=O)Nc2ccc(C)nc2OC(F)F)(c2ccccc2C(C)C)C1 ; Cc1ccc(NC(=O)C2(c3ccccc3C(C)C)CC(O)(C(F)(F)C(=O)O)C2)c(OC(F)F)n1 ; COc1ccc(NC(=O)C2(c3ccccc3C(C)C)CC(O)(C(F)(F)C(=O)O)C2)c(OC(F)F)n1 ; CC(C)c1ccccc1C1(C(=O)Nc2ncc(Cl)cc2OC(F)F)CC(O)(C(F)(F)C(=O)O)C1]

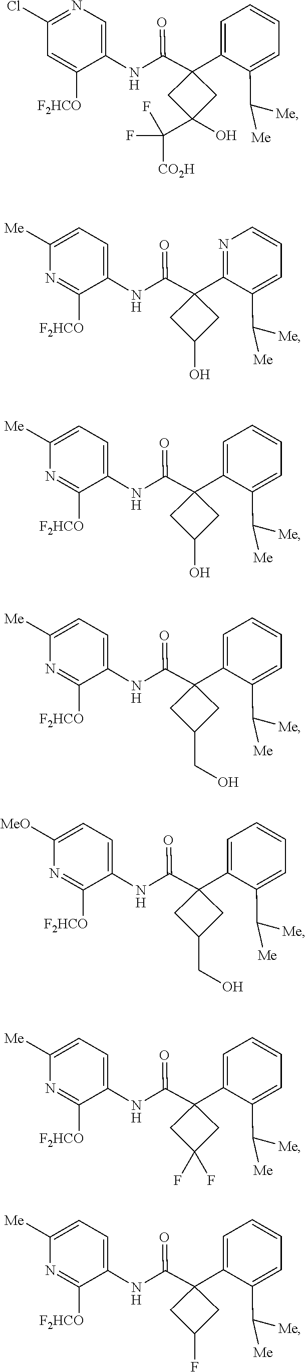

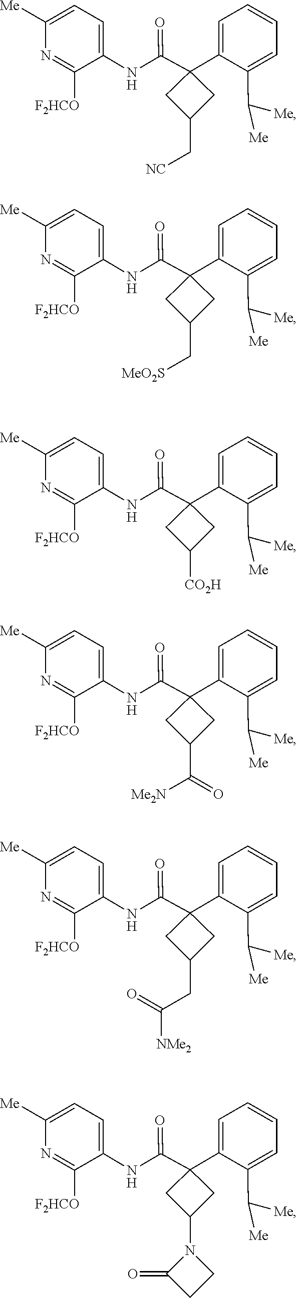 [SMILES: Cc1ccc(NC(=O)C2(c3ccccc3C(C)C)CC(CC#N)C2)c(OC(F)F)n1 ; Cc1ccc(NC(=O)C2(c3ccccc3C(C)C)CC(CS(C)(=O)=O)C2)c(OC(F)F)n1 ; Cc1ccc(NC(=O)C2(c3ccccc3C(C)C)CC(C(=O)O)C2)c(OC(F)F)n1 ; Cc1ccc(NC(=O)C2(c3ccccc3C(C)C)CC(C(=O)N(C)C)C2)c(OC(F)F)n1 ; Cc1ccc(NC(=O)C2(c3ccccc3C(C)C)CC(CC(=O)N(C)C)C2)c(OC(F)F)n1 ; Cc1ccc(NC(=O)C2(c3ccccc3C(C)C)CC(N3CCC3=O)C2)c(OC(F)F)n1]

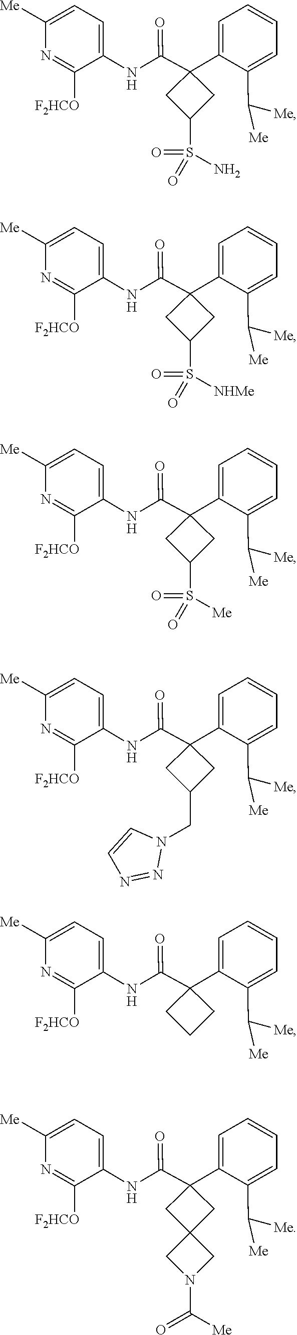 [SMILES: Cc1ccc(NC(=O)C2(c3ccccc3C(C)C)CC(S(N)(=O)=O)C2)c(OC(F)F)n1 ; CNS(=O)(=O)C1CC(C(=O)Nc2ccc(C)nc2OC(F)F)(c2ccccc2C(C)C)C1 ; Cc1ccc(NC(=O)C2(c3ccccc3C(C)C)CC(S(C)(=O)=O)C2)c(OC(F)F)n1 ; Cc1ccc(NC(=O)C2(c3ccccc3C(C)C)CC(Cn3ccnn3)C2)c(OC(F)F)n1 ; Cc1ccc(NC(=O)C2(c3ccccc3C(C)C)CCC2)c(OC(F)F)n1 ; CC(=O)N1CC2(C1)CC(C(=O)Nc1ccc(C)nc1OC(F)F)(c1ccccc1C(C)C)C2]

Embodiment P34. A pharmaceutical composition comprising the compound of one of embodiments P1 to P33, or a pharmaceutically acceptable salt or solvate thereof, and a pharmaceutically acceptable excipient.

Embodiment P35. A method of treating a neurodegenerative disorder in a subject in need thereof, said method comprising administering to the subject in need thereof a therapeutically effective amount of a compound of one of embodiments P1 to P33, or a pharmaceutically acceptable salt or solvate thereof.

Embodiment P36. A method of treating an inflammatory disease in a subject in need thereof, said method comprising administering to the subject in need thereof a therapeutically effective amount of a compound of one of embodiments P1 to P33, or a pharmaceutically acceptable salt or solvate thereof.

Embodiment P37. The method of embodiment P36, wherein the inflammatory disease is encephalitis.

Embodiment P38. The method of embodiment P37, wherein the encephalitis is post-hemorrhagic encephalitis.

Embodiment P39. A method of treating a demyelinating disease in a subject in need thereof, said method comprising administering to the subject in need thereof a therapeutically effective amount of a compound of one of embodiments P1 to P33, or a pharmaceutically acceptable salt or solvate thereof.

Embodiment P40. The method of embodiment P39, wherein the demyelinating disease is a demyelinating disease of the central nervous system.

Embodiment P41. The method of embodiment P40, wherein the demyelinating disease is multiple sclerosis.

Embodiment P42. The method of embodiment P39, wherein the demyelinating disease is a demyelinating disease of the peripheral nervous system.

Embodiment P43. A method of treating a fibrotic disease in a subject in need thereof, said method comprising administering to the subject in need thereof a therapeutically effective amount of a compound of one of embodiments P1 to P33, or a pharmaceutically acceptable salt or solvate thereof.

Embodiment P44. The method of embodiment P43, wherein the fibrotic disease is pulmonary fibrosis, skin fibrosis, liver fibrosis, or ocular fibrosis.

Embodiment P45. The method of embodiment P43, wherein the fibrotic disease is idiopathic pulmonary fibrosis, scleroderma, nonalcoholic steatohepatitis, or ocular fibrosis.

Embodiment P46. A method of treating cancer in a subject in need thereof, said method comprising administering to the subject in need thereof a therapeutically effective amount of a compound of one of embodiments P1 to P33, or a pharmaceutically acceptable salt or solvate thereof.

Embodiment P47. The method of embodiment P46, wherein the cancer is brain cancer.

Embodiment P48. The method of embodiment P47, wherein the cancer is glioblastoma.

Embodiment P49. A method of modulating LPAR1 activity in a subject, said method comprising administering to the subject a compound of one of embodiments P1 to P33, or a pharmaceutically acceptable salt or solvate thereof.

### VI. Additional Embodiments

Embodiment 1. A compound, or a pharmaceutically acceptable salt or solvate thereof, having the formula:

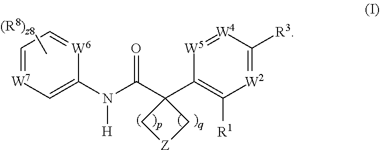

wherein
R¹ is unsubstituted C₂-C₅ alkyl;
W² is N or C(R²);
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁴ is N or C(R⁴);
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁵ is N or C(R⁵);
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted cycloalkyl, substituted or unsubstituted heterocycloalkyl, substituted or unsubstituted aryl, or substituted or unsubstituted heteroaryl;
R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl;
W⁶ is N or C(R⁶)
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR⁶CNR^{6A}R^{6B}, —NHC(O)NR^{6A}R^{6B}, —N(O)ₘ₆, —N^{6A}R^{6B}, —C(O)R^{6C}, —C(O)OR^{6C}, —C(O)NR^{6A}R^{6B}, —OR^{6D}, —SR^{6D}, —N⁶ASO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁷ is N, N⁺—O⁻, or C(R⁷);
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —N_{7A}R^{7B}, —C(O)R^{7C}, —C(O)OR^{7C}, —C(O)NR^{7A}R^{7B}, —OR^{7D}, —SR^{7D}, —N_{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl;
Z is O, C(R¹¹)(R¹²), or C(O)N(R¹³) R¹¹ is hydrogen, halogen, —CX¹¹₃, —CHX¹¹₂, —CH₂X¹¹, —OCX¹¹₃, —OCH₂X¹¹, —OCHX¹¹₂, —CN, —SOₙ₁₁R^{11D}, —SOᵥ₁₁NR^{11A}R^{11B}, —NR^{11C}NR^{11A}R^{11B}, —ONR^{11A}R^{11B}, —NHC(O)NR^{11C}NR^{11A}R^{11B}, —NHC(O)NR^{11A}R^{11B}, —N(O)min, —NR^{11A}R^{11B}, —C(O)R^{11C}, —C(O)OR^{11C}—C(O)NR^{11A}R^{11B}, —OR^{11D}, —SR^{11D}, —NR^{11A}SO₂R^{11D}, —NR^{11A} C(O)R^{11C}, —NR^{11A} C(O)OR^{11C}, —NR^{11A} OR^{11C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —CN, —SOₙ₁₂R₁₂R^{12D}, —SOᵥ₂NR^{12A}R^{12B}, —NR^{12C}NR^{12A}R^{12B} ONR^{12A}R^{12B}, —NHC(O)NR^{12C}NR^{12A}R^{12B}, —NHC(O)NR^{12A}R^{12B}, —N(O)ₘ₁₂, —NR^{12A}R^{12B}, —C(O)R^{12C}, —C(O)OR^{12C}, —C(O)NR^{12A}R^{12B}, —OR^{12D}, —SR^{12D}, —NR^{12A}SO₂R^{12D}, —NR^{12A}C(O)R^{12C}, —NR^{12A}C(O)OR^{12C}, —NR^{12A} OR^{12C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹¹ and R¹² substituents may optionally be joined to form an oxo, substituted or unsubstituted cycloalkyl, or substituted or unsubstituted heterocycloalkyl;
R¹³ is hydrogen, halogen, —CX¹³₃, —CHX¹³₂, —CH₂X¹³, —OCX¹³₃, —OCH₂X¹³, —OCHX¹³₂, —SOₙ₁₃R^{13D}, —SOᵥ₁₃NR^{13A}R^{13B}, —C(O)R^{13C}, —C(O)OR^{13C}, —C(O)NR^{13A}R^{13B}, —OR^{13D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, R^{8D}, R^{11A}, R^{11B}, R^{11C}, R^{11D}, R^{12A}, R^{12B}, R^{12C}, R^{12D}R^{13A}, R^{13B}, R^{13C}, and R^{13D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} and R^{2B} substituents bonded to the same nitrogen atom may optionally be joined to form a substituted or unsubstituted heterocycloalkyl or substituted or unsubstituted heteroaryl; R^{3A} and R^{3B}substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{12A} and R^{12B} substituents bonded to the same nitrogen atom may optionally be joined to form a substituted or unsubstituted heterocycloalkyl or substituted or unsubstituted heteroaryl; R^{13A} and R^{13B} substituents bonded to the same nitrogen atom may optionally be joined to form a substituted or unsubstituted heterocycloalkyl or substituted or unsubstituted heteroaryl;
X², X³, X⁴, X⁵, X⁶, X⁷, X⁸, X¹¹, X¹², and X¹³ are independently —F, —Cl, —Br, or —I;
n2, n3, n4, n5, n6, n7, n8, n11, n12, and n13 are independently an integer from 0 to 4;
m2, m3, m4, m5, m6, m7, m8,m11, m12, v2, v3, v4, v5, v6, v7, v8, v11, v12, and v13 are independently 1 or 2;
z8 is an integer from 0 to 3;
p is 1 or 2; and
q is 1 or 2.

Embodiment 2. The compound of embodiment 1, having the formula:

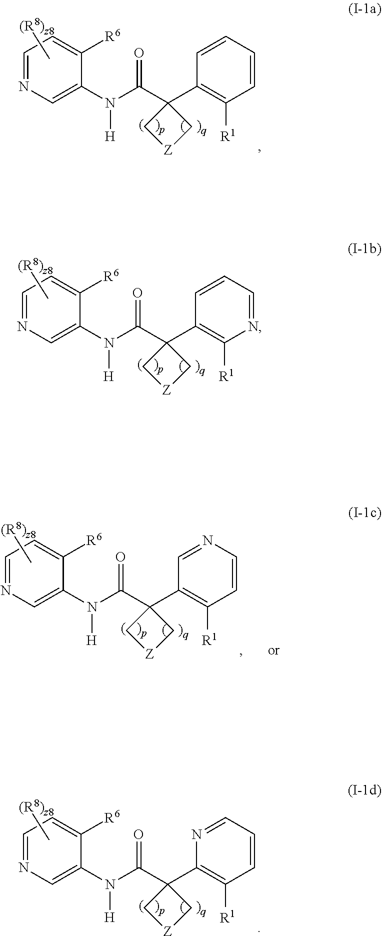

Embodiment 3. The compound of embodiment 1, having the formula:

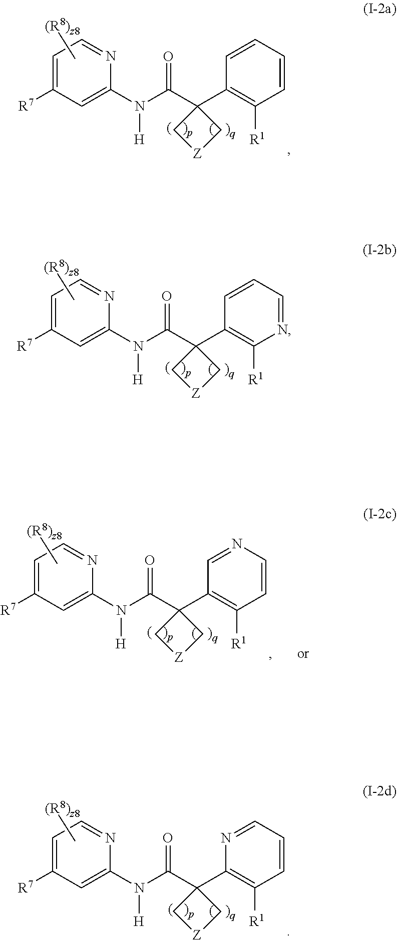

Embodiment 4. The compound of embodiment 1, having the formula:

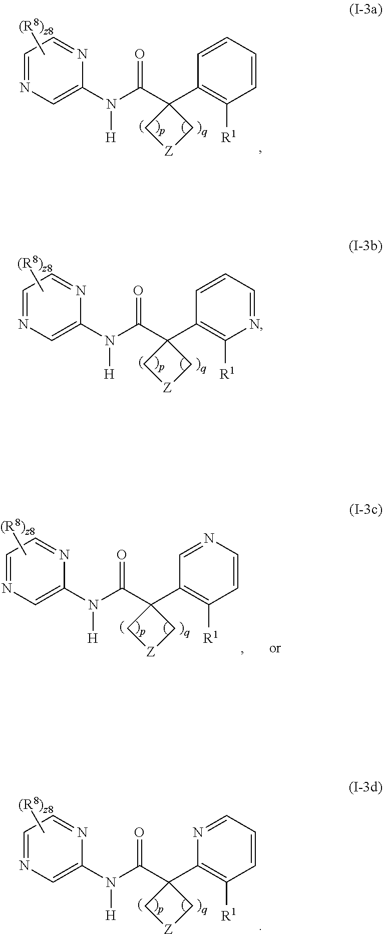

Embodiment 5. The compound of embodiment 1, having the formula:

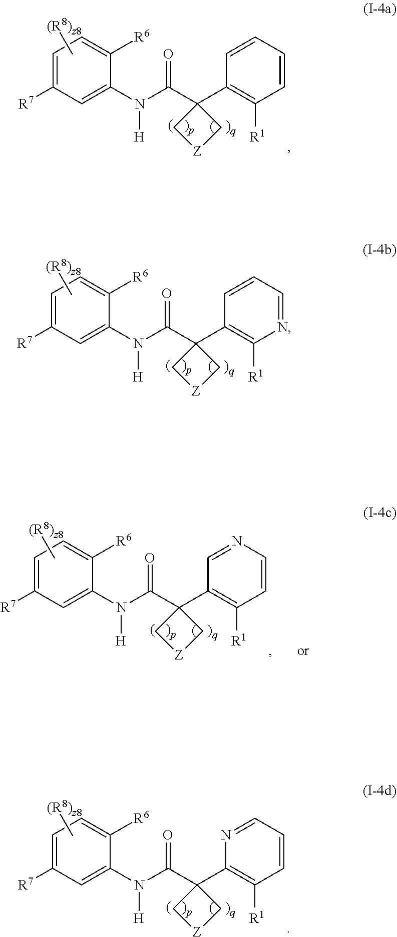

Embodiment 6. The compound of one of embodiments 1 to 5, wherein R¹ is unsubstituted C₃ alkyl.

Embodiment 7. The compound of one of embodiments 1 to 5, wherein R¹ is isopropyl.

Embodiment 8. A compound, or a pharmaceutically acceptable salt or solvate thereof, having the formula:

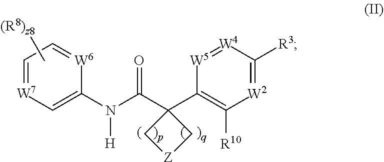

wherein
W² is N or C(R²)
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —N^{2A}R^{2B}, —C(O)R^{2C}, —C(O)OR^{2C}, —C(O)NR^{2A}R^{2B}, —OR^{2D}, —SR^{2D}—NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁴ is N or C(R⁴);
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —N^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁵ is N or C(R⁵);
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted cycloalkyl, substituted or unsubstituted heterocycloalkyl, substituted or unsubstituted aryl, or substituted or unsubstituted heteroaryl;
R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl;
W⁶ is N or C(R⁶)
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR⁶CNR^{6A}R^{6B}, —NHC(O)NR^{6A}R^{6B}, —N(O)ₘ₆, —N^{6A}R^{6B}, —C(O)R^{6C}, —C(O)OR^{6C}, —C(O)NR^{6A}R^{6B}, —OR^{6D}, —SR^{6D}, —N⁶ASO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁷ is N, N⁺—O⁻, or C(R⁷);
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —N₇^{A}R^{7B}, —C(O)R^{7C}, —C(O)OR^{7C}, —C(O)NR^{7A}R^{7B}, —OR^{7D}, —SR^{7D}, —N_{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX⁸₃, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —NR^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl;
R¹⁰ is hydrogen, halogen, —CX¹⁰₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R² substituents may optionally be joined to form a substituted or unsubstituted cycloalkyl, substituted or unsubstituted heterocycloalkyl, substituted or unsubstituted aryl, or substituted or unsubstituted heteroaryl;
Z is O, C(R¹¹)(R¹²), or C(O)N(R¹³)
R¹¹ is hydrogen, halogen, —CX¹¹₃, —CHX¹¹₂, —CH₂X¹¹, —OCX¹¹₃, —OCH₂X¹¹, —OCHX¹¹₂, —CN, —SOₙ₁₁R^{11D}, —SOᵥ₁₁NR^{11A}R^{11B}, —NR^{11C}NR^{11A}R^{11B}, —ONR^{11A}R^{11B}, —NHC(O)NR^{11C}NR^{11A}R^{11B}, —NHC(O)NR^{11A}R^{11B}, —N(O)ₘ₁₁, —NR^{11A}R^{11B}, —C(O)R^{11C}, —C(O)OR^{11C}, —C(O)NR^{11A}R^{11B}, —OR^{11D}, —SR^{11D}, —NR^{11A} SO₂R^{11D}, —NR^{11A} C(O)R^{11C}, —NR^{11A} C(O)OR^{11C}, —NR^{11A} OR^{11C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —CN, —SOᵥ₁₂R^{12D}, —SOᵥ₁₂NR^{12A}R^{12B}, —NR^{12C}NR^{12A}R^{12B} ONR^{12A}R^{12B}, —NHC(O)NR^{12C}NR^{12A}R^{12B}, —NHC(O)NR^{12A}R^{12B}, —N(O)ₘ₁₂, —NR^{12A}R^{12B}, —C(O)R^{12C}, —C(O)OR^{12C}, —C(O)NR^{12A}R^{12B}, —OR^{12D}, —SR^{12D}, —NR^{12A}SO₂R^{12D}, —NR^{12A}C(O)R^{12C}, —NR^{12A}C(O)OR^{12C}, —NR^{12A} OR^{12C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹¹ and R¹² substituents may optionally be joined to form an oxo, substituted or unsubstituted cycloalkyl, or substituted or unsubstituted heterocycloalkyl;
R¹³ is hydrogen, halogen, —CX¹³₃, —CHX¹³₂, —CH₂X¹³, —OCX¹³₃, —OCH₂X¹³, —OCHX¹³₂, —SOₙ₁₃R^{13D}, —SOᵥ₁₃NR^{13A}R^{13B}, —C(O)R^{13C}, —C(O)OR^{13C}, —C(O)NR^{13A}R^{13B}, —OR^{13D} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, R^{8D}, R^{10A}, R^{10B}, R^{10C}, R^{10D}, R^{11A}, R^{11B}, R^{11C}, R^{11D}R^{12A}, R^{12B}, R^{12C}, R^{12D}, R^{13A}, R^{13B}, R^{13C}, and R^{13D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{12A} and R^{12B} substituents bonded to the same nitrogen atom may optionally be joined to form a substituted or unsubstituted heterocycloalkyl or substituted or unsubstituted heteroaryl; R^{13A} and R^{13B} substituents bonded to the same nitrogen atom may optionally be joined to form a substituted or unsubstituted heterocycloalkyl or substituted or unsubstituted heteroaryl;
X², X³, X⁴, X⁵, X⁶, X⁷, X⁸, X¹⁰, X¹¹, X¹², and X¹³ are independently —F, —Cl, —Br, or —I;
n2, n3, n4, n5, n6, n7, n8, n10, n11, n12, and n13 are independently an integer from 0 to 4;
m2, m3, m4, m5, m6, m7, m8, m10, m11, m12, v2, v3, v4, v5, v6, v7, v8, v10, v11, v12, and v13 are independently 1 or 2;
z8 is an integer from 0 to 3;
p is 1 or 2; and
q is 1 or 2;
wherein at least one of W⁶ or W⁷ is N;
wherein if W⁶ is N and W⁷ is C(R⁷), then R¹⁰ is not hydrogen, —F, —Cl, —CH₃, or —OCH₃;
wherein if W⁶ is C(R⁶) and W⁷ is N, then R¹⁰ is not hydrogen, —F, —Cl, —CH₃, or —OCH₃; and
wherein if W⁶ and W⁷ are N and Z is O or CH₂, then R¹⁰ is not hydrogen, —F, —Cl, —CH₃ or —OCH₃.

Embodiment 9. The compound of embodiment 8, having the formula:

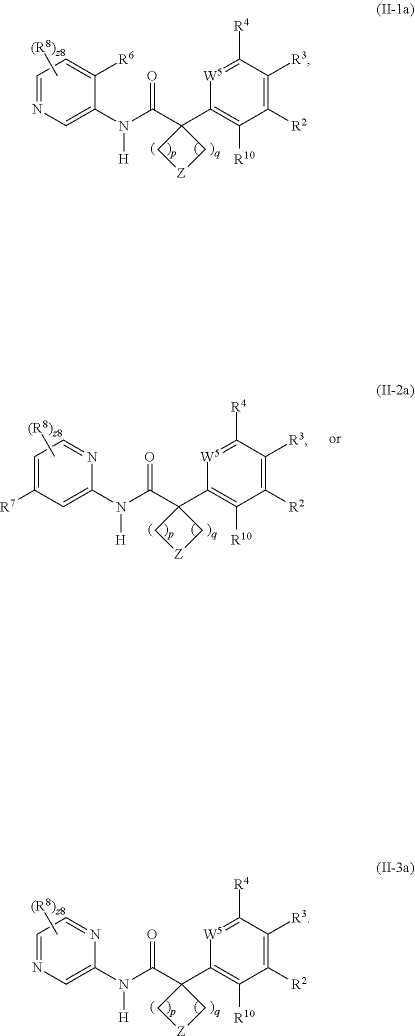

Embodiment 10. The compound of embodiment 8, having the formula:

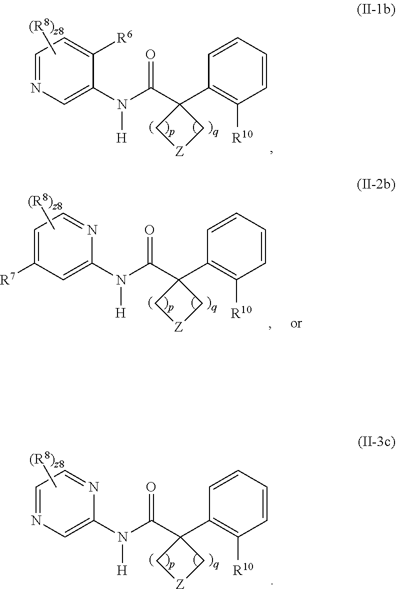

Embodiment 11. The compound of one of embodiments 8 to 10, wherein R¹⁰ is hydrogen or unsubstituted C₁-C₆ alkyl.

Embodiment 12. The compound of one of embodiments 8 to 10, wherein R¹⁰ is isopropyl.

Embodiment 13. The compound of one of embodiments 1 to 12, wherein R⁸ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —SO₃H, —OSO₃H, —SO₂NH₂, —NHNH₂, —ONH₂, —NHC(O)NHNH₂, —NHC(O)NH₂, —NO₂, —NH₂, —C(O)H, —C(O)OH, —CONH₂, —OH, —SH, —NHSO₂H, —NHC(O)H, —NHC(O)OH, —NHOH, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 14. The compound of one of embodiments 1 to 12, wherein R⁸ is independently halogen, —CF₃, —CHF₂, —OCHF₂, —OR^{8D}, unsubstituted C₁-C₆ alkyl, or unsubstituted 2 to 8 membered heteroalkyl.

Embodiment 15. The compound of embodiment 14, wherein R^{8D} is independently unsubstituted C₁-C₆ alkyl or unsubstituted C₃-C₈ cycloalkyl.

Embodiment 16. The compound of one of embodiments 1 to 12, wherein R⁸ is independently —F, —Cl, —Br, —CH₂F, —CHF₂, —OCHF₂, —OCH₃, —OCH(CH₃)₂, —OCH(CH₃)CH₂OCH₃, —O-(unsubstituted cyclopropyl), or substituted or unsubstituted methyl.

Embodiment 17. The compound of one of embodiments 1 to 16, wherein

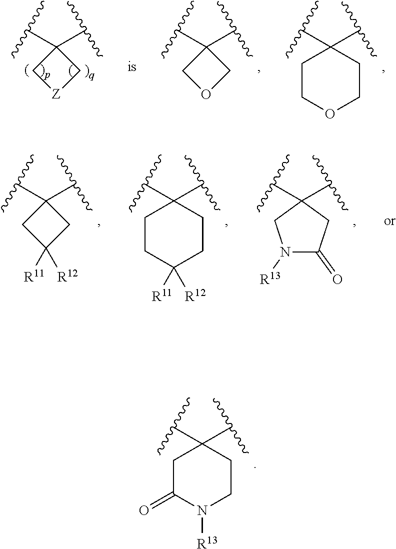

Embodiment 18. The compound of one of embodiments 1 to 17, wherein

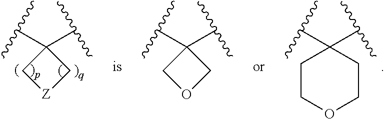

Embodiment 19. The compound of embodiment 17, wherein R¹¹ is hydrogen, halogen, —SOₙ₁₁R^{11D}, —SOᵥ₁₁NR^{11A}R^{11B}, —NR^{11A}R^{11B}, —C(O)OR^{11C}, —C(O)NR^{11A}R^{11B}, —OR^{11D}, —NR^{11A} SO₂R^{11D}, —NR^{11A} C(O)R^{11C}, substituted or unsubstituted C₁-C₆ alkyl, substituted or unsubstituted 2 to 6 membered heteroalkyl, substituted or unsubstituted 3 to 8 membered heterocycloalkyl, or substituted or unsubstituted 5 to 6 membered heteroaryl.

Embodiment 20. The compound of embodiment 19, wherein R^{11A} and R^{11B} are independently hydrogen, unsubstituted C₁-C₆ alkyl, or unsubstituted C₃-C₈ cycloalkyl.

Embodiment 21. The compound of embodiment 19, wherein R^{11C} is hydrogen or unsubstituted C₁-C₆ alkyl.

Embodiment 22. The compound of embodiment 19, wherein R^{11D} is hydrogen or unsubstituted C₁-C₆ alkyl.

Embodiment 23. The compound of embodiment 17, wherein R¹¹ is hydrogen, —F, —OH, —CN, —CH₃, —CH₂CH₃, —NHCH₃, —N(CH₃)₂, —C(O)OH, —C(O)NH₂, —C(O)N(CH₃)₂, —S(O)₂CH₃, —S(O)₂NH₂, —S(O)₂NH(CH₃), —S(O)₂N(CH₃)₂, —N₃,

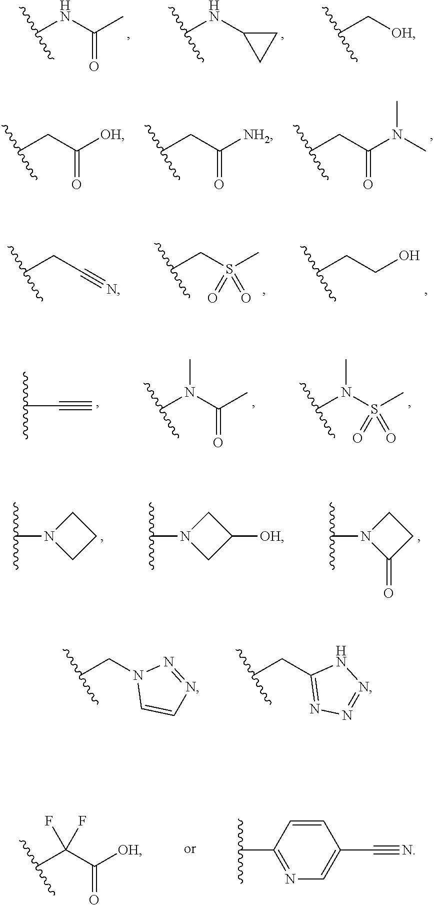

Embodiment 24. The compound of embodiment 17, wherein R¹² is hydrogen, halogen, —SOₙ₁₂R^{12D}, —SOᵥ₁₂NR^{12A}R^{12B}, —NR^{12A}R^{12B}, —C(O)OR^{12C}, —C(O)NR^{12A}R^{12B}, —OR^{12D}, —NR^{12A} SO₂R^{12D}, —NR^{12A}C(O)R^{12C}, substituted or unsubstituted C₁-C₆ alkyl, substituted or unsubstituted 2 to 6 membered heteroalkyl, substituted or unsubstituted 3 to 8 membered heterocycloalkyl, or substituted or unsubstituted 5 to 6 membered heteroaryl.

Embodiment 25. The compound of embodiment 24, wherein R^{12A} and R^{12B} are independently hydrogen, unsubstituted C₁-C₆ alkyl, or unsubstituted C₃-C₈ cycloalkyl.

Embodiment 26. The compound of embodiment 24, wherein R^{12C} is hydrogen or unsubstituted C₁-C₆ alkyl.

Embodiment 27. The compound of embodiment 24, wherein R^{12D} is hydrogen or unsubstituted C₁-C₆ alkyl.

Embodiment 28. The compound of embodiment 17, wherein R¹² is hydrogen, —F, —OH, —CN, —CH₃, —CH₂CH₃, —NHCH₃, —N(CH₃)₂, —C(O)OH, —C(O)NH₂, —C(O)N(CH₃)₂, —S(O)₂CH₃, —S(O)₂NH₂, —S(O)₂NH(CH₃), —S(O)₂N(CH₃)₂, —N₃,

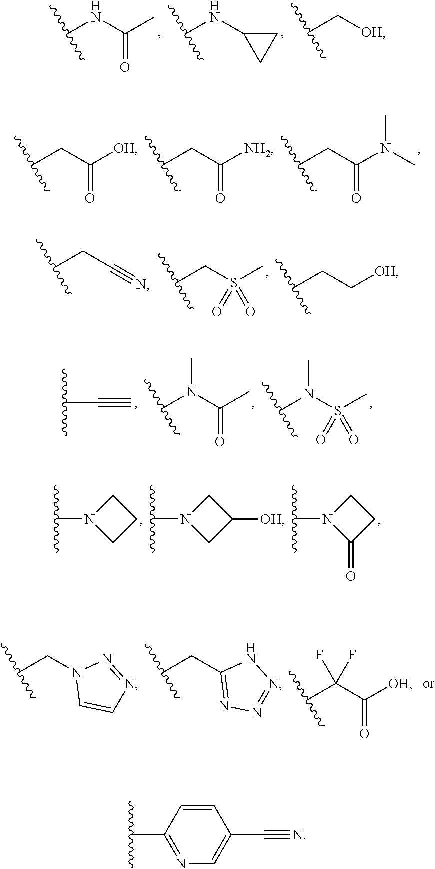

Embodiment 29. The compound of embodiment 17, wherein R¹¹ and R¹² substituents are joined to form a substituted or unsubstituted C₃-C₈ cycloalkyl or substituted or unsubstituted 3 to 8 membered heterocycloalkyl.

Embodiment 30. The compound of embodiment 17, wherein R¹¹ and R¹² substituents are joined to form an oxo.

Embodiment 31. The compound of one of embodiments 1 to 17, wherein

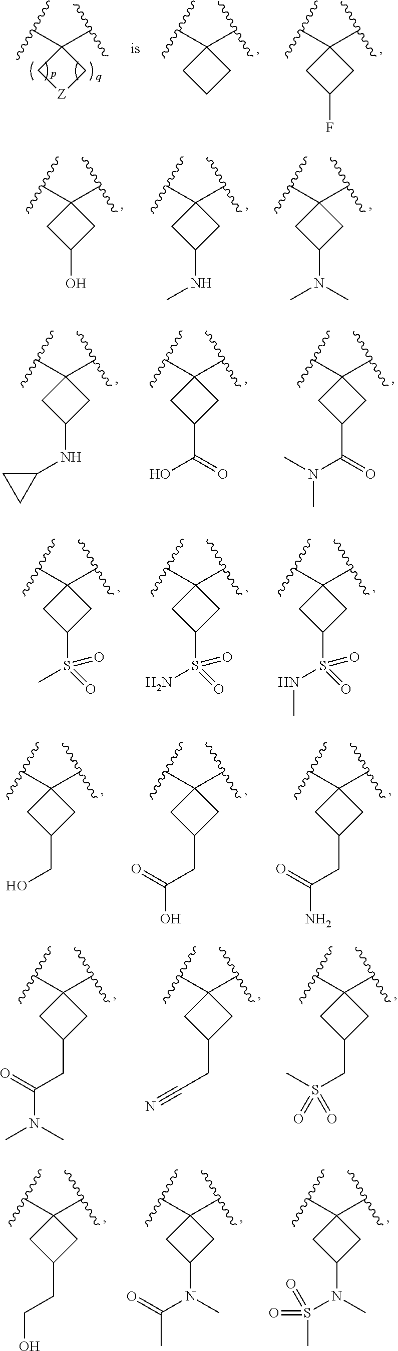

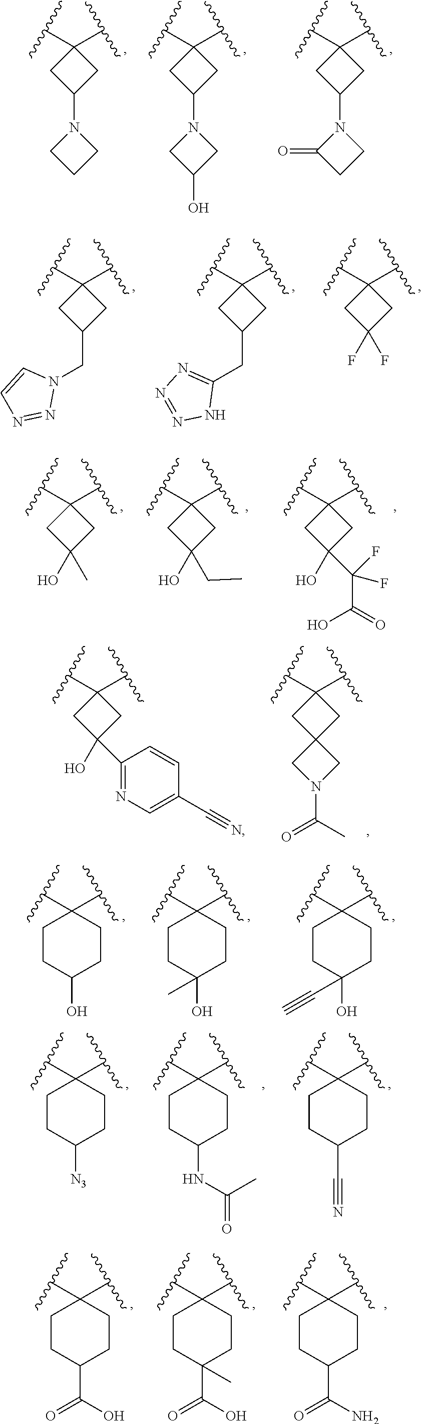

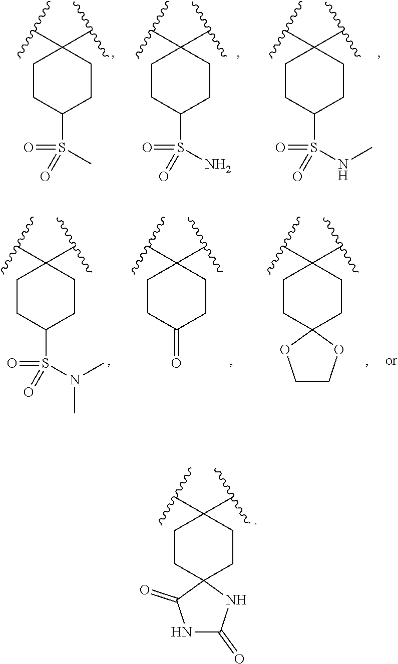

Embodiment 32. The compound of embodiment 17, wherein R¹³ is hydrogen, unsubstituted C₁-C₆ alkyl, or unsubstituted 2 to 6 membered heteroalkyl.

Embodiment 33. The compound of one of embodiments 1 to 17, wherein

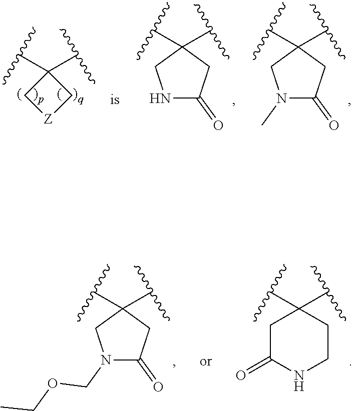

Embodiment 34. A compound, or a pharmaceutically acceptable salt or solvate thereof, having the formula:

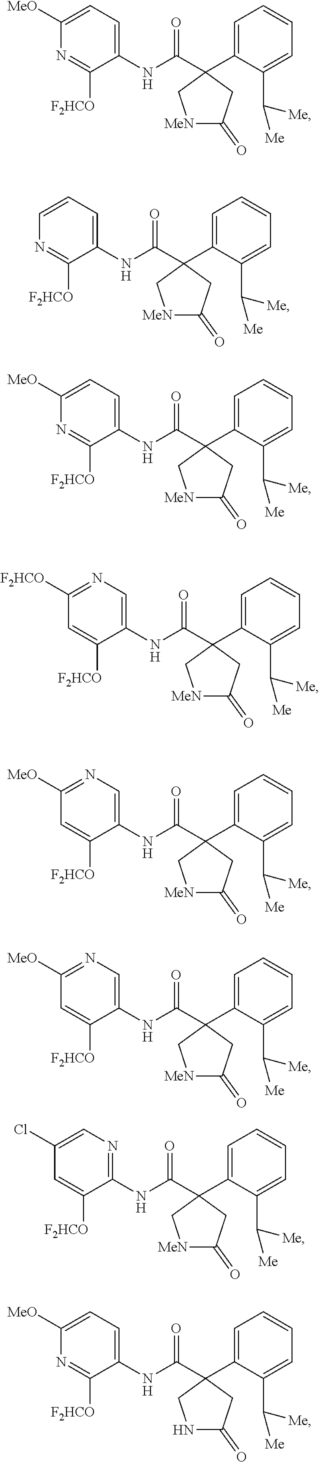

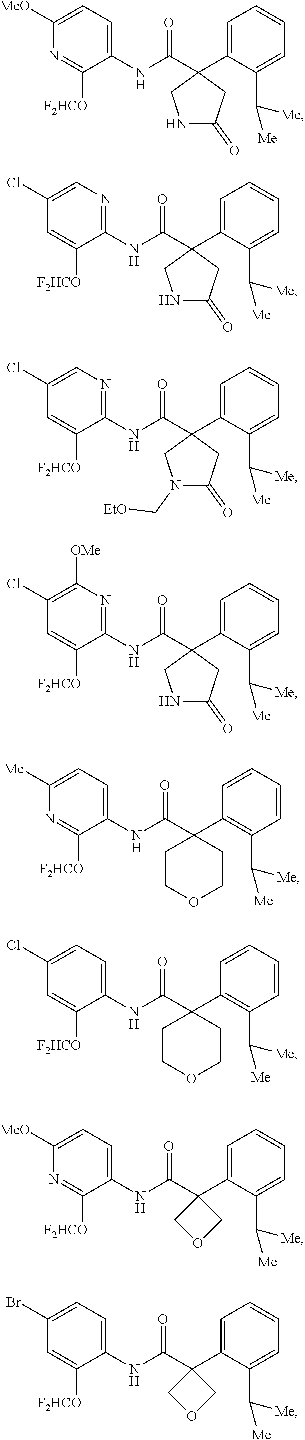

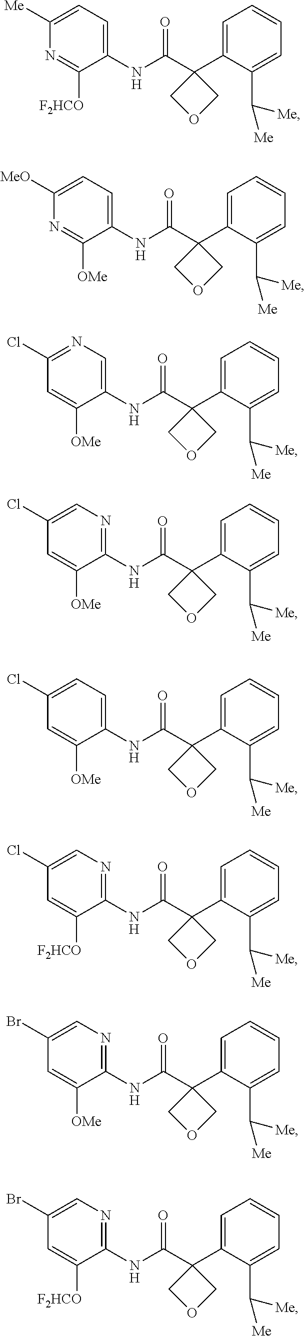

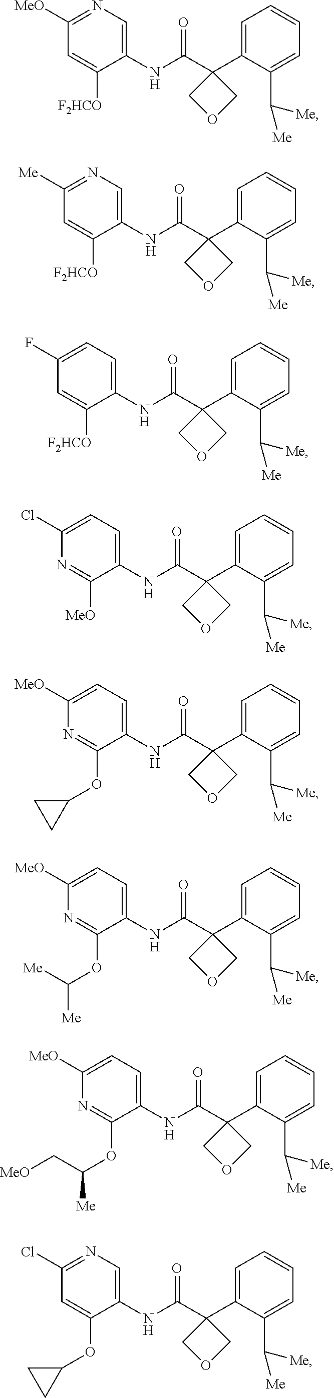

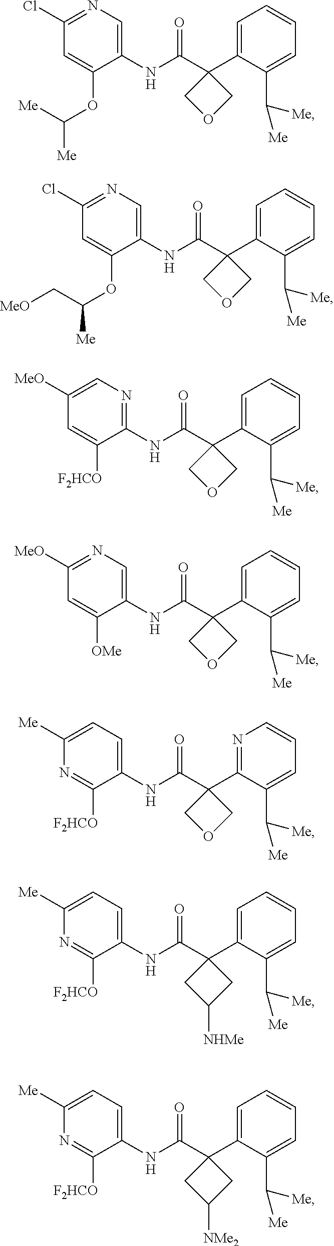

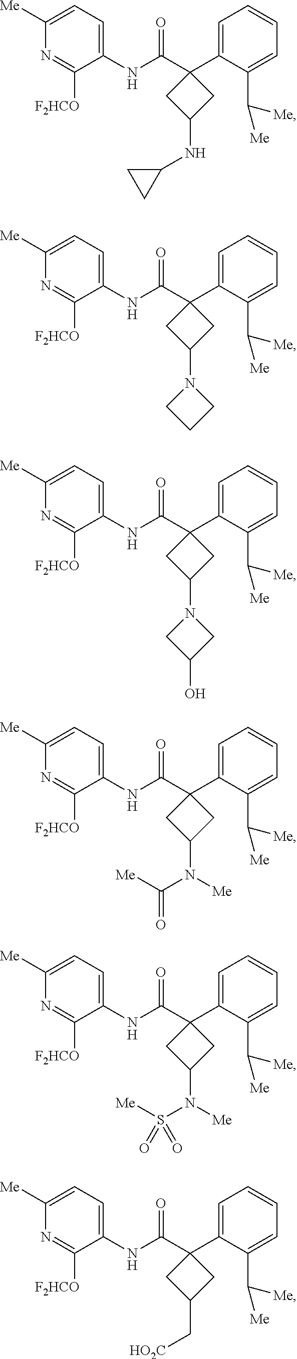 [SMILES: Cc1ccc(NC(=O)C2(c3ccccc3C(C)C)CC(NC3CC3)C2)c(OC(F)F)n1 ; Cc1ccc(NC(=O)C2(c3ccccc3C(C)C)CC(N3CCC3)C2)c(OC(F)F)n1 ; Cc1ccc(NC(=O)C2(c3ccccc3C(C)C)CC(N3CC(O)C3)C2)c(OC(F)F)n1 ; CC(=O)N(C)C1CC(C(=O)Nc2ccc(C)nc2OC(F)F)(c2ccccc2C(C)C)C1 ; Cc1ccc(NC(=O)C2(c3ccccc3C(C)C)CC(N(C)S(C)(=O)=O)C2)c(OC(F)F)n1 ; Cc1ccc(NC(=O)C2(c3ccccc3C(C)C)CC(CC(=O)O)C2)c(OC(F)F)n1]

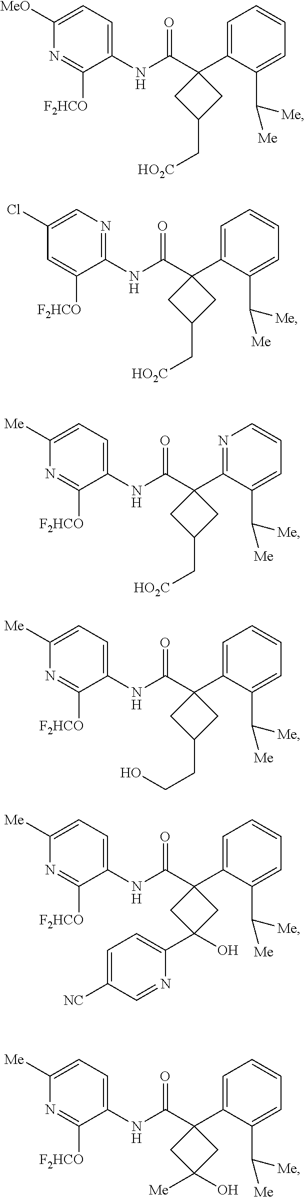 [SMILES: COc1ccc(NC(=O)C2(c3ccccc3C(C)C)CC(CC(=O)O)C2)c(OC(F)F)n1 ; CC(C)c1ccccc1C1(C(=O)Nc2ncc(Cl)cc2OC(F)F)CC(CC(=O)O)C1 ; Cc1ccc(NC(=O)C2(c3ncccc3C(C)C)CC(CC(=O)O)C2)c(OC(F)F)n1 ; Cc1ccc(NC(=O)C2(c3ccccc3C(C)C)CC(CCO)C2)c(OC(F)F)n1 ; Cc1ccc(NC(=O)C2(c3ccccc3C(C)C)CC(O)(c3ccc(C#N)cn3)C2)c(OC(F)F)n1 ; Cc1ccc(NC(=O)C2(c3ccccc3C(C)C)CC(C)(O)C2)c(OC(F)F)n1]

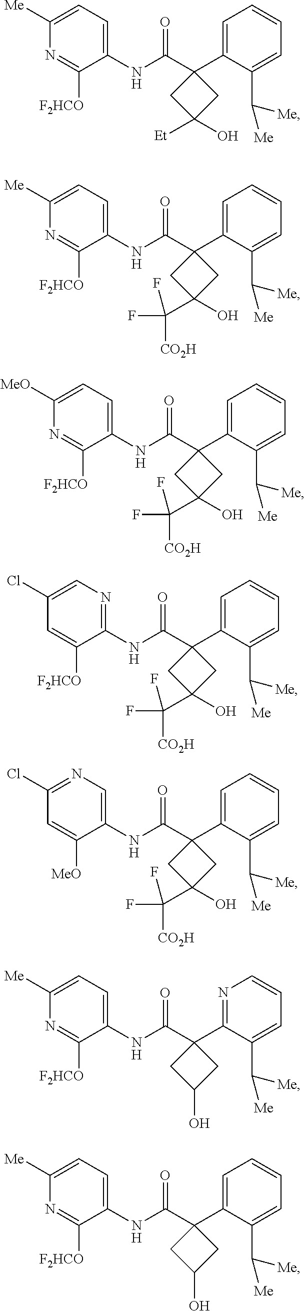

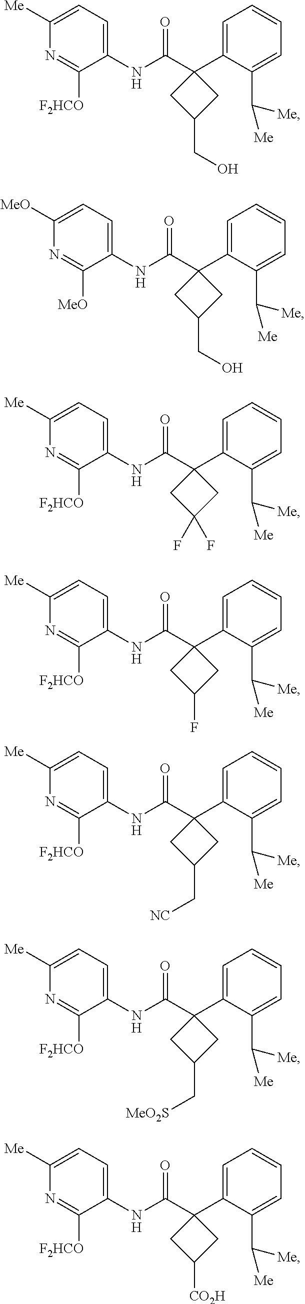

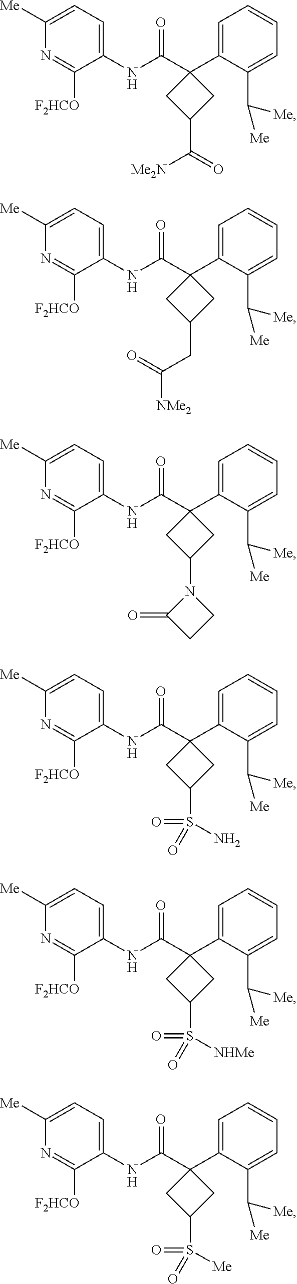 [SMILES: Cc1ccc(NC(=O)C2(c3ccccc3C(C)C)CC(C(=O)N(C)C)C2)c(OC(F)F)n1 ; Cc1ccc(NC(=O)C2(c3ccccc3C(C)C)CC(CC(=O)N(C)C)C2)c(OC(F)F)n1 ; Cc1ccc(NC(=O)C2(c3ccccc3C(C)C)CC(N3CCC3=O)C2)c(OC(F)F)n1 ; Cc1ccc(NC(=O)C2(c3ccccc3C(C)C)CC(S(N)(=O)=O)C2)c(OC(F)F)n1 ; CNS(=O)(=O)C1CC(C(=O)Nc2ccc(C)nc2OC(F)F)(c2ccccc2C(C)C)C1 ; Cc1ccc(NC(=O)C2(c3ccccc3C(C)C)CC(S(C)(=O)=O)C2)c(OC(F)F)n1]

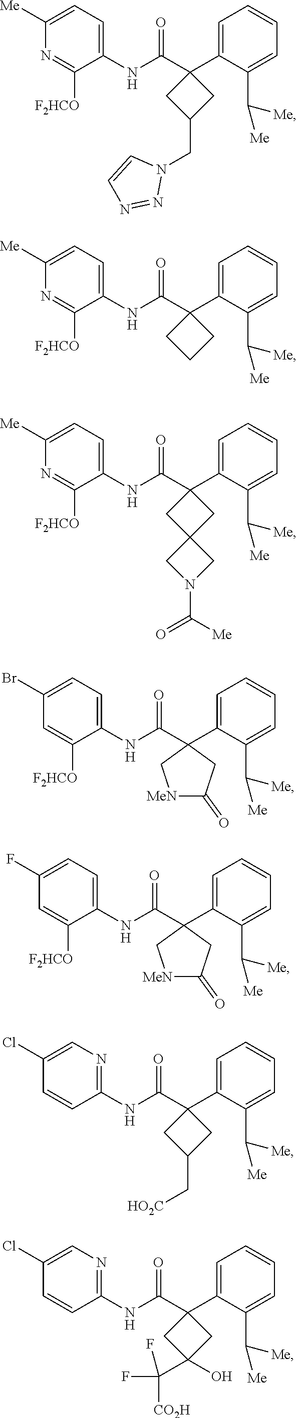

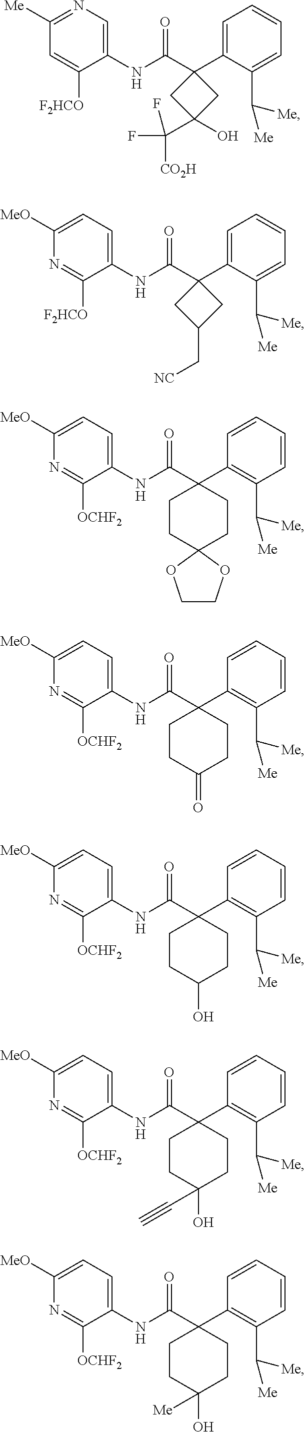

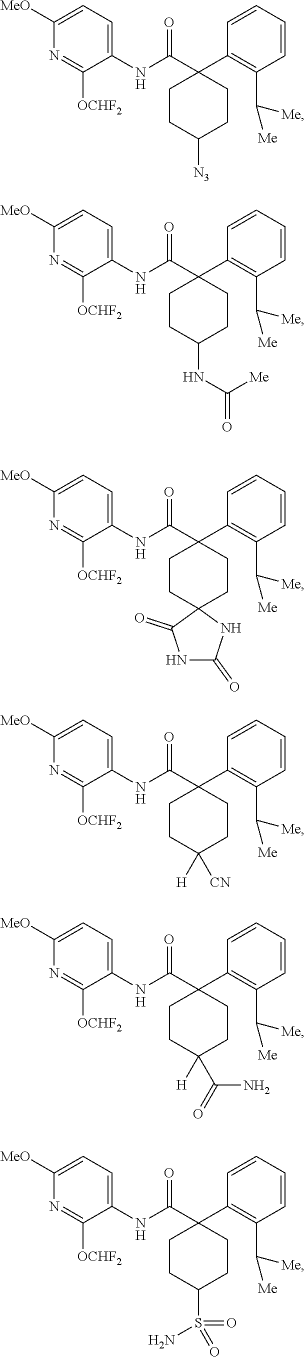

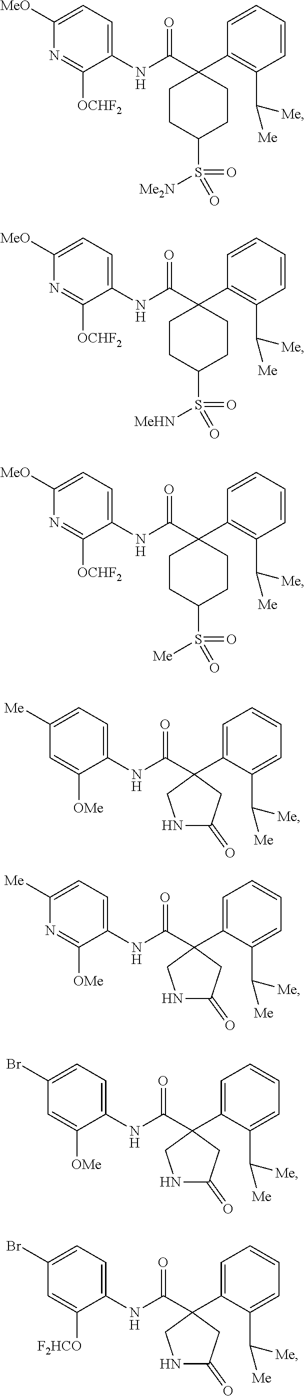

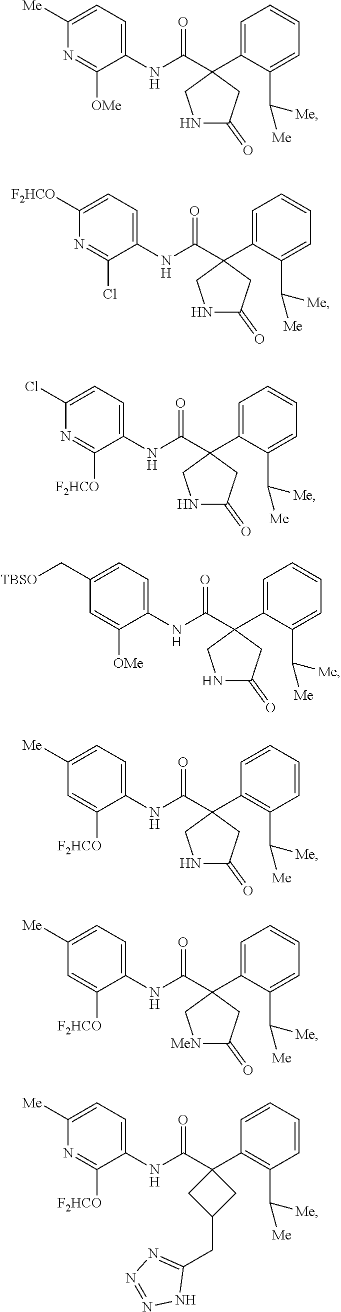

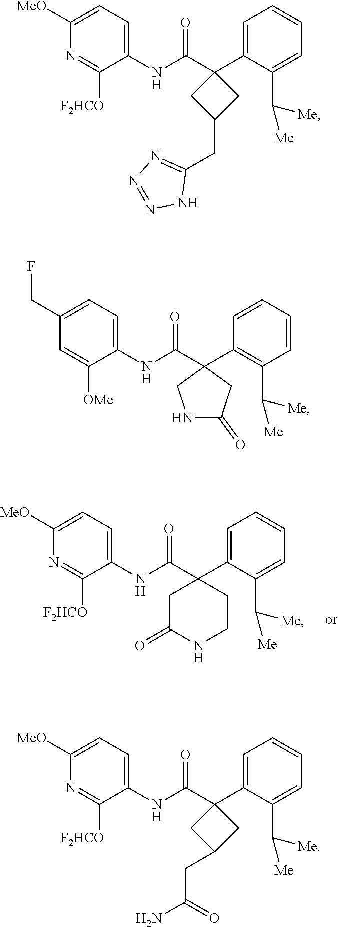 [SMILES: COc1ccc(NC(=O)C2(c3ccccc3C(C)C)CC(Cc3nnn[nH]3)C2)c(OC(F)F)n1 ; COc1cc(CF)ccc1NC(=O)C1(c2ccccc2C(C)C)CNC(=O)C1 ; COc1ccc(NC(=O)C2(c3ccccc3C(C)C)CCNC(=O)C2)c(OC(F)F)n1 ; "or" ; COc1ccc(NC(=O)C2(c3ccccc3C(C)C)CC(CC(N)=O)C2)c(OC(F)F)n1]

Embodiment 35. A compound, or a pharmaceutically acceptable salt or solvate thereof, having the formula:

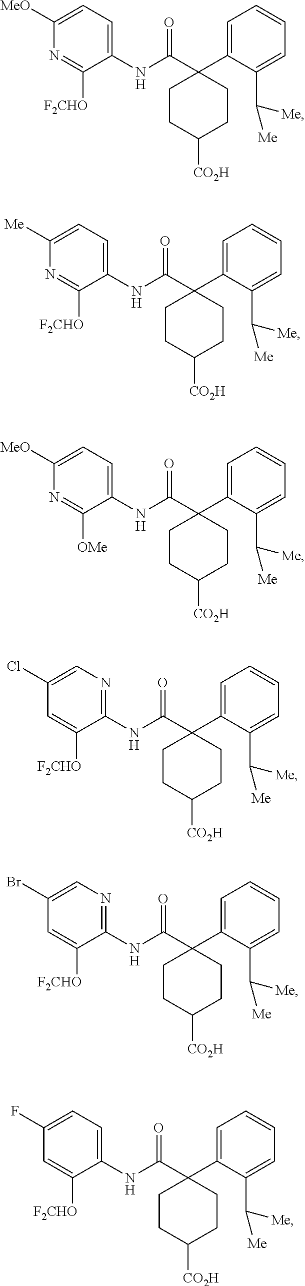

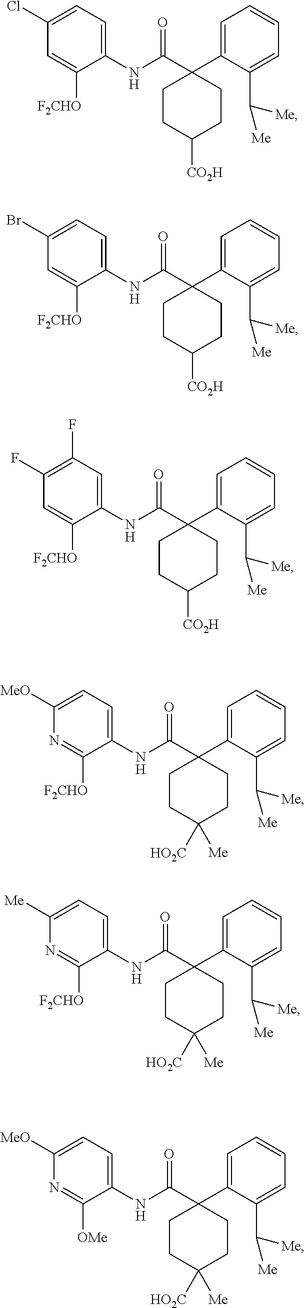

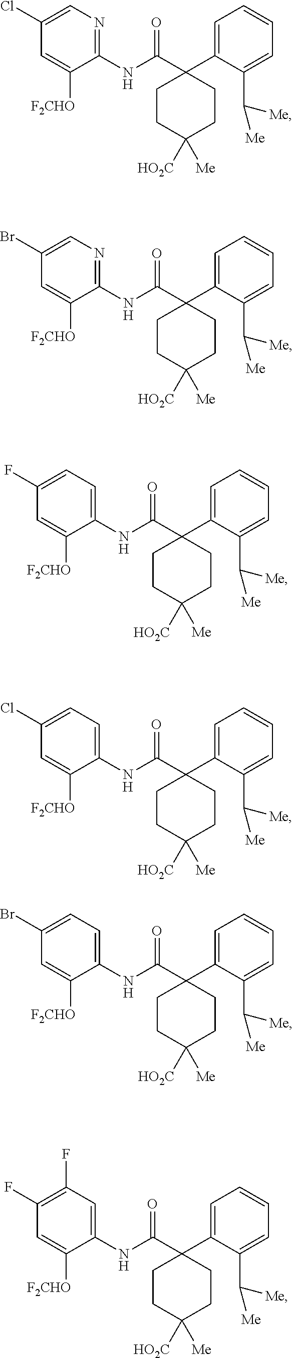

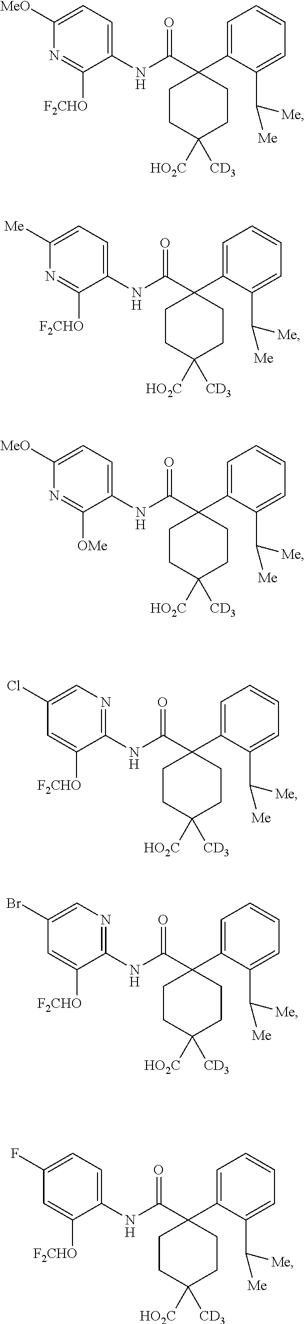

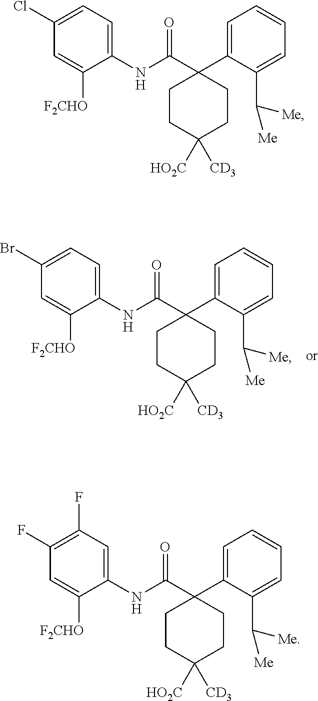

Embodiment 36. A pharmaceutical composition comprising the compound of one of embodiments 1 to 35, or a pharmaceutically acceptable salt or solvate thereof, and a pharmaceutically acceptable excipient.

Embodiment 37. A method of treating a neurodegenerative disorder in a subject in need thereof, said method comprising administering to the subject in need thereof a therapeutically effective amount of a compound of one of embodiments 1 to 35, or a pharmaceutically acceptable salt or solvate thereof.

Embodiment 38. A method of treating an inflammatory disease in a subject in need thereof, said method comprising administering to the subject in need thereof a therapeutically effective amount of a compound of one of embodiments 1 to 35, or a pharmaceutically acceptable salt or solvate thereof.

Embodiment 39. The method of embodiment 38, wherein the inflammatory disease is encephalitis.

Embodiment 40. The method of embodiment 39, wherein the encephalitis is post-hemorrhagic encephalitis.

Embodiment 41. A method of treating a demyelinating disease in a subject in need thereof, said method comprising administering to the subject in need thereof a therapeutically effective amount of a compound of one of embodiments 1 to 35, or a pharmaceutically acceptable salt or solvate thereof.

Embodiment 42. The method of embodiment 41, wherein the demyelinating disease is a demyelinating disease of the central nervous system.

Embodiment 43. The method of embodiment 42, wherein the demyelinating disease is multiple sclerosis.

Embodiment 44. The method of embodiment 41, wherein the demyelinating disease is a demyelinating disease of the peripheral nervous system.

Embodiment 45. A method of treating a fibrotic disease in a subject in need thereof, said method comprising administering to the subject in need thereof a therapeutically effective amount of a compound of one of embodiments 1 to 35, or a pharmaceutically acceptable salt or solvate thereof.

Embodiment 46. The method of embodiment 45, wherein the fibrotic disease is pulmonary fibrosis, skin fibrosis, liver fibrosis, or ocular fibrosis.

Embodiment 47. The method of embodiment 45, wherein the fibrotic disease is idiopathic pulmonary fibrosis, scleroderma, nonalcoholic steatohepatitis, or ocular fibrosis.

Embodiment 48. A method of treating cancer in a subject in need thereof, said method comprising administering to the subject in need thereof a therapeutically effective amount of a compound of one of embodiments 1 to 35, or a pharmaceutically acceptable salt or solvate thereof.

Embodiment 49. The method of embodiment 48, wherein the cancer is brain cancer.

Embodiment 50. The method of embodiment 49, wherein the cancer is glioblastoma.

Embodiment 51. A method of modulating LPAR1 activity in a subject, said method comprising administering to the subject a compound of one of embodiments 1 to 35, or a pharmaceutically acceptable salt or solvate thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

Multiple sclerosis (MS), a chronic progressive disorder, is an inflammatory autoimmune disease whereby the dysregulated immune system attacks the patient's own protective myelin sheath. If left unchecked, the signal transduction along denuded axons can become increasingly deteriorated, eventually leading to the permanent damage of the affected nerve fibers. Recent studies have shown that, when compared to patients with non-inflammatory, non-vascular neurological diseases, MS patients have increased levels of LPAs in the serum (Balood et al., Hum. Immunol., 2014, 75, 411-413). Furthermore, in MS patients, the LPA levels in serum and cerebral spinal fluid (CSF) are even further elevated during disease flares when compared to periods of remission (Jiang et al., Neurol Res., 2018, 40, 335-339). This is consistent with the increased autotaxin activity, an enzyme known to be important for the generation of LPA from lysophosphatidylcholine, observed in MS patients (Zahednasab et al., J. Neuroimmunol., 2014, 273, 120-123). Indeed, it has been established in cellular assays that LPA can induce a pro-inflammatory response through the activation of LPAR1 found in MS patient monocyte-derived macrophages by stimulating the production of pro-inflammatory cytokines such as interleukin-1β (IL-1β) and tumor necrosis factor alpha (TNFα), as well as suppressing the production of anti-inflammatory cytokines such as interleukin-2 (IL-2). Furthermore, in the mouse experimental autoimmune encephalomyelitis (EAE) model of MS, blockade of LPA signaling through genetic deletion of LPA1 was found to decrease the severity of the disease (Fransson et al., Mol. Neurobiol., 2021, 58, 470-482). While dampening the inflammatory immune attack on the myelin sheath can constitute an effective approach to the treatment of MS, promoting the remyelination of damaged, denuded axons would represent another attractive approach (Deshmukh et al., Nature, 2013, 502, 327-332). In this regard, neuronal remyelination can be achieved by driving the differentiation of oligodendrocyte precursor cells (OPCs), an endogenous stem cell, to myelin-producing oligodendrocytes (Najm et al., Nature, 2015, 52, 216-220). It has been demonstrated that LPAR1 is highly expressed in OPCs. Furthermore, a direct role of LPAR1 in OPC differentiation in mice has been established whereby the genetic knockout of LPAR1 led to an enrichment of oligodendrocytes and overexpression of myelin-protein positive (MBP) cells in the mouse cortex (Lorrain et al., Society for Neuroscience Conference Proceedings, 2017, “LPA receptors modulate oligodendrocyte differentiation and maturation”). Since LPA-LPAR1 signaling axis has been shown to play an important role in both demyelination (i.e., promotion of an inflammatory environment) and remyelination (i.e., prevention of OPC differentiation into oligodendrocyte) of axons, the identification of a potent and selective LPAR1 antagonists would thus be of significant relevance in the treatment of MS and other inflammatory demyelination disorders.

### 1. Experimental Procedures and Characterization Data

The compounds used in the reactions described herein are made according to known organic synthesis techniques, starting from commercially available chemicals and/or from compounds described in the chemical literature. “Commercially available chemicals” are obtained from standard commercial sources including Acros Organics (Geel, Belgium), Aldrich Chemical (Milwaukee, WI, including Sigma Chemical and Fluka), Apin Chemicals Ltd. (Milton Park, UK), Ark Pharm, Inc. (Libertyville, IL), Avocado Research (Lancashire, U.K.), BDH Inc. (Toronto, Canada), Bionet (Cornwall, U.K.), Chemservice Inc. (West Chester, PA), Combi-blocks (San Diego, CA), Crescent Chemical Co. (Hauppauge, NY), eMolecules (San Diego, CA), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Matrix Scientific, (Columbia, SC), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Ryan Scientific, Inc. (Mount Pleasant, SC), Spectrum Chemicals (Gardena, CA), Sundia Meditech, (Shanghai, China), TCI America (Portland, OR), Trans World Chemicals, Inc. (Rockville, MD), and WuXi (Shanghai, China).

Suitable reference books and treatises that detail the synthesis of reactants useful in the preparation of compounds described herein, or provide references to articles that describe the preparation, include for example, “Synthetic Organic Chemistry,” New York: John Wiley & Sons, Inc., 1982; Sandler S. R. et al., “Organic Functional Group Preparations,” 2^{nd} ed., New York: Academic Press, 1983; House, H. O., “Modern Synthetic Reactions,” 2^{nd}ed., Menlo Park: W. A. Benjamin, Inc., 1972; Gilchrist, T. L., “Heterocyclic Chemistry,” 2^{nd} ed., New York: Wiley, 1992; March, J., “Advanced Organic Chemistry: Reactions, Mechanisms and Structure,” 4^{th} ed., New York: Wiley, 1992. Additional suitable reference books and treatises that detail the synthesis of reactants useful in the preparation of compounds described herein, or provide references to articles that describe the preparation, include for example, Fuhrhop, J., Penzlin, G., “Organic Synthesis: Concepts, Methods, Starting Materials,” 2^{nd} ed., New York: Wiley, 1994; Hoffman, R. V., “Organic Chemistry, An Intermediate Text,” Oxford: Oxford University Press, 1996; Larock, R. C., “Comprehensive Organic Transformations: A Guide to Functional Group Preparations,” 2^{nd} ed., New York: Wiley, 1999; Otera, J., “Modem Carbonyl Chemistry,” New York: Wiley, 2000; Solomons, T. W. G., “Organic Chemistry,” 7^{th} ed., New York: Wiley, 2000; Stowell, J. C., “Intermediate Organic Chemistry,” 2^{nd} ed., New York: Wiley, 1993; “Industrial Organic Chemicals: Starting Materials and Intermediates: An Ullmann's Encyclopedia,” New York: Wiley, in 8 volumes; “Organic Reactions,” New York: Wiley, in over 55 volumes; and “Chemistry of Functional Groups,” New York: Wiley, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is Stahl, P. H., Wermuth, C. G., “Handbook of Pharmaceutical Salts,” Zurich: Verlag Helvetica Chimica Acta, 2002.

### LIST OF ABBREVIATIONS

As used above, and throughout the description of the invention, the following abbreviations, unless otherwise indicated, shall be understood to have the following meanings:
ACN or MeCN acetonitrile
aq aqueous
Bu butyl
BOC or Boc tert-butyl carbamate
BrettPhos Pd G3 [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate
BSA bovine serum albumin
CDI 1,1′-carbonyldiimidazole
CHO Chinese hamster ovary
Cy cyclohexyl
dba dibenzylideneacetone
DAST diethylaminosulfur trifluoride
DCC N,N′-dicyclohexylcarbodiimide
DCE dichloroethane (C₁CH₂CH₂Cl)
DCM dichloromethane (CH₂Cl₂)
DIPEA or DIEA diisopropylethylamine
DMAP 4-(N,N-dimethylamino)pyridine
DME 1,2-dimethoxyethane
DMEM Dulbecco's modified eagle medium
DMF N,N-dimethylformamide
DMA N,N-dimethylacetamide
DMSO dimethylsulfoxide
EDC N-(3-dimethylaminopropyl)-N′-ethylcarbodiimide
equiv equivalent(s)
Et ethyl
EtOH ethanol
EtOAc ethyl acetate
FBS fetal bovine serum
h hour(s)
HATU 1-[bis(dimethylamino)methylene]-1H-1,2,3-triazolo[4,5-b]pyridinium 3-oxide hexafluorophosphate
HEPES 4-(2-hydroxyethyl)-1-piperazineethanesulfonic acid
Hex hexanes
HPLC high performance liquid chromatography
LCMS or LC-MS liquid chromatography-mass spectrometry
LG leaving group
M molar
mCPBA meta-chloroperoxybenzoic acid
Me methyl
MeOH methanol
min minute(s)
MS mass spectroscopy
NMI N-methylimidazole
NMP N-methyl-2-pyrrolidone
NMR nuclear magnetic resonance
Pd/C palladium on carbon
PG protecting group
RT room temperature
T3P propylphosphonic anhydride
TBAF tetrabutylammonium fluoride
TCFH chloro-N,N,N′,N′-tetramethylformamidinium hexafluorophosphate
TEA triethylamine
TFA trifluoroacetic acid
TFAA trifluoroacetic anhydride
THF tetrahydrofuran
v/v volume per volume
w/w weight per weight
XantPhos 4,5-bis(diphenylphosphino)-9,9-dimethylxanthene
XPhos 2-dicyclohexylphosphino-2′4′6′-triisopropylbiphenyl
XtalFluor-E (diethylamino)difluorosulfonium tetrafluoroborate

### General Synthetic Schemes

Compounds of Formula (I) and Formula (II) of the present disclosure may be prepared, for example from (hetero)aryl amine (2), or its corresponding (hetero)aryl ammonium salt, and a carboxylic acid (1a) or (1b) in the presence of an appropriate coupling reagent such as HATU, CDI, T3P, TCFH, or the like, and an appropriate base such as TEA, DIEA, NMI, or the like (Scheme 1). Alternatively, the acid may be pre-activated via its conversion into the corresponding acid chloride using an agent such as thionyl chloride, oxalyl chloride, or the like. The product amide (3), which itself may be a compound of Formula (I) or (II), can be further functionalized using synthetic methodologies known to those skilled in the art to deliver another compound of Formula (I) or (II). Examples of such transformations include, but are not limited to:
(a) reductive amination of a ketone or an aldehyde present in (3), with a suitable amine in the presence of a suitable a reducing agent such as sodium cyanoborohydride, sodium triacetoxyborohydride, or the like, and a suitable additive such as acetic acid, titanium(IV) isopropoxide, or the like;
(b) capping of an amine or an alcohol present in (3), with a suitable electrophile such as acyl chloride, sulfonyl chloride, alkyl halide, or the like, in the presence of a suitable base such as TEA, sodium hydride, cesium carbonate, or the like;
(c) olefination of a ketone or an aldehyde present in (3), with a suitable Wittig reagent, Horner-Wadsworth-Emmons reagent, Tebbe's reagent, or the like;
(d) hydrogenation of an alkene or an alkyne present in (3), in the presence of a catalyst such as Pd/C, Pd(OH)₂, or the like, and a reducing agent such as hydrogen gas, deuterium gas, or the like;
(e) reduction of a ketone, an aldehyde, or an ester present in (3), with a suitable a reducing agent such as lithium aluminum hydride, sodium borohydride, lithium borohydride, or the like;
(f) hydrolysis of a nitrile or an ester present in (3), with a suitable reagent such as lithium hydroxide, sodium hydroxide, or potassium hydroxide, or the like;
(g) reaction of a ketone, an aldehyde, or an ester present in (3), with a suitable organometallic reagent such as organolithiums, Grignard reagents, organozincates, or the like;
(h) deoxyfluorination of a ketone, an aldehyde, or an alcohol present in (3), with a suitable reagent such as DAST, XtalFluor-E, or the like;
(i) oxidation of an alcohol or an aldehyde present in (3), with a suitable oxidant such as Jones reagent, sodium chlorite, potassium permanganate, or the like;
(j) coupling of a carboxylic acid present in (3), with a suitable amine in the presence of a coupling reagent such as HATU, CDI, T3P, TCFH, or the like, and a base such as TEA, DIEA, NMI, or the like;
(k) conversion of an alcohol present in (3), to a suitable leaving group such as iodide, mesylate, or the like, and its subsequent displacement by a suitable nucleophile such as potassium thioacetate, sodium thiomethoxide, sodium azide, or the like;
(l) oxidation of a sulfide present in (3), with a suitable oxidant such as oxone, mCPBA, N-chlorosuccinimide, or the like;
(m) coupling of a sulfonyl chloride present in (3) with a suitable amine;
(n) Huisgen cycloaddition of an azide present in (3), with a suitable alkyne in the presence of an appropriate promoter such as a copper catalyst, a ruthenium catalyst, heat, or the like;
(o) Coupling of a (hetero)aryl halide present in (3), with a suitable organotin reagent, organoboron reagent, organosilicon reagent, or the like, in the presence of an appropriate promoter such as a palladium catalyst, an iron catalyst, a nickel catalyst, or the like, and an appropriate base such as triethylamine, cesium carbonate, potassium phosphate, sodium bicarbonate, tetrabutylammonium fluoride, or the like; and
(o) separation of a mixture of stereoisomers into its stereochemically-enriched constituents utilizing an appropriate chiral column such as ChiralPAK IF, CHIRAL ART Amylose SA, CHIRAL ART Cellulose SB, or the like.

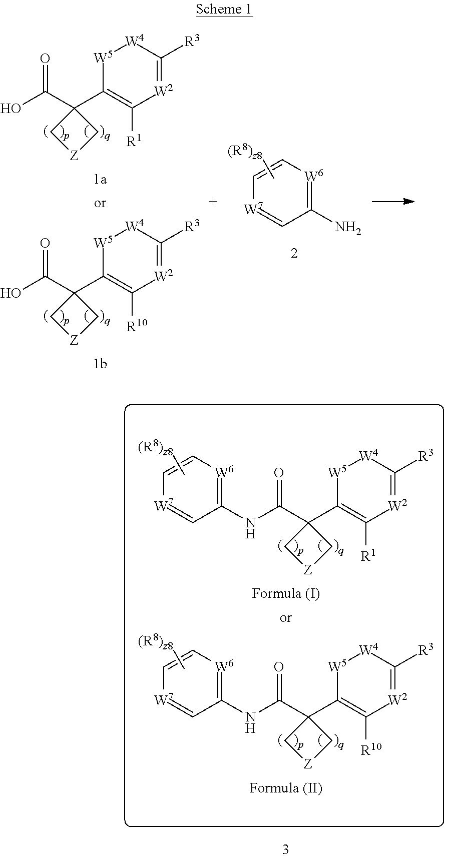

Alternatively, it may be more advantageous to instead prepare compounds of Formula (I) or (II) via the copper- or palladium-mediated coupling (Scheme 2) of a primary amide (4a) or (4b), itself accessible from carboxylic acid (1a) or (1b) and ammonia using conditions well known to those skilled in the art, and a (hetero)aryl halide (5).

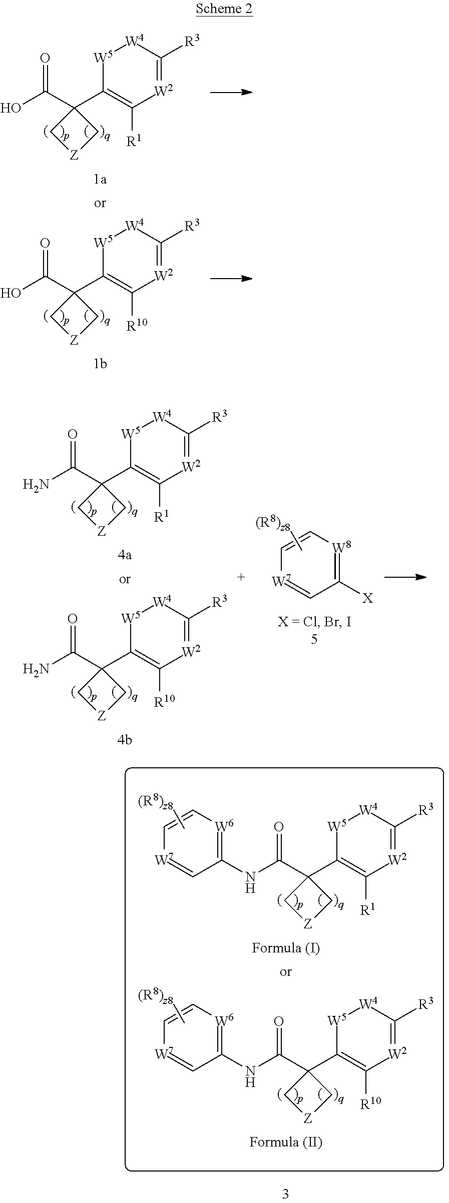

For certain embodiments, a person skilled in the art can access the carboxylic acid used for the coupling depicted in Scheme 1 by an initial C-arylation of a suitable cyanoacetate (6) with an appropriately functionalized (hetero)aryl halide (7) in the presence of a promoter such as a palladium catalyst, a copper catalyst, heat, or the like, and a base such as potassium phosphate, TEA, potassium bis(trimethylsilyl)amide, or the like (Scheme 3). The resulting (hetero)arylated nitrile (8) can then be further alkylated with an appropriate electrophile, such as an alpha-halogenated ester, an acrylate ester, or the like, to furnish nitrile (9). Reduction of the nitrile in the presence of a catalyst such as Pd/C, Pd(OH)₂, or the like, and a reducing agent such as hydrogen gas, deuterium gas, or the like, would then trigger an intramolecular cyclization event to afford lactam ester (10). This lactam ester can be further functionalized using techniques known to a person skilled in the art, including, for example, N-alkylation with an alkyl halide, prior to its hydrolysis to the requisite carboxylic acid (11).

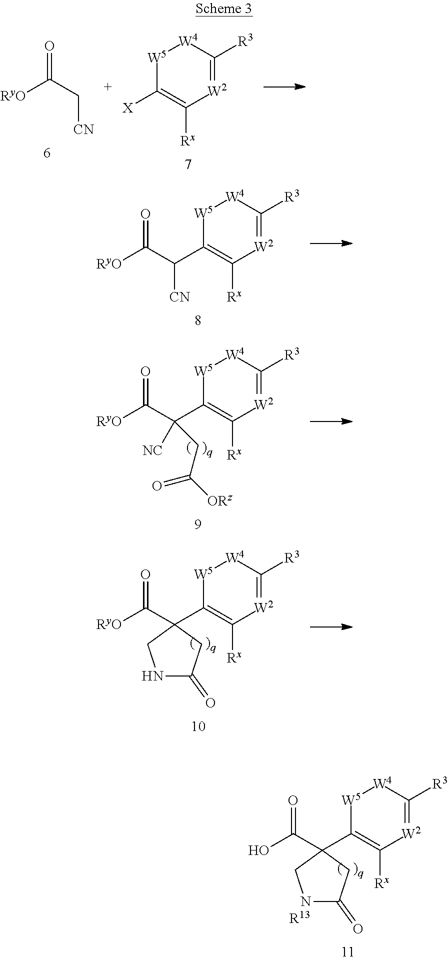

For certain embodiments, a person skilled in the art can also access the carboxylic acid used for the coupling depicted in Scheme 1 by an initial union of a suitable ketone (12) with an appropriately functionalized (hetero)aryl halide (7), in the presence of a promoter such as n-butyl lithium, Turbo Grignard, or the like (Scheme 4). The resulting (hetero)arylated alcohol (13) can then be made to undergo a Friedel-Crafts alkylation of 2-methylfuran, a known carboxylic acid surrogate, to furnish furan (14). Finally, oxidation of the furan ring in the presence of a suitable oxidant such as ozone, ruthenium(IV) salts, or the like, would furnish the requisite carboxylic acid (15).

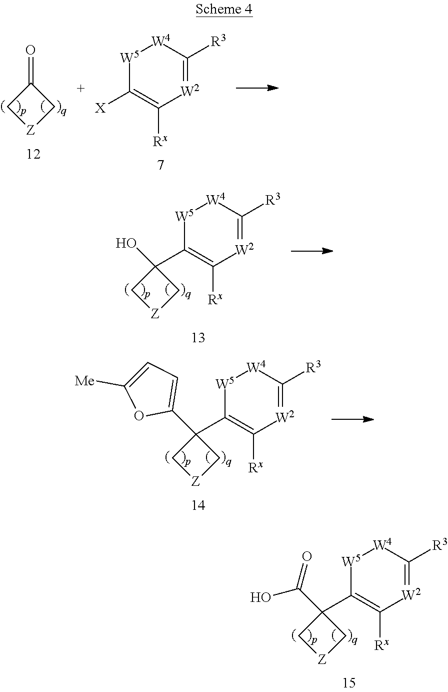

For certain embodiments, it may prove advantageous to instead convert (hetero)arylated alcohol (13) to nitrile (16) in the presence of a suitable Lewis acid, such as Indium(III) salts, aluminum(III) salts, or the like, and a suitable cyanide source, such as potassium cyanide, trimethylsilyl cyanide, or the like (Scheme 5). Nitrile (16) can in turn be converted, through the judicious choice of hydrolysis reaction conditions known to those skilled in the art, to either acid (15), for participation in the coupling event depicted in Scheme 1, or to primary amide (17), for participation in the coupling event depicted in Scheme 2.

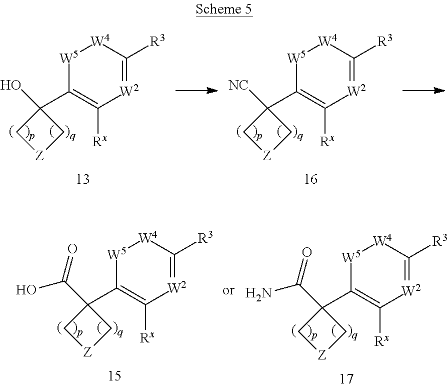

For certain embodiments, a person skilled in the art can also access nitrile (16) by reacting precursor nitrile (18) with an appropriately functionalized bis-electrophile (19), in the presence of a suitable base such as sodium hydride, potassium tert-butoxide, or the like, as depicted in Scheme 6. Alternatively, in instances where precursor nitrile (20) is more readily accessible, C-arylation with an appropriately functionalized (hetero)aryl halide (7), in the presence of a promoter such as a palladium catalyst, a copper catalyst, heat, or the like, and a base such as potassium phosphate, TEA, potassium bis(trimethylsilyl)amide, or the like, could instead be executed to access nitrile (16).

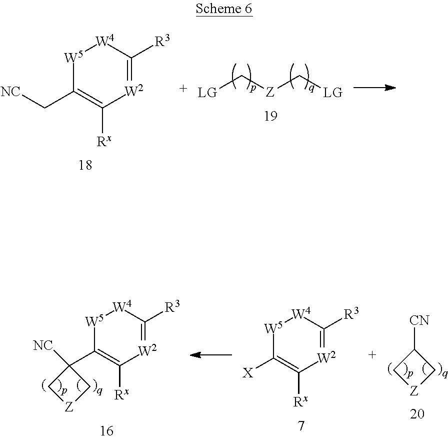

The general synthetic schemes above have been described in an illustrative manner and is intended to be in the nature of description rather than of limitation. It will also be appreciated that many of the reagents provided in the following examples may be substituted with other suitable reagents (see, e.g., is Fieser, L., et al., “Encyclopedia of Reagents for Organic Synthesis,” 2^{d} ed., New York: Wiley, 2009). In addition, it will be appreciated that conditions such as choice of solvent, temperature of reaction, volumes and reaction time may vary while still producing the desired compounds. Such changes and modifications, including without limitation, those relating to the chemical structures, substituents, derivatives, intermediates and/or syntheses provided herein, may be made without departing from the spirit and scope thereof.

### EXAMPLES

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography was performed on silica gel unless otherwise noted.

### Intermediate Acid 1: Preparation of 3-(2-isopropylphenyl)-1-methyl-5-oxopyrrolidine-3-carboxylic acid

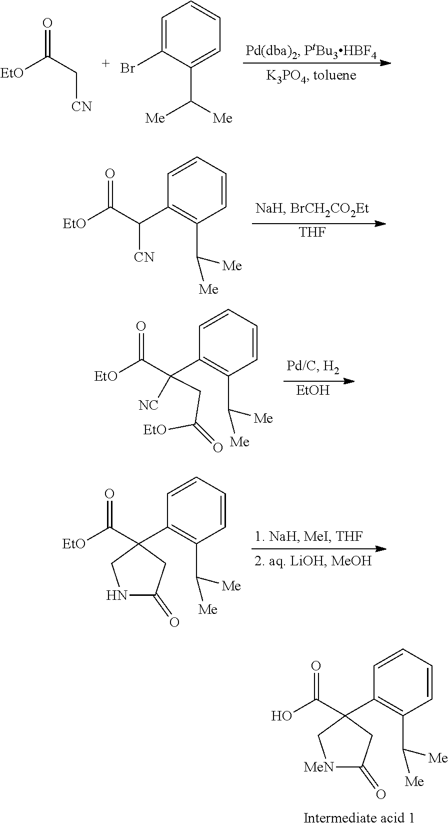

Step 1: In a thick-walled glass reaction vessel equipped with a magnetic stirrer and a Teflon screwcap was combined ethyl 2-cyanoacetate (2 equiv, Acros), 1-bromo-2-isopropylbenzene (1 equiv, TCI), bis(dibenzylidineacetone)palladium(0) (0.02 equiv, Sigma-Aldrich), tri-tert-butylphosphonium tetrafluoroborate (0.04 equiv, Strem), and potassium phosphate (3 equiv, Sigma-Aldrich) in toluene (0.4 M). The resulting dark purple suspension was then deoxygenated via subsurface purging with nitrogen for 10 min before the reaction vessel was tightly sealed and heated at 100° C. for 20 h. The resulting orange suspension was cooled to RT, diluted with tert-butyl methyl ether, and washed sequentially with water and brine. The organic extract thus obtained was then dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded ethyl 2-cyano-2-(2-isopropylphenyl)acetate as a colorless oil (46% yield).

Step 2: In a dried, round-bottom flask equipped with a magnetic stirrer was dissolved ethyl 2-cyano-2-(2-isopropylphenyl)acetate (1 equiv) from the previous step in anhydrous THE (0.28 M). To this was then added at 0° C. sodium hydride (60% w/w dispersion in paraffin oil, 1.5 equiv, Sigma-Aldrich) in one rapid portion. The resulting grey suspension was first stirred at 0° C. for 15 min and then at RT for 30 min. The now yellow suspension was then added ethyl 2-bromoacetate (1.5 equiv, Sigma-Aldrich), neat and dropwise, over a period of 5 min. Finally, a reflux condenser was attached, and the reaction mixture was heated at reflux for 16 h. The resulting pink reaction suspension was cooled to RT and carefully quenched with saturated aq. NH₄C₁. The biphasic mixture was then poured into a separatory funnel and extracted with tert-butyl methyl ether. The combined organic extracts were then washed further with water and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded diethyl 2-cyano-2-(2-isopropylphenyl)succinate as a pale-yellow oil (86% yield).

Step 3: In a dried, round-bottom flask equipped with a magnetic stirrer was dissolved diethyl 2-cyano-2-(2-isopropylphenyl)succinate (1 equiv) from the previous step in absolute ethanol (0.06 M). The resulting colorless solution was then deoxygenated via subsurface purging with nitrogen for 10 min before palladium (10% w/w over activated carbon, dry, 0.2 equiv, Sigma-Aldrich) was added in one rapid portion. The resulting black suspension was then subsurface purged with hydrogen for 10 min before it was stirred under a static hydrogen atmosphere (maintained with multiple balloons) at RT for 72 h. The reaction was subsequently quenched with dichloromethane and filtered through a bed of dichloromethane-wetted celite. The insolubles were washed further with dichloromethane and the filtrate was concentrated in vacuo. The solid residue thus obtained was then triturated in a 1:1 (v/v) solution of hexanes and tert-butyl methyl ether to afford ethyl 3-(2-isopropylphenyl)-5-oxopyrrolidine-3-carboxylate as a white solid (64% yield).

Step 4: In a thick-walled glass reaction vessel equipped with a magnetic stirrer and a Teflon screwcap was dissolved ethyl 3-(2-isopropylphenyl)-5-oxopyrrolidine-3-carboxylate (1 equiv) from the previous step in anhydrous THF (0.11 M). To this was then added at 0° C. sodium hydride (60% w/w dispersion in paraffin oil, 1.5 equiv, Sigma-Aldrich) in one rapid portion. The resulting grey suspension was first stirred at 0° C. for 15 min and then at RT for 30 min. The now pale-yellow suspension was then added iodomethane (1.5 equiv, Sigma-Aldrich), neat and dropwise, over a period of 5 min. After 2 h of stirring at RT, the reaction mixture was then added methanol (0.05 M) and lithium hydroxide (1 M solution in water, 6 equiv). The reaction vessel was then sealed and heated at 50° C. for 2 h. Upon cooling to RT, the reaction mixture was carefully quenched with HCl (1 M solution in water, 6 equiv) and the volatiles were then removed in vacuo. The resulting suspension was back extracted with EtOAc. The combined organic extracts were washed sequentially with water and brine, dried over MgSO₄, filtered, and the filtrate concentrated in vacuo. The crude product thus obtained was then triturated in a 1:1 (v/v) solution of EtOAc and tert-butyl methyl ether to afford the title compound as a white, crystalline solid (69% yield).

### Intermediate Acid 2: Preparation of 3-(2-isopropylphenyl)-5-oxo-1-((2-(trimethylsilyl)ethoxy)methyl)pyrrolidine-3-carboxylic acid

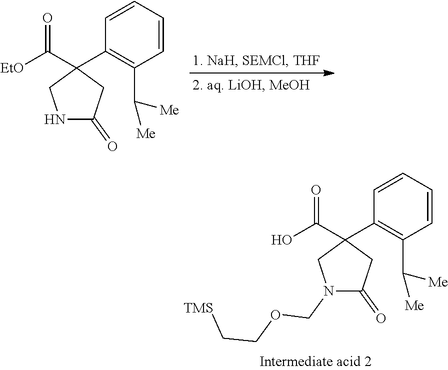 [SMILES: CCOC(=O)C1(c2ccccc2C(C)C)CNC(=O)C1 ; CC(C)c1ccccc1C1(C(=O)O)CC(=O)N(COCC[Si](C)(C)C)C1]

Step 1: In a dried, round-bottom flask equipped with a magnetic stirrer was dissolved ethyl 3-(2-isopropylphenyl)-5-oxopyrrolidine-3-carboxylate (1 equiv, Intermediate acid 1, Step 3) in anhydrous THE (0.11 M). To this was then added at 0° C. sodium hydride (60% w/w dispersion in paraffin oil, 1.5 equiv, Sigma-Aldrich) in one rapid portion. The resulting grey suspension was first stirred at 0° C. for 15 min and then at RT for 30 min. The now pale-yellow suspension was then added 2-(trimethylsilyl)ethoxymethyl chloride (2.2 equiv, technical grade, Sigma-Aldrich), neat and dropwise, over a period of 5 min. After 48 h of stirring at RT, the reaction mixture was diluted with tert-butyl methyl ether and washed sequentially with 1 M aq. NaOH, water and brine. The organic extract was then dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded ethyl 3-(2-isopropylphenyl)-5-oxo-1-((2-(trimethylsilyl)ethoxy)methyl)pyrrolidine-3-carboxylate as a colorless oil (77% yield).

Step 2: In a dried, round-bottom flask equipped with a magnetic stirrer was dissolved ethyl 3-(2-isopropylphenyl)-5-oxo-1-((2-(trimethylsilyl)ethoxy)methyl)pyrrolidine-3-carboxylate (1 equiv) from the previous step in a 3:2 (v/v) solution of THE and methanol (0.055 M). To this colorless solution was then added lithium hydroxide (1 M solution in water, 3 equiv) and the resulting mixture was heated at 50° C. for 18 h. Upon cooling to RT, the reaction mixture was carefully quenched with HCl (1 M solution in water, 3 equiv) and the volatiles were removed in vacuo. The resulting suspension was back extracted with EtOAc. The combined organic extracts were washed sequentially with water and brine, dried over MgSO₄, filtered, and the filtrate concentrated in vacuo. The crude product thus obtained was then triturated in a 1:1 (v/v) solution of hexanes and tert-butyl methyl ether to afford the title compound as a white, crystalline solid (95% yield).

### Intermediate Acid 3: Preparation of 3-(2-isopropylphenyl)oxetane-3-carboxylic acid

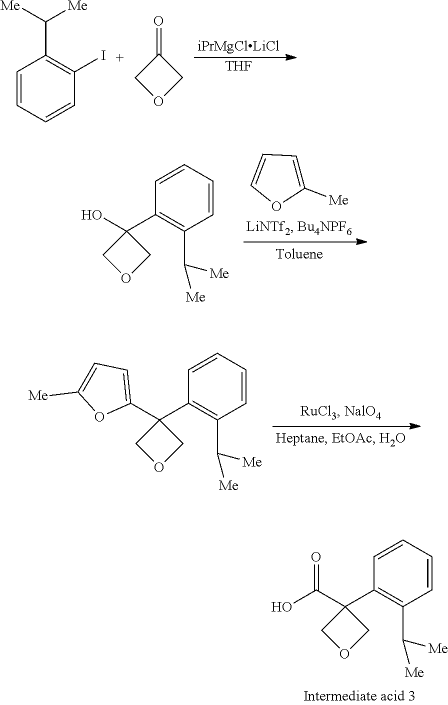

Step 1: In a dried, round-bottom flask equipped with a magnetic stirrer was dissolved 1-iodo-2-isopropylbenzene (1 equiv, Combi-Blocks) in THE (0.30 M). The resulting solution was then cooled to 0° C. before Turbo Grignard (1.3 M solution in THF, 1.2 equiv, Sigma-Aldrich) was added dropwise over 5 min. After 60 min of stirring at 0° C., oxetan-3-one (1.2 equiv, Combi-Blocks) was then added neat and dropwise over 5 min. The cooling bath was then removed, and the reaction mixture was stirred at RT for 30 min. Finally, the reaction was quenched with the addition of saturated aq. NH₄Cl and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded 3-(2-isopropylphenyl)oxetan-3-ol as a white solid (47% yield).

Step 2: In a dried, round-bottom flask equipped with a magnetic stirrer was combined 3-(2-isopropylphenyl)oxetan-3-ol (1 equiv) from the previous step, bis(trifluoromethane)sulfonimide lithium salt (0.11 equiv, Sigma-Aldrich), tetrabutylammonium hexafluorophosphate (0.055 equiv, Sigma-Aldrich), and 2-methylfuran (5 equiv, Sigma-Aldrich) in toluene (0.4 M). The resulting reaction mixture was then heated at 60° C. for 16 h. The reaction was then quenched with the addition of water and extracted with EtOAc. The combined organic extracts were dried over MgSO₄, filtered, and the filtrate concentrated in vacuo. Purification of the crude product thus obtained by way of column chromatography (SiO₂, gradient elution: Hex→4:1 (v/v) Hex: EtOAc) afforded 2-(3-(2-isopropylphenyl)oxetan-3-yl)-5-methylfuran as a colorless oil (23% yield).

Step 3: In round-bottom flask equipped with a magnetic stirrer was dissolved 2-(3-(2-isopropylphenyl)oxetan-3-yl)-5-methylfuran (1 equiv) from the previous step in a 1:1:2 (v/v/v) solution of heptane, ethyl acetate, and water. To this was then added sodium periodate (7 equiv, Sigma-Aldrich) and the resulting biphasic mixture was vigorously stirred at RT for 5 min. Upon cooling to 0° C., ruthenium(III) chloride hydrate (0.03 equiv, Sigma-Aldrich) was added in one rapid portion. The reaction mixture was then warmed slowly to RT over 16 h and diluted further with water. The aqueous layer was separated and back extracted with EtOAc. The combined organic extracts were dried over MgSO₄, filtered, and the filtrate concentrated in vacuo. Purification of the crude product thus obtained by way of column chromatography (SiO₂, gradient elution: 7:3 (v/v) Hex: EtOAc→EtOAc) afforded the title compound as a pale-yellow solid (47% yield).

The following acid was prepared in an analogous fashion to Intermediate acid 3, but substituting 1-iodo-2-isopropylbenzene in step 1 with the requisite (hetero)aryl halide.

| Starting Material | Product |
|---|---|
| 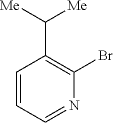 | 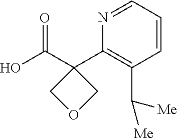 [SMILES: CC(C)c1cccnc1C1(C(=O)O)COC1] |
| (Ambeed) | Intermediate acid 4 |

Intermediate acid 5: Preparation of 3-(2-isopropylphenyl)-5-oxopyrrolidine-3-carboxylic acid

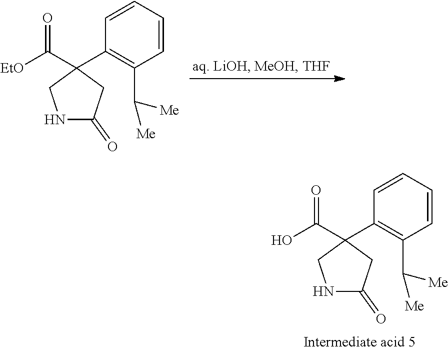 [SMILES: CCOC(=O)C1(c2ccccc2C(C)C)CNC(=O)C1 ; CC(C)c1ccccc1C1(C(=O)O)CNC(=O)C1]

In a dried, round-bottom flask equipped with a magnetic stirrer was dissolved ethyl 3-(2-isopropylphenyl)-5-oxopyrrolidine-3-carboxylate (1 equiv, Intermediate acid 1, Step 3) in a 2:1 (v/v) solution of THF and methanol (0.077 M). To this colorless solution was then added lithium hydroxide (1 M solution in water, 3 equiv) and the resulting mixture was heated at 50° C. for 18 h. Upon cooling to RT, the reaction mixture was carefully quenched with HCl (1 M solution in water, 3 equiv) and the volatiles were removed in vacuo. The resulting suspension was back extracted with EtOAc. The combined organic extracts were washed sequentially with water and brine, dried over MgSO₄, filtered, and the filtrate concentrated in vacuo. The crude product thus obtained was then triturated in a 1:1:1 (v/v/v) solution of EtOAc, hexanes, and tert-butyl methyl ether to afford the title compound as a white, crystalline solid (98% yield). The racemic Intermediate acid 5 can be further resolved into its enantiomerically pure antipodes, Intermediate acid 5-isomer A and Intermediate acid 5-isomer B, via chiral column chromatography.

| Starting Mixture | Separation Conditions | RT (min) |
|---|---|---|
| 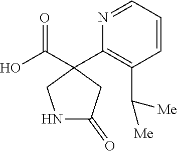 [SMILES: CC(C)c1cccnc1C1(C(=O)O)CNC(=O)C1] | Column: CHIRALPAK IH (5 × 25 cm; 5 μm) Mobile phase: 60:40 (v/v) supercritical CO₂: MeOH Additive: 0.5% 2 M NH₃ in MeOH Flow rate: 20 mL/min Injection volume: 4 mL in MeOH | Intermediate acid 5-isomer A 8.49 5-isomer B 10.93 |
| Intermediate acid 5 | | |

Intermediate acid 6: Preparation of 8-(2-isopropylphenyl)-1,4-dioxaspiro[4.5]decane-8-carboxylic acid

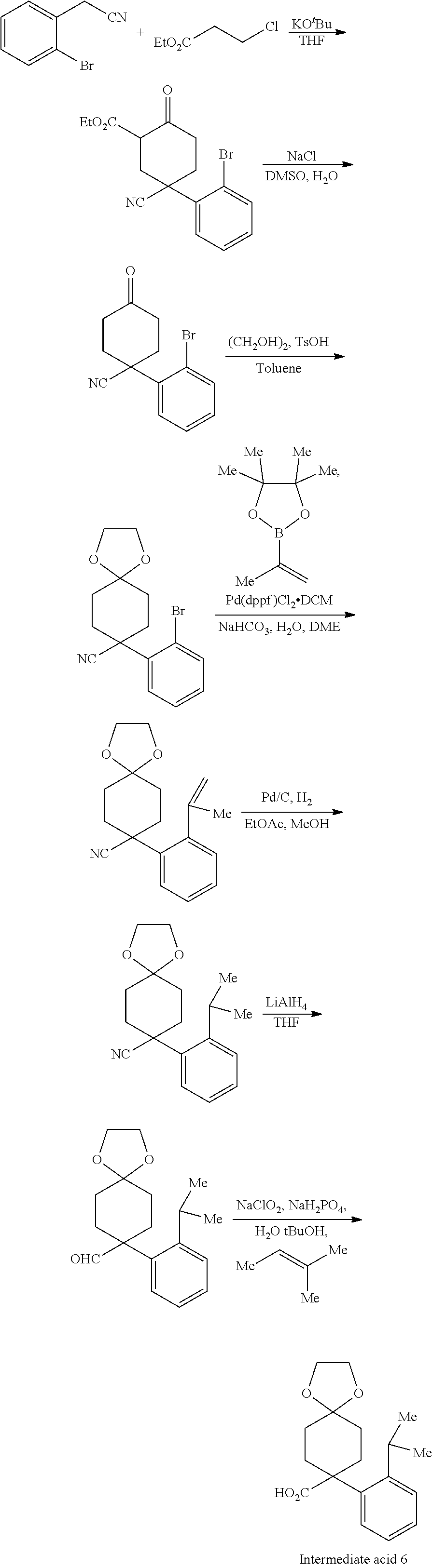

Step 1: In a dried, round-bottom flask equipped with a magnetic stirrer was suspended potassium tert-butoxide (5 equiv, Sigma-Aldrich) in THE (1.5 M). The resulting white suspension was then cooled to 0° C. before 2-(2-bromophenyl)acetonitrile (1.2 equiv, AmBeed) was added neat and dropwise over 5 min. The cooling bath was then removed, and the resulting yellow suspension was stirred at RT for 15 min. The reaction mixture thus obtained was re-cooled to 0° C. before ethyl 3-chloropropanoate (2 equiv, Acros) was added neat and dropwise over 5 min. The resulting orange suspension was then stirred at 0° C. for 45 min and then at RT for 4 h. The crude reaction mixture was then diluted with tert-butyl methyl ether and water before the reaction was carefully quenched with HCl (1 M solution in water, 5 equiv). The aqueous layer was separated and back extracted with tert-butyl methyl ether. The combined organic extracts were washed further with water and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded ethyl 5-(2-bromophenyl)-5-cyano-2-oxocyclohexane-1-carboxylate as a pale-yellow oil (64% yield).

Step 2: In a dried, round-bottom flask equipped with a magnetic stirrer was combined ethyl 5-(2-bromophenyl)-5-cyano-2-oxocyclohexane-1-carboxylate (1 equiv) from the previous step and sodium chloride (1 equiv, Acros) in an 8:1 (v/v) solution of dimethylsulfoxide and water (0.28 M). The resulting reaction solution was then heated at 140° C. under a nitrogen atmosphere for 18 h. The reaction mixture was then cooled to RT, diluted with dichloromethane, and washed sequentially with 1 M aq. HCl, water and brine. The organic extract was then dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded 1-(2-bromophenyl)-4-oxocyclohexane-1-carbonitrile as a pale-yellow oil (74% yield).

Step 3: In a round-bottom flask equipped with a magnetic stirrer and a Dean-Stark apparatus was combined 1-(2-bromophenyl)-4-oxocyclohexane-1-carbonitrile (1 equiv) from the previous step, ethylene glycol (4.4 equiv, Sigma-Aldrich), and p-toluenesulfonic acid (a few crystals, Combi-Blocks) in toluene (0.09 M). The resulting solution was heated at reflux for 48 h. The volatiles were then removed in vacuo and the crude product thus obtained was purified by way of column chromatography (SiO₂, gradient elution: 9:1 (v/v) Hex: EtOAc→EtOAc) to afford 8-(2-bromophenyl)-1,4-dioxaspiro[4.5]decane-8-carbonitrile as an off-white solid (65% yield).

Step 4: In a thick-walled glass reaction vessel equipped with a magnetic stirrer and a Teflon screwcap was combined 8-(2-bromophenyl)-1,4-dioxaspiro[4.5]decane-8-carbonitrile (1 equiv) from the previous step, 4,4,5,5-tetramethyl-2-(prop-1-en-2-yl)-1,3,2-dioxaborolane (3 equiv, Frontier Scientific), [1,1′-bis(diphenylphosphino)ferrocene]dichloropalladium(II) dichloromethane complex (0.1 equiv, Sigma-Aldrich), and sodium bicarbonate (3 equiv, Alfa Aesar) in a 3:1 (v/v) solution of 1,2-dimethoxyethane and water (0.15 M). The resulting mixture was deoxygenated via subsurface purging with nitrogen for 10 min before the reaction vessel was tightly sealed and heated at 95° C. for 18 h. The now deep brown suspension was cooled to RT, quenched with water, and extracted with EtOAc. The combined organic extracts were washed further with water and brine, dried over MgSO₄, filtered, and the filtrate concentrated in vacuo. Purification of the crude product thus obtained by way of column chromatography (SiO₂, Hex→1:1 (v/v) Hex: EtOAc) afforded 8-(2-(prop-1-en-2-yl)phenyl)-1,4-dioxaspiro[4.5]decane-8-carbonitrile as a colorless oil (80% yield).

Step 5: In a dried, round-bottom flask equipped with a magnetic stirrer was dissolved 8-(2-(prop-1-en-2-yl)phenyl)-1,4-dioxaspiro[4.5]decane-8-carbonitrile (1 equiv) from the previous step in a 2:1 (v/v) solution of EtOAc and methanol (0.064 M). The resulting solution was then deoxygenated via subsurface purging with nitrogen for 10 min before palladium (10% w/w over activated carbon, dry, 0.15 equiv, Sigma-Aldrich) was added in one rapid portion. The resulting black suspension was then subsurface purged with hydrogen for 10 min before it was stirred under a static hydrogen atmosphere at RT for 2 h. The reaction was subsequently quenched with dichloromethane and filtered through a bed of dichloromethane-wetted celite. The insolubles were washed further with dichloromethane and the filtrate thus obtained was concentrated in vacuo to afford 8-(2-isopropylphenyl)-1,4-dioxaspiro[4.5]decane-8-carbonitrile as a white, crystalline solid (99% yield).

Step 6: In a dried, round-bottom flask equipped with a magnetic stirrer was suspended lithium aluminum hydride (1 equiv, Sigma-Aldrich) in anhydrous THF (1 M). To this grey suspension was then added, at 0° C., 8-(2-isopropylphenyl)-1,4-dioxaspiro[4.5]decane-8-carbonitrile (1 equiv) from the previous step as a THE solution (0.2 M) dropwise over a period of 10 min. The resulting suspension was stirred at 0° C. for 30 min and then at RT for 4 h. The crude reaction mixture was then re-cooled to 0° C. and carefully quenched with NaOH (0.5 M solution in water, 1 equiv). The resulting white suspension was vigorously stirred at 0° C. for 10 min before tert-butyl methyl ether was added. The biphasic mixture thus obtained was stirred vigorously at RT for an additional 40 min before the organic layer was separated and concentrated in vacuo. The resulting residue was taken up again in THF (0.33 M), added HCl (1 M solution in water, 1 equiv), and stirred at RT for an additional 20 min. The reaction mixture was then carefully quenched with saturated aq. NaHCO₃ and back extracted with tert-butyl methyl ether. The combined organic extracts were washed further with water and brine, dried over MgSO₄, filtered, and the filtrate concentrated in vacuo. Purification of the crude product thus obtained by way of column chromatography (SiO₂, gradient elution: Hex→1:2 (v/v) Hex: EtOAc) afforded 8-(2-isopropylphenyl)-1,4-dioxaspiro[4.5]decane-8-carbaldehyde as a white crystalline solid (74% yield).

Step 7: In a dried, round-bottom flask equipped with a magnetic stirrer was combined 8-(2-isopropylphenyl)-1,4-dioxaspiro[4.5]decane-8-carbaldehyde (1 equiv) from the previous step and sodium hydrogen phosphate (15 equiv, Sigma-Aldrich) in a 2:2:1 (v/v/v) solution of tert-butanol, water, and 2-methylbut-2-ene (0.01 M). To this colorless solution was then added sodium chlorite (30 equiv, Sigma-Aldrich) and the resulting mixture was stirred at RT for 96 h. The volatiles were then removed in vacuo, and the resulting residue was partitioned between saturated aq. NH₄Cl and EtOAc. The aqueous layer was separated and back extracted with EtOAc. The combined organic extracts were washed further with water and brine, dried over MgSO₄, filtered, and the filtrate concentrated in vacuo. The crude product thus obtained was then triturated in a 1:1 (v/v) solution of hexanes and tert-butyl methyl ether to afford the title compound as a white, crystalline solid (68% yield).

### Intermediate Acid 7 & Intermediate acid 8: Preparation of cis-3-(2-ethoxy-2-oxoethyl)-1-(2-isopropylphenyl)cyclobutane-1-carboxylic acid and trans-3-(2-ethoxy-2-oxoethyl)-1-(2-isopropylphenyl)cyclobutane-1-carboxylic acid

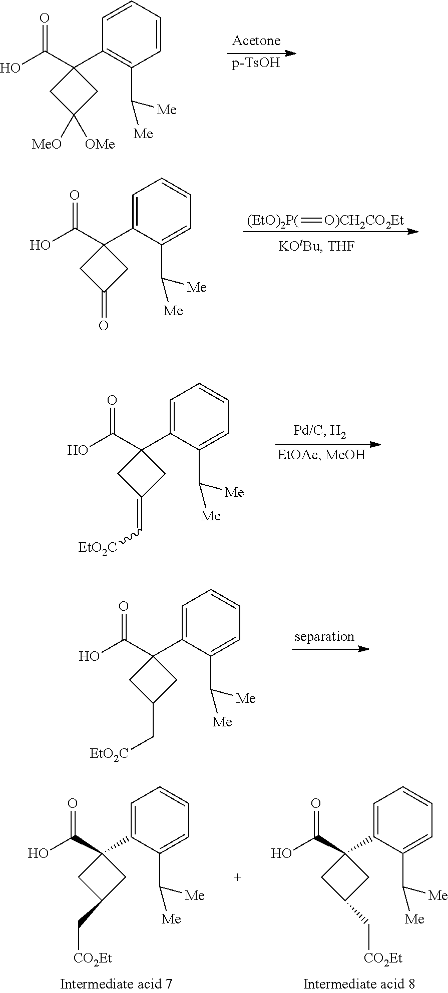

Step 1: In a dried, round-bottom flask equipped with a magnetic stirrer was dissolved 1-(2-isopropylphenyl)-3,3-dimethoxycyclobutane-1-carboxylic acid (1 equiv, Intermediate ketone 1, step 4) in acetone (0.36 M). 4-Methylbenzenesulfonic acid (0.5 equiv, Combi-Blocks) was added and the resulting mixture was stirred at RT for 4 h. The volatiles were then concentrated in vacuo and the resulting residue was partitioned between EtOAc and saturated aqueous NH₄C₁. The organic layer was separated, washed further with saturated aqueous NH₄C₁, and brine, dried over MgSO₄ and filtered. Concentration of the filtrate thus obtained in vacuo afforded crude 1-(2-isopropylphenyl)-3-oxocyclobutane-1-carboxylic acid (92% yield) as a white solid.

Step 2: In a dried, round-bottom flask equipped with a magnetic stirrer was dissolved ethyl 2-(diethoxyphosphoryl)acetate (2.2 equiv, Combi-Blocks) in anhydrous THF (0.73 M). To this solution was then added potassium tert-butoxide (1 M solution in THF, 2.2 equiv, Sigma-Aldrich) dropwise over a period of 5 min and the resulting mixture was stirred at RT for an additional 30 min. Finally, 1-(2-isopropylphenyl)-3-oxocyclobutane-1-carboxylic acid (1 equiv) from the previous step was added as a solution in THE (0.33 M) and the resulting mixture was stirred at RT for an additional 14 h. The reaction was then quenched with the careful addition of 2 M aqueous HCl and extracted with EtOAc. The combined organic extracts were washed further with brine, dried over MgSO₄, filtered, and the filtrate concentrated in vacuo. Purification of the crude product thus obtained by way of column chromatography (SiO₂, gradient elution: 95:5 (v/v) Hex: EtOAc→35:65 (v/v) Hex: EtOAc) afforded 3-(2-ethoxy-2-oxoethylidene)-1-(2-isopropylphenyl)cyclobutane-1-carboxylic acid as a viscous oil (78% yield).

Step 3: In a dried, round-bottom flask equipped with a magnetic stirrer was dissolved 3-(2-ethoxy-2-oxoethylidene)-1-(2-isopropylphenyl)cyclobutane-1-carboxylic acid (1 equiv) from the previous step in a 2:1 (v/v) solution of EtOAc and methanol (0.030 M). The resulting solution was then deoxygenated via subsurface purging with nitrogen for 10 min before palladium (10% w/w over activated carbon, dry, 0.15 equiv, Sigma-Aldrich) was added in one rapid portion. The resulting black suspension was then subsurface purged with hydrogen for 10 min before it was stirred under a static hydrogen atmosphere at RT for 2 h. The reaction was subsequently quenched with dichloromethane and filtered through a bed of dichloromethane-wetted celite. The insolubles were washed further with dichloromethane and the filtrate thus obtained was concentrated in vacuo to afford 3-(2-ethoxy-2-oxoethyl)-1-(2-isopropylphenyl)cyclobutane-1-carboxylic acid as a 2.5:1 mixture of diastereomers. The isomers were separated via chiral supercritical fluid column chromatography (ChiralPal AD: 85:15 (v/v) supercritical CO₂: EtOH) to afford the two title compounds.

Intermediate amide 1: Preparation of 3-(2-isopropylphenyl)-5-oxo-1-((2-(trimethylsilyl)ethoxy)methyl)pyrrolidine-3-carboxamide

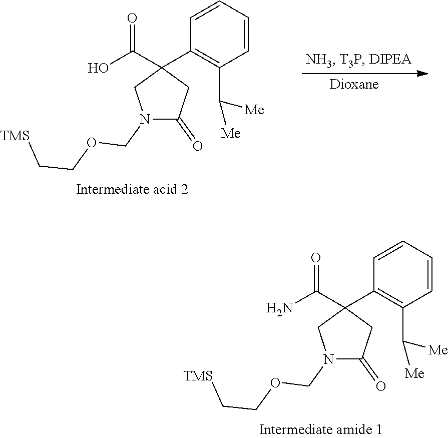 [SMILES: CC(C)c1ccccc1C1(C(=O)O)CC(=O)N(COCC[Si](C)(C)C)C1 ; CC(C)c1ccccc1C1(C(N)=O)CC(=O)N(COCC[Si](C)(C)C)C1]

In a dried, round-bottom flask equipped with a magnetic stirrer was combined Intermediate acid 2 (1 equiv), ammonia (0.4 M in 1,4-dioxane, 5 equiv, Sigma-Aldrich), and N,N-diisopropylethylamine (1.5 equiv, Sigma-Aldrich) in 1,4-dioxane (0.05 M). To this reaction mixture was then added propylphosphonic anhydride (50% w/w solution in EtOAc, 1.5 equiv, Sigma-Aldrich) and the resulting solution was stirred at RT for 18 h. The now white suspension was then quenched with water and extracted with EtOAc. The combined organic extracts were washed further with 1 M aq. NaOH, water and brine, dried over MgSO₄, filtered, and the filtrate concentrated in vacuo. Purification of the crude product thus obtained by way of column chromatography (SiO₂, gradient elution: 9:1 (v/v) Hex: EtOAc→EtOAc) afforded the title compound as a white solid (73% yield).

The following amides were prepared in an analogous fashion to Intermediate amide 1, but substituting Intermediate acid 2 with the requisite acid.

| Starting Material | Product | Starting Material | Product |
|---|---|---|---|
| 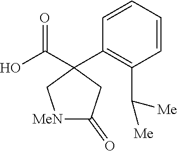 [SMILES: CC(C)c1ccccc1C1(C(=O)O)CC(=O)N(C)C1] | 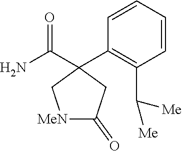 [SMILES: CC(C)c1ccccc1C1(C(N)=O)CC(=O)N(C)C1] | 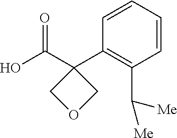 [SMILES: CC(C)c1ccccc1C1(C(=O)O)COC1] | 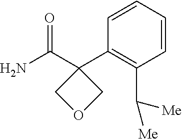 [SMILES: CC(C)c1ccccc1C1(C(N)=O)COC1] |
| Intermedate acid 1 | Intermedate amide 2 | Intermediate acid 3 | Intermediate amide 3 |

Intermediate amide 4: Preparation of 4-(2-isopropylphenyl)tetrahydro-2H-pyran-4-carboxamide

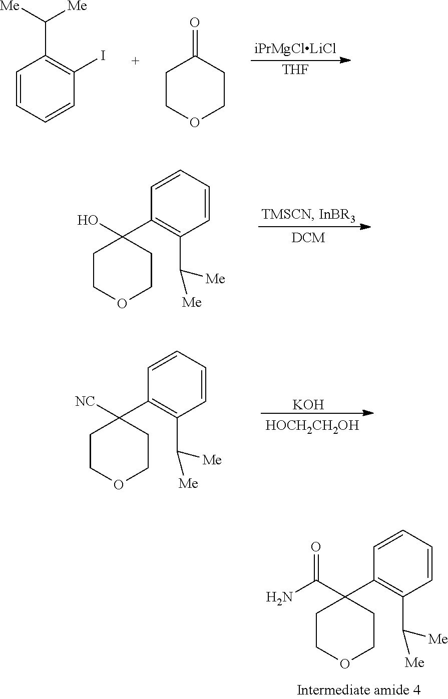

Step 1: In a dried, round-bottom flask equipped with a magnetic stirrer was dissolved 1-iodo-2-isopropylbenzene (1 equiv, Combi-Blocks) in THE (0.30 M). The resulting solution was then cooled to 0° C. before Turbo Grignard (1.3 M solution in THF, 1.2 equiv, Sigma-Aldrich) was added dropwise over 5 min. After 60 min of stirring at 0° C., tetrahydro-4H-pyran-4-one (1.2 equiv, Combi-Blocks) was then added neat and dropwise over 5 min. The cooling bath was then removed, and the reaction mixture was stirred at RT for 30 min. Finally, the reaction was quenched with the addition of saturated aq. NH₄Cl and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: Hex 4 3:2 (v/v) Hex: EtOAc) afforded 4-(2-isopropylphenyl)tetrahydro-2H-pyran-4-ol as a white solid (55% yield).

Step 2: In a dried, round-bottom flask equipped with a magnetic stirrer was suspended indium(III) bromide (0.2 equiv, Sigma-Aldrich) in dichloromethane (0.2 M). To this suspension was then added trimethylsilyl cyanide (3 equiv, TCI) neat and dropwise over a period of 10 min. Finally, 4-(2-isopropylphenyl)tetrahydro-2H-pyran-4-ol (1 equiv) from the previous step in dichloromethane (0.2 M) was added dropwise over a period of 10 min. Following the completion of addition, the reaction mixture was sonicated briefly and then stirred at RT for 1 h. The reaction was then quenched with the addition of water and extracted with dichloromethane. The combined organic extracts were washed further with water and brine, dried over MgSO₄, filtered and the filtrate concentrated in vacuo. Purification of the crude product thus obtained by way of column chromatography (SiO₂, gradient elution: Hex 4 7:3 (v/v) Hex: EtOAc) afforded 4-(2-isopropylphenyl)tetrahydro-2H-pyran-4-carbonitrile as a white, crystalline solid (71% yield).

Step 3: In a thick-walled glass reaction vessel equipped with a magnetic stirrer and a Teflon screwcap was combined 4-(2-isopropylphenyl)tetrahydro-2H-pyran-4-carbonitrile (1 equiv) from the previous step and potassium hydroxide (10 equiv, Alfa Aesar) in ethylene glycol (0.25 M). The vessel was then tightly sealed and heated at 180° C. for 48 h. The reaction mixture was then cooled to RT, neutralized with HCl (1 M solution in water, 10 equiv), and extracted with dichloromethane. The combined organic extracts were washed further with water and brine, dried over MgSO₄, filtered and the filtrate concentrated in vacuo. Purification of the crude product thus obtained by way of column chromatography (SiO₂, gradient elution: 7:3 (v/v) Hex: EtOAc→EtOAc) afforded the title compound as a white, crystalline solid (85% yield).

Intermediate halide 1: Preparation of 5-chloro-3-(difluoromethoxy)-2-iodopyridine

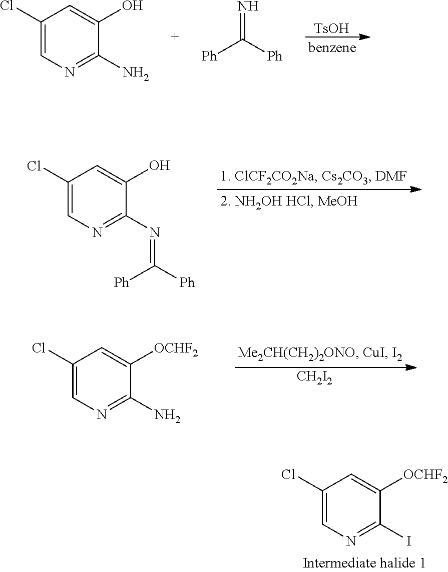

Step 1: In a dried, round-bottom flask equipped with a magnetic stirrer was suspended 2-amino-5-chloropyridin-3-ol (1 equiv, Combi-Blocks), benzophenone imine (1 equiv, Combi-Blocks), and p-toluenesulfonic acid (a few crystals, Combi-Blocks) in benzene (0.1 M). A Dean-Stark trap was attached, and the resulting mixture was heated to reflux for 16 h. The now yellow suspension was cooled to RT, diluted with dichloromethane, and washed with water. The organic extract thus obtained was then dried over MgSO₄, filtered, and the filtrate concentrated in vacuo. The crude product thus obtained was then triturated in hexanes to afford 5-chloro-2-((diphenylmethylene)amino)pyridin-3-ol as a yellow solid (40% yield).

Step 2: In a dried, round-bottom flask equipped with a magnetic stirrer was suspended 5-chloro-2-((diphenylmethylene)amino)pyridin-3-ol (1 equiv) from the previous step and cesium carbonate (1.1 equiv, Sigma-Aldrich) in DMF (0.19 M). The resulting suspension was heated to 90° C. before sodium chlorodifluoroacetate (1.1 equiv, TCI) was added as a DMF solution (0.2 M), dropwise over a period of 30 min. Following the completion of addition, the now dark reaction solution was heated at 90° C. for an extra 30 min before it was cooled to RT, diluted with tert-butyl methyl ether, and washed sequentially with 1 M aq. NaOH, water and brine. The organic layer was then dried over MgSO₄, filtered, and the filtrate concentrated in vacuo. The resulting residue was then taken up in methanol (0.06 M) and added hydroxylamine hydrochloride (2 equiv, Sigma-Aldrich). After 3 h of stirring at RT, the volatiles were then removed in vacuo and the resulting residue partitioned between EtOAc and water. The organic layer was separated, washed further with water and brine, dried over MgSO₄, filtered, and the filtrate concentrated in vacuo. Purification of the crude product thus obtained by way of column chromatography (SiO₂, gradient elution: 9:1 (v/v) Hex: EtOAc→EtOAc) afforded 5-chloro-3-(difluoromethoxy)pyridin-2-amine as an off-white solid (32% yield).

Step 3: In a dried, round-bottom flask equipped with a magnetic stirrer was suspended 5-chloro-3-(difluoromethoxy)pyridin-2-amine (1 equiv) from the previous step, copper(I) iodide (1 equiv, Sigma-Aldrich), and iodine (1 equiv, Sigma-Aldrich) in diiodomethane (0.23 M). The resulting purple suspension was vigorously stirred at 85° C. before isopentyl nitrite (3 equiv, Sigma-Aldrich) was added neat and dropwise over a period of 3 min. Following the completion of addition, the reaction suspension was heated at 85° C. for an extra 10 min before it was cooled to RT, diluted with tert-butyl methyl ether, and washed sequentially with 10% aq. Na₂S₂O₃, 1 M aq. HCl, water and brine. The organic layer was then dried over MgSO₄, filtered, and the filtrate concentrated in vacuo. Purification of the crude product thus obtained by way of reverse phase column chromatography (Cis, gradient elution: 9:1 (v/v) H₂O: MeCN+0.1% formic acid 4 MeCN+0.1% formic acid) afforded the title compound as a pale-yellow oil (58% yield).

Intermediate halide 2: Preparation of 2-bromo-5-chloro-3-(difluoromethoxy)-6-methoxypyridine

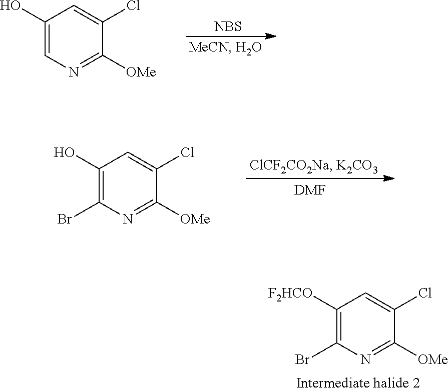

Step 1: In a dried, round-bottom flask equipped with a magnetic stirrer was dissolved 5-chloro-6-methoxypyridin-3-ol (1 equiv, Frontier Specialty Chemicals) in a 5:1 (v/v) solution of acetonitrile and water (0.63 M). To this solution was then added at 0° C. N-bromosuccinimide (1.1 equiv, Acros) portion-wise over a period of 5 min. The resulting pink solution was then stirred at 0° C. for 15 min and finally at RT for 45 min. The crude reaction mixture thus obtained was diluted with EtOAc, washed sequentially with water and brine, dried over MgSO₄, filtered, and the filtrate concentrated in vacuo. Purification of the crude product thus obtained by way of column chromatography (SiO₂, gradient elution: 9:1 (v/v) Hex: EtOAc→EtOAc) afforded 2-bromo-5-chloro-6-methoxypyridin-3-ol as a white solid (82% yield).

Step 2: In a dried, round-bottom flask equipped with a magnetic stirrer was suspended potassium carbonate (4 equiv, Sigma-Aldrich) in DMF (0.68 M). The reaction suspension was heated to 100° C. before a DMF solution (0.25 M) of 2-bromo-5-chloro-6-methoxypyridin-3-ol (1 equiv) from the previous step and sodium chlorodifluoroacetate (1.5 equiv, TCI) was added dropwise over a period of 10 min. Following the completion of addition, the now orange reaction solution was heated at 100° C. for an extra 45 min before it was cooled to RT, diluted with tert-butyl methyl ether, and washed sequentially with water and brine. The organic layer was then dried over MgSO₄, filtered, and the filtrate concentrated in vacuo. The resulting residue was then triturated in a 1:1 (v/v) solution of hexanes and tert-butyl methyl ether and filtered. Concentration of the filtrate in vacuo afforded the title compound as a yellow oil (70% yield).

Intermediate halide 3: Preparation of 3-bromo-2-(difluoromethoxy)-6-methylpyridine

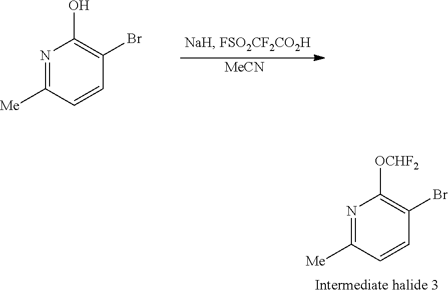 [SMILES: "MeCN" ; Cc1ccc(Br)c(O)n1 ; Cc1ccc(Br)c(OC(F)F)n1]

In a dried, round-bottom flask equipped with a magnetic stirrer was dissolved 3-bromo-6-methylpyridin-2-ol (1 equiv, Combi-Blocks) in acetonitrile (0.10 M). To this was then added sodium hydride (60% w/w dispersion in paraffin oil, 2.4 equiv, Sigma-Aldrich) in one rapid portion and the resulting mixture was stirred at RT for 10 min. Then, 2,2-difluoro-2-(fluorosulfonyl)acetic acid (1.4 equiv, Sigma-Aldrich) was added neat and dropwise over a period of 5 min, during which time a mild exotherm was observed. After 24 h of stirring at RT, the crude reaction mixture was carefully quenched with water, and then diluted with a 1:1 (v/v) solution of ethyl acetate and hexanes. The organic layer was then separated and washed sequentially with saturated aq. NaHCO₃, water and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded the title compound as a yellow oil (62% yield).

Intermediate halide 4: Preparation of 4-chloro-2-(difluoromethoxy)-1-iodobenzene

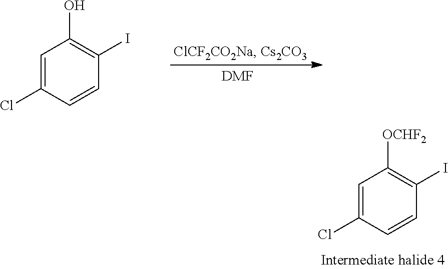

In a dried, round-bottom flask equipped with a magnetic stirrer was combined 5-chloro-2-iodophenol (1 equiv, Combi-Blocks), cesium carbonate (2 equiv, Sigma-Aldrich), and sodium chlorodifluoroacetate (1.5 equiv, TCI) in DMF (0.25 M). The reaction mixture was heated at 110° C. for 10 min during which time vigorous gas evolution could be discerned. The now orange reaction mixture was cooled to RT, diluted with tert-butyl methyl ether, and washed sequentially with water and brine. The organic layer was then dried over MgSO₄, filtered, and the filtrate concentrated in vacuo. Purification of the crude product thus obtained by way of column chromatography (SiO₂, gradient elution: Hex 4 9:1 (v/v) Hex: EtOAc) afforded the title compound as a colorless oil (88% yield).

Intermediate amine 1: Preparation of 2-(difluoromethoxy)-6-methoxypyridin-3-amine

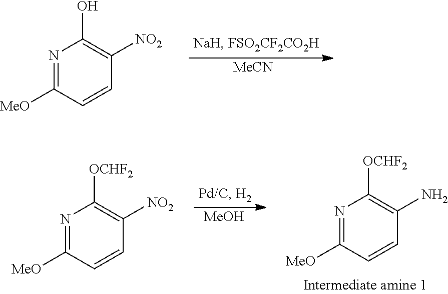 [SMILES: "MeCN" ; "MeOH" ; COc1ccc([N+](=O)[O-])c(O)n1 ; COc1ccc([N+](=O)[O-])c(OC(F)F)n1 ; COc1ccc(N)c(OC(F)F)n1]

Step 1: In a dried, round-bottom flask equipped with a magnetic stirrer was suspended 6-methoxy-3-nitropyridin-2-ol (1 equiv, Combi-Blocks) in acetonitrile (0.10 M). To this was then added sodium hydride (60% w/w dispersion in paraffin oil, 2.8 equiv, Sigma-Aldrich) in one rapid portion and the resulting mixture was stirred at RT for 10 min to afford a brownish, yellow suspension. Then, 2,2-difluoro-2-(fluorosulfonyl)acetic acid (1.8 equiv, Sigma-Aldrich) was added neat and dropwise over a period of 5 min, during which time a mild exotherm was observed. After 16 h of stirring, another aliquot of 2,2-difluoro-2-(fluorosulfonyl)acetic acid (1.8 equiv, Sigma-Aldrich) was added neat and dropwise over a period of 5 min. After another 48 h of stirring at RT, the crude reaction mixture was carefully quenched with water, and then diluted with a 1:1 (v/v) solution of ethyl acetate and hexanes. The organic layer was then separated and washed sequentially with saturated aq. NaHCO₃, water and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded 2-(difluoromethoxy)-6-methoxy-3-nitropyridine as a yellow solid (75% yield).

Step 2: In a dried, round-bottom flask equipped with a magnetic stirrer was dissolved 2-(difluoromethoxy)-6-methoxy-3-nitropyridine (1 equiv) from the previous step in methanol (0.17 M). The resulting yellow solution was then deoxygenated via subsurface purging with nitrogen for 10 min before palladium (10% w/w over activated carbon, dry, 0.08 equiv, Sigma-Aldrich) was added in one rapid portion. The resulting black suspension was then subsurface purged with hydrogen for 10 min before it was stirred under a static hydrogen atmosphere (maintained with a balloon) at RT for 90 min. The reaction was subsequently diluted with EtOAc and filtered through a bed of dichloromethane-wetted celite. The insolubles were washed further with EtOAc. Concentration of the filtrate thus obtained in vacuo afforded the title compound as a reddish, brown solid (>99% yield).

Intermediate amine 2: Preparation of 4-(difluoromethoxy)-6-(difluoromethyl)pyridin-3-amine hydrochloride

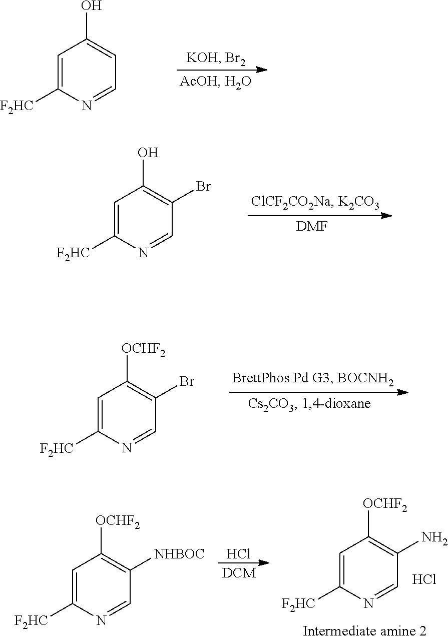

Step 1: In a dried, round-bottom flask equipped with a magnetic stirrer was dissolved 2-(difluoromethyl)pyridin-4-ol (1 equiv, Apollo Scientific) in acetic acid (0.63 M). The resulting solution was then cooled to 0° C. before potassium hydroxide (1 M solution in water, 3 equiv, Fisher Scientific) and neat bromine (1.2 equiv, Sigma-Aldrich) were added sequentially and dropwise. The resulting golden yellow solution was stirred at 0° C. for 1 h and then at RT for an additional 18 h. The now pale-yellow suspension was filtered, the insolubles washed further with dichloromethane, and the filtrate concentrated in vacuo. The residue thus obtained was then partitioned between EtOAc and water. The organic layer was separated, washed further with water and brine, dried over MgSO₄, filtered, and the filtrate concentrated in vacuo. Purification of the crude product thus obtained by way of column chromatography (SiO₂, gradient elution: 4:1 (v/v) Hex: EtOAc→EtOAc) afforded 5-bromo-2-(difluoromethyl)pyridin-4-ol as a white solid (16% yield).

Step 2: In a dried, round-bottom flask equipped with a magnetic stirrer was suspended potassium carbonate (4 equiv, Sigma-Aldrich) in DMF (0.71 M). The reaction suspension was heated to 100° C. before a DMF solution (0.19 M) of 5-bromo-2-(difluoromethyl)pyridin-4-ol (1 equiv) from the previous step and sodium chlorodifluoroacetate (1.5 equiv, TCI) was added dropwise over a period of 10 min. Following the completion of addition, the now orange reaction solution was heated at 100° C. for an extra 90 min before it was cooled to RT, diluted with tert-butyl methyl ether, and washed sequentially with water and brine. The organic layer was then dried over MgSO₄, filtered, and the filtrate concentrated in vacuo. Purification of the crude product thus obtained by way of column chromatography (SiO₂, gradient elution: 9:1 (v/v) Hex: EtOAc 4 3:7 (v/v) Hex: EtOAc) afforded 5-bromo-4-(difluoromethoxy)-2-(difluoromethyl)pyridine as a colorless oil (47% yield).

Step 3: In a thick-walled glass reaction vessel equipped with a magnetic stirrer and a Teflon screwcap was combined 5-bromo-4-(difluoromethoxy)-2-(difluoromethyl)pyridine (1 equiv) from the previous step, cesium carbonate (1 equiv, Sigma-Aldrich), [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate (0.1 equiv, Sigma-Aldrich), and tert-butyl carbamate (10 equiv, Combi-Blocks) in 1,4-dioxane (0.06 M). The resulting yellow suspension was deoxygenated via subsurface purging with nitrogen for 10 min before the reaction vessel was tightly sealed and heated at 100° C. for 16 h. The reaction mixture was then cooled to RT, diluted with EtOAc, and washed sequentially with water and brine. The organic layer was then dried over MgSO₄, filtered, and the filtrate concentrated in vacuo. Purification of the crude product thus obtained by way of column chromatography (SiO₂, gradient elution: 9:1 (v/v) Hex: EtOAc→EtOAc) afforded tert-butyl (4-(difluoromethoxy)-6-(difluoromethyl)pyridin-3-yl)carbamate as a white solid (45% yield).

Step 4: In a dried, round-bottom flask equipped with a magnetic stirrer was dissolved tert-butyl (4-(difluoromethoxy)-6-(difluoromethyl)pyridin-3-yl)carbamate (1 equiv) from the previous step in dichloromethane (0.11M). To this solution was then added HCl (4 M solution in 1,4-dioxane, 4 equiv, Sigma-Aldrich) and the resulting reaction mixture was stirred at RT for 16 h. The now white suspension was then diluted with a 1:1 (v/v) solution of hexanes and tert-butyl methyl ether, sonicated for 10 min, and filtered. The insolubles were washed further with cold tert-butyl methyl ether and air-dried. The crude title compound thus obtained was used without further purification (74% yield).

The following amines were prepared in an analogous fashion to Intermediate amine 2, but substituting 2-(difluoromethyl)pyridin-4-ol in step 1 with the requisite, commercially available (hetero)aryl alcohol. In the case of Intermediate amine 4, step 1 was obviated and 5-bromo-2-(difluoromethyl)pyridin-4-ol in step 2 was substituted with the requisite commercially available (hetero)aryl alcohol.

| Starting Material | Product | Starting Material | Product |
|---|---|---|---|
| 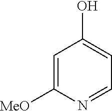 | 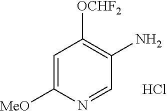 [SMILES: COc1cc(OC(F)F)c(N)cn1 ; Cl] | 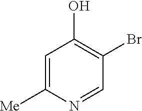 | 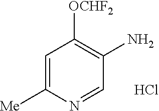 [SMILES: Cc1cc(OC(F)F)c(N)cn1 ; Cl] |
| AmBeed | Intermedate amine 3 | Combi-Blocks | Intermedate amine 4 |

Intermediate amine 5: Preparation of 2-cyclopropoxy-6-methoxypyridin-3-amine

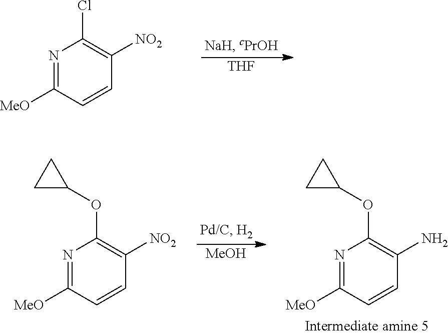 [SMILES: "MeOH" ; COc1ccc([N+](=O)[O-])c(Cl)n1 ; COc1ccc([N+](=O)[O-])c(OC2CC2)n1 ; COc1ccc(N)c(OC2CC2)n1 ; "THF"]

Step 1: In a dried, round-bottom flask equipped with a magnetic stirrer was diluted cyclopropanol (1.2 equiv, Sigma-Aldrich) with anhydrous THE (0.34 M). To this was then added sodium hydride (60% w/w dispersion in paraffin oil, 2 equiv, Sigma-Aldrich) in one rapid portion at 0° C. and the resulting mixture was stirred at 0° C. for 1 h. Then, 2-chloro-6-methoxy-3-nitropyridine (1 equiv, Enamine) was added at 0° C. portion-wise over a period of 5 min and the resulting mixture was allowed to warm to RT. After 2 h of stirring at RT, the crude reaction mixture was carefully quenched with ice water and then extracted with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution, 7:1 (v/v) Hex: EtOAc→1:4 (v/v) Hex: EtOAc) afforded 2-cyclopropoxy-6-methoxy-3-nitropyridine as a yellow solid (22% yield).

Step 2: In a dried, round-bottom flask equipped with a magnetic stirrer was dissolved 2-cyclopropoxy-6-methoxy-3-nitropyridine (1 equiv) from the previous step in methanol (0.038 M). The resulting yellow solution was then deoxygenated via subsurface purging with nitrogen for 10 min before palladium (10% w/w over activated carbon, dry, 0.08 equiv, Sigma-Aldrich) was added in one rapid portion. The resulting black suspension was then subsurface purged with hydrogen for 10 min before it was stirred under a static hydrogen atmosphere (maintained with a balloon) at RT for 1 h. The reaction was subsequently diluted with EtOAc and filtered through a bed of dichloromethane-wetted celite. The insolubles were washed further with EtOAc. Concentration of the filtrate thus obtained in vacuo afforded the title compound as a yellow solid (87% yield).

The following amines were prepared in an analogous fashion to Intermediate amine 5, but substituting cyclopropanol in step 1 with the requisite, commercially available alcohol.

| Starting Material | Product | Starting Material | Product |
|---|---|---|---|
| 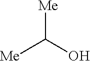 | 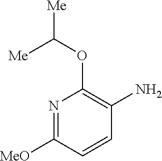 [SMILES: COc1ccc(N)c(OC(C)C)n1] | 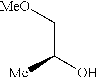 | 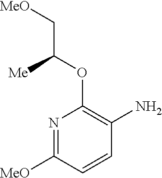 [SMILES: COC[C@H](C)Oc1nc(OC)ccc1N] |
| Sigma-Aldrich | Intermediate amine 6 | Enamine | Intermediate amine 7 |

Intermediate amine 8: Preparation of 6-chloro-4-cyclopropoxypyridin-3-amine

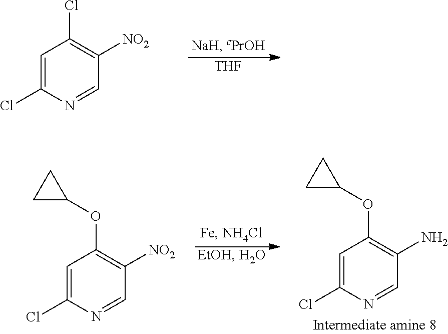

Step 1: In a dried, round-bottom flask equipped with a magnetic stirrer was diluted cyclopropanol (1.2 equiv, Sigma-Aldrich) with anhydrous THE (0.2 M). To this was then added sodium hydride (60% w/w dispersion in paraffin oil, 2 equiv, Sigma-Aldrich) in one rapid portion at 0° C. and the resulting mixture was stirred at 0° C. for 1 h. Then, 2,4-dichloro-5-nitropyridine (1 equiv, AmBeed) was added at 0° C. portion-wise over a period of 5 min and the resulting mixture was allowed to warm to RT. After 2 h of stirring at RT, the crude reaction mixture was carefully quenched with ice water and then extracted with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution, 7:1 (v/v) Hex: EtOAc→1:4 (v/v) Hex: EtOAc) afforded 2-chloro-4-cyclopropoxy-5-nitropyridine as a yellow solid (27% yield).

Step 2: In a dried, round-bottom flask equipped with a magnetic stirrer was combined 2-chloro-4-cyclopropoxy-5-nitropyridine (1 equiv) from the previous step and ammonium chloride (10 equiv, Fisher) in a 5:3 (v/v) solution of ethanol and water (0.028 M). To this was then added iron powder (10 equiv, Sigma-Aldrich) in one rapid portion and the resulting grey suspension was heated at 90° C. under a nitrogen atmosphere for 1 h. The reaction suspension was then cooled to RT, filtered, and the insolubles washed with EtOAc. The filtrate was then diluted with water and extracted further with EtOAc. The combined organic extracts were washed further with water and brine, dried over Na₂SO₄, filtered, and the filtrate concentrated in vacuo. Purification of the crude product thus obtained by way of column chromatography (SiO₂, 1:3 (v/v) Hex: EtOAc) afforded the title compound as a yellow solid (94% yield).

The following amines were prepared in an analogous fashion to Intermediate amine 8, but substituting cyclopropanol in step 1 with the requisite, commercially available alcohol.

| Starting Material | Product | Starting Material | Product |
|---|---|---|---|
| 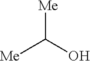 | 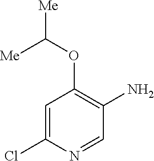 [SMILES: CC(C)Oc1cc(Cl)ncc1N] | 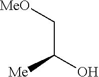 | 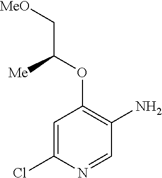 [SMILES: COC[C@H](C)Oc1cc(Cl)ncc1N] |
| Sigma-Aldrich | Intermediate amine 9 | Enamine | Intermediate amine 10 |

Intermediate amine 11: Preparation of 3-(difluoromethoxy)-5-methoxypyridin-2-amine

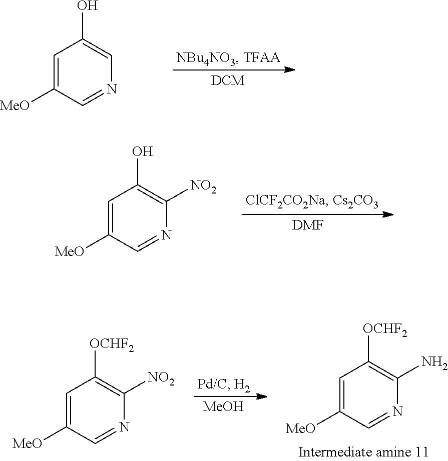

Step 1: In a dried, round-bottom flask equipped with a magnetic stirrer was combined 5-methoxypyridin-3-ol (1 equiv, Combi-Blocks) and tetrabutylammonium nitrate (1.6 equiv, Sigma-Aldrich) in dichloromethane (0.16 M). The resulting solution was then cooled to 0° C. before trifluoroacetic anhydride (1.6 equiv, Sigma-Aldrich) was added neat and dropwise. The resulting mixture was stirred at 0° C. for 2 h and then at RT for an additional 30 min. The reaction was then quenched with water and extracted further with dichloromethane. The combined organic extracts were washed further with water and brine, dried over Na₂SO₄, filtered, and the filtrate concentrated in vacuo. Purification of the crude product thus obtained by way of column chromatography (SiO₂, 3:1 (v/v) Hex: EtOAc) afforded 5-methoxy-2-nitropyridin-3-ol as a yellow oil (31% yield).

Step 2: In a dried, round-bottom flask equipped with a magnetic stirrer was suspended cesium carbonate (3 equiv, Sigma-Aldrich) in DMF (0.42 M). The reaction suspension was heated to 80° C. before a DMF solution (0.28 M) of 5-methoxy-2-nitropyridin-3-ol (1 equiv) from the previous step and sodium chlorodifluoroacetate (1.5 equiv, TCI) was added dropwise over a period of 10 min. Following the completion of addition, the now orange reaction solution was heated at 80° C. for an extra 30 min before it was cooled to RT, diluted with EtOAc, and washed sequentially with water and brine. The organic layer was then dried over Na₂SO₄, filtered, and the filtrate concentrated in vacuo. Purification of the crude product thus obtained by way of column chromatography (SiO₂, 7:1 (v/v) Hex: EtOAc) afforded 3-(difluoromethoxy)-5-methoxy-2-nitropyridine as a yellow oil (32% yield).

Step 3: In a dried, round-bottom flask equipped with a magnetic stirrer was dissolved 3-(difluoromethoxy)-5-methoxy-2-nitropyridine (1 equiv) from the previous step in methanol (0.023 M). The resulting yellow solution was then deoxygenated via subsurface purging with nitrogen for 10 min before palladium (10% w/w over activated carbon, dry, 0.2 equiv, Sigma-Aldrich) was added in one rapid portion. The resulting black suspension was then subsurface purged with hydrogen for 10 min before it was stirred under a static hydrogen atmosphere at RT for 1 h. The reaction was subsequently quenched with dichloromethane and filtered through a bed of dichloromethane-wetted celite. Concentration of the filtrate in vacuo afforded the title compound as a yellow solid (97% yield).

Intermediate amine 12 & Intermediate amine 13: Preparation of 2-chloro-6-(difluoromethoxy)pyridin-3-amine and 6-chloro-2-(difluoromethoxy)pyridin-3-amine

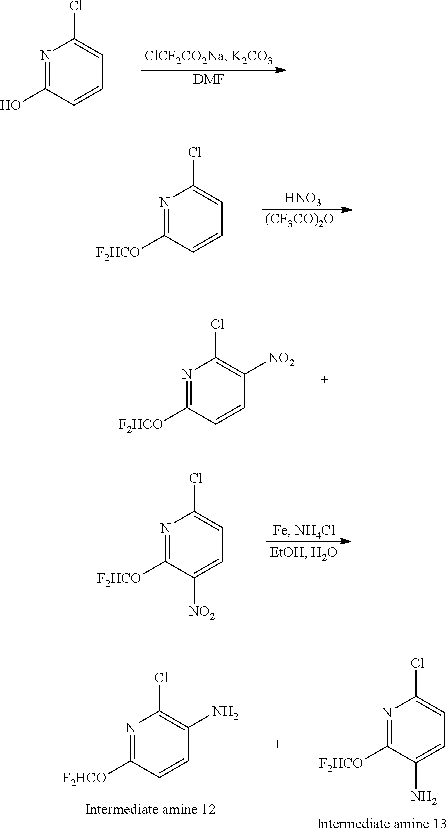

Step 1: In a dried, round-bottom flask equipped with a magnetic stirrer was suspended 6-chloropyridin-2-ol (1 equiv, Combi-Blocks) and potassium carbonate (1 equiv, Sigma-Aldrich) in DMF (0.10 M). The resulting suspension was heated to 85° C. before sodium chlorodifluoroacetate (1.2 equiv, TCI) was added as a DMF solution (0.2 M), dropwise, over a period of 10 min. Following the completion of addition, the now dark reaction mixture was heated at 85° C. for an extra 50 min and then at 100° C. for 2 h. The reaction suspension was then cooled RT, diluted with EtOAc, and washed sequentially with water and brine. The organic layer was then dried over MgSO₄ and filtered. Concentration of the filtrate thus obtained in vacuo afforded crude 2-chloro-6-(difluoromethoxy)pyridine as a white solid (14% yield).

Step 2: In a dried, round-bottom flask equipped with a magnetic stirrer was dissolved 2-chloro-6-(difluoromethoxy)pyridine (1 equiv) from the previous step in trifluoroacetic anhydride (1.8 M). To this mixture was then added, at 0° C., fuming nitric acid (1.1 equiv, Fisher Scientific) over a period of 5 min. The resulting yellow reaction solution was stirred at 0° C. for 2 h, before it was carefully quenched with ice water and extracted with tert-butyl methyl ether. The combined organic extracts were washed further with water and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded 2-chloro-6-(difluoromethoxy)-3-nitropyridine and 6-chloro-2-(difluoromethoxy)-3-nitropyridine as a mixture (90% combined yield).

Step 3: In a dried, round-bottom flask equipped with a magnetic stirrer was combined the inseparable mixture of 2-chloro-6-(difluoromethoxy)-3-nitropyridine and 6-chloro-2-(difluoromethoxy)-3-nitropyridine (1 equiv) from the previous step, and ammonium chloride (5 equiv, Fisher Scientific) in a 2:1 (v/v) solution of ethanol and water (0.67 M). To this solution was then added iron powder (5 equiv, Sigma-Aldrich) in one rapid portion and the resulting grey suspension was heated at 80° C. under a nitrogen atmosphere for 2 h. The reaction suspension was then cooled to RT, filtered through a bed of celite, and the insolubles washed with EtOAc. The filtrate thus obtained was then diluted with water and extracted further with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: Hex 4 EtOAc) afforded Intermediate amine 12 as the faster eluting compound, and Intermediate amine 13 as the slower eluting compound (91% combined yield).

Intermediate amine 14: Preparation of 4-(((tert-butyldimethylsilyl)oxy)methyl)-2-methoxyaniline

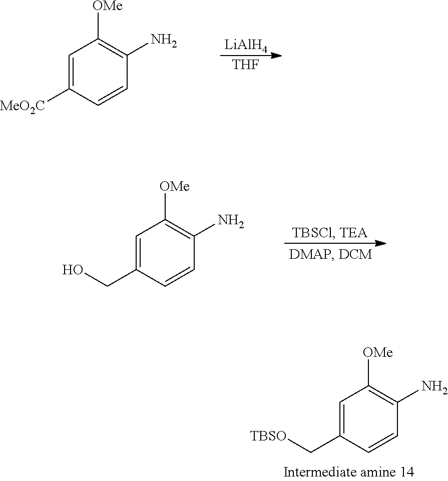 [SMILES: COC(=O)c1ccc(N)c(OC)c1 ; "LiAlH4" ; COc1cc(CO)ccc1N ; COc1cc(CO[Si](C)(C)C(C)(C)C)ccc1N ; "THF"]

Step 1: In a dried, round-bottom flask equipped with a magnetic stirrer was dissolved methyl 4-amino-3-methoxybenzoate (1 equiv, Sigma-Aldrich) in THF (0.22 M). To the resulting solution was then added lithium aluminum hydride (1.0 M solution in THF, 2 equiv, Sigma-Aldrich) dropwise at RT over a period of 20 min. Following the completion of addition, the now grey-white suspension was stirred at RT for another 2 h before the reaction was carefully quenched with the addition of water. The resulting emulsion was then extracted with tert-butyl methyl ether. The combined organic extracts were washed further with water and brine, dried over MgSO₄, and filtered. Concentration of the filtrate thus obtained in vacuo afforded crude (4-amino-3-methoxyphenyl)methanol as a viscous oil (78% yield).

Step 2: In a dried, round-bottom flask equipped with a magnetic stirrer was combined (4-amino-3-methoxyphenyl)methanol (1 equiv), tert-butyldimethylsilyl chloride (1.2 equiv, Sigma-Aldrich), and triethylamine (1.8 equiv, Sigma-Aldrich) in dichloromethane (0.22 M). To the resulting solution was then added 4-dimethylaminopyridine (0.1 equiv, Sigma-Aldrich) in one rapid portion and the reaction mixture was allowed to stir at RT for 2 h. The reaction was quenched with the addition of water and extracted with dichloromethane. The combined organic extracts were washed further with brine, dried over MgSO₄, filtered, and the filtrate concentrated in vacuo. Purification of the crude product thus obtained by way of column chromatography (SiO₂, gradient elution: Hex→3:1 (v/v) Hex: EtOAc) afforded the title compound as a colorless oil (83% yield).

Intermediate ketone 1: Preparation of N-(2-(difluoromethoxy)-6-methylpyridin-3-yl)-1-(2-isopropylphenyl)-3-oxocyclobutane-1-carboxamide

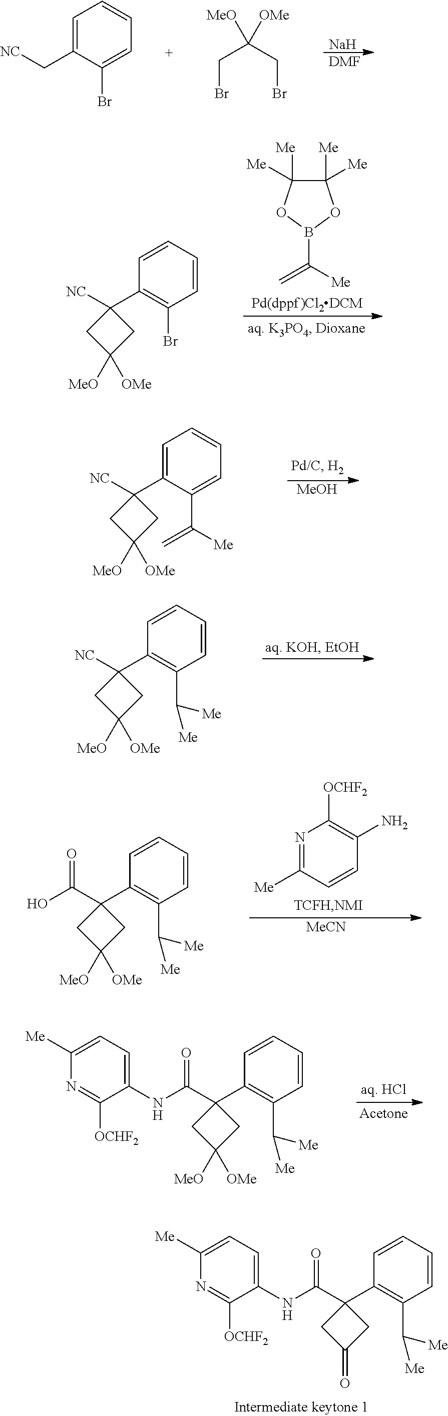

Step 1: In a dried, round-bottom flask equipped with a magnetic stirrer was dissolved 2-(2-bromophenyl)acetonitrile (1 equiv, Acros) in anhydrous DMF (0.43 M). To this was then added at 0° C. sodium hydride (60% w/w dispersion in paraffin oil, 2 equiv, Sigma-Aldrich) in one rapid portion and the resulting mixture was stirred at 0° C. for 1 h. Finally, 1,3-dibromo-2,2-dimethoxypropane (1.5 equiv, Sigma-Aldrich) was added to the reaction mixture in one rapid portion at 0° C. The cooling bath was then removed, and the reaction mixture was heated at 60° C. for 16 h. The reaction was then carefully quenched with ice water and extracted with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: Hex 4 EtOAc) afforded 1-(2-bromophenyl)-3,3-dimethoxycyclobutane-1-carbonitrile as a yellow oil (48% yield).

Step 2: In a dried, round-bottom flask equipped with a magnetic stirrer was combined 1-(2-bromophenyl)-3,3-dimethoxycyclobutane-1-carbonitrile (1 equiv) from the previous step, 4,4,5,5-tetramethyl-2-(prop-1-en-2-yl)-1,3,2-dioxaborolane (3 equiv, Frontier Scientific), potassium phosphate (3 equiv, Sigma-Aldrich), and [1,1′-bis(diphenylphosphino)ferrocene]dichloropalladium(II) dichloromethane complex (0.1 equiv, Sigma-Aldrich) in a 10:1 (v/v) solution of 1,4-dioxane and water (0.18 M). The resulting mixture was deoxygenated via subsurface purging with nitrogen for 10 min before it was heated at 80° C. for 2 h. The now dark brown suspension was cooled to RT, quenched with water, and extracted with EtOAc. The combined organic extracts were washed further with water and brine, dried over Na₂SO₄, filtered, and the filtrate concentrated in vacuo. Purification of the crude product thus obtained by way of column chromatography (SiO₂, gradient elution: Hex 4 EtOAc) afforded 3,3-dimethoxy-1-(2-(prop-1-en-2-yl)phenyl)cyclobutane-1-carbonitrile as an off-white solid (78% yield).

Step 3: In a dried, round-bottom flask equipped with a magnetic stirrer was dissolved 3,3-dimethoxy-1-(2-(prop-1-en-2-yl)phenyl)cyclobutane-1-carbonitrile (1 equiv) from the previous step in methanol (0.36 M). The resulting colorless solution was then deoxygenated via subsurface purging with nitrogen for 10 min before palladium (10% w/w over activated carbon, dry, 0.2 equiv, Sigma-Aldrich) was added in one rapid portion. The resulting black suspension was then subsurface purged with hydrogen for 10 min before it was stirred under a static hydrogen atmosphere at RT for 12 h. The reaction was subsequently quenched with dichloromethane and filtered through a bed of dichloromethane-wetted celite. The insolubles were washed further with dichloromethane and the filtrate was concentrated in vacuo. Purification of the crude product thus obtained by way of column chromatography (SiO₂, gradient elution: Hex→EtOAc) afforded 1-(2-isopropylphenyl)-3,3-dimethoxycyclobutane-1-carbonitrile as a yellow oil (95% yield).

Step 4: In a thick-walled reaction vessel equipped with a magnetic stirrer and a Teflon screwcap was combined 1-(2-isopropylphenyl)-3,3-dimethoxycyclobutane-1-carbonitrile (1 equiv) from the previous step and potassium hydroxide (20 equiv, Acros) in a 2:1 (v/v) solution of ethanol and water (0.16 M). The reaction vessel was then tightly sealed and heated at 140° C. for 48 h. Upon cooling to RT, the reaction mixture was carefully quenched with HCl (1 M solution in water, 20 equiv) and the volatiles were removed in vacuo. The resulting suspension was back extracted with EtOAc. The combined organic extracts were washed sequentially with water and brine, dried over Na₂SO₄, and filtered. Concentration of the filtrate in vacuo afforded 1-(2-isopropylphenyl)-3,3-dimethoxycyclobutane-1-carboxylic acid as a yellow oil (65% yield).

Step 5: In a dried, round-bottom flask equipped with a magnetic stirrer was combined 1-(2-isopropylphenyl)-3,3-dimethoxycyclobutane-1-carboxylic acid (1.8 equiv) from the previous step, 2-(difluoromethoxy)-6-methylpyridin-3-amine (1 equiv, synthesized as per WO2019/234115), and 1-methylimidazole (3.5 equiv, Sigma-Aldrich) in anhydrous acetonitrile (0.19 M). To this was then added chloro-N,N,N′,N′-tetramethylformamidinium hexafluorophosphate (1.9 equiv, Chem-Impex) in one rapid portion and the resulting mixture was stirred at RT for 2 h. The reaction was subsequently quenched with saturated aq. NH₄Cl and extracted with EtOAc. The combined organic extracts were washed further with water and brine, dried over Na₂SO₄, filtered, and the filtrate concentrated in vacuo. Purification of the crude product thus obtained by way of column chromatography (SiO₂, 2:1 (v/v) Hex: EtOAc) afforded N-(2-(difluoromethoxy)-6-methylpyridin-3-yl)-1-(2-isopropylphenyl)-3,3-dimethoxycyclobutane-1-carboxamide as a yellow oil (83% yield).

Step 6: In a round-bottom flask equipped with a magnetic stirrer was dissolved N-(2-(difluoromethoxy)-6-methylpyridin-3-yl)-1-(2-isopropylphenyl)-3,3-dimethoxycyclobutane-1-carboxamide (1 equiv) from the previous step in acetone (0.3 M). To this solution was then added HCl (6 M solution in water, 20 equiv) and the resulting reaction mixture was heated at 90° C. for 5 h. Upon cooling to RT, the reaction mixture was carefully quenched with NaOH (2 M solution in water, 20 equiv) and the volatiles were removed in vacuo. The resulting suspension was back extracted with EtOAc. The combined organic extracts were washed further with water and brine, dried over Na₂SO₄, filtered, and the filtrate concentrated in vacuo. Purification of the crude product thus obtained by way of column chromatography (SiO₂, 1:1 (v/v) Hex: EtOAc) afforded the title compound as a yellow solid (86% yield).

The following ketone was prepared in an analogous fashion to Intermediate ketone 1, but substituting 2-(difluoromethoxy)-6-methylpyridin-3-amine in step 5 with the requisite amine.

| Starting Material | Product |
|---|---|
| 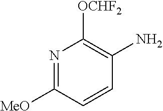 [SMILES: COc1ccc(N)c(OC(F)F)n1] Intermedate amine 1 | 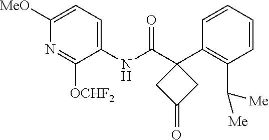 [SMILES: COc1ccc(NC(=O)C2(c3ccccc3C(C)C)CC(=O)C2)c(OC(F)F)n1] |
| | Intermediate ketone 2 |
| 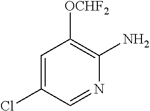 [SMILES: Nc1ncc(Cl)cc1OC(F)F] Intermedate halide 1, step 2 | 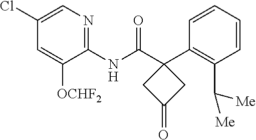 [SMILES: CC(C)c1ccccc1C1(C(=O)Nc2ncc(Cl)cc2OC(F)F)CC(=O)C1] |
| | Intermediate ketone 3 |
| 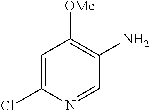 (Combi-Blocks) | 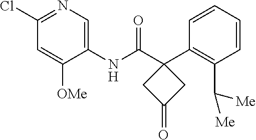 [SMILES: COc1cc(Cl)ncc1NC(=O)C1(c2ccccc2C(C)C)CC(=O)C1] |
| | Intermediate ketone 4 |
| 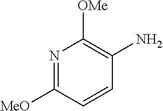 [SMILES: COc1ccc(N)c(OC)n1] (AmBeed) | 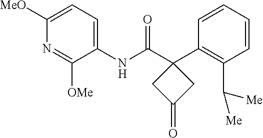 [SMILES: COc1ccc(NC(=O)C2(c3ccccc3C(C)C)CC(=O)C2)c(OC)n1] |
| | Intermediate ketone 5 |
| 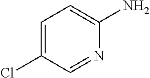 (Combi-Blocks) | 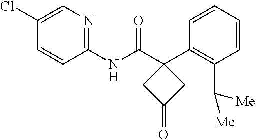 [SMILES: CC(C)c1ccccc1C1(C(=O)Nc2ccc(Cl)cn2)CC(=O)C1] |
| | Intermediate ketone 7 |
| 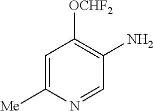 [SMILES: Cc1cc(OC(F)F)c(N)cn1] Intermediate amine 4 | 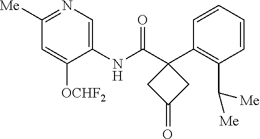 [SMILES: Cc1cc(OC(F)F)c(NC(=O)C2(c3ccccc3C(C)C)CC(=O)C2)cn1] |
| | Intermediate ketone 8 |

Intermediate ketone 6: Preparation of N-(2-(difluoromethoxy)-6-methylpyridin-3-yl)-1-(3-isopropylpyridin-2-yl)-3-oxocyclobutane-1-carboxamide

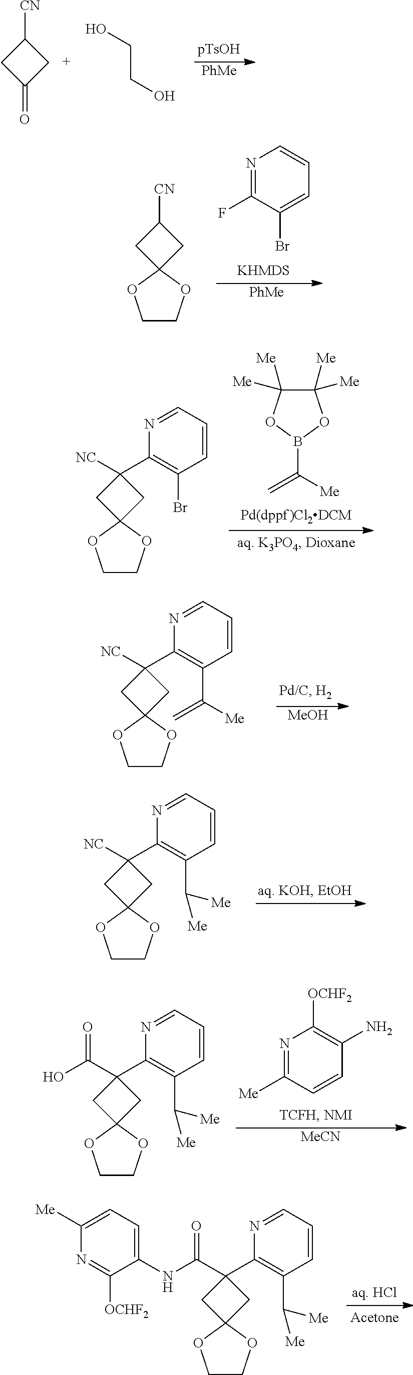

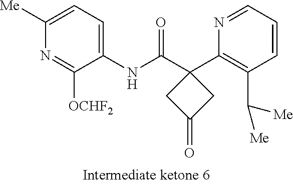 [SMILES: Cc1ccc(NC(=O)C2(c3ncccc3C(C)C)CC(=O)C2)c(OC(F)F)n1]

Step 1: In a dried, round-bottom flask equipped with a magnetic stirrer was combined 3-oxocyclobutane-1-carbonitrile (1 equiv, AstaTech), ethylene glycol (1.5 equiv, Sigma-Aldrich), and p-toluenesulfonic acid (0.1 equiv, Sigma-Aldrich) in anhydrous toluene (0.53 M). To this was attached a Dean-Stark apparatus and the reaction mixture was at 110° C. for 16 h. The resulting mixture was then cooled to RT and concentrated in vacuo. The residue thus obtained was then partitioned between saturated aq. NaHCO₃ and EtOAc. The organic layer was separated and washed further with water and brine, dried over Na₂SO₄, filtered, and the filtrate concentrated in vacuo. Purification of the crude product thus obtained by way of column chromatography (SiO₂, 12:1 (v/v) Hex: EtOAc) afforded 5,8-dioxaspiro[3.4]octane-2-carbonitrile as a light yellow oil (68% yield).

Step 2: In a dried, round-bottom flask equipped with a magnetic stirrer was combined 5,8-dioxaspiro[3.4]octane-2-carbonitrile (1 equiv) from the previous step and 3-bromo-2-fluoropyridine (1 equiv, Sigma-Aldrich) in anhydrous toluene (0.29 M). To this was then added at RT potassium bis(trimethylsilyl)amide (1.1 equiv, Sigma-Aldrich) portion-wise and the resulting mixture was heated at 60° C. for 4 h. The reaction was then carefully quenched with saturated aq. NH₄Cl at RT and extracted with EtOAc. The combined organic extracts were washed further with water and brine, dried over Na₂SO₄, filtered, and the filtrate concentrated in vacuo. Purification of the crude product thus obtained by way of column chromatography (SiO₂, 5:1 (v/v) Hex: EtOAc) afforded 2-(3-bromopyridin-2-yl)-5,8-dioxaspiro[3.4]octane-2-carbonitrile as a yellow solid (64% yield).

Step 3: In a dried, round-bottom flask equipped with a magnetic stirrer was combined 2-(3-bromopyridin-2-yl)-5,8-dioxaspiro[3.4]octane-2-carbonitrile (1 equiv) from the previous step, 4,4,5,5-tetramethyl-2-(prop-1-en-2-yl)-1,3,2-dioxaborolane (3 equiv, Frontier Scientific), potassium phosphate (3 equiv, Sigma-Aldrich), and [1,1′-bis(diphenylphosphino)ferrocene]dichloropalladium(II) dichloromethane complex (0.1 equiv, Sigma-Aldrich) in a 10:1 (v/v) solution of 1,4-dioxane and water (0.17 M). The resulting mixture was deoxygenated via subsurface purging with nitrogen for 10 min before it was heated at 80° C. for 2 h. The now dark brown suspension was cooled to RT, quenched with water, and extracted with EtOAc. The combined organic extracts were washed further with water and brine, dried over Na₂SO₄, filtered, and the filtrate concentrated in vacuo. Purification of the crude product thus obtained by way of column chromatography (SiO₂, 4:1 (v/v) Hex: EtOAc) afforded 2-(3-(prop-1-en-2-yl)pyridin-2-yl)-5,8-dioxaspiro[3.4]octane-2-carbonitrile as a yellow solid (94% yield).

Step 4: In a dried, round-bottom flask equipped with a magnetic stirrer was dissolved 2-(3-(prop-1-en-2-yl)pyridin-2-yl)-5,8-dioxaspiro[3.4]octane-2-carbonitrile (1 equiv) from the previous step in methanol (0.18 M). The resulting colorless solution was then deoxygenated via subsurface purging with nitrogen for 10 min before palladium (10% w/w over activated carbon, dry, 0.16 equiv, Sigma-Aldrich) was added in one rapid portion. The resulting black suspension was then subsurface purged with hydrogen for 10 min before it was stirred under a static hydrogen atmosphere at RT for 8 h. The reaction was subsequently quenched with dichloromethane and filtered through a bed of dichloromethane-wetted celite. The insolubles were washed further with dichloromethane. Concentration of the filtrate thus obtained in vacuo afforded 2-(3-isopropylpyridin-2-yl)-5,8-dioxaspiro[3.4]octane-2-carbonitrile as a yellow oil (97% yield).

Step 5: In a thick-walled reaction vessel equipped with a magnetic stirrer and a Teflon screwcap was combined 2-(3-isopropylpyridin-2-yl)-5,8-dioxaspiro[3.4]octane-2-carbonitrile (1 equiv) from the previous step and potassium hydroxide (20 equiv, Acros) in a 2:1 (v/v) solution of ethanol and water (0.17 M). The reaction vessel was then tightly sealed and heated at 100° C. for 16 h. Upon cooling to RT, the reaction mixture was carefully quenched with HCl (1 M solution in water, 20 equiv) and the volatiles were removed in vacuo. The resulting suspension was back extracted with EtOAc. The combined organic extracts were washed sequentially with water and brine, dried over Na₂SO₄, and filtered. Concentration of the filtrate in vacuo afforded 2-(3-isopropylpyridin-2-yl)-5,8-dioxaspiro[3.4]octane-2-carboxylic acid as a white solid (2.3% yield).

Step 6: In a dried, round-bottom flask equipped with a magnetic stirrer was combined 2-(3-isopropylpyridin-2-yl)-5,8-dioxaspiro[3.4]octane-2-carboxylic acid (1 equiv) from the previous step, 2-(difluoromethoxy)-6-methylpyridin-3-amine (1.2 equiv, synthesized as per WO2019/234115), and 1-methylimidazole (7 equiv, Sigma-Aldrich) in anhydrous acetonitrile (0.036 M). To this was then added chloro-N,N,N′,N′-tetramethylformamidinium hexafluorophosphate (5 equiv, Chem-Impex) in one rapid portion and the resulting mixture was stirred at RT for 16 h. The volatiles were then removed in vacuo and the crude product thus obtained was purified by way of reverse-phase column chromatography (C₁₈, gradient elution: 95:5 (v/v) H₂O: MeCN→MeCN) to afford N-(2-(difluoromethoxy)-6-methylpyridin-3-yl)-2-(3-isopropylpyridin-2-yl)-5,8-dioxaspiro[3.4]octane-2-carboxamide as a yellow oil (38% yield).

Step 7: In a round-bottom flask equipped with a magnetic stirrer was dissolved N-(2-(difluoromethoxy)-6-methylpyridin-3-yl)-2-(3-isopropylpyridin-2-yl)-5,8-dioxaspiro[3.4]octane-2-carboxamide (1 equiv) from the previous step in acetone (0.14 M). To this solution was then added HCl (6 M solution in water, 45 equiv) and the resulting reaction mixture was heated at 90° C. for 4 h. Upon cooling to RT, the reaction mixture was carefully quenched with NaOH (2 M solution in water, 45 equiv) and the volatiles were removed in vacuo. The resulting suspension was back extracted with EtOAc. The combined organic extracts were washed further with brine, dried over Na₂SO₄, filtered, and the filtrate concentrated in vacuo. Purification of the crude product thus obtained by way of column chromatography (SiO₂, gradient elution, 1:1 (v/v) Hex: EtOAc→EtOAc) afforded the title compound as a yellow oil (74% yield).

Intermediate aldehyde 1: Preparation of N-(2-(difluoromethoxy)-6-methylpyridin-3-yl)-3-formyl-1-(2-isopropylphenyl)cyclobutane-1-carboxamide

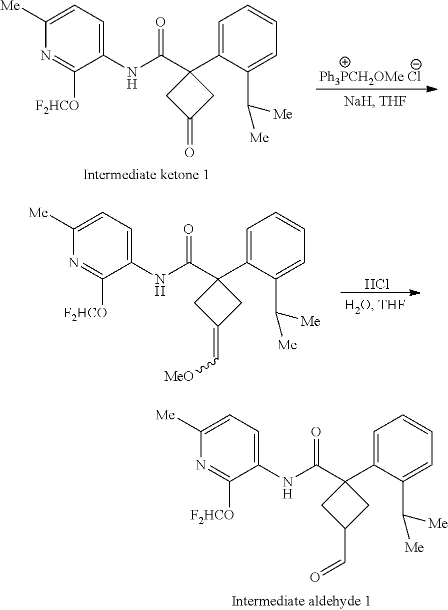

Step 1: In a dried, round-bottom flask equipped with a magnetic stirrer was dissolved (methoxymethyl)triphenylphosphonium chloride (1.1 equiv, Sigma-Aldrich) in THF (0.28 M). To this was then added at 0° C. sodium hydride (60% w/w dispersion in paraffin oil, 1.1 equiv, Sigma-Aldrich) in one rapid portion. After 1 h of stirring at 0° C., Intermediate ketone 1 (1 equiv) was then added dropwise as a solution in THE (0.16 M) over a period of 5 min. Following the completion of addition, the reaction mixture was stirred at 0° C. for 30 min and then at RT for 1 h. The reaction was then carefully quenched with the dropwise addition of saturated aq. NH₄C₁ and extracted with EtOAc. The combined organic extracts were washed further with water and brine, dried over Na₂SO₄, filtered, and the filtrate concentrated in vacuo. Purification of the crude product thus obtained by way of column chromatography (SiO₂, 5:1 (v/v) Hex: EtOAc) afforded N-(2-(difluoromethoxy)-6-methylpyridin-3-yl)-1-(2-isopropylphenyl)-3-(methoxymethylene)cyclobutane-1-carboxamide as a yellow oil (79% yield).

Step 2: In a thick-walled glass reaction vessel equipped with a magnetic stirrer and a Teflon screwcap was dissolved N-(2-(difluoromethoxy)-6-methylpyridin-3-yl)-1-(2-isopropylphenyl)-3-(methoxymethylene)cyclobutane-1-carboxamide (1 equiv) from the previous step in THE (0.027 M). To this solution was then added HCl (2 M solution in water, 25 equiv) and the resulting reaction mixture was heated at 70° C. for 2 h. Upon cooling to RT, the reaction mixture was carefully quenched with NaOH (2 M solution in water, 25 equiv) and then extracted with EtOAc. The combined organic extracts were washed further with brine, dried over Na₂SO₄, and filtered. Concentration of the filtrate in vacuo afforded the title compound as a white solid (89% yield).

The following aldehyde was prepared in an analogous fashion to Intermediate aldehyde 1, but substituting Intermediate ketone 1 in step 1 with the requisite ketone.

| Starting Material | Product |
|---|---|
| 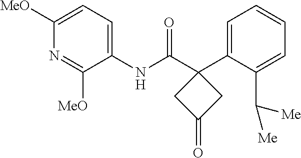 [SMILES: COc1ccc(NC(=O)C2(c3ccccc3C(C)C)CC(=O)C2)c(OC)n1] Intermediate ketone 5 | 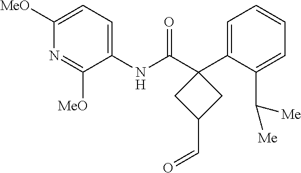 [SMILES: COc1ccc(NC(=O)C2(c3ccccc3C(C)C)CC(C=O)C2)c(OC)n1] Intermediate aldehyde 2 |

### Example 1: Preparation of N-(2-(difluoromethoxy)-6-methoxypyridin-3-yl)-3-(2-isopropylphenyl)-1-methyl-5-oxopyrrolidine-3-carboxamide

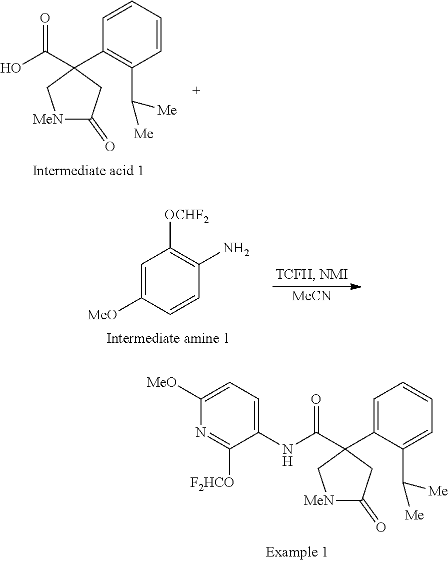

In a dried, round-bottom flask equipped with a magnetic stirrer was combined Intermediate acid 1 (1 equiv), Intermediate amine 1 (1.3 equiv), and 1-methylimidazole (4 equiv, Sigma-Aldrich) in anhydrous acetonitrile (0.04 M). To this was then added chloro-N,N,N′,N′-tetramethylformamidinium hexafluorophosphate (1.3 equiv, Chem-Impex) in one rapid portion and the resulting orange solution was stirred at RT for 24 h. The volatiles were then removed in vacuo and the residue thus obtained was subjected first to reverse-phase column chromatography (C₁₈, gradient elution: 9:1 (v/v) H₂O: MeCN+0.1% formic acid 4 MeCN+0.1% formic acid), and then to regular phase column chromatography (SiO₂, gradient elution: EtOAc→10:1 (v/v) EtOAc: MeOH), to afford the title compound as a white solid (31% yield). LCMS: m z=434.1 [M+H]⁺; ¹H NMR (CDCl₃): δ=8.43 (d, J=9 Hz, 1H), 7.46-7.28 (m, 4H), 7.19 (t, J=72 Hz, 1H), 6.96 (s, 1H), 6.53 (d, J=9 Hz, 1H), 4.26 (d, J=10 Hz, 1H), 3.81 (s, 3H), 3.75 (d, J=10 Hz, 1H), 3.29 (d, J=16 Hz, 1H), 3.01 (d, J=16 Hz, 1H), 2.92 (s, 3H), 2.83 (sept, J=6.6 Hz, 1H), 1.17 (d, J=6.6 Hz, 3H), 1.14 (d, J=6.6 Hz, 3H).

The following examples were prepared in an analogous fashion to Example 1, but substituting Intermediate amine 1 with the requisite amine.

| | | | LCMS |
|---|---|---|---|
| Example | Starting Amine | Structure | (m/z) |
| 2 | 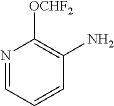 [SMILES: Nc1cccnc1OC(F)F] (Enamine) | 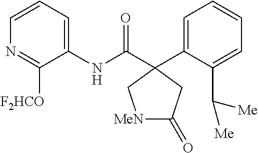 [SMILES: CC(C)c1ccccc1C1(C(=O)Nc2cccnc2OC(F)F)CC(=O)N(C)C1] | 404.1 [M + H]⁺ |
| | | N-(2-(difluoromethoxy)pyridin-3-yl)-3-(2- | |
| | | isopropylphenyl)-1-methyl-5- | |
| | | oxopyrrolidine-3-carboxamide | |
| 3 | 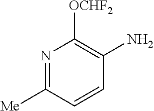 [SMILES: Cc1ccc(N)c(OC(F)F)n1] (WO2019/234115) | 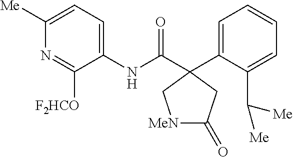 [SMILES: Cc1ccc(NC(=O)C2(c3ccccc3C(C)C)CC(=O)N(C)C2)c(OC(F)F)n1] | 418.1 [M + H]⁺ |
| | | N-(2-(difluoromethoxy)-6-methylpyridin- | |
| | | 3-yl)-3-(2-isopropylphenyl)-1-methyl-5- | |
| | | oxopyrrolidine-3-carboxamide | |
| 4 | 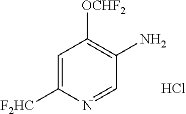 [SMILES: Nc1cnc(C(F)F)cc1OC(F)F ; Cl] Intermedate amine 2 | 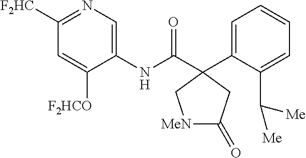 [SMILES: CC(C)c1ccccc1C1(C(=O)Nc2cnc(C(F)F)cc2OC(F)F)CC(=O)N(C)C1] | 454.1 [M + H]⁺ |
| | | N-(4-(difluoromethoxy)-6- | |
| | | (difluoromethyl)pyridin-3-yl)-3-(2- | |
| | | isopropylphenyl)-1-methyl-5- | |
| | | oxopyrrolidine-3-carboxamide | |
| 5 | 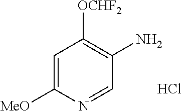 [SMILES: COc1cc(OC(F)F)c(N)cn1 ; Cl] Intermedate amine 3 | 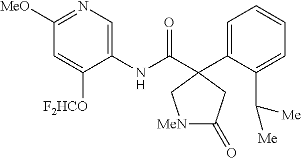 [SMILES: COc1cc(OC(F)F)c(NC(=O)C2(c3ccccc3C(C)C)CC(=O)N(C)C2)cn1] | 434.1 [M + H]⁺ |
| | | N-(4-(difluoromethoxy)-6- | |
| | | methoxypyridin-3-yl)-3-(2- | |
| | | isopropylphenyl)-1-methyl-5- | |
| | | oxopyrrolidine-3-carboxamide | |
| 6 | 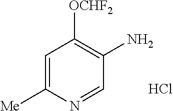 [SMILES: Cc1cc(OC(F)F)c(N)cn1 ; Cl] Intermedate amine 4 | 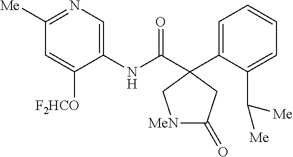 [SMILES: Cc1cc(OC(F)F)c(NC(=O)C2(c3ccccc3C(C)C)CC(=O)N(C)C2)cn1] | 418.1 [M + H]⁺ |
| | | N-(4-(difluoromethoxy)-6-methylpyridin- | |
| | | 3-yl)-3-(2-isopropylphenyl)-1-methyl-5- | |
| | | oxopyrrolidine-3-carboxamide | |
| 87 | 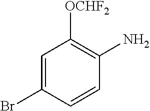 [SMILES: Nc1ccc(Br)cc1OC(F)F] (Combi-Blocks) | 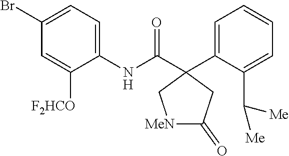 [SMILES: CC(C)c1ccccc1C1(C(=O)Nc2ccc(Br)cc2OC(F)F)CC(=O)N(C)C1] | 480.1, 483.0 [M + H]⁺ |
| | | N-(4-bromo-2-(difluoromethoxy)phenyl)- | |
| | | 3-(2-isopropylphenyl)-1-methyl-5- | |
| | | oxopyrrolidine-3-carboxamide | |
| 88 | 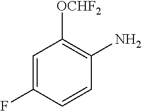 [SMILES: Nc1ccc(F)cc1OC(F)F] (Apollo) | 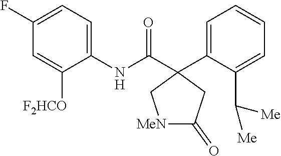 [SMILES: CC(C)c1ccccc1C1(C(=O)Nc2ccc(F)cc2OC(F)F)CC(=O)N(C)C1] | 421.1 [M + H]⁺ |
| | | N-(2-(difluoromethoxy)-4-fluorophenyl)-3- | |
| | | (2-isopropylphenyl)-1-methyl-5- | |
| | | oxopyrrolidine-3-carboxamide | |

### Example 7: Preparation of N-(5-chloro-3-(difluoromethoxy)pyridin-2-yl)-3-(2-isopropylphenyl)-1-methyl-5-oxopyrrolidine-3-carboxamide

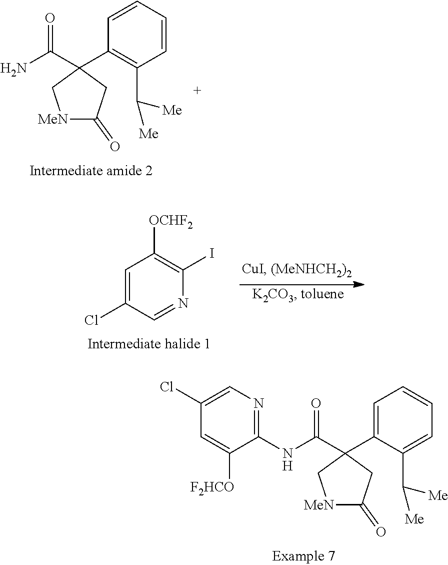

In a thick-walled glass reaction vessel equipped with a magnetic stirrer and a Teflon screwcap was combined Intermediate amide 2 (1 equiv), Intermediate halide 1 (1.1 equiv), copper(I) iodide (0.5 equiv Sigma-Aldrich), N,N′-dimethylethylenediamine (1 equiv, Combi-Blocks), and potassium carbonate (2.1 equiv, Sigma-Aldrich) in toluene (0.074 M). The resulting blue suspension was deoxygenated via subsurface purging with nitrogen for 10 min before the reaction vessel was tightly sealed and heated at 120° C. for 16 h. The now dark brown suspension was cooled to RT and filtered through a pad of celite. The insolubles were rinsed further with EtOAc and the filtrate thus obtained was washed sequentially with water and brine. The organic layer was then dried over MgSO₄, filtered, and the filtrate concentrated in vacuo. Purification of the crude product thus obtained by way of column chromatography (SiO₂, gradient elution: 9:1 (v/v) Hex: EtOAc→EtOAc) afforded the title compound as a white solid (74% yield). LCMS: m z=438.0, 440.0 [M+H]⁺; ¹H NMR (CDCl₃): δ=8.21 (d, J=2 Hz, 1H), 7.54 (d, J=2 Hz, 1H), 7.53˜7.25 (m, 5H), 6.45 (t, J=73 Hz, 1H), 4.27 (d, J=10 Hz, 1H), 3.76 (d, J=10 Hz, 1H), 3.31 (d, J=16.5 Hz, 1H), 2.97 (d, J=16.5 Hz, 1H), 2.90 (s, 3H), 2.83 (sept, J=6 Hz, 1H), 1.25 (d, J=6 Hz, 3H), 1.21 (d, J=6 Hz, 3H).

### Example 8: Preparation of N-(2-(difluoromethoxy)-6-methoxypyridin-3-yl)-3-(2-isopropylphenyl)-5-oxopyrrolidine-3-carboxamide

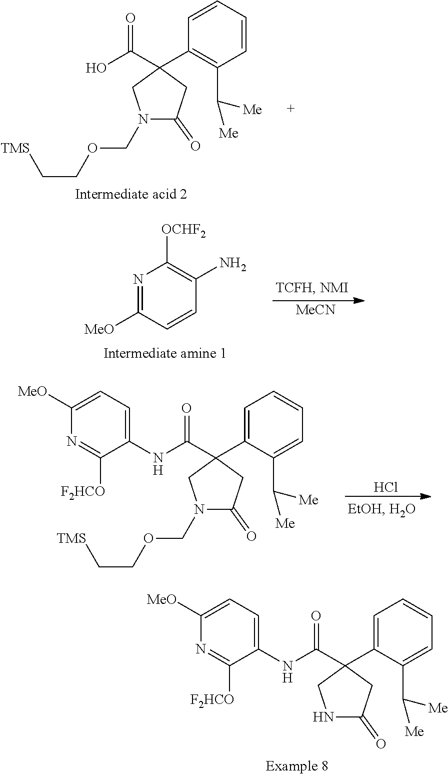 [SMILES: "MeCN" ; CC(C)c1ccccc1C1(C(=O)O)CC(=O)N(COCC[Si](C)(C)C)C1 ; COc1ccc(N)c(OC(F)F)n1 ; COc1ccc(NC(=O)C2(c3ccccc3C(C)C)CC(=O)N(COCC[Si](C)(C)C)C2)c(OC(F)F)n1 ; Cl ; COc1ccc(NC(=O)C2(c3ccccc3C(C)C)CNC(=O)C2)c(OC(F)F)n1]

Step 1: In a dried, round-bottom flask equipped with a magnetic stirrer was combined Intermediate acid 2 (1 equiv), Intermediate amine 1 (1.3 equiv), and 1-methylimidazole (4 equiv, Sigma-Aldrich) in anhydrous acetonitrile (0.04 M). To this was then added chloro-N,N,N′,N′-tetramethylformamidinium hexafluorophosphate (1.3 equiv, Chem-Impex) in one rapid portion and the resulting orange solution was stirred at RT for 24 h. The volatiles were then removed in vacuo and the residue thus obtained was subjected to reverse-phase column chromatography (C₁₈, gradient elution: 9:1 (v/v) H₂O: MeCN+0.1% formic acid 4 MeCN+0.1% formic acid) to afford N-(2-(difluoromethoxy)-6-methoxypyridin-3-yl)-3-(2-isopropylphenyl)-5-oxo-1-((2-(trimethylsilyl)ethoxy)methyl)pyrrolidine-3-carboxamide as a colorless oil (63% yield).

Step 2: In a round-bottom flask equipped with a magnetic stirrer was dissolved N-(2-(difluoromethoxy)-6-methoxypyridin-3-yl)-3-(2-isopropylphenyl)-5-oxo-1-((2-(trimethylsilyl)ethoxy)methyl)pyrrolidine-3-carboxamide (1 equiv) from the previous step in absolute ethanol (0.04 M). To this solution was then added HCl (1 M solution in water, 60 equiv) and the resulting reaction mixture was heated at 80° C. for 6 days. The volatiles were then removed in vacuo and the residue thus obtained was partitioned between 1 M aq. NaOH and EtOAc. The aqueous layer was separated and back extracted with EtOAc. The combined organic extracts were washed further with water and brine, dried over MgSO₄, filtered, and the filtrate concentrated in vacuo. The crude product thus obtained was subjected first to reverse-phase column chromatography (C₁₈, gradient elution: 9:1 (v/v) H₂O: MeCN+0.1% formic acid 4 MeCN+0.1% formic acid), and then to regular phase column chromatography (SiO₂, gradient elution: EtOAc→10:1 (v/v) EtOAc: MeOH), to afford the title compound as a white solid (24% yield). LCMS: m/z=420.0 [M+H]⁺; ¹H NMR (CDCl₃): δ=8.45 (d, J=9 Hz, 1H), 7.45˜7.29 (m, 4H), 7.19 (t, J=72 Hz, 1H), 6.97 (s, 1H), 6.53 (d, J=9 Hz, 1H), 5.87 (br s, 1H), 4.26 (d, J=10 Hz, 1H), 3.85 (s, 3H), 3.50 (d, J=10 Hz, 1H), 3.30 (d, J=16 Hz, 1H), 2.88 (d, J=16 Hz, 1H), 2.84 (sept, J=6.6 Hz, 1H), 1.15 (d, J=6.6 Hz, 6H).

The following example was prepared in an analogous fashion to Example 8, but substituting Intermediate amine 1 in step 1 with the requisite amine.

| | | | LCMS |
|---|---|---|---|
| Example | Starting Amine | Structure | (m/z) |
| 9 | 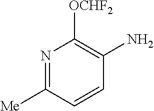 [SMILES: Cc1ccc(N)c(OC(F)F)n1] (WO2019/234115) | 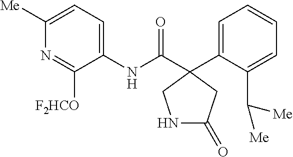 [SMILES: Cc1ccc(NC(=O)C2(c3ccccc3C(C)C)CNC(=O)C2)c(OC(F)F)n1] | 403.8 [M + H]⁺ |
| | | N-(2-(difluoromethoxy)-6-methylpyridin- | |
| | | 3-yl)-3-(2-isopropylphenyl)-5- | |
| | | oxopyrrolidine-3-carboxamide | |

Examples 10 & 11: Preparation of N-(5-chloro-3-(difluoromethoxy)pyridin-2-yl)-3-(2-isopropylphenyl)-5-oxopyrrolidine-3-carboxamide & N-(5-chloro-3-(difluoromethoxy)pyridin-2-yl)-1-(ethoxymethyl)-3-(2-isopropylphenyl)-5-oxopyrrolidine-3-carboxamide

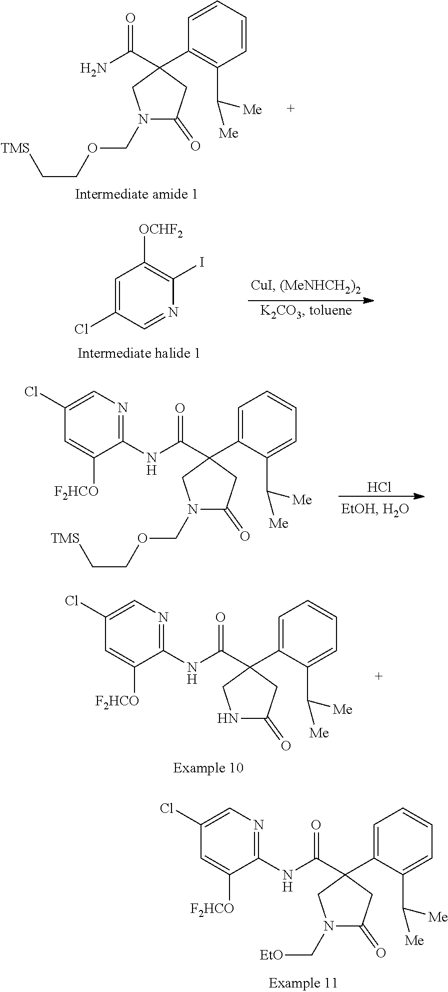

Step 1: In a thick-walled glass reaction vessel equipped with a magnetic stirrer and a Teflon screwcap was combined Intermediate amide 1 (1 equiv), Intermediate halide 1 (1 equiv), copper(I) iodide (0.5 equiv Sigma-Aldrich), N,N′-dimethylethylenediamine (1 equiv, 10 Combi-Blocks), and potassium carbonate (2.1 equiv, Sigma-Aldrich) in toluene (0.053 M). The resulting blue suspension was deoxygenated via subsurface purging with nitrogen for 10 min before the reaction vessel was tightly sealed and heated at 120° C. for 60 h. The now dark brown suspension was cooled to RT and filtered through a pad of celite. The insolubles were rinsed further with EtOAc and the filtrate thus obtained was washed sequentially with water and brine. The organic layer was then dried over MgSO₄, filtered, and the filtrate concentrated in vacuo. Purification of the crude product thus obtained by way of column chromatography (SiO₂, gradient elution: 9:1 (v/v) Hex: EtOAc→EtOAc) afforded N-(5-chloro-3-(difluoromethoxy)pyridin-2-yl)-3-(2-isopropylphenyl)-5-oxo-1-((2-(trimethylsilyl)ethoxy)methyl)pyrrolidine-3-carboxamide as a white solid (55% yield).

Step 2: In a dried, round-bottom flask equipped with a magnetic stirrer was dissolved N-(5-chloro-3-(difluoromethoxy)pyridin-2-yl)-3-(2-isopropylphenyl)-5-oxo-1-((2-(trimethylsilyl)ethoxy)methyl)pyrrolidine-3-carboxamide (1 equiv) from the previous step in absolute ethanol (0.06 M). To this solution was then added HCl (1 M solution in water, 60 equiv) and the resulting reaction mixture was heated at 80° C. for 6 days. The volatiles were then removed in vacuo and the residue thus obtained was partitioned between 1 M aq. NaOH and ethyl acetate. The aqueous layer was separated and back extracted with EtOAc. The combined organic extracts were washed further with water and brine, dried over MgSO₄, filtered, and the filtrate concentrated in vacuo. The crude product thus obtained was subjected first to reverse-phase column chromatography (C₁₈, gradient elution: 9:1 (v/v) H₂O: MeCN+0.1% formic acid 4 MeCN+0.1% formic acid), and then to regular phase column chromatography (SiO₂, gradient elution: EtOAc→10:1 (v/v) EtOAc: MeOH), to afford the two title compounds. Example 10 (white solid, 36% yield): LCMS: m/z=424.0, 426.0 [M+H]⁺; ¹H NMR (CDCl₃): δ=8.21 (d, J=2 Hz, 1H), 7.53 (d, J=2 Hz, 1H), 7.53˜7.27 (m, 5H), 6.44 (t, J=73 Hz, 1H), 6.13 (br s, 1H), 4.27 (d, J=10 Hz, 1H), 3.84 (d, J=10 Hz, 1H), 3.31 (d, J=16.5 Hz, 1H), 2.91˜2.82 (m, 2H), 1.21 (d, J=6 Hz, 6H). Example 11 (white solid, 21% yield): LCMS: m/z=504.0, 506.0 [M+Na]+; ¹H NMR (CDCl₃): δ=8.22 (d, J=2 Hz, 1H), 7.53 (d, J=2 Hz, 1H), 7.49˜7.29 (m, 5H), 6.43 (t, J=73 Hz, 1H), 4.85 (d, J=11 Hz, 1H), 4.70 (d, J=11 Hz, 1H), 4.31 (d, J=10 Hz, 1H), 3.94 (d, J=10 Hz, 1H), 3.52˜3.33 (m, 3H), 3.00 (d, J=17 Hz, 1H), 2.88 (sept, J=6.5 Hz, 1H), 1.23 (d, J=6.5 Hz, 3H), 1.21 (d, J=6.5 Hz, 3H), 1.11 (t, J=7 Hz, 3H).

The following example was prepared in an analogous fashion to Example 10, but substituting Intermediate halide 1 in step 1 with the requisite halide.

| | | | LCMS |
|---|---|---|---|
| Example | Starting Halide | Structure | (m/z) |
| 12 | 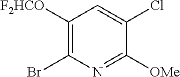 [SMILES: COc1nc(Br)c(OC(F)F)cc1Cl] Intermediate halide 2 | 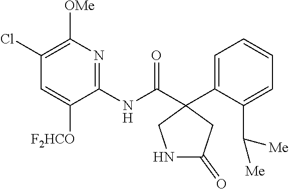 [SMILES: COc1nc(NC(=O)C2(c3ccccc3C(C)C)CNC(=O)C2)c(OC(F)F)cc1Cl] | 454.0, 456.0 [M + H]⁺ |
| | | N-(5-chloro-3-(difluoromethoxy)-6- | |
| | | methoxypyridin-2-yl)-3-(2- | |
| | | isopropylphenyl)-5-oxopyrrolidine-3- | |
| | | carboxamide | |

### Example 13: Preparation of N-(2-(difluoromethoxy)-6-methylpyridin-3-yl)-4-(2-isopropylphenyl)tetrahydro-2H-pyran-4-carboxamide

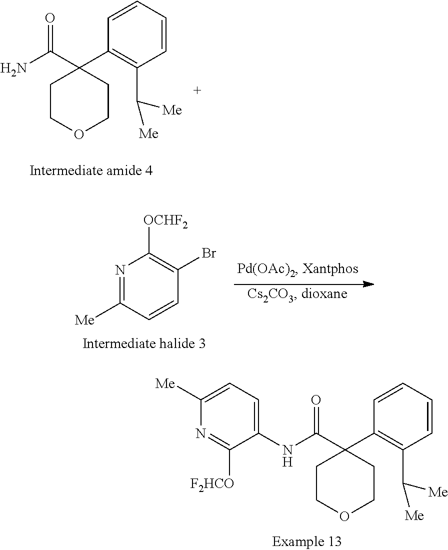

In a thick-walled glass reaction vessel equipped with a magnetic stirrer and a Teflon screwcap was combined Intermediate amide 4 (1 equiv), Intermediate halide 3 (1 equiv), palladium(II) acetate (0.1 equiv, Sigma-Aldrich), 4,5-bis(diphenylphosphino)-9,9-dimethylxanthene (0.1 equiv, Combi-Blocks), and cesium carbonate (2.5 equiv, Sigma-Aldrich) in 1,4-dioxane (0.12 M). The resulting suspension was deoxygenated via subsurface purging with nitrogen for 10 min before the reaction vessel was tightly sealed and heated at 100° C. for 16 h. The now dark brown suspension was cooled to RT, quenched with water, and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: Hex→75:35 (v/v) Hex: EtOAc) afforded the title compound as a white solid (71% yield). LCMS: m/z=404.9 [M+H]⁺; ¹H NMR (DMSO-d₆): δ=8.13 (s, 1H), 7.87 (d, J=8 Hz, 1H), 7.52 (t, J=73.5 Hz, 1H), 7.50 (d, J=1.5 Hz, 1H), 7.41 (dd, J=8, 1.5 Hz, 1H), 7.35˜7.26 (m, 2H), 7.08 (d, J=8 Hz, 1H), 3.91˜3.86 (m, 2H), 3.69˜3.65 (m, 2H), 3.22 (sept, J=6.5 Hz, 1H), 2.39˜2.36 (m, 5H), 2.06˜2.01 (m, 2H), 1.06 (d, J=6.5 Hz, 6H).

The following example was prepared in an analogous fashion to Example 13, but substituting Intermediate halide 3 with the requisite halide.

| | | | LCMS |
|---|---|---|---|
| Example | Starting Halide | Structure | (m/z) |
| 14 | 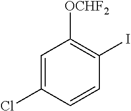 [SMILES: FC(F)Oc1cc(Cl)ccc1I] Intermediate halide 4 | 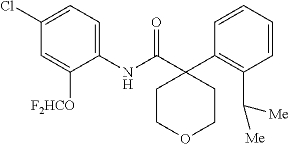 [SMILES: CC(C)c1ccccc1C1(C(=O)Nc2ccc(Cl)cc2OC(F)F)CCOCC1] | 423.8, 425.8 [M + H]⁺ |
| | | N-(4-chloro-2-(difluoromethoxy)phenyl)- | |
| | | 4-(2-isopropylphenyl)tetrahydro-2H- | |
| | | pyran-4-carboxamide | |

### Example 15: Preparation of N-(2-(difluoromethoxy)-6-methoxypyridin-3-yl)-3-(2-isopropylphenyl)oxetane-3-carboxamide

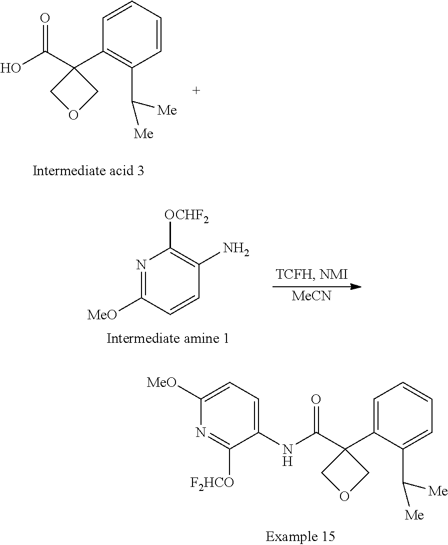

In a dried, round-bottom flask equipped with a magnetic stirrer was combined Intermediate acid 3 (1 equiv), Intermediate amine 1 (1.3 equiv), and 1-methylimidazole (4 equiv, Sigma-Aldrich) in anhydrous acetonitrile (0.04 M). To this was then added chloro-N,N,N′,N′-tetramethylformamidinium hexafluorophosphate (1.3 equiv, Chem-Impex) in one rapid portion and the resulting orange solution was stirred at RT for 24 h. The volatiles were then removed in vacuo and the residue thus obtained was subjected to reverse-phase column chromatography (C₁₈, gradient elution: 9:1 (v/v) H₂O: MeCN+0.1% formic acid→MeCN+0.1% formic acid) to afford the title compound as a white solid (76% yield). LCMS: m/z=393.1 [M+H]⁺; ¹H NMR (DMSO-d₆): δ=8.57 (s, 1H), 7.81 (d, J=8 Hz, 1H), 7.63 (t, J=73 Hz, 1H), 7.40˜7.34 (m, 4H), 6.67 (d, J=8 Hz, 1H), 5.19 (d, J=6 Hz, 2H), 4.99 (d, J=6 Hz, 2H), 3.83 (s, 3H), 2.39 (sept, J=7 Hz, 1H), 1.10 (d, J=7 Hz, 6H).

The following examples were prepared in an analogous fashion to Example 15, but substituting Intermediate amine 1 with the requisite amine.

| | | | LCMS |
|---|---|---|---|
| Example | Starting Amine | Structure | (m/z) |
| 16 | 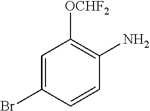 [SMILES: Nc1ccc(Br)cc1OC(F)F] (Combi-Blocks) | 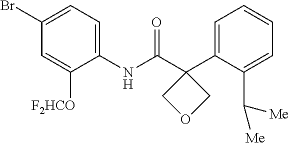 [SMILES: CC(C)c1ccccc1C1(C(=O)Nc2ccc(Br)cc2OC(F)F)COC1] | 440.9, 442.9 [M + H]⁺ |
| | | N-(4-bromo-2-(difluoromethoxy)phenyl)-3- | |
| | | (2-isopropylphenyl)oxetane-3-carboxamide | |
| 17 | 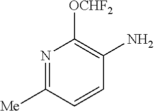 [SMILES: Cc1ccc(N)c(OC(F)F)n1] (WO2019/234115) | 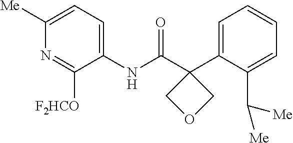 [SMILES: Cc1ccc(NC(=O)C2(c3ccccc3C(C)C)COC2)c(OC(F)F)n1] | 377.1 [M + H]⁺ |
| | | N-(2-(difluoromethoxy)-6-methylpyridin-3- | |
| | | yl)-3-(2-isopropylphenyl)oxetane-3- | |
| | | carboxamide | |
| 18 | 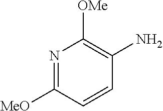 [SMILES: COc1ccc(N)c(OC)n1] (Ambeed) | 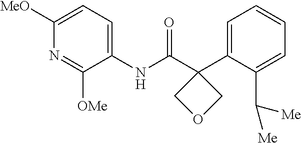 [SMILES: COc1ccc(NC(=O)C2(c3ccccc3C(C)C)COC2)c(OC)n1] | 357.2 [M + H]⁺ |
| | | N-(2,6-dimethoxypyridin-3-yl)-3-(2- | |
| | | isopropylphenyl)oxetane-3-carboxamide | |
| 19 | 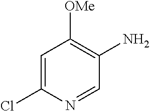 (Combi-Blocks) | 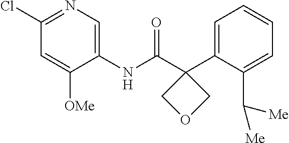 [SMILES: COc1cc(Cl)ncc1NC(=O)C1(c2ccccc2C(C)C)COC1] | 360.9, 362.9 [M + H]⁺ |
| | | N-(6-chloro-4-methoxypyridin-3-yl)-3-(2- | |
| | | isopropylphenyl)oxetane-3-carboxamide | |
| 20 | 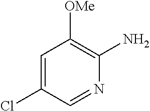 (Combi-Blocks) | 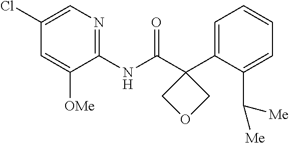 [SMILES: COc1cc(Cl)cnc1NC(=O)C1(c2ccccc2C(C)C)COC1] | 360.9, 362.9 [M + H]⁺ |
| | | N-(5-chloro-3-methoxypyridin-2-yl)-3-(2- | |
| | | isopropylphenyl)oxetane-3-carboxamide | |
| 21 | 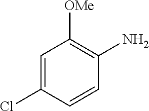 (Combi-Blocks) | 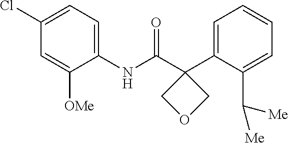 [SMILES: COc1cc(Cl)ccc1NC(=O)C1(c2ccccc2C(C)C)COC1] | 359.9, 361.9 [M + H]⁺ |
| | | N-(4-chloro-2-methoxyphenyl)-3-(2- | |
| | | isopropylphenyl)oxetane-3-carboxamide | |
| 22 | 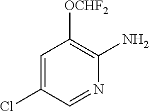 [SMILES: Nc1ncc(Cl)cc1OC(F)F] Intermedate halide 1 Step 2 | 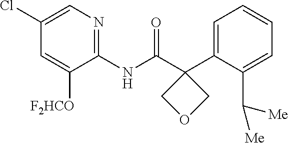 [SMILES: CC(C)c1ccccc1C1(C(=O)Nc2ncc(Cl)cc2OC(F)F)COC1] | 396.8, 398.8 [M + H]⁺ |
| | | N-(5-chloro-3-(difluoromethoxy)pyridin-2- | |
| | | yl)-3-(2-isopropylphenyl)oxetane-3- | |
| | | carboxamide | |
| 23 | 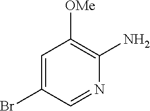 (Combi-Blocks) | 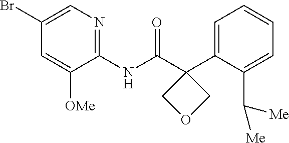 [SMILES: COc1cc(Br)cnc1NC(=O)C1(c2ccccc2C(C)C)COC1] | 404.7, 406.7 [M + H]⁺ |
| | | N-(5-bromo-3-methoxypyridin-2-yl)-3-(2- | |
| | | isopropylphenyl)oxetane-3-carboxamide | |
| 24 | 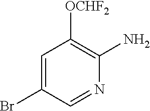 [SMILES: Nc1ncc(Br)cc1OC(F)F] (Ambeed) | 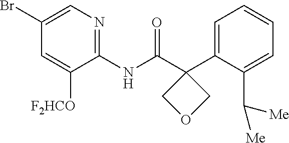 [SMILES: CC(C)c1ccccc1C1(C(=O)Nc2ncc(Br)cc2OC(F)F)COC1] | 441.5, 443.5 [M + H]⁺ |
| | | N-(5-bromo-3-(difluoromethoxy)pyridin-2- | |
| | | yl)-3-(2-isopropylphenyl)oxetane-3- | |
| | | carboxamide | |
| 25 | 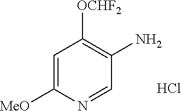 [SMILES: COc1cc(OC(F)F)c(N)cn1 ; Cl] Intermediate amine 3 | 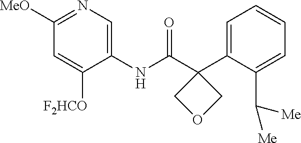 [SMILES: COc1cc(OC(F)F)c(NC(=O)C2(c3ccccc3C(C)C)COC2)cn1] | 393.2 [M + H]⁺ |
| | | N-(4-(difluoromethoxy)-6-methoxypyridin- | |
| | | 3-yl)-3-(2-isopropylphenyl)oxetane-3- | |
| | | carboxamide | |
| 26 | 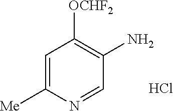 [SMILES: Cc1cc(OC(F)F)c(N)cn1 ; Cl] Intermediate amine 4 | 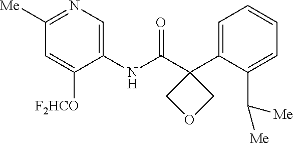 [SMILES: Cc1cc(OC(F)F)c(NC(=O)C2(c3ccccc3C(C)C)COC2)cn1] | 377.2 [M + H]⁺ |
| | | N-(4-(difluoromethoxy)-6-methylpyridin-3- | |
| | | yl)-3-(2-isopropylphenyl)oxetane-3- | |
| | | carboxamide | |
| 27 | 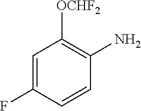 [SMILES: Nc1ccc(F)cc1OC(F)F] (Enamine) | 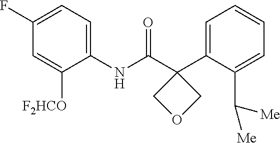 [SMILES: CC(C)c1ccccc1C1(C(=O)Nc2ccc(F)cc2OC(F)F)COC1] | 380.1 [M + H]⁺ |
| | | N-(2-(difluoromethoxy)-4-fluorophenyl)-3- | |
| | | (2-isopropylphenyl)oxetane-3-carboxamide | |
| 28 | 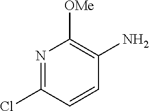 (Enamine) | 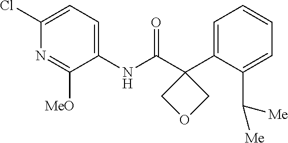 [SMILES: COc1nc(Cl)ccc1NC(=O)C1(c2ccccc2C(C)C)COC1] | 361.1, 363.1 [M + H]⁺ |
| | | N-(6-chloro-2-methoxypyridin-3-yl)-3-(2- | |
| | | isopropylphenyl)oxetane-3-carboxamide | |
| 29 | 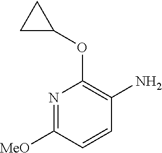 [SMILES: COc1ccc(N)c(OC2CC2)n1] | 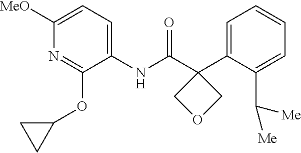 [SMILES: COc1ccc(NC(=O)C2(c3ccccc3C(C)C)COC2)c(OC2CC2)n1] | 383.3 [M + H]⁺ |
| | Intermediate amine 5 | N-(2-cyclopropoxy-6-methoxypyridin-3-yl)- | |
| | | 3-(2-isopropylphenyl)oxetane-3- | |
| | | carboxamide | |
| 30 | 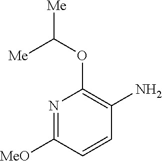 [SMILES: COc1ccc(N)c(OC(C)C)n1] | 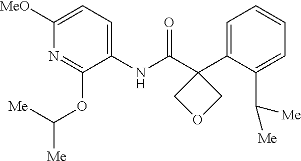 [SMILES: COc1ccc(NC(=O)C2(c3ccccc3C(C)C)COC2)c(OC(C)C)n1] | 385.2 [M + H]⁺ |
| | Intermediate amine 6 | N-(2-isopropoxy-6-methoxypyridin-3-yl)-3- | |
| | | (2-isopropylphenyl)oxetane-3-carboxamide | |
| 31 | 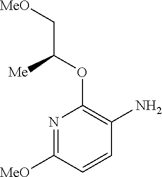 [SMILES: COC[C@H](C)Oc1nc(OC)ccc1N] | 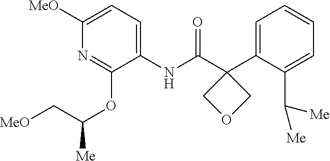 [SMILES: COC[C@H](C)Oc1nc(OC)ccc1NC(=O)C1(c2ccccc2C(C)C)COC1] | 415.2 [M + H]⁺ |
| | | (S)-3-(2-isopropylphenyl)-N-(6-methoxy-2- | |
| | Intermediate amine 7 | ((1-methoxypropan-2-yl)oxy)pyridin-3- | |
| | | yl)oxetane-3-carboxamide | |
| 32 | 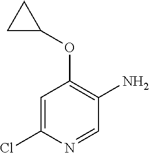 [SMILES: Nc1cnc(Cl)cc1OC1CC1] | 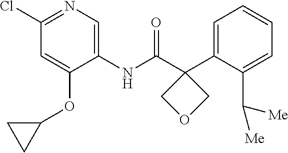 [SMILES: CC(C)c1ccccc1C1(C(=O)Nc2cnc(Cl)cc2OC2CC2)COC1] | 387.1, 389.1 [M + H]⁺ |
| | Intermediate amine 8 | N-(6-chloro-4-cyclopropoxypyridin-3-yl)-3- | |
| | | (2-isopropylphenyl)oxetane-3-carboxamide | |
| 33 | 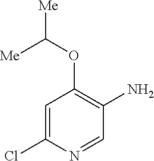 [SMILES: CC(C)Oc1cc(Cl)ncc1N] | 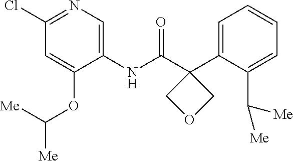 [SMILES: CC(C)Oc1cc(Cl)ncc1NC(=O)C1(c2ccccc2C(C)C)COC1] | 389.2 391.2 [M + H]⁺ |
| | Intermediate amine 9 | N-(6-chloro-4-isopropoxypyridin-3-yl)-3-(2- | |
| | | isopropylphenyl)oxetane-3-carboxamide | |
| 34 | 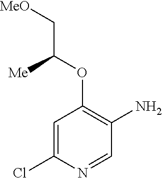 [SMILES: COC[C@H](C)Oc1cc(Cl)ncc1N] | 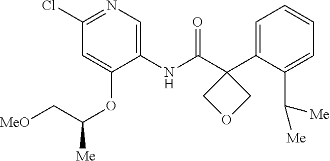 [SMILES: COC[C@H](C)Oc1cc(Cl)ncc1NC(=O)C1(c2ccccc2C(C)C)COC1] | 419.2 421.2 [M + H]⁺ |
| | | (S)-N-(6-chloro-4-((1-methoxypropan-2- | |
| | Intermediate amine 10 | yl)oxy)pyridin-3-yl)-3-(2- | |
| | | isopropylphenyl)oxetane-3-carboxamide | |
| 35 | 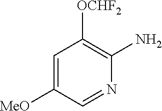 [SMILES: COc1cnc(N)c(OC(F)F)c1] Intermediate amine 11 | 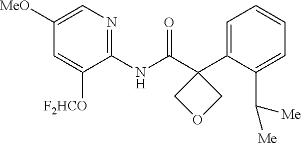 [SMILES: COc1cnc(NC(=O)C2(c3ccccc3C(C)C)COC2)c(OC(F)F)c1] | 393.2 [M + H]⁺ |
| | | N-(3-(difluoromethoxy)-5-methoxypyridin- | |
| | | 2-yl)-3-(2-isopropylphenyl)oxetane-3- | |
| | | carboxamide | |
| 36 | 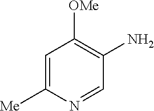 (Nanjing Gerhouse Pharmtech) | 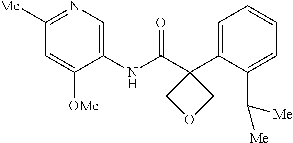 [SMILES: COc1cc(C)ncc1NC(=O)C1(c2ccccc2C(C)C)COC1] | 341.2 [M + H]⁺ |
| | | 3-(2-isopropylphenyl)-N-(4-methoxy-6- | |
| | | methylpyridin-3-yl)oxetane-3-carboxamide | |

The following example was prepared in an analogous fashion to Example 15, but substituting Intermediate acid 3 with Intermediate acid 4, and Intermediate amine 1 with the requisite amine.

| | | | LCMS |
|---|---|---|---|
| Example | Starting Amine | Structure | (m/z) |
| 37 | 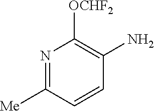 [SMILES: Cc1ccc(N)c(OC(F)F)n1] (WO2019/234115) | 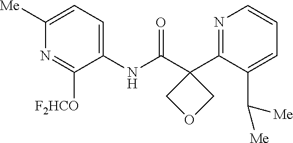 [SMILES: Cc1ccc(NC(=O)C2(c3ncccc3C(C)C)COC2)c(OC(F)F)n1] | 377.9 [M + H]⁺ |
| | | N-(2-(difluoromethoxy)-6-methylpyridin-3- | |
| | | yl)-3-(3-isopropylpyridin-2-yl)oxetane-3- | |
| | | carboxamide | |

### Example 38: Preparation of N-(2-(difluoromethoxy)-6-methylpyridin-3-yl)-1-(2-isopropylphenyl)-3-(methylamino)cyclobutane-1-carboxamide

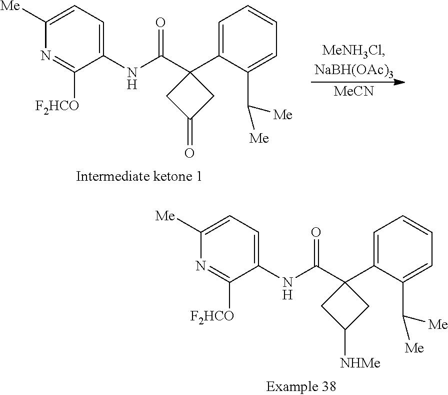

In a dried, round-bottom flask equipped with a magnetic stirrer was combined Intermediate ketone 1 (1 equiv) and methylamine hydrochloride (2 equiv, Sigma-Aldrich) in anhydrous acetonitrile (0.065 M). To this was then added sodium triacetoxyborohydride (5 equiv, Sigma-Aldrich) in one rapid portion and the resulting mixture was stirred at RT for 24 h. The volatiles were then removed in vacuo and the residue thus obtained was subjected to reverse-phase column chromatography (C₁₈, gradient elution: 95:5 (v/v) H₂O: MeCN→5:95 (v/v) H₂O: MeCN) to afford the title compound as a mixture of diastereomers (20% yield). LCMS: m/z=404.2 [M+H]⁺; ¹H NMR (methanol-d₄): δ=8.35˜8.26 (m, 1H), 7.57˜7.21 (m, 5H), 7.06˜7.03 (m, 1H), 4.26˜4.05 (m, 0.3H, diastereomer A), 3.74˜3.70 (m, 0.7H, diastereomer B), 3.16˜3.09 (m, 4H), 2.86˜2.63 (m, 4H), 2.40 (s, 3H), 1.19 (d, J=6.8 Hz, 6H).

The following examples were prepared in an analogous fashion to Example 38, but substituting methylamine hydrochloride with the requisite amine. In the case of Examples 40 and 42, acetic acid (2.5 equiv, Sigma-Aldrich) was included as an additive. In the case of Example 41, titanium(IV) isopropoxide (5 equiv, Sigma-Aldrich) was included as an additive. In the case of Examples 41 and 42, sodium triacetoxyborohydride was also substituted with sodium cyanoborohydride (Sigma-Aldrich).

| | | | LCMS |
|---|---|---|---|
| Example | Starting Amine | Structure | (m/z) |
| 39 | 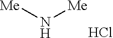 [SMILES: CNC ; Cl] (Sigma-Aldrich) | 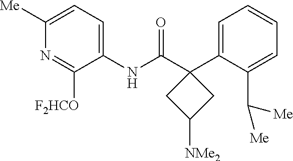 [SMILES: Cc1ccc(NC(=O)C2(c3ccccc3C(C)C)CC(N(C)C)C2)c(OC(F)F)n1] | 418.2 [M + H]⁺ |
| | | N-(2-(difluoromethoxy)-6-methylpyridin-3- | |
| | | yl)-3-(dimethylamino)-1-(2- | |
| | | isopropylphenyl)cyclobutane-1-carboxamide | |
| 40- isomer A | 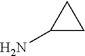 (Sigma-Aldrich) | 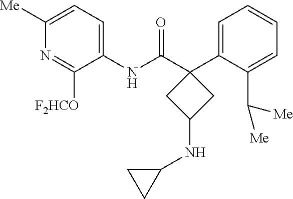 [SMILES: Cc1ccc(NC(=O)C2(c3ccccc3C(C)C)CC(NC3CC3)C2)c(OC(F)F)n1] | 430.2 [M + H]⁺ |
| | | 3-(cyclopropylamino)-N-(2- | |
| | | (difluoromethoxy)-6-methylpyridin-3-yl)-1-(2- | |
| | | isopropylphenyl)cyclobutane-1-carboxamide | |
| 40- isomer B | 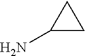 (Sigma-Aldrich) | 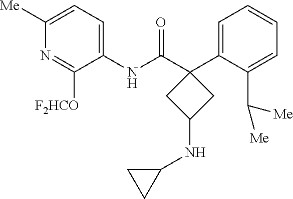 [SMILES: Cc1ccc(NC(=O)C2(c3ccccc3C(C)C)CC(NC3CC3)C2)c(OC(F)F)n1] | 430.2 [M + H]⁺ |
| | | 3-(cyclopropylamino)-N-(2- | |
| | | (difluoromethoxy)-6-methylpyridin-3-yl)-1-(2- | |
| | | isopropylphenyl)cyclobutane-1-carboxamide | |
| 41 | 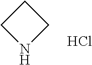 (Sigma-Aldrich) | 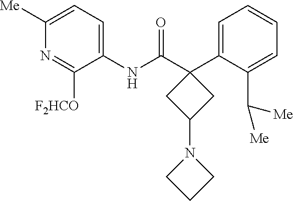 [SMILES: Cc1ccc(NC(=O)C2(c3ccccc3C(C)C)CC(N3CCC3)C2)c(OC(F)F)n1] | 430.2 [M + H]⁺ |
| | | 3-(azetidin-1-yl)-N-(2-(difluoromethoxy)-6- | |
| | | methylpyridin-3-yl)-1-(2- | |
| | | isopropylphenyl)cyclobutane-1-carboxamide | |
| 42 | 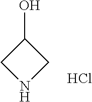 (Sigma-Aldrich) | 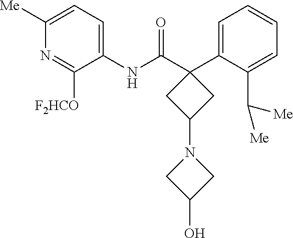 [SMILES: Cc1ccc(NC(=O)C2(c3ccccc3C(C)C)CC(N3CC(O)C3)C2)c(OC(F)F)n1] | 446.2 [M + H]⁺ |
| | | N-(2-(difluoromethoxy)-6-methylpyridin-3- | |
| | | yl)-3-(3-hydroxyazetidin-1-yl)-1-(2- | |
| | | isopropylphenyl)cyclobutane-1-carboxamide | |

### Example 43: Preparation of N-(2-(difluoromethoxy)-6-methylpyridin-3-yl)-1-(2-isopropylphenyl)-3-(N-methylacetamido)cyclobutane-1-carboxamide

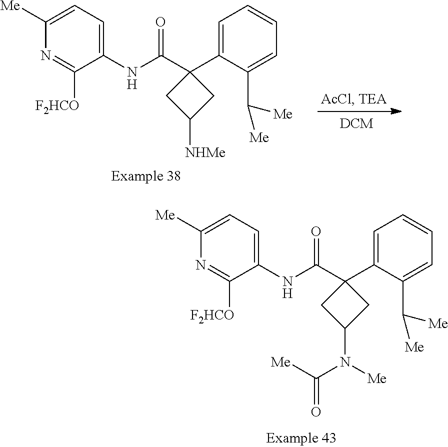 [SMILES: CNC1CC(C(=O)Nc2ccc(C)nc2OC(F)F)(c2ccccc2C(C)C)C1 ; CC(=O)N(C)C1CC(C(=O)Nc2ccc(C)nc2OC(F)F)(c2ccccc2C(C)C)C1]

In a dried, round-bottom flask equipped with a magnetic stirrer was combined Example 38 (1 equiv), acetyl chloride (1.5 equiv, Sigma-Aldrich), and triethylamine (3 equiv, Sigma-Aldrich) in anhydrous dichloromethane (0.06 M). The resulting reaction mixture was stirred at RT for 30 min. The volatiles were then removed in vacuo and the residue thus obtained was subjected to reverse-phase column chromatography (C₁₈, gradient elution: 95:5 (v/v) H₂O: MeCN→5:95 (v/v) H₂O: MeCN) to afford the title compound as a mixture of diastereomers (20% yield). LCMS: m/z=446.2 [M+H]⁺; ¹H NMR (CDCl₃): δ=8.54 (d, J=8.0 Hz, 1H), 7.60 (d, J=7.4 Hz, 1H), 7.50˜6.91 (m, 6H), 5.07 (br s, 0.7H, diastereomer A), 4.30 (br s, 0.3H, diastereomer B), 3.21˜2.57 (m, 7H), 2.38 (s, 3H), 2.11 (s, 3H), 1.89˜1.85 (m, 1H), 1.11 (d, J=6.8 Hz, 6H).

The following example was prepared in an analogous fashion to Example 43, but substituting acetyl chloride with the requisite electrophile.

| | Starting | | LCMS |
|---|---|---|---|
| Example | Electrophile | Structure | (m/z) |
| 44 | 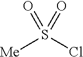 (Sigma- Aldrich) | 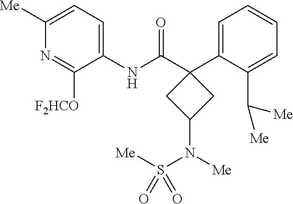 [SMILES: Cc1ccc(NC(=O)C2(c3ccccc3C(C)C)CC(N(C)S(C)(=O)=O)C2)c(OC(F)F)n1] | 482.2 [M + H]⁺ |
| | | N-(2-(difluoromethoxy)-6-methylpyridin-3- | |
| | | yl)-1-(2-isopropylphenyl)-3-(N- | |
| | | methylmethylsulfonamido)cyclobutane-1- | |
| | | carboxamide | |

### Example 45: Preparation of 2-(3-((2-(difluoromethoxy)-6-methylpyridin-3-yl)carbamoyl)-3-(2-isopropylphenyl)cyclobutyl)acetic acid

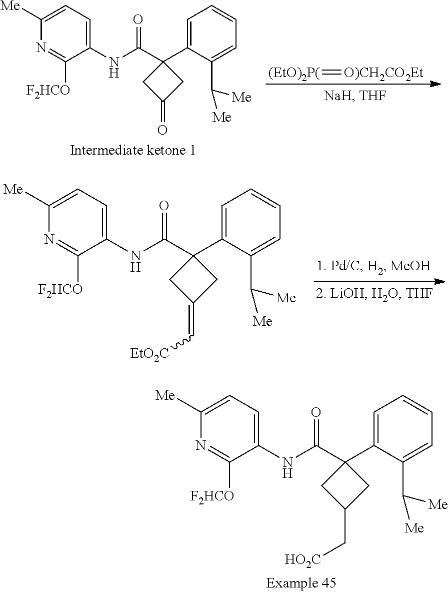

Step 1: In a dried, round-bottom flask equipped with a magnetic stirrer was dissolved triethyl phosphonoacetate (1.1 equiv, Sigma-Aldrich) in THE (0.10 M). To this was then added at 0° C. sodium hydride (60% w/w dispersion in paraffin oil, 1.1 equiv, Sigma-Aldrich) in one rapid portion. After 1 h of stirring at 0° C., Intermediate ketone 1 (1 equiv) was then added dropwise as a solution in THE (0.15 M) over a period of 5 min. Following the completion of addition, the reaction mixture was stirred at 0° C. for 30 min and then at RT for 1 h. The reaction was then carefully quenched with the dropwise addition of saturated aq. NH₄Cl and extracted with EtOAc. The combined organic extracts were washed further with water and brine, dried over Na₂SO₄, filtered, and the filtrate concentrated in vacuo. Purification of the crude product thus obtained by way of column chromatography (SiO₂, 5:1 (v/v) Hex: EtOAc) afforded ethyl 2-(3-((2-(difluoromethoxy)-6-methylpyridin-3-yl)carbamoyl)-3-(2-isopropylphenyl)cyclobutylidene)acetate as a yellow oil (70% yield).

Step 2: In a dried, round-bottom flask equipped with a magnetic stirrer was dissolved ethyl 2-(3-((2-(difluoromethoxy)-6-methylpyridin-3-yl)carbamoyl)-3-(2-isopropylphenyl)cyclobutylidene)acetate (1 equiv) from the previous step in methanol (0.065 M). The resulting pale-yellow solution was then deoxygenated via subsurface purging with nitrogen for 10 min before palladium (10% w/w over activated carbon, dry, 0.15 equiv, Sigma-Aldrich) was added in one rapid portion. The resulting black suspension was then subsurface purged with hydrogen for 10 min before it was stirred under a static hydrogen atmosphere at RT for 1 h. The reaction was subsequently quenched with dichloromethane and filtered through a bed of dichloromethane-wetted celite. The insolubles were washed further with dichloromethane and the filtrate was concentrated in vacuo. The crude ethyl 2-(3-((2-(difluoromethoxy)-6-methylpyridin-3-yl)carbamoyl)-3-(2-isopropylphenyl)cyclobutyl)acetate thus obtained was then taken up in THE (0.043 M). To this colorless solution was then added lithium hydroxide (0.4 M solution in water, 3 equiv) and the resulting mixture was stirred at RT for 2 h. Finally, the reaction mixture was carefully quenched with HCl (1 M solution in water, 3 equiv) and extracted with EtOAc. The combined organic extracts were washed sequentially with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN→5:95 (v/v) H₂O: MeCN) to afford the title compound as a mixture of diastereomers (4700 yield). LCMS: m/z=433.2 [M+H]⁺; ¹H NMR (DMSO-d₆): δ=12.09 (s, br, 1H), 8.11-8.03 (m, 1H), 7.77-7.24 (m, 6H), 7.10-7.07 (m, 1H), 2.76-2.27 (m, 11H), 1.06 (d, J=6.0 Hz, 3H), 1.05 (d, J=6.0 Hz, 3H).

The following examples were prepared in an analogous fashion to Example 45, but substituting Intermediate ketone 1 with the requisite ketone.

| | | | LCMS |
|---|---|---|---|
| Example | Starting Ketone | Structure | (m/z) |
| 46 | 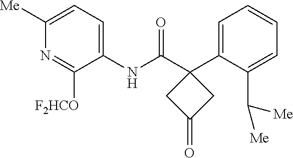 [SMILES: Cc1ccc(NC(=O)C2(c3ccccc3C(C)C)CC(=O)C2)c(OC(F)F)n1] Intermediate ketone 2 | 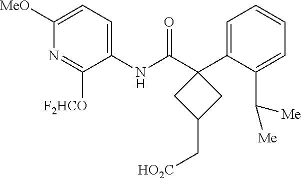 [SMILES: COc1ccc(NC(=O)C2(c3ccccc3C(C)C)CC(CC(=O)O)C2)c(OC(F)F)n1] | 449.2 [M + H]⁺ |
| | | 2-(3-((2-(difluoromethoxy)-6- | |
| | | methoxypyridin-3-yl)carbamoyl)-3- | |
| | | (2-isopropylphenyl)cyclobutyl)acetic | |
| | | acid | |
| 47 | 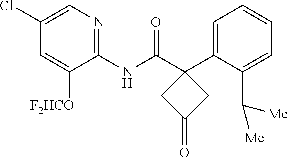 [SMILES: CC(C)c1ccccc1C1(C(=O)Nc2ncc(Cl)cc2OC(F)F)CC(=O)C1] Intermediate ketone 3 | 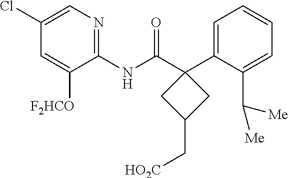 [SMILES: CC(C)c1ccccc1C1(C(=O)Nc2ncc(Cl)cc2OC(F)F)CC(CC(=O)O)C1] | 453.1, 455.1 [M + H]⁺ |
| | | 2-(3-((5-chloro-3- | |
| | | (difluoromethoxy)pyridin-2- | |
| | | yl)carbamoyl)-3-(2- | |
| | | isopropylphenyl)cyclobutyl)acetic | |
| | | acid | |
| 48 | 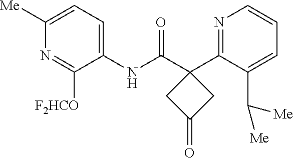 [SMILES: Cc1ccc(NC(=O)C2(c3ncccc3C(C)C)CC(=O)C2)c(OC(F)F)n1] Intermediate ketone 6 | 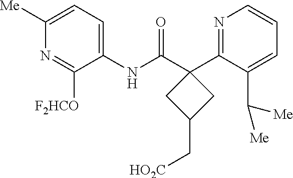 [SMILES: Cc1ccc(NC(=O)C2(c3ncccc3C(C)C)CC(CC(=O)O)C2)c(OC(F)F)n1] | 434.2 [M + H]⁺ |
| | | 2-(3-((2-(difluoromethoxy)-6- | |
| | | methylpyridin-3-y1)carbamoyl)-3-(3- | |
| | | isopropylpyridin-2- | |
| | | yl)cyclobutyl)acetic acid | |
| 91 | 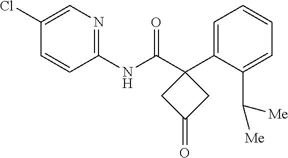 [SMILES: CC(C)c1ccccc1C1(C(=O)Nc2ccc(Cl)cn2)CC(=O)C1] Intermediate ketone 7 | 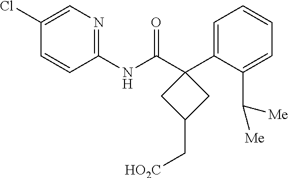 [SMILES: CC(C)c1ccccc1C1(C(=O)Nc2ccc(Cl)cn2)CC(CC(=O)O)C1] | 387.2, 389.2 [M + H]⁺ |
| | | 2-(3-((5-chloropyridin-2- | |
| | | yl)carbamoyl)-3-(2- | |
| | | isopropylphenyl)cyclobutyl)acetic | |
| | | acid | |

### Example 49: Preparation of N-(2-(difluoromethoxy)-6-methylpyridin-3-yl)-3-(2-hydroxyethyl)-1-(2-isopropylphenyl)cyclobutane-1-carboxamide

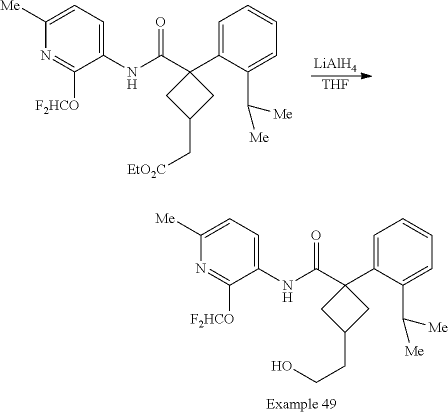

In a dried, round-bottom flask equipped with a magnetic stirrer was dissolved ethyl 2-(3-((2-(difluoromethoxy)-6-methylpyridin-3-yl)carbamoyl)-3-(2-isopropylphenyl)cyclobutyl)acetate (1 equiv, Example 45, step 2) in anhydrous THE (0.022 M). To this colorless solution was then added lithium aluminum hydride (1 M solution in THF, 2 equiv, Sigma-Aldrich) dropwise and the resulting reaction mixture was stirred at RT for 2 h. The reaction was then carefully quenched with saturated aq. NH₄Cl at 0° C. and extracted with EtOAc. The combined organic extracts were washed further with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN 4 5:95 (v/v) H₂O: MeCN) to afford the title compound as a mixture of diastereomers (62% yield). LCMS: m/z=419.2 [M+H]⁺; ¹H NMR (DMSO-d₆): δ=8.11 8.04 (m, 1H), 7.77˜7.24 (m, 6H), 7.10˜7.07 (m, 1H), 3.38˜3.34 (m, 2H), 2.95˜2.50 (m, 4H), 2.35 (s, 3H), 2.29˜2.16 (m, 2H), 1.65˜1.49 (m, 2H), 1.06 (d, J=6.0 Hz, 3H), 1.04 (d, J=6.0 Hz, 3H).

### Example 50: Preparation of 3-(5-cyanopyridin-2-yl)-N-(2-(difluoromethoxy)-6-methylpyridin-3-yl)-3-hydroxy-1-(2-isopropylphenyl)cyclobutane-1-carboxamide

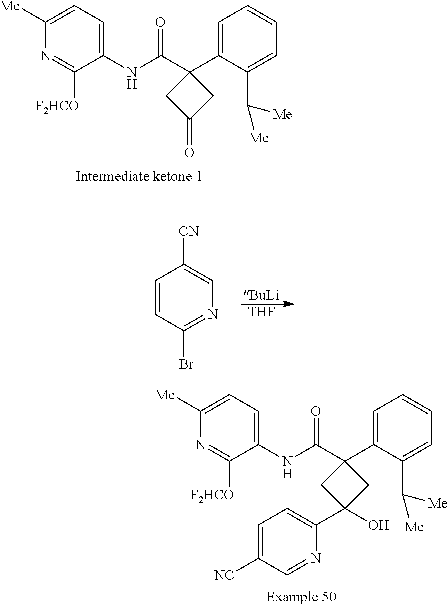

In a dried, round-bottom flask equipped with a magnetic stirrer was dissolved 6-bromonicotinonitrile (1 equiv, Alfa Aesar) in a 3:1 (v/v) solution of THE and hexanes (0.045 M). To this was then added dropwise at −100° C. n-butyl lithium (1.6 M solution in hexanes, 1.2 equiv, 1 equiv, Sigma-Aldrich) over a period of 10 min, and the resulting orange solution was stirred at −100° C. for another 10 min. Finally, Intermediate ketone 1 (1 equiv) was added dropwise at −100° C. as a solution in THF (0.16 M) over a period of 5 min. Following the completion of addition, the reaction mixture was stirred at −100° C. for 30 min and then at RT for 1 h. The reaction was then carefully quenched with ice water and extracted with EtOAc. The combined organic extracts were washed further with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN 4 5:95 (v/v) H₂O: MeCN) to afford the title compound as a white solid (4.5% yield). LCMS: m/z=493.2 [M+H]⁺; ¹H NMR (methanol-d₄): δ=8.80 (dd, J=2.1, 0.9 Hz, 1H), 8.43 (d, J=8.1 Hz, 1H), 8.11 (dd, J=8.4, 2.1 Hz, 1H), 7.88 (dd, J=8.1, 0.9 Hz, 1H), 7.63˜7.26 (m, 5H), 7.04 (d, J=8.4 Hz, 1H), 3.54˜3.51 (m, 2H), 3.16˜3.04 (m, 2H), 2.82 (sept, J=6.9 Hz, 1H), 2.39 (s, 3H), 1.15 (d, J=6.9 Hz, 6H).

The following examples were prepared in an analogous fashion to Example 50, but substituting the aryl lithium generated in situ with the requisite commercially available organometallic reagent.

| | Starting | | |
|---|---|---|---|
| | Organometallic | | LCMS |
| Example | Reagent | Structure | (m/z) |
| 51-isomer A | MeLi (Sigma-Aldrich) | 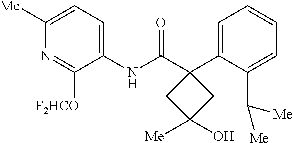 [SMILES: Cc1ccc(NC(=O)C2(c3ccccc3C(C)C)CC(C)(O)C2)c(OC(F)F)n1] | 405.2 [M + H]⁺ |
| | | N-(2-(difluoromethoxy)-6-methylpyridin-3- | |
| | | yl)-3-hydroxy-1-(2-isopropylphenyl)-3- | |
| | | methylcyclobutane-1-carboxamide | |
| 51-isomer B | MeLi (Sigma-Aldrich) | 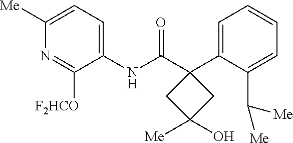 [SMILES: Cc1ccc(NC(=O)C2(c3ccccc3C(C)C)CC(C)(O)C2)c(OC(F)F)n1] | 405.2 [M + H]⁺ |
| | | N-(2-(difluoromethoxy)-6-methylpyridin-3- | |
| | | yl)-3-hydroxy-1-(2-isopropylphenyl)-3- | |
| | | methylcyclobutane-1-carboxamide | |
| 52 | EtMgBr (Sigma-Aldrich) | 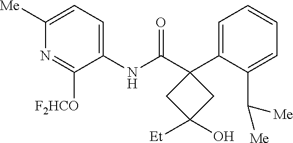 [SMILES: CCC1(O)CC(C(=O)Nc2ccc(C)nc2OC(F)F)(c2ccccc2C(C)C)C1] | 419.2 [M + H]⁺ |
| | | N-(2-(difluoromethoxy)-6-methylpyridin-3- | |
| | | yl)-3-ethyl-3-hydroxy-1-(2- | |
| | | isopropylphenyl)cyclobutane-1-carboxamide | |

### Example 53: Preparation of 2-(3-((2-(difluoromethoxy)-6-methylpyridin-3-yl)carbamoyl)-1-hydroxy-3-(2-isopropylphenyl)cyclobutyl)-2,2-difluoroacetic acid

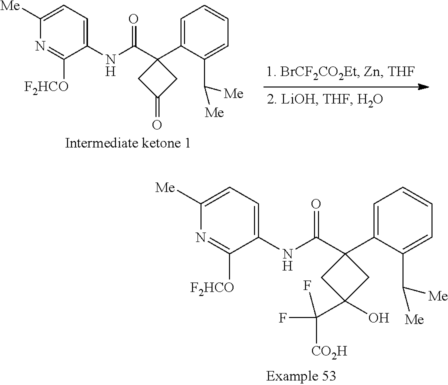 [SMILES: Cc1ccc(NC(=O)C2(c3ccccc3C(C)C)CC(=O)C2)c(OC(F)F)n1 ; Cc1ccc(NC(=O)C2(c3ccccc3C(C)C)CC(O)(C(F)(F)C(=O)O)C2)c(OC(F)F)n1]

Step 1: In a dried, round-bottom flask equipped with a magnetic stirrer was combined Intermediate ketone 1 (1 equiv), ethyl bromodifluoroacetate (2 equiv, Sigma-Aldrich), and zinc (3 equiv, Strem) in THE (0.033 M). The resulting suspension was sonicated under nitrogen for 5 h. The reaction was then quenched with saturated aq. NH₄Cl and extracted with EtOAc. The combined organic extracts were washed further with water and brine, dried over Na₂SO₄, filtered, and the filtrate concentrated in vacuo. Purification of the crude product thus obtained by way of column chromatography (SiO₂, 5:1 (v/v) Hex: EtOAc) afforded ethyl 2-(3-((2-(difluoromethoxy)-6-methylpyridin-3-yl)carbamoyl)-1-hydroxy-3-(2-isopropylphenyl)cyclobutyl)-2,2-difluoroacetate as a colorless oil (94% yield).

Step 2: In a dried, round-bottom flask equipped with a magnetic stirrer was dissolved ethyl 2-(3-((2-(difluoromethoxy)-6-methylpyridin-3-yl)carbamoyl)-1-hydroxy-3-(2-isopropylphenyl)cyclobutyl)-2,2-difluoroacetate (1 equiv) from the previous step in THE (0.033 M). To this colorless solution was then added lithium hydroxide (0.25 M solution in water, 2.5 equiv) and the resulting mixture was stirred at RT for 16 h. Finally, the reaction mixture was carefully quenched with HCl (1 M solution in water, 2.5 equiv) and extracted with EtOAc. The combined organic extracts were washed sequentially with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN→5:95 (v/v) H₂O: MeCN) to afford the title compound as a mixture of diastereomers (56% yield). LCMS: m/z=485.2 [M+H]⁺; ¹H NMR (methanol-d₄): δ=8.80 (d, J=8.4 Hz, 0.85H, diastereomer A), 8.25 (d, J=8.4 Hz, 0.15H, diastereomer B), 7.80˜7.09 (m, 5H), 7.00 (d, J=8.4 Hz, 0.85H, diastereomer A), 6.98 (d, J=8.4 Hz, 0.15H, diastereomer B), 3.58 (d, J=12.6 Hz, 0.3H, diastereomer B), 3.26 (d, J=12.6 Hz, 1.7H, diastereomer A), 2.95 (d, J=12.6 Hz, 1.7H, diastereomer A), 2.89˜2.77 (m, 1H), 2.65 (d, J=12.6 Hz, 0.3H, diastereomer B), 2.37 (s, 2.55H, diastereomer A), 2.36 (s, 0.45H, diastereomer B), 1.13 (d, J=6.6 Hz, 5.1H, diastereomer A), 1.11 (d, J=6.6 Hz, 0.9H, diastereomer B).

The following examples were prepared in an analogous fashion to Example 53, but substituting Intermediate ketone 1 with the requisite ketone.

| | | | LCMS |
|---|---|---|---|
| Example | Starting Ketone | Structure | (m/z) |
| 54- isomer A | 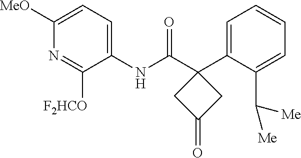 [SMILES: COc1ccc(NC(=O)C2(c3ccccc3C(C)C)CC(=O)C2)c(OC(F)F)n1] Intermediate ketone 2 | 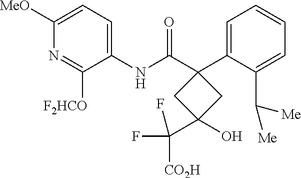 [SMILES: COc1ccc(NC(=O)C2(c3ccccc3C(C)C)CC(O)(C(F)(F)C(=O)O)C2)c(OC(F)F)n1] | 501.2 [M + H]⁺ |
| | | 2-(3-((2-(difluoromethoxy)-6- | |
| | | methoxypyridin-3-yl)carbamoyl)-1- | |
| | | hydroxy-3-(2- | |
| | | isopropylphenyl)cyclobutyl)-2,2- | |
| | | difluoroacetic acid | |
| 54- isomer B | 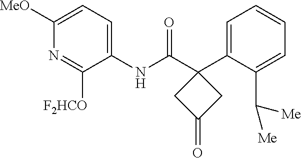 [SMILES: COc1ccc(NC(=O)C2(c3ccccc3C(C)C)CC(=O)C2)c(OC(F)F)n1] Intermediate ketone 2 | 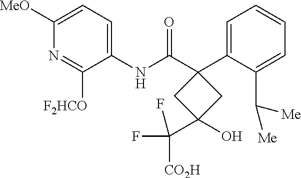 [SMILES: COc1ccc(NC(=O)C2(c3ccccc3C(C)C)CC(O)(C(F)(F)C(=O)O)C2)c(OC(F)F)n1] | 501.2 [M + H]⁺ |
| | | 2-(3-((2-(difluoromethoxy)-6- | |
| | | methoxypyridin-3-yl)carbamoyl)-1- | |
| | | hydroxy-3-(2- | |
| | | isopropylphenyl)cyclobutyl)-2,2- | |
| | | difluoroacetic acid | |
| 55- isomer A | 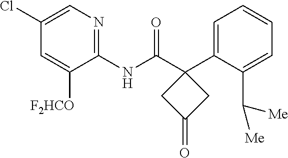 [SMILES: CC(C)c1ccccc1C1(C(=O)Nc2ncc(Cl)cc2OC(F)F)CC(=O)C1] Intermediate ketone 3 | 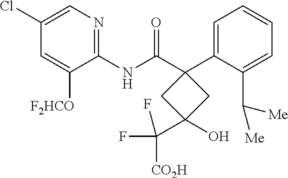 [SMILES: CC(C)c1ccccc1C1(C(=O)Nc2ncc(Cl)cc2OC(F)F)CC(O)(C(F)(F)C(=O)O)C1] | 505.2, 507.2 [M + H]⁺ |
| | | 2-(3-((5-chloro-3- | |
| | | (difluoromethoxy)pyridin-2- | |
| | | yl)carbamoyl)-1-hydroxy-3-(2- | |
| | | isopropylphenyl)cyclobutyl)-2,2- | |
| | | difluoroacetic acid | |
| 55- isomer B | 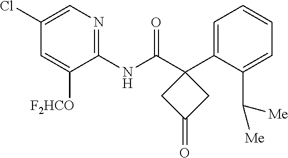 [SMILES: CC(C)c1ccccc1C1(C(=O)Nc2ncc(Cl)cc2OC(F)F)CC(=O)C1] Intermediate ketone 3 | 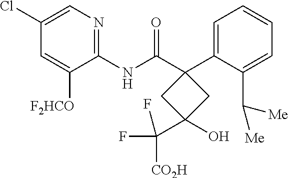 [SMILES: CC(C)c1ccccc1C1(C(=O)Nc2ncc(Cl)cc2OC(F)F)CC(O)(C(F)(F)C(=O)O)C1] | 505.2, 507.2 [M + H]⁺ |
| | | 2-(3-((5-chloro-3- | |
| | | (difluoromethoxy)pyridin-2- | |
| | | yl)carbamoyl)-1-hydroxy-3-(2- | |
| | | isopropylphenyl)cyclobutyl)-2,2- | |
| | | difluoroacetic acid | |
| 56 | 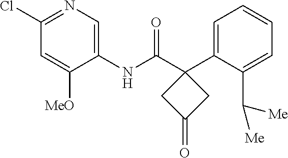 [SMILES: COc1cc(Cl)ncc1NC(=O)C1(c2ccccc2C(C)C)CC(=O)C1] Intermediate ketone 4 | 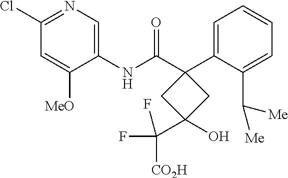 [SMILES: COc1cc(Cl)ncc1NC(=O)C1(c2ccccc2C(C)C)CC(O)(C(F)(F)C(=O)O)C1] | 469.1, 471.1 [M + H]⁺ |
| | | 2-(3-((6-chloro-4-methoxypyridin-3- | |
| | | yl)carbamoyl)-1-hydroxy-3-(2- | |
| | | isopropylphenyl)cyclobutyl)-2,2- | |
| | | difluoroacetic acid | |
| 93- isomer A | 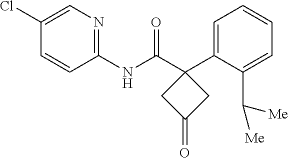 [SMILES: CC(C)c1ccccc1C1(C(=O)Nc2ccc(Cl)cn2)CC(=O)C1] Intermediate ketone 7 | 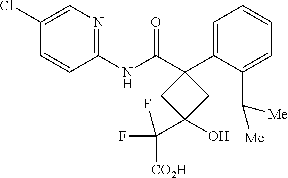 [SMILES: CC(C)c1ccccc1C1(C(=O)Nc2ccc(Cl)cn2)CC(O)(C(F)(F)C(=O)O)C1] | 439.1, 441.1 [M + H]⁺ |
| | | 2-(3-((5-chloropyridin-2- | |
| | | yl)carbamoyl)-1-hydroxy-3-(2- | |
| | | isopropylphenyl)cyclobutyl)-2,2- | |
| | | difluoroacetic acid | |
| 93- isomer B | 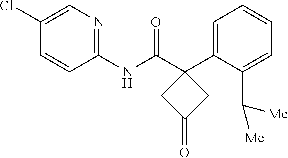 [SMILES: CC(C)c1ccccc1C1(C(=O)Nc2ccc(Cl)cn2)CC(=O)C1] Intermediate ketone 7 | 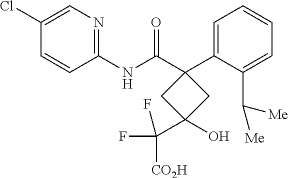 [SMILES: CC(C)c1ccccc1C1(C(=O)Nc2ccc(Cl)cn2)CC(O)(C(F)(F)C(=O)O)C1] | 439.1, 441.1 [M + H]⁺ |
| | | 2-(3-((5-chloropyridin-2- | |
| | | y1)carbamoyl)-1-hydroxy-3-(2- | |
| | | isopropylphenyl)cyclobutyl)-2,2- | |
| | | difluoroacetic acid | |
| 94- isomer A | 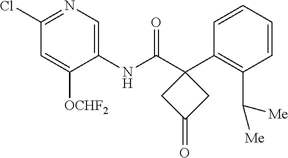 [SMILES: CC(C)c1ccccc1C1(C(=O)Nc2cnc(Cl)cc2OC(F)F)CC(=O)C1] Intermediate ketone 8 | 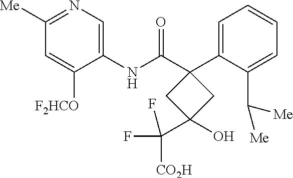 [SMILES: Cc1cc(OC(F)F)c(NC(=O)C2(c3ccccc3C(C)C)CC(O)(C(F)(F)C(=O)O)C2)cn1] | 485.2 [M + H]⁺ |
| | | 2-(3-((4-(difluoromethoxy)-6- | |
| | | methylpyridin-3-yl)carbamoyl)-1- | |
| | | hydroxy-3-(2- | |
| | | isopropylphenyl)cyclobutyl)-2,2- | |
| | | difluoroacetic acid | |
| 94- isomer B | 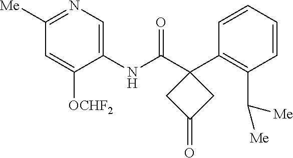 [SMILES: Cc1cc(OC(F)F)c(NC(=O)C2(c3ccccc3C(C)C)CC(=O)C2)cn1] Intermediate ketone 8 | 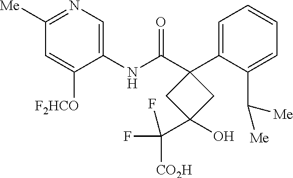 [SMILES: Cc1cc(OC(F)F)c(NC(=O)C2(c3ccccc3C(C)C)CC(O)(C(F)(F)C(=O)O)C2)cn1] | 485.2 [M + H]⁺ |
| | | 2-(3-((4-(difluoromethoxy)-6- | |
| | | methylpyridin-3-yl)carbamoyl)-1- | |
| | | hydroxy-3-(2- | |
| | | isopropylphenyl)cyclobutyl)-2,2- | |
| | | difluoroacetic acid | |

### Example 57: N-(2-(difluoromethoxy)-6-methylpyridin-3-yl)-3-hydroxy-1-(3-isopropylpyridin-2-yl)cyclobutane-1-carboxamide

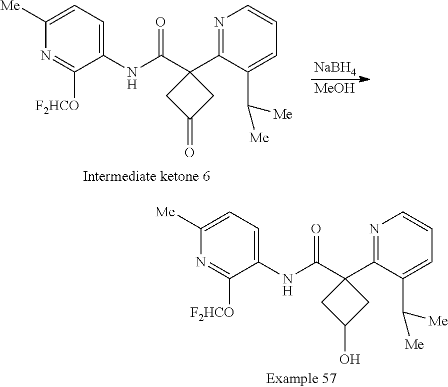

In a dried, round-bottom flask equipped with a magnetic stirrer was dissolved Intermediate ketone 6 (1 equiv) in methanol (0.033 M). To this colorless solution was then added sodium borohydride (4 equiv, Sigma-Aldrich) and the resulting mixture was stirred at RT for 1 h. Finally, the reaction mixture was carefully quenched with water and extracted with EtOAc. The combined organic extracts were washed further with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN→5:95 (v/v) H₂O: MeCN) to afford the title compound as a mixture of diastereomers (5600 yield). LCMS: m/z=392.1 [M+H]⁺; ¹H NMR (methanol-d₄): δ=8.50 (d, J=4.2 Hz, 1H), 8.28 (d, J=8.0 Hz, 1H), 7.82 (d, J=8.0 Hz, 1H), 7.69-7.14 (m, 2H), 7.02 (d, J=8.0 Hz, 1H), 4.49-4.42 (mn, 0.4H, diastereomer A), 4.14-4.05 (mn, 0.6H, diastereomner B), 3.25-2.60 (mn, 5H), 2.39 (s, 3H), 1.15 (d, J=6.6 Hz, 2.4H, diastereomner B), 1.13 (d, J=6.6 Hz, 3.6H, diastereomner A).

The following examples were prepared in an analogous fashion to Example 57, but substituting Intermediate ketone 6 with the requisite ketone or aldehyde.

| | Starting Ketone/ | | LCMS |
|---|---|---|---|
| Example | Aldehyde | Structure | (m/z) |
| 58 | 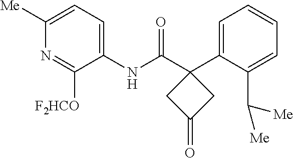 [SMILES: Cc1ccc(NC(=O)C2(c3ccccc3C(C)C)CC(=O)C2)c(OC(F)F)n1] Intermediate ketone 1 | 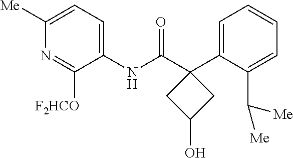 [SMILES: Cc1ccc(NC(=O)C2(c3ccccc3C(C)C)CC(O)C2)c(OC(F)F)n1] N-(2-(difluoromethoxy)-6- methylpyridin-3-yl)-3-hydroxy-1-(2- isopropylphenyl)cyclobutane-1- carboxamide | 391.1 [M + H]⁺ |
| 59 | 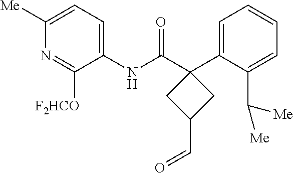 [SMILES: Cc1ccc(NC(=O)C2(c3ccccc3C(C)C)CC(C=O)C2)c(OC(F)F)n1] Intermediate aldehyde 1 | 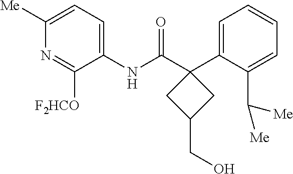 [SMILES: Cc1ccc(NC(=O)C2(c3ccccc3C(C)C)CC(CO)C2)c(OC(F)F)n1] N-(2-(difluoromethoxy)-6- methylpyridin-3-yl)-3-(hydroxymethyl)- 1-(2-isopropylphenyl)cyclobutane-1- carboxamide | 405.2 [M + H]⁺ |
| 60 | 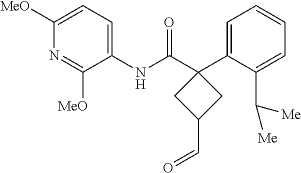 [SMILES: COc1ccc(NC(=O)C2(c3ccccc3C(C)C)CC(C=O)C2)c(OC)n1] Intermediate aldehyde 2 | 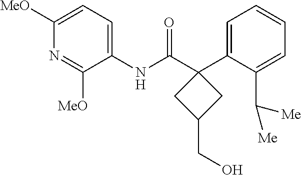 [SMILES: COc1ccc(NC(=O)C2(c3ccccc3C(C)C)CC(CO)C2)c(OC)n1] N-(2,6-dimethoxypyridin-3-yl)-3- (hydroxymethyl)-1-(2- isopropylphenyl)cyclobutane-1- carboxamide | 385.2 [M + H]⁺ |

### Example 61: N-(2-(difluoromethoxy)-6-methylpyridin-3-yl)-3,3-difluoro-1-(2-isopropylphenyl)cyclobutane-1-carboxamide

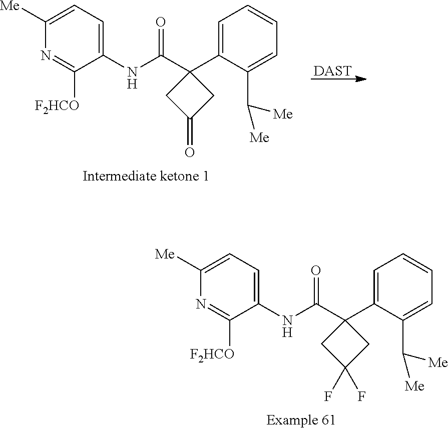

In a Nalgene reaction vessel equipped with a magnetic stirrer was reacted Intermediate ketone 1 (1 equiv) with diethylaminosulfur trifluoride (250 equiv, Sigma-Aldrich) at RT for 1 h. The reaction was then carefully quenched with ice water and extracted with dichloromethane. The combined organic extracts were washed further with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN→5:95 (v/v) H₂O: MeCN) to afford the title compound as a white solid (15% yield). LCMS: m/z=411.2 [M+H]⁺; ¹H NMR (methanol-d₄): δ=8.24 (d, J=8.4 Hz, 1H), 7.57˜7.19 (m, 5H), 7.03 (d, J=8.4 Hz, 1H), 3.65˜3.50 (m, 2H), 3.21˜3.12 (m, 2H), 2.75 (sept, J=6.8 Hz, 1H), 2.39 (s, 3H), 1.14 (d, J=6.8 Hz, 6H).

### Example 62: N-(2-(difluoromethoxy)-6-methylpyridin-3-yl)-3-fluoro-1-(2-isopropylphenyl)cyclobutane-1-carboxamide

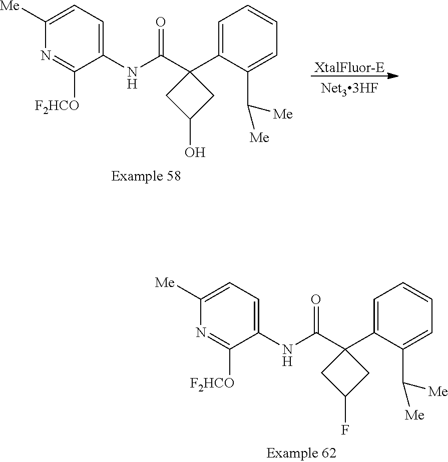

In a Nalgene reaction vessel equipped with a magnetic stirrer was combined Example 58 (1 equiv) and (diethylamino)difluorosulfonium tetrafluoroborate (2.1 equiv, Sigma-Aldrich) in 1,2-dichloroethane (0.065 M). To this solution was then added triethylamine trishydrofluoride (2.1 equiv, Sigma-Aldrich) and the resulting mixture was stirred at RT for 1 h. The reaction was then carefully quenched with ice water and extracted with dichloromethane. The combined organic extracts were washed further with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN→5:95 (v/v) H₂O: MeCN) to afford the title compound as a white solid (6.2% yield). LCMS: m/z=393.2 [M+H]⁺; ¹H NMR (methanol-d₄): δ=8.32 (d, J=8.4 Hz, 1H), 7.53˜7.17 (m, 5H), 7.02 (d, J=8.4 Hz, 1H), 5.46˜5.28 (m, 1H), 3.36˜3.27 (m, 2H), 2.81˜2.70 (m, 3H), 2.38 (s, 3H), 1.25 (d, J=6.8 Hz, 6H).

### Example 63: Preparation of 3-(cyanomethyl)-N-(2-(difluoromethoxy)-6-methylpyridin-3-yl)-1-(2-isopropylphenyl)cyclobutane-1-carboxamide

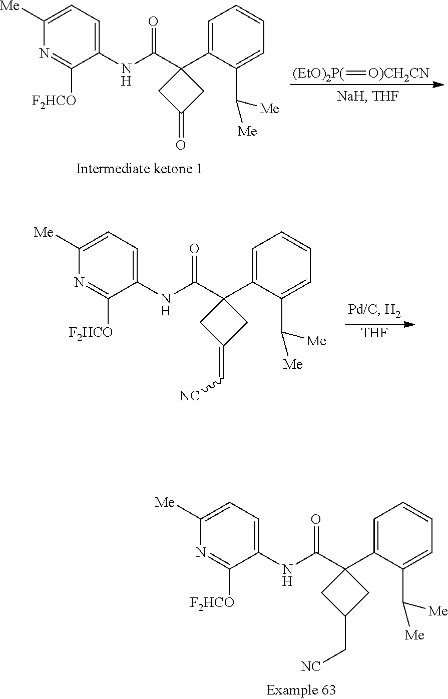

Step 1: In a dried, round-bottom flask equipped with a magnetic stirrer was dissolved diethyl cyanomethylphosphonate (1.1 equiv, Sigma-Aldrich) in THF (0.034 M). To this was then added at 0° C. sodium hydride (60% w/w dispersion in paraffin oil, 1.1 equiv, Sigma-Aldrich) in one rapid portion. After 1 h of stirring at 0° C., Intermediate ketone 1 (1 equiv) was then added dropwise as a solution in THE (0.11 M) over a period of 5 min. Following the completion of addition, the reaction mixture was stirred at 0° C. for 30 min and then at RT for 1 h. The reaction was then carefully quenched with the dropwise addition of water and extracted with EtOAc. The combined organic extracts were washed further with water and brine, dried over Na₂SO₄, filtered, and the filtrate concentrated in vacuo. Purification of the crude product thus obtained by way of column chromatography (SiO₂, 5:1 (v/v) Hex: EtOAc) afforded 3-(cyanomethylene)-N-(2-(difluoromethoxy)-6-methylpyridin-3-yl)-1-(2-isopropylphenyl)cyclobutane-1-carboxamide as a yellow oil (86% yield).

Step 2: In a dried, round-bottom flask equipped with a magnetic stirrer was dissolved 3-(cyanomethylene)-N-(2-(difluoromethoxy)-6-methylpyridin-3-yl)-1-(2-isopropylphenyl)cyclobutane-1-carboxamide (1 equiv) from the previous step in THF (0.06 M). The resulting solution was then deoxygenated via subsurface purging with nitrogen for 10 min before palladium (10% w/w over activated carbon, dry, 0.4 equiv, Sigma-Aldrich) was added in one rapid portion. The resulting black suspension was then subsurface purged with hydrogen for 10 min before it was stirred under a static hydrogen atmosphere at RT for 1 h. The reaction was subsequently quenched with dichloromethane and filtered through a bed of dichloromethane-wetted celite. The insolubles were washed further with dichloromethane and the filtrate was concentrated in vacuo. The crude product thus obtained was subjected to reverse-phase column chromatography (C₁₈, gradient elution: 95:5 (v/v) H₂O: MeCN→5:95 (v/v) H₂O: MeCN) to afford the title compound as a mixture of diastereomers (47% yield). LCMS: m/z=414.2 [M+H]⁺; ¹H NMR (DMSO-d₆): δ=8.05 (d, J=7.8 Hz, 0.29H, diastereomer A), 8.00 (d, J=7.8 Hz, 0.71H, diastereomer B), 7.83˜7.25 (m, 6H), 7.09 (d, J=8.1 Hz, 1H), 3.01˜2.63 (m, 6H), 2.48˜2.27 (m, 5H), 1.06 (d, J=6.0 Hz, 1.74H, diastereomer A), 1.05 (d, J=6.0 Hz, 4.26H, diastereomer B).

The following example was prepared in an analogous fashion to Example 63, but substituting diethyl cyanomethylphosphonate with the requisite phosphonate ester.

| | Starting Phosphonate | | LCMS |
|---|---|---|---|
| Example | Ester | Structure | (m/z) |
| 64 | 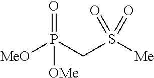 [SMILES: COP(=O)(CS(C)(=O)=O)OC] (Sigma-Aldrich) | 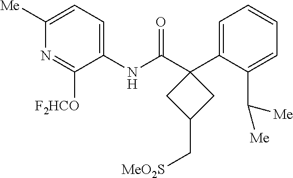 [SMILES: Cc1ccc(NC(=O)C2(c3ccccc3C(C)C)CC(CS(C)(=O)=O)C2)c(OC(F)F)n1] N-(2-(difluoromethoxy)-6- methylpyridin-3-yl)-1-(2- isopropylphenyl)-3- ((methylsulfonyl)methyl)cyclobutane-1- carboxamide | 467.1 [M + H]⁺ |

The following example was prepared in an analogous fashion to Example 63, but substituting Intermediate ketone 1 with the requisite ketone.

| | | | LCMS |
|---|---|---|---|
| Example | Starting Ketone | Structure | (m/z) |
| 111 | 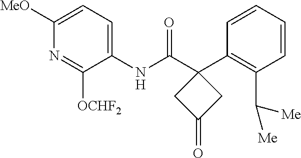 [SMILES: COc1ccc(NC(=O)C2(c3ccccc3C(C)C)CC(=O)C2)c(OC(F)F)n1] Intermediate ketone 2 | 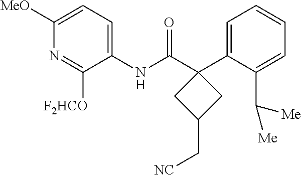 [SMILES: COc1ccc(NC(=O)C2(c3ccccc3C(C)C)CC(CC#N)C2)c(OC(F)F)n1] 3-(cyanomethyl)-N-(2- (difluoromethoxy)-6- methoxypyridin-3-yl)-1-(2- isopropylphenyl)cyclobutane-1- carboxamide | 430.2 [M + H]⁺ |

### Example 65: 3-((2-(difluoromethoxy)-6-methylpyridin-3-yl)carbamoyl)-3-(2-isopropylphenyl)cyclobutane-1-carboxylic acid

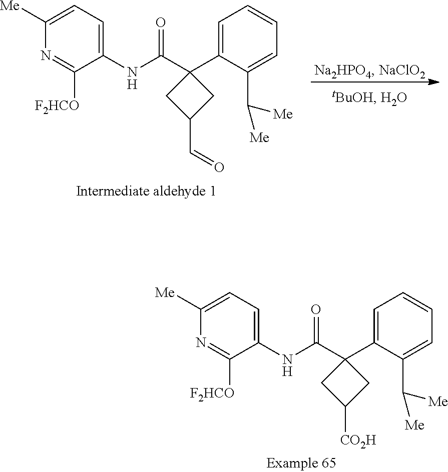

In a round-bottom flask equipped with a magnetic stirrer was combined Intermediate aldehyde 1 (1 equiv) and sodium hydrogen phosphate (10 equiv, Sigma-Aldrich) in a 3:1 (v/v) solution of tert-butanol and water (0.02 M). To this colorless solution was then added sodium chlorite (6 equiv, Sigma-Aldrich) and the resulting mixture was heated at 70° C. for 48 h. The volatiles were then removed in vacuo and the crude product thus obtained was subjected to reverse-phase column chromatography (C₁₈, gradient elution: 95:5 (v/v) H₂O: MeCN→5:95 (v/v) H₂O: MeCN) to afford the title compound as a white solid (34% yield). LCMS: m/z=419.2 [M+H]⁺; ¹H NMR (DMSO-d₆): δ=12.22 (s, 1H), 8.05 (d, J=7.8 Hz, 1H), 7.73 (s, 1H), 7.63˜7.25 (m, 5H), 7.09 (d, J=7.8 Hz, 1H), 3.04˜2.92 (m, 3H), 2.74˜2.51 (m, 3H), 2.35 (s, 3H), 1.06 (d, J=6.9 Hz, 6H).

### Example 66: N¹-(2-(difluoromethoxy)-6-methylpyridin-3-yl)-1-(2-isopropylphenyl)-N³,N³-dimethylcyclobutane-1,3-dicarboxamide

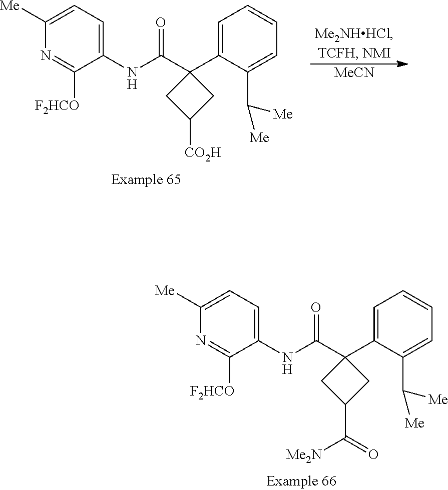

In a dried, round-bottom flask equipped with a magnetic stirrer was combined Example 65 (1 equiv), dimethylamine hydrochloride (1.3 equiv, Sigma-Aldrich), and 1-methylimidazole (5 equiv, Sigma-Aldrich) in anhydrous acetonitrile (0.028 M). To this was then added chloro-N,N,N′,N′-tetramethylformamidinium hexafluorophosphate (1.3 equiv, Chem-Impex) in one rapid portion and the resulting solution was stirred at RT for 2 h. The reaction was subsequently quenched with saturated aq. NH₄Cl and extracted with EtOAc. The combined organic extracts were washed further with water and brine, dried over Na₂SO₄, filtered, and the filtrate concentrated in vacuo. The crude product thus obtained was subjected 10 to reverse-phase column chromatography (C₁₈, gradient elution: 95:5 (v/v) H₂O: MeCN→5:95 (v/v) H₂O: MeCN) to afford the title compound as a white solid (38% yield). LCMS: m z=446.2 [M+H]⁺; ¹H NMR (DMSO-d₆): δ=8.08 (d, J=8.1 Hz, 1H), 7.75˜7.69 (m, 2H), 7.49˜7.25 (m, 4H), 7.09 (d, J=8.1 Hz, 1H), 3.19˜3.01 (m, 3H), 2.87 (s, 3H), 2.83 (s, 3H), 2.80˜2.65 (m, 3H), 2.35 (s, 3H), 1.06 (d, J=6.6 Hz, 6H).

The following example was prepared in an analogous fashion to Example 66, but substituting Example 65 with the requisite carboxylic acid.

| | | | LCMS |
|---|---|---|---|
| Example | Starting Acid | Structure | (m/z) |
| 67 | 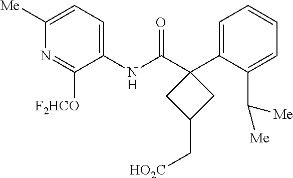 [SMILES: Cc1ccc(NC(=O)C2(c3ccccc3C(C)C)CC(CC(=O)O)C2)c(OC(F)F)n1] Example 45 | 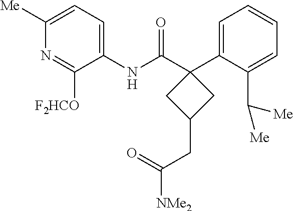 [SMILES: Cc1ccc(NC(=O)C2(c3ccccc3C(C)C)CC(CC(=O)N(C)C)C2)c(OC(F)F)n1] N-(2-(difluoromethoxy)-6- methylpyridin-3-yl)-3-(2- (dimethylamino)-2-oxoethyl)-1-(2- isopropylphenyl)cyclobutane-1- carboxamide | 460.1 [M + H]⁺ |

### Example 68: Preparation of N-(2-(difluoromethoxy)-6-methylpyridin-3-yl)-1-(2-isopropylphenyl)-3-(2-oxoazetidin-1-yl)cyclobutane-1-carboxamide

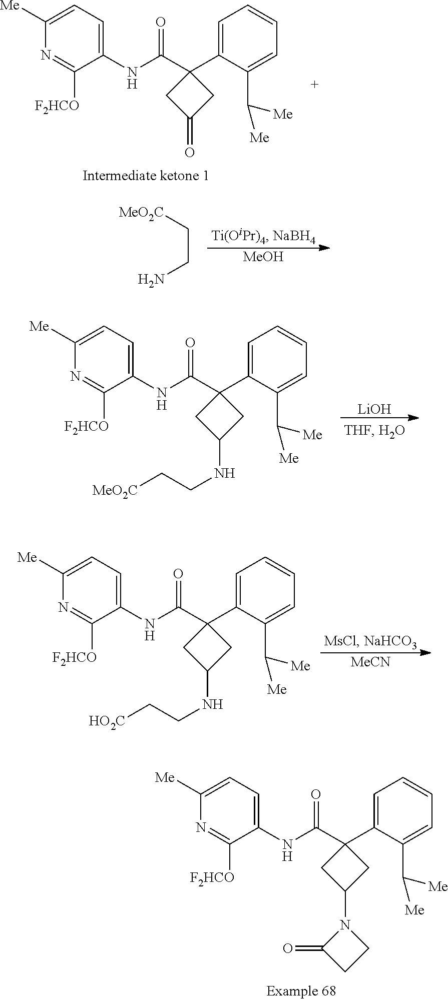

Step 1: In a dried, round-bottom flask equipped with a magnetic stirrer was combined Intermediate ketone 1 (1 equiv) and methyl 3-aminopropanoate (1.2 equiv, Matrix Scientific) in methanol (0.09 M). To this was then added titanium(IV) isopropoxide (2 equiv, Sigma-Aldrich) dropwise over a period of 1 min and the resulting mixture was stirred at RT for 2 h. Finally, with the reaction mixture cooled to 0° C., sodium borohydride (3 equiv, Sigma-Aldrich) was added portion-wise over a period of 5 min. Following the completion of addition, the reaction mixture was stirred at 0° C. for 30 min and then at RT for 3 h. The reaction was then carefully quenched with the dropwise addition of water and extracted with EtOAc. The combined organic extracts were washed further with water and brine, dried over Na₂SO₄, filtered, and the filtrate concentrated in vacuo. Purification of the crude product thus obtained by way of reverse-phase column chromatography (C₁₈, gradient elution: 9:1 (v/v) H₂O: MeCN→1:1 (v/v) H₂O: MeCN) afforded methyl 3-((3-((2-(difluoromethoxy)-6-methylpyridin-3-yl)carbamoyl)-3-(2-isopropylphenyl)cyclobutyl)amino)propanoate as a yellow solid (70% yield).

Step 2: In a dried, round-bottom flask equipped with a magnetic stirrer was dissolved methyl 3-((3-((2-(difluoromethoxy)-6-methylpyridin-3-yl)carbamoyl)-3-(2-isopropylphenyl)cyclobutyl)amino)propanoate (1 equiv) from the previous step in THE (0.084 M). To this colorless solution was then added lithium hydroxide (0.35 M solution in water, 2 equiv) and the resulting mixture was stirred at RT for 1 h. Finally, the reaction mixture was carefully quenched with HCl (1 M solution in water, 1 equiv) and extracted with EtOAc. The combined organic extracts were washed sequentially with water and brine, dried over Na₂SO₄, and filtered. Concentration of the filtrate in vacuo afforded 3-((3-((2-(difluoromethoxy)-6-methylpyridin-3-yl)carbamoyl)-3-(2-isopropylphenyl)cyclobutyl)amino)propanoic acid as a white solid (95% yield).

Step 3: In a dried, round-bottom flask equipped with a magnetic stirrer was combined 3-((3-((2-(difluoromethoxy)-6-methylpyridin-3-yl)carbamoyl)-3-(2-isopropylphenyl)cyclobutyl)amino)propanoic acid (1 equiv) from the previous step and sodium bicarbonate (6 equiv, Sigma-Aldrich) in acetonitrile (0.021 M). To this was then added methanesulfonyl chloride (1.2 equiv, Sigma-Aldrich) and the resulting mixture was heated at 60° C. for 3 h. Finally, the reaction was quenched with water and extracted with EtOAc. The combined organic extracts were washed sequentially with water and brine, dried over Na₂SO₄, filtered, and the filtrate concentrated in vacuo. The crude product thus obtained was subjected to reverse-phase column chromatography (C₁₈, gradient elution: 9:1 (v/v) H₂O: MeCN→1:1 (v/v) H₂O: MeCN) to afford the title compound as a mixture of diastereomers (37% yield). LCMS: m/z=444.2 [M+H]⁺; ¹H NMR (methanol-d₄): δ=8.31 (d, J=8.1 Hz, 0.2H, diastereomer A), 8.27 (d, J=7.8 Hz, 0.8H, diastereomer B), 7.67˜7.32 (m, 5H), 7.11 7.00 (m, 1H), 4.70˜4.53 (m, 0.2H, diastereomer A), 4.25˜4.09 (m, 0.8H, diastereomer B), 3.51˜3.48 (m, 2H), 3.21˜3.08 (m, 2H), 2.90˜2.68 (m, 5H), 2.37 (s, 3H), 1.13 (d, J=6.6 Hz, 1.2H, diastereomer A), 1.11 (d, J=6.6 Hz, 4.8H, diastereomer B).

### Example 69: Preparation of N-(2-(difluoromethoxy)-6-methylpyridin-3-yl)-1-(2-isopropylphenyl)-3-sulfamoylcyclobutane-1-carboxamide

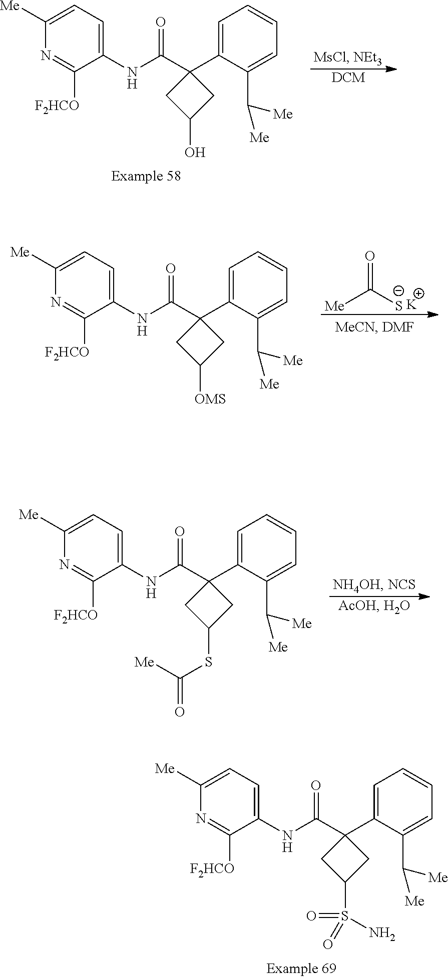

Step 1: In a dried, round-bottom flask equipped with a magnetic stirrer was combined Example 58 (1 equiv) and triethylamine (3 equiv, Sigma-Aldrich) in dichloromethane (0.051 M). To this was added methanesulfonyl chloride (1.5 equiv, Sigma-Aldrich) neat and dropwise at 0° C., and the resulting mixture was then warmed to RT over 1 h. Finally, the reaction was quenched with water and extracted with DCM. The combined organic extracts were dried over Na₂SO₄ and filtered. Concentration of the filtrate in vacuo afforded crude 3-((2-(difluoromethoxy)-6-methylpyridin-3-yl)carbamoyl)-3-(2-isopropylphenyl)cyclobutyl methanesulfonate as a yellow oil (83% yield).

Step 2: In a dried, round-bottom flask equipped with a magnetic stirrer was dissolved 3-((2-(difluoromethoxy)-6-methylpyridin-3-yl)carbamoyl)-3-(2-isopropylphenyl)cyclobutyl methanesulfonate (1 equiv) from the previous step in a 4:1 (v/v) solution of acetonitrile and DMF (0.017 M). To this solution was then added potassium thioacetate (2 equiv, Sigma-Aldrich) at RT and the resulting mixture was heated at 100° C. for 16 h. Finally, the reaction mixture was diluted with water and extracted with EtOAc. The combined organic extracts were washed further with water and brine, dried over Na₂SO₄, filtered and the filtrate concentrated in vacuo. Purification of the crude product thus obtained by way of column chromatography (SiO₂, gradient elution, Hex→1:1 (v/v) Hex: EtOAc) afforded S-(3-((2-(difluoromethoxy)-6-methylpyridin-3-yl)carbamoyl)-3-(2-isopropylphenyl)cyclobutyl) ethanethioate as a yellow oil (47% yield).

Step 3: In a dried, round-bottom flask equipped with a magnetic stirrer was dissolved S-(3-((2-(difluoromethoxy)-6-methylpyridin-3-yl)carbamoyl)-3-(2-isopropylphenyl)cyclobutyl) ethanethioate (1 equiv) from the previous step in a 5:1 (v/v) solution of acetic acid and water (0.18 M). To this was added at 0° C. N-chlorosuccinimide (3.25 equiv, Sigma-Aldrich) and the resulting mixture was then warmed to RT over 2 h. Finally, ammonium hydroxide (25% w/w solution in water, 40 equiv, J. T. Baker) was added and the resulting mixture was stirred at RT for 16 h. The reaction was then quenched with water and extracted with DCM. The combined organic extracts were washed sequentially with water and brine, dried over Na₂SO₄, filtered, and the filtrate concentrated in vacuo. The crude product thus obtained was subjected to reverse-phase column chromatography (Cis, gradient elution: 95:5 (v/v) H₂O: MeCN 4 5:95 (v/v) H₂O: MeCN) to afford the title compound as a mixture of diastereomers (35% yield). LCMS: m/z=454.2 [M+H]⁺; ¹H NMR (methanol-d₄): δ=8.34˜8.30 (m, 1H), 7.73˜7.18 (m, 5H), 7.04 (d, J=7.6 Hz, 0.5H, diastereomer A), 7.03 (d, J=7.6 Hz, 0.5H, diastereomer B), 4.23˜4.14 (m, 0.5H, diastereomer A), 3.77˜3.68 (m, 0.5H, diastereomer B), 3.39˜2.72 (m, 5H), 2.39 (s, 3H), 1.16 (d, J=6.8 Hz, 3H, diastereomer A), 1.13 (d, J=6.8 Hz, 3H, diastereomer B).

The following example was prepared in an analogous fashion to Example 69, but substituting ammonium hydroxide in step 3 with the requisite amine.

| | | | LCMS |
|---|---|---|---|
| Example | Starting Amine | Structure | (m/z) |
| 70 | MeNH₂ (Sigma-Aldrich) | 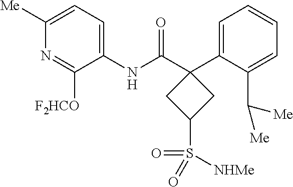 [SMILES: CNS(=O)(=O)C1CC(C(=O)Nc2ccc(C)nc2OC(F)F)(c2ccccc2C(C)C)C1] N-(2-(difluoromethoxy)-6-methylpyridin-3-yl)- 1-(2-isopropylphenyl)-3-(N- methylsulfamoyl)cyclobutane-1-carboxamide | 468.2 [M + H]⁺ |

### Example 71: Preparation of N-(2-(difluoromethoxy)-6-methylpyridin-3-yl)-1-(2-isopropylphenyl)-3-(methylsulfonyl)cyclobutane-1-carboxamide

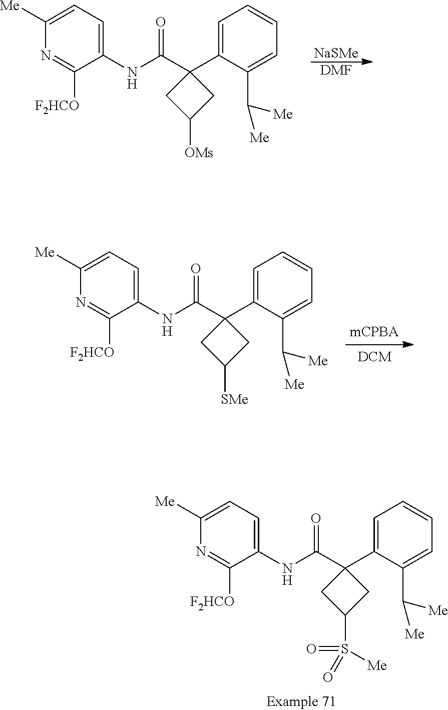

Step 1: In a dried, round-bottom flask equipped with a magnetic stirrer was combined 3-((2-(difluoromethoxy)-6-methylpyridin-3-yl)carbamoyl)-3-(2-isopropylphenyl)cyclobutyl methanesulfonate (1 equiv, Example 69, step 1) and sodium thiomethoxide (1.1 equiv, Sigma-Aldrich) in DMF (0.05 M). The resulting mixture was heated at 110° C. for 2 h, diluted with water, and then extracted with EtOAc. The combined organic extracts were washed further with water and brine, dried over Na₂SO₄, filtered and the filtrate concentrated in vacuo. Purification of the crude product thus obtained by way of column chromatography (SiO₂, gradient elution, Hex→1:1 (v/v) Hex: EtOAc) afforded N-(2-(difluoromethoxy)-6-methylpyridin-3-yl)-1-(2-isopropylphenyl)-3-(methylthio)cyclobutane-1-carboxamide as a white solid (80% yield).

Step 2: In a dried, round-bottom flask equipped with a magnetic stirrer was dissolved N-(2-(difluoromethoxy)-6-methylpyridin-3-yl)-1-(2-isopropylphenyl)-3-(methylthio)cyclobutane-1-carboxamide (1 equiv) from the previous step in dichloromethane (0.06 M). To this was then added m-chloroperoxybenzoic acid (2 equiv, Sigma-Aldrich) and the resulting mixture was stirred at RT for 2 h. The reaction was then quenched with water and extracted with DCM. The combined organic extracts were washed sequentially with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN 4 5:95 (v/v) H₂O: MeCN) to afford the title compound as a white solid (10% yield). LCMS: m/z=453.1 [M+H]⁺; ¹H NMR (methanol-d₄): δ=8.34 (d, J=8.0 Hz, 1H), 7.55˜7.18 (m, 5H), 7.04 (d, J=8.4 Hz, 1H), 4.35˜4.27 (m, 1H), 3.21˜3.16 (m, 2H), 3.04˜3.01 (m, 2H), 2.92 (s, 3H), 2.74 (sept, J=6.8 Hz, 1H), 2.39 (s, 3H), 1.18 (d, J=6.8 Hz, 6H).

### Example 72: Preparation of 3-((1H-1,2,3-triazol-1-yl)methyl)-N-(2-(difluoromethoxy)-6-methylpyridin-3-yl)-1-(2-isopropylphenyl)cyclobutane-1-carboxamide

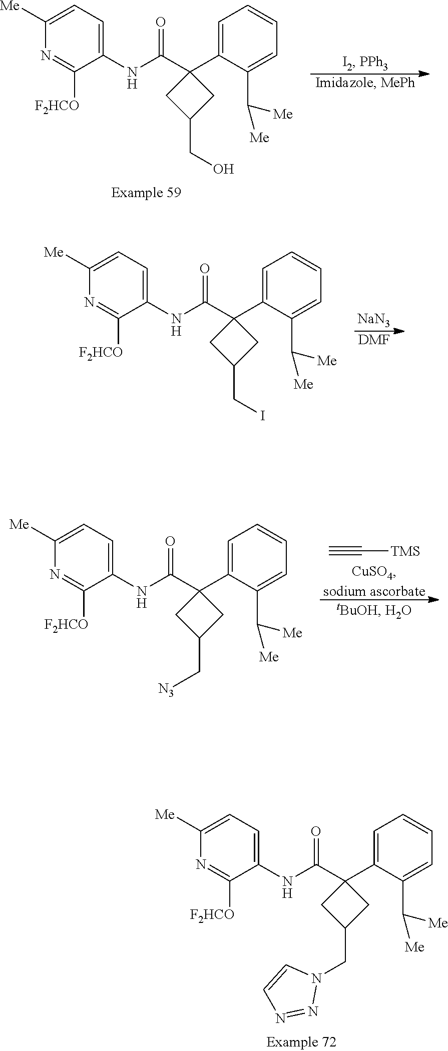

Step 1: In a dried, round-bottom flask equipped with a magnetic stirrer was combined Example 59 (1 equiv), imidazole (2 equiv, Sigma-Aldrich), and triphenylphosphine (2 equiv, Sigma-Aldrich) in toluene (0.047 M). To this solution was added iodine (2 equiv, Sigma-Aldrich) in one rapid portion, and the resulting mixture was then heated at 90° C. for 2 h. Finally, the reaction was quenched with water and extracted with EtOAc. The combined organic extracts were washed with brine, dried over Na₂SO₄, filtered, and the filtrate concentrated in vacuo. Purification of the crude product thus obtained by way of column chromatography (SiO₂, 2:1 (v/v) Hex: EtOAc) afforded N-(2-(difluoromethoxy)-6-methylpyridin-3-yl)-3-(iodomethyl)-1-(2-isopropylphenyl)cyclobutane-1-carboxamide as a colorless oil (41% yield).

Step 2: In a dried, round-bottom flask equipped with a magnetic stirrer was dissolved N-(2-(difluoromethoxy)-6-methylpyridin-3-yl)-3-(iodomethyl)-1-(2-isopropylphenyl)cyclobutane-1-carboxamide (1 equiv) from the previous step in DMF (0.11 M). To this solution was added sodium azide (1.5 equiv, Sigma-Aldrich) in one rapid portion, and the resulting mixture was heated at 45° C. for 18 h. Finally, the reaction mixture was diluted with water and extracted with EtOAc. The combined organic extracts were washed further with water and brine, dried over Na₂SO₄, filtered and the filtrate concentrated in vacuo. Purification of the crude product thus obtained by way of column chromatography (SiO₂, 10:1 (v/v) Hex: EtOAc) afforded 3-(azidomethyl)-N-(2-(difluoromethoxy)-6-methylpyridin-3-yl)-1-(2-isopropylphenyl)cyclobutane-1-carboxamide as a white solid (96% yield).

Step 3: In a dried, round-bottom flask equipped with a magnetic stirrer was combined 3-(azidomethyl)-N-(2-(difluoromethoxy)-6-methylpyridin-3-yl)-1-(2-isopropylphenyl)cyclobutane-1-carboxamide (1 equiv) from the previous step and trimethylsilylacetylene (1.2 equiv, Sigma-Aldrich) in tert-butanol (0.035 M). In a separate reaction vessel was dissolved copper(II) sulfate pentahydrate (0.15 equiv, Sigma-Aldrich) in water (0.005 M). To this blue aqueous solution was then added sodium ascorbate (0.45 equiv, Sigma-Aldrich) and the resulting brown solution was shaken until a golden, yellow suspension was obtained. This golden catalyst suspension was then immediately added to the tert-butanol reaction solution of 3-(azidomethyl)-N-(2-(difluoromethoxy)-6-methylpyridin-3-yl)-1-(2-isopropylphenyl)cyclobutane-1-carboxamide and trimethylsilylacetylene. The resulting reaction suspension was then stirred at RT for 48 h. Finally, the reaction was quenched with water and extracted with EtOAc. The combined organic extracts were washed sequentially with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN→5:95 (v/v) H₂O: MeCN) to afford the title compound as a white solid (27% yield). LCMS: m/z=456.1 [M+H]⁺; ¹H NMR (methanol-d₄): δ=8.32 (d, J=8.0 Hz, 1H), 8.02 (s, 1H), 7.74 (s, 1H), 7.60˜7.19 (m, 5H), 7.03 (d, J=8.0 Hz, 1H), 4.70 (d, J=6.0 Hz, 2H), 2.83˜2.70 (m, 6H), 2.39 (s, 3H), 1.11 (d, J=6.8 Hz, 6H).

### Example 73: Preparation of N-(2-(difluoromethoxy)-6-methylpyridin-3-yl)-1-(2-isopropylphenyl)cyclobutane-1-carboxamide

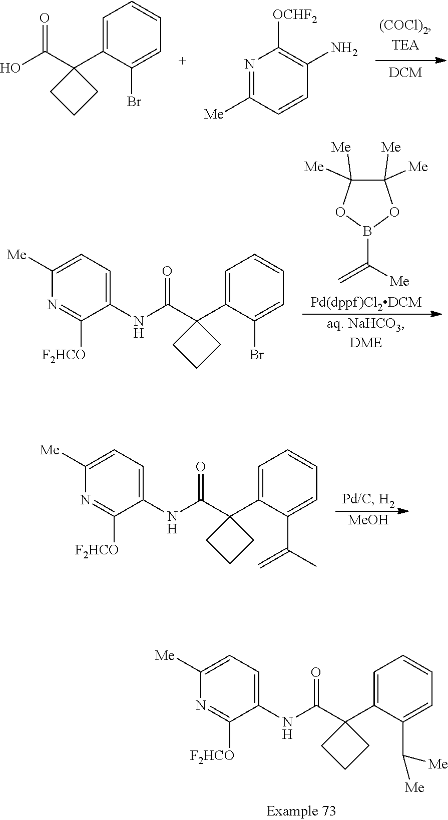

Step 1: In a dried, round-bottom flask equipped with a magnetic stirrer was dissolved 1-(2-bromophenyl)cyclobutane-1-carboxylic acid (1 equiv, Combi-Blocks) in dichloromethane (0.08 M). To this was then added at 0° C. oxalyl chloride (3 equiv, Sigma-Aldrich) neat and dropwise over 5 min. Then, a couple of drops of DMF was added and the resulting mixture was allowed to warm to RT over 30 min. The volatiles were then removed in vacuo and the resulting residue was further azeotroped with toluene and heptane. The crude 1-(2-bromophenyl)cyclobutane-1-carbonyl chloride thus obtained was then retaken up in dichloromethane (0.08 M) and added, sequentially at 0° C., triethylamine (6 equiv, Sigma-Aldrich) and 2-(difluoromethoxy)-6-methylpyridin-3-amine (1 equiv, synthesized as per WO2019/234115). Following the completion of addition, the reaction mixture was warmed to RT and stirred at RT for 16 h. The reaction mixture was then diluted with water and extracted with EtOAc. The combined organic extracts were washed further with water and brine, dried over Na₂SO₄, filtered, and the filtrate concentrated in vacuo. Purification of the crude product thus obtained by way of column chromatography (SiO₂, 4:1 (v/v) Hex: EtOAc) afforded 1-(2-bromophenyl)-N-(2-(difluoromethoxy)-6-methylpyridin-3-yl)cyclobutane-1-carboxamide as a yellow oil (71% yield).

Step 2: In a thick-walled glass reaction vessel equipped with a magnetic stirrer and a Teflon screwcap was combined 1-(2-bromophenyl)-N-(2-(difluoromethoxy)-6-methylpyridin-3-yl)cyclobutane-1-carboxamide (1 equiv) from the previous step, 4,4,5,5-tetramethyl-2-(prop-1-en-2-yl)-1,3,2-dioxaborolane (4 equiv, Frontier Scientific), [1,1′-bis(diphenylphosphino)ferrocene]dichloropalladium(II) dichloromethane complex (0.1 equiv, Sigma-Aldrich), and sodium bicarbonate (3 equiv, Alfa Aesar) in a 3:1 (v/v) solution of 1,2-dimethoxyethane and water (0.020 M). The resulting mixture was deoxygenated via subsurface purging with nitrogen for 10 min before the reaction vessel was tightly sealed and heated at 90° C. for 16 h. The now dark brown suspension was cooled to RT, quenched with water, and extracted with EtOAc. The combined organic extracts were washed further with water and brine, dried over Na₂SO₄, filtered, and the filtrate concentrated in vacuo. Purification of the crude product thus obtained by way of column chromatography (SiO₂, 5:1 (v/v) Hex: EtOAc) afforded N-(2-(difluoromethoxy)-6-methylpyridin-3-yl)-1-(2-(prop-1-en-2-yl)phenyl)cyclobutane-1-carboxamide as a yellow oil (88% yield).

Step 3: In a dried, round-bottom flask equipped with a magnetic stirrer was dissolved N-(2-(difluoromethoxy)-6-methylpyridin-3-yl)-1-(2-(prop-1-en-2-yl)phenyl)cyclobutane-1-carboxamide (1 equiv) from the previous step in methanol (0.031 M). The resulting solution was then deoxygenated via subsurface purging with nitrogen for 10 min before palladium (10% w/w over activated carbon, dry, 0.35 equiv, Sigma-Aldrich) was added in one rapid portion. The resulting black suspension was then subsurface purged with hydrogen for 10 min before it was stirred under a static hydrogen atmosphere at RT for 30 min. The reaction was subsequently quenched with dichloromethane and filtered through a bed of dichloromethane-wetted celite. The insolubles were washed further with dichloromethane and the filtrate was concentrated in vacuo. The crude product thus obtained was subjected to reverse-phase column chromatography (C₁₈, gradient elution: 95:5 (v/v) H₂O: MeCN→5:95 (v/v) H₂O: MeCN) to afford the title compound as a white solid (38% yield). LCMS: m/z=375.2 [M+H]⁺; ¹H NMR (DMSO-d₆): δ=8.09 (d, J=7.8 Hz, 1H), 7.73˜7.24 (m, 6H), 7.09 (d, J=8.1 Hz, 1H), 2.83˜2.53 (m, 3H), 2.60˜2.51 (m, 2H), 2.35 (s, 3H), 2.12 (sept, J=6.6 Hz, 1H), 1.85˜1.80 (m, 1H), 1.06 (d, J=6.6 Hz, 6H).

### Example 74: Preparation of 2-acetyl-N-(2-(difluoromethoxy)-6-methylpyridin-3-yl)-6-(2-isopropylphenyl)-2-azaspiro[3.3]heptane-6-carboxamide

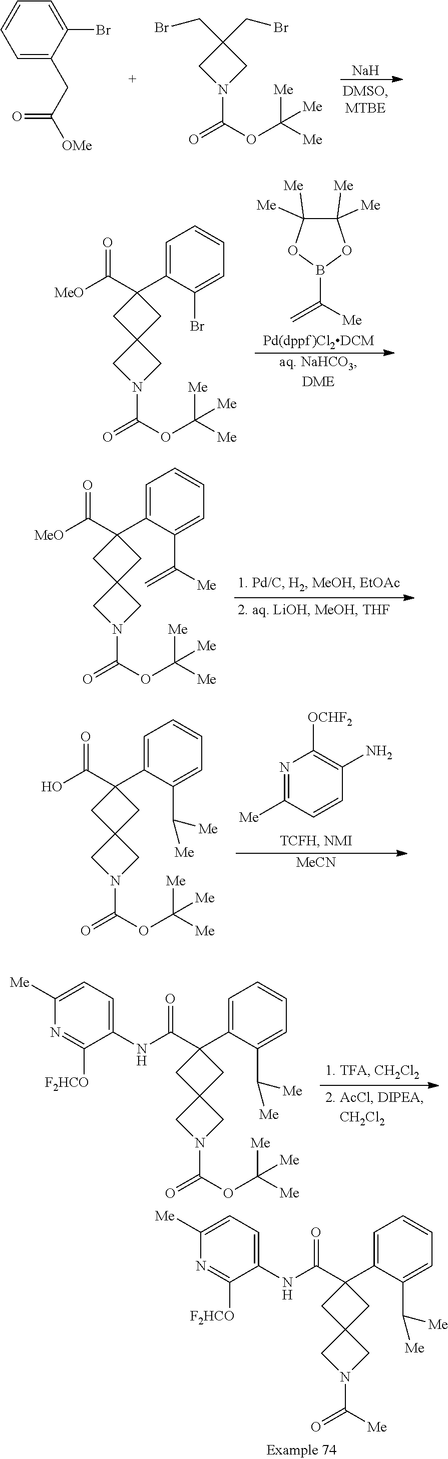

Step 1: In a dried, round-bottom flask equipped with a magnetic stirrer was suspended sodium hydride (60% w/w dispersion in paraffin oil, 2.2 equiv, Sigma-Aldrich) in anhydrous dimethylsulfoxide (0.23 M). To this was then added at RT, a tert-butyl methyl ether solution (0.092 M) of methyl 2-(2-bromophenyl)acetate (1 equiv, Combi-Blocks) and tert-butyl 3,3-bis(bromomethyl)azetidine-1-carboxylate (1 equiv, Combi-Blocks) dropwise over a period of 90 min. Following the completion of addition, the reaction mixture was stirred at RT for another 4 h. The reaction was then carefully quenched with the dropwise addition of 1 M aq. HCl and extracted with a 1:1 (v/v) solution of hexanes and EtOAc. The combined organic extracts were washed further with water and brine, dried over MgSO₄, filtered, and the filtrate concentrated in vacuo. Purification of the crude product thus obtained by way of column chromatography (SiO₂, gradient elution: Hex→3:2 (v/v) Hex: EtOAc) afforded 2-(tert-butyl) 6-methyl 6-(2-bromophenyl)-2-azaspiro[3.3]heptane-2,6-dicarboxylate as a viscous oil (48% yield).

Step 2: In a thick-walled glass reaction vessel equipped with a magnetic stirrer and a Teflon screwcap was combined 2-(tert-butyl) 6-methyl 6-(2-bromophenyl)-2-azaspiro[3.3]heptane-2,6-dicarboxylate (1 equiv) from the previous step, 4,4,5,5-tetramethyl-2-(prop-1-en-2-yl)-1,3,2-dioxaborolane (4 equiv, Frontier Scientific), [1,1′-bis(diphenylphosphino)ferrocene]dichloropalladium(II) dichloromethane complex (0.1 equiv, Sigma-Aldrich), and sodium bicarbonate (3 equiv, Alfa Aesar) in a 5:2 (v/v) solution of 1,2-dimethoxyethane and water (0.095 M). The resulting mixture was deoxygenated via subsurface purging with nitrogen for 10 min before the reaction vessel was tightly sealed and heated at 85° C. for 4 h. The now dark brown suspension was cooled to RT, quenched with water, and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: Hex→1:1 (v/v) Hex: EtOAc) afforded 2-(tert-butyl) 6-methyl 6-(2-(prop-1-en-2-yl)phenyl)-2-azaspiro[3.3]heptane-2,6-dicarboxylate as a foam (69% yield).

Step 3: In a dried, round-bottom flask equipped with a magnetic stirrer was dissolved 2-(tert-butyl) 6-methyl 6-(2-(prop-1-en-2-yl)phenyl)-2-azaspiro[3.3]heptane-2,6-dicarboxylate (1 equiv) from the previous step in a 2:1 (v/v) solution of ethyl acetate and methanol (0.061 M). The resulting colorless solution was then deoxygenated via subsurface purging with nitrogen for 10 min before palladium (10% w/w over activated carbon, dry, 0.05 equiv, Sigma-Aldrich) was added in one rapid portion. The resulting black suspension was then subsurface purged with hydrogen for 10 min before it was stirred under a static hydrogen atmosphere (maintained with multiple balloons) at RT for 30 min. The reaction was subsequently quenched with dichloromethane and filtered through a bed of dichloromethane-wetted celite. The insolubles were washed further with dichloromethane and the filtrate was concentrated in vacuo. The residue thus obtained was then taken up in a 1:1 (v/v) solution of THF and methanol. To this colorless solution was then added lithium hydroxide (1 M solution in water, 5 equiv) and the resulting mixture was heated at 50° C. for 18 h. Upon cooling to RT, the reaction mixture was carefully quenched with HCl (1 M solution in water, 5 equiv) and the volatiles were removed in vacuo. The resulting suspension was back extracted with EtOAc. The combined organic extracts were washed sequentially with water and brine, dried over MgSO₄, and filtered. Concentration of the filtrate in vacuo afforded 2-(tert-butoxycarbonyl)-6-(2-isopropylphenyl)-2-azaspiro[3.3]heptane-6-carboxylic acid as a white foam (92% yield).

Step 4: In a dried, round-bottom flask equipped with a magnetic stirrer was combined 2-(tert-butoxycarbonyl)-6-(2-isopropylphenyl)-2-azaspiro[3.3]heptane-6-carboxylic acid (1 equiv) from the previous step, 2-(difluoromethoxy)-6-methylpyridin-3-amine (1.2 equiv, synthesized as per WO2019/234115), and 1-methylimidazole (5 equiv, Sigma-Aldrich) in anhydrous acetonitrile (0.077 M). To this was then added chloro-N,N,N′,N′-tetramethylformamidinium hexafluorophosphate (2 equiv, Chem-Impex) in one rapid portion and the resulting mixture was stirred at RT for 48 h. The volatiles were then removed in vacuo and the residue thus obtained was subjected to reverse-phase column chromatography (C₁₈, gradient elution: 9:1 (v/v) H₂O: MeCN+0.1% formic acid 4 MeCN+0.1% formic acid) to afford tert-butyl 6-((2-(difluoromethoxy)-6-methylpyridin-3-yl)carbamoyl)-6-(2-isopropylphenyl)-2-azaspiro[3.3]heptane-2-carboxylate as a viscous oil (50% yield).

Step 5: In a round-bottom flask equipped with a magnetic stirrer was dissolved tert-butyl 6-((2-(difluoromethoxy)-6-methylpyridin-3-yl)carbamoyl)-6-(2-isopropylphenyl)-2-azaspiro[3.3]heptane-2-carboxylate (1 equiv) from the previous step in dichloromethane (0.04 M). To this solution was then added trifluoroacetic acid (70 equiv, Sigma-Aldrich) and the resulting reaction mixture was stirred at RT for 20 min. The volatiles were then removed in vacuo via sequential azeotropic distillation with toluene and heptane. The residue thus obtained was then taken up in dichloromethane (0.04 M), and added sequentially N,N-diisopropylethylamine (1.3 equiv, Sigma-Aldrich) and acetyl chloride (1.3 equiv, Sigma-Aldrich). After 5 min of stirring at RT, the volatiles were quickly removed in vacuo. The crude product thus obtained was subjected first to reverse-phase column chromatography (C₁₈, gradient elution: 9:1 (v/v) H₂O: MeCN+0.1% formic acid 4 MeCN+0.1% formic acid), and then to regular phase column chromatography (SiO₂, gradient elution: EtOAc 4 10:1 (v/v) EtOAc: MeOH), to afford the title compound as a foam (17% yield). LCMS: m/z=458.1 [M+H]⁺; ¹H NMR (DMSO-d₆): δ=7.99 (t, J=7.5 Hz, 1H), 7.58 (t, J=72 Hz, 1H), 7.30˜7.10 (m, 3H), 7.05 (d, J=7.5 Hz, 1H), 6.99 (dd, J=8.5, 1 Hz, 1H), 6.95 (d, J=7.5 Hz, 1H), 4.19 (d, J=4 Hz, 2H), 3.16 (t, J=6 Hz, 2H), 3.04 (sept, J=6.5 Hz, 1H), 2.48 2.24 (m, 7H), 1.82 (s, 3H), 1.17 (d, J=6.5 Hz, 3H), 1.14 (d, J=6.5 Hz, 3H).

The following examples were prepared via chiral chromatographic separation of the corresponding mixture of stereoisomers.

| | | | RT |
|---|---|---|---|
| Example | Starting Mixture | Separation Conditions | (min) |
| 75 isomer A 75 isomer B | 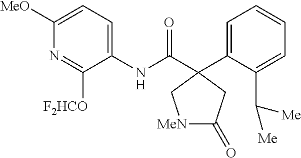 [SMILES: COc1ccc(NC(=O)C2(c3ccccc3C(C)C)CC(=O)N(C)C2)c(OC(F)F)n1] Example 1 | Column: CHIRAL ART Amylose SA (2 × 25 cm; 5 μm) Mobile phase: 70:30 (v/v) Hex: EtOH Additive: 0.5% 2M NH₃ in MeOH Flow rate: 20 mL/min | 8.25 10.02 |
| 76 isomer A 76 isomer B | 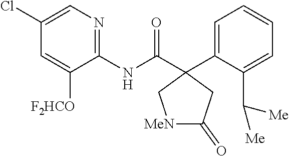 [SMILES: CC(C)c1ccccc1C1(C(=O)Nc2ncc(Cl)cc2OC(F)F)CC(=O)N(C)C1] Example 7 | Column: CHIRALPAK IF (2 × 25 cm; 5 μm) Mobile phase: 90:10 (v/v) Hex: EtOH Additive: 0.5% 2M NH₃ in MeOH Flow rate: 20 mL/min | 17.35 22.2 |
| 77 isomer A 77 isomer B | 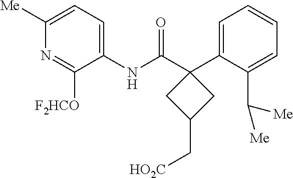 [SMILES: Cc1ccc(NC(=O)C2(c3ccccc3C(C)C)CC(CC(=O)O)C2)c(OC(F)F)n1] Example 45 | Column: CHIRAL ART Cellulose SB (2 × 25 cm; 5 μm) Mobile phase: 95:5 (v/v) Hex: ⁱPrOH Additive: 0.1% formic acid Flow rate: 20 mL/min | 10.39 13.06 |
| 78 isomer A 78 isomer B | 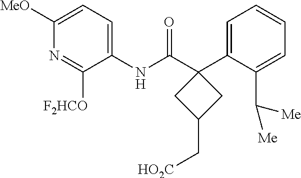 [SMILES: COc1ccc(NC(=O)C2(c3ccccc3C(C)C)CC(CC(=O)O)C2)c(OC(F)F)n1] Example 46 | Column: CHIRAL ART Cellulose SC (2 × 25 cm; 5 μm) Mobile phase: 97:3 (v/v) Hex: EtOH Additive: 0.1% formic acid Flow rate: 20 mL/min | 10.81 13.40 |
| 89 isomer A 89 isomer B | 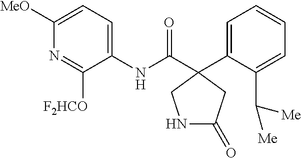 [SMILES: COc1ccc(NC(=O)C2(c3ccccc3C(C)C)CNC(=O)C2)c(OC(F)F)n1] Example 8 | Column: CHIRALPAK IH (2 × 25 cm; 5 μm) Mobile phase: 50:50 (v/v) Hex: EtOH Additive: 0.5% 2M NH₃ in methanol Flow rate: 20 mL/min | 9.45 19.96 |
| 90 isomer A 90 isomer B | 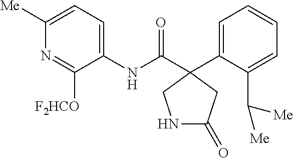 [SMILES: Cc1ccc(NC(=O)C2(c3ccccc3C(C)C)CNC(=O)C2)c(OC(F)F)n1] Example 9 | Column: CHIRALPAK IH-3 (4.6 × 5 cm; 3 μm) Mobile phase: Hex Additive: 0.5% diethylamine Flow rate: 1 mL/min | 2.14 4.59 |
| 92 isomer A 92 isomer B | 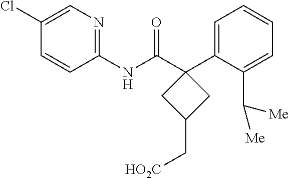 [SMILES: CC(C)c1ccccc1C1(C(=O)Nc2ccc(Cl)cn2)CC(CC(=O)O)C1] Example 91 | Column: Lux Cellulose-2 (2.12 × 25 cm; 5 μm) Mobile phase: 95:5 (v/v) Hex: ⁱPrOH Additive: 0.1% formic acid Flow rate: 20mL/min | 11.40 17.40 |

### Example 79: N-(2-(difluoromethoxy)-6-methoxypyridin-3-yl)-8-(2-isopropylphenyl)-1,4-dioxaspiro[4.5]decane-8-carboxamide

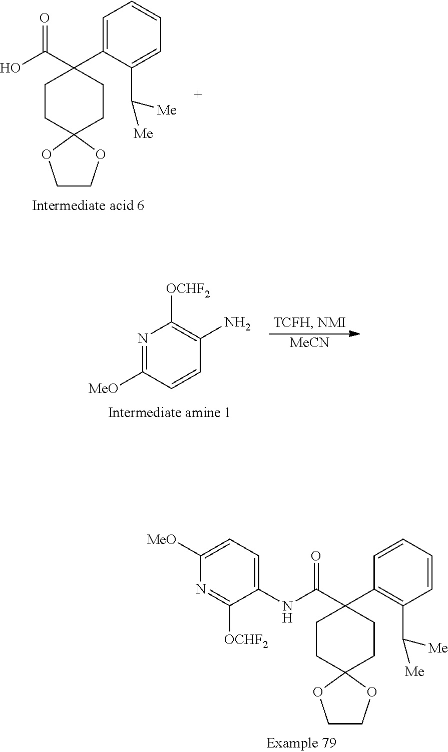

In a dried, round-bottom flask equipped with a magnetic stirrer was combined Intermediate acid 6 (1 equiv), Intermediate amine 1 (1.3 equiv), and 1-methylimidazole (4 equiv, Sigma-Aldrich) in anhydrous acetonitrile (0.053 M). To this was then added chloro-N,N,N′,N′-tetramethylformamidinium hexafluorophosphate (1.3 equiv, Chem-Impex) in one rapid portion and the resulting mixture was heated at 60° C. for 72 h. Upon cooling to RT, the volatiles were removed in vacuo and the crude product thus obtained was purified first by way of reverse-phase column chromatography (C₁₈, gradient elution: 95:5 (v/v) H₂O: MeCN+0.1% formic acid 4 MeCN+0.1% formic acid), and then by regular-phase column chromatography (SiO₂, gradient elution: Hex 4 3:7 (v/v) Hex: EtOAc), to afford the title compound as a pale-yellow oil (38% yield). LCMS: m/z=477.1 [M+H]⁺; ¹H NMR (CDCl₃): δ=8.48 (d, J=8.7 Hz, 1H), 7.56 (dd, J=8.0, 1.1 Hz, 1H), 7.41˜7.25 (m, 3H), 7.20 (t, J=72.7 Hz, 1H), 6.91 (s, 1H), 6.53 (d, J=8.7 Hz, 1H). 4.63˜3.93 (m, 4H), 3.81 (s, 3H), 3.24 (sept, J=6.7 Hz, 1H), 2.54˜2.46 (m, 2H), 2.31˜2.22 (m, 2H), 2.15˜2.05 (m, 2H), 1.79 1.72 (m, 2H), 1.12 (d, J=6.7 Hz, 6H).

### Example 80: N-(2-(difluoromethoxy)-6-methoxypyridin-3-yl)-1-(2-isopropylphenyl)-4-oxocyclohexane-1-carboxamide

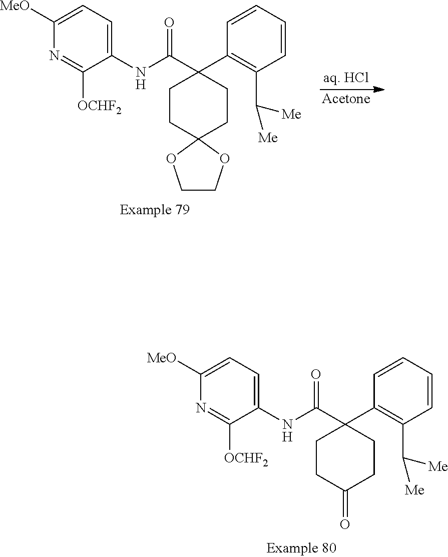

In a round-bottom flask equipped with a magnetic stirrer was dissolved Example 79 (1 equiv) in acetone (0.1 M). To this solution was then added HCl (3 M solution in water, 1 equiv), and the resulting reaction mixture was stirred at RT for 16 h. The reaction mixture was then carefully quenched with saturated aq. NaHCO₃ and extracted with EtOAc. The combined organic extracts were washed further with water and brine, dried over MgSO₄, filtered, and the filtrate concentrated in vacuo. The crude product thus obtained was subjected to reverse-phase column chromatography (C₁₈, gradient elution: 95:5 (v/v) H₂O: MeCN+0.1% formic acid→MeCN+0.1% formic acid) to afford the title compound as a white foam (73% yield). LCMS: m/z=432.8 [M+H]⁺; ¹H NMR (CDCl₃): δ=8.42 (d, J=8.7 Hz, 1H), 7.52˜7.28 (m, 4H), 7.21 (t, J=72.7 Hz, 1H), 6.96 (s, 1H), 6.54 (d, J=8.7 Hz, 1H). 3.82 (s, 3H), 3.21 (sept, J=6.7 Hz, 1H), 2.89˜2.69 (m, 4H), 2.49˜2.33 (m, 4H), 1.15 (d, J=6.7 Hz, 6H).

### Example 81: N-(2-(difluoromethoxy)-6-methoxypyridin-3-yl)-4-hydroxy-1-(2-isopropylphenyl)cyclohexane-1-carboxamide

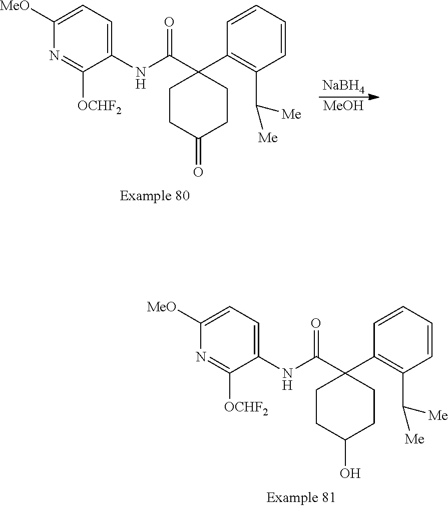 [SMILES: "MeOH" ; COc1ccc(NC(=O)C2(c3ccccc3C(C)C)CCC(=O)CC2)c(OC(F)F)n1 ; "NaBH4" ; COc1ccc(NC(=O)C2(c3ccccc3C(C)C)CCC(O)CC2)c(OC(F)F)n1]

In a round-bottom flask equipped with a magnetic stirrer was dissolved Example 80 (1 equiv) in a 1:1 (v/v) solution of THF and methanol (0.09 M). To this solution was then added sodium borohydride (1.5 equiv, Sigma-Aldrich) in one rapid portion, and the resulting reaction mixture was stirred at RT for 1 h. The volatiles were then removed in vacuo and the resulting residue was partitioned between EtOAc and 1 M aq. HCl. The aqueous layer was separated and back extracted with EtOAc. The combined organic extracts were washed further with water and brine, dried over MgSO₄, filtered, and the filtrate concentrated in vacuo. The crude product thus obtained was subjected to column chromatography (SiO₂, gradient elution: Hex→3:7 (v/v) Hex: EtOAc) to afford the title compound as a 3.3:1 mixture of diastereomers (72% yield). LCMS: m/z=434.8 [M+H]⁺; ¹H NMR (CDCl₃): δ=8.45 (d, J=8.7 Hz, 0.23H, diastereomer A), 8.44 (d, J=8.7 Hz, 0.77H, diastereomer B), 7.58 (dd, J=7.7, 1.2 Hz, 0.23H, diastereomer A), 7.51 (dd, J=7.7, 1.2 Hz, 0.77H, diastereomer B), 7.59˜7.23 (m, 3H), 7.20 (t, J=72.7 Hz, 0.77H, diastereomer B), 7.18 (t, J=72.7 Hz, 0.23H, diastereomer A), 6.94 (s, 0.23H, diastereomer A), 6.85 (s, 0.77H, diastereomer B), 6.52 (d, J=8.7 Hz, 0.77H, diastereomer B), 6.51 (d, J=8.7 Hz, 0.23H, diastereomer A), 3.94 3.86 (m, 0.23H, diastereomer A), 3.81 (s, 2.31H, diastereomer B), 3.80 (s, 0.69H, diastereomer A), 3.79˜3.72 (m, 0.77H, diastereomer B), 3.29 (sept, J=6.7 Hz, 0.77H, diastereomer B), 3.11 (sept, J=6.7 Hz, 0.23H, diastereomer A), 2.56˜1.59 (m, 9H), 1.11 (d, J=6.7 Hz, 6H).

### Example 82: N-(2-(difluoromethoxy)-6-methoxypyridin-3-yl)-4-ethynyl-4-hydroxy-1-(2-isopropylphenyl)cyclohexane-1-carboxamide

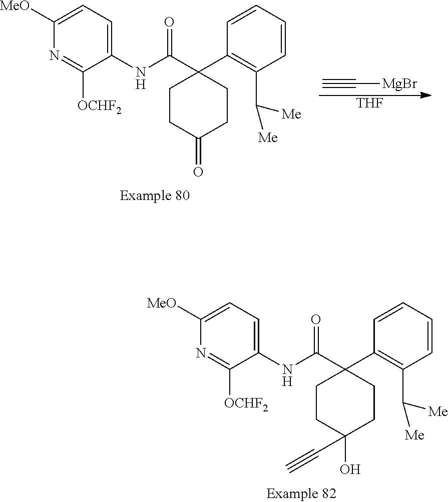

In a round-bottom flask equipped with a magnetic stirrer was dissolved Example 80 (1 equiv) in THE (0.03 M). To this solution was then added, at −78° C., ethynylmagnesium bromide (0.5 M solution in THF, 3 equiv, Sigma-Aldrich) dropwise over a period of 10 min. The resulting reddish-orange reaction mixture was first stirred at −78° C. for 1 h and then allowed to warm slowly to RT over 4 h. The reaction mixture was then diluted with tert-butyl methyl ether and carefully quenched with saturated aq. NH₄C₁. The aqueous layer was separated and back extracted with tert-butyl methyl ether. The combined organic extracts were washed further with water, saturated aq. NaHCO₃, and brine, dried over MgSO₄, filtered, and the filtrate concentrated in vacuo. The crude product thus obtained was subjected to column chromatography (SiO₂, gradient elution: 9:1 (v/v) Hex: EtOAc→3:7 (v/v) Hex: EtOAc) to afford the title compound as a white foam (2000 yield). LCMS: m/z=458.9 [M+H]⁺; ¹H NMR (CDCl₃): δ=8.43 (d, J=8.7 Hz, 1H), 7.55 (dd, J=7.8, 1.2 Hz, 1H), 7.42-7.28 (m, 3H), 7.19 (t, J=72.7 Hz, 1H), 6.85 (s, 1H), 6.52 (d, J=8.7 Hz, 1H). 3.81 (s, 3H), 3.25 (sept, J=6.7 Hz, 1H), 2.55-2.49 (m, 2H), 2.46 (s, 1H), 2.32-2.17 (m, 4H), 2.02˜1.99 (m, 3H), 1.12 (d, J=6.7 Hz, 6H).

The following examples were prepared in an analogous fashion to Example 82 but substituting ethynylmagnesium bromide (3 equiv) with the requisite commercially available organometallic reagent (10 equiv).

| | Starting | | |
|---|---|---|---|
| | Organometallic | | LCMS |
| Example | Reagent | Structure | (m/z) |
| 83-isomer A | MeLi (Sigma-Aldrich) | 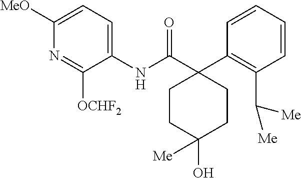 [SMILES: COc1ccc(NC(=O)C2(c3ccccc3C(C)C)CCC(C)(O)CC2)c(OC(F)F)n1] N-(2-(difluoromethoxy)-6-methoxypyridin-3- yl)-4-hydroxy-1-(2-isopropylphenyl)-4- methylcyclohexane-1-carboxamide | 471.2 [M + Na]⁺ |
| 83-isomer B | MeLi (Sigma-Aldrich) | 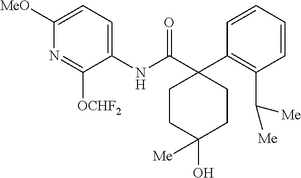 [SMILES: COc1ccc(NC(=O)C2(c3ccccc3C(C)C)CCC(C)(O)CC2)c(OC(F)F)n1] N-(2-(difluoromethoxy)-6-methoxypyridin-3- yl)-4-hydroxy-1-(2-isopropylphenyl)-4- methylcyclohexane-1-carboxamide | 471.2 [M + Na]⁺ |

### Example 84: 4-azido-N-(2-(difluoromethoxy)-6-methoxypyridin-3-yl)-1-(2-isopropylphenyl)cyclohexane-1I-carboxamide

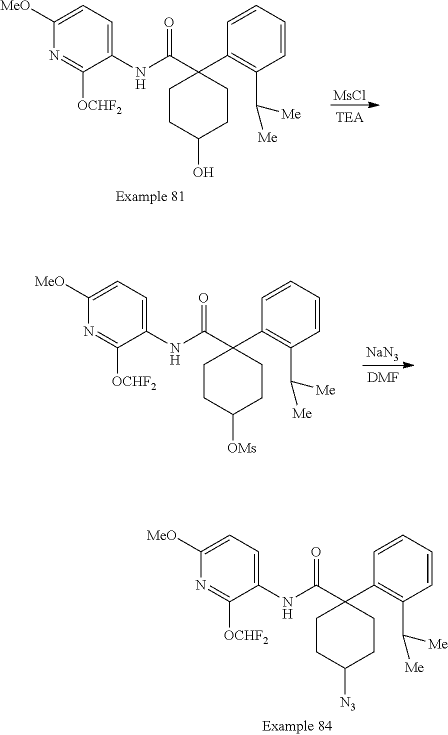

Step 1: In a dried, round-bottom flask equipped with a magnetic stirrer was combined Example 81 (1 equiv) and triethylamine (3 equiv, Sigma-Aldrich) in dichloromethane (0.043 M). To this was added methanesulfonyl chloride (1.5 equiv, Sigma-Aldrich) neat and dropwise at 0° C., and the resulting mixture was then warmed to RT over 4 h. Finally, the reaction was quenched with water and extracted with tert-butyl methyl ether. The combined organic extracts were dried over MgSO₄ and filtered. Concentration of the filtrate in vacuo afforded crude 4-((2-(difluoromethoxy)-6-methoxypyridin-3-yl)carbamoyl)-4-(2-isopropylphenyl)cyclohexyl methanesulfonate as a white foam (98% yield).

Step 2: In a dried, round-bottom flask equipped with a magnetic stirrer was dissolved 4-((2-(difluoromethoxy)-6-methoxypyridin-3-yl)carbamoyl)-4-(2-isopropylphenyl)cyclohexyl methanesulfonate (1 equiv) from the previous step in DMF (0.042 M). To this solution was then added sodium azide (4 equiv, Sigma-Aldrich) at RT, and the resulting mixture was heated at 80° C. for 4 h. Finally, the reaction mixture was cooled to RT, diluted with water, and extracted with tert-butyl ether. The combined organic extracts were washed further with water and brine, dried over MgSO₄, filtered, and the filtrate concentrated in vacuo. The crude product thus obtained was subjected to column chromatography (SiO₂, gradient elution: 9:1 (v/v) Hex: EtOAc→3:7 (v/v) Hex: EtOAc) to afford the title compound as a 3.3:1 mixture of diastereomers (43% yield). LCMS: m/z=460.1 [M+H]⁺; ¹H NMR (CDCl₃): δ=8.44 (d, J=8.6 Hz, 0.23H, diastereomer A), 8.42 (d, J=8.6 Hz, 0.77H, diastereomer B), 7.52 (dd, J=7.5, 1.2 Hz, 0.77H, diastereomer B), 7.49 (dd, J=7.5, 1.2 Hz, 0.23H, diastereomer A), 7.42˜7.27 (m, 3H), 7.19 (t, J=72.7 Hz, 0.23H, diastereomer A), 7.18 (t, J=72.7 Hz, 0.77H, diastereomer B), 6.90 (s, 0.77H, diastereomer B), 6.84 (s, 0.23H, diastereomer A), 6.53 (d, J=8.6 Hz, 0.23H, diastereomer A), 6.52 (d, J=8.6 Hz, 0.77H, diastereomer B), 3.89 (s, 3H), 3.74˜3.66 (m, 0.77H, diastereomer B), 3.55˜3.46 (m, 0.23H, diastereomer A), 3.25 (sept, J=6.7 Hz, 0.23H, diastereomer A), 3.13 (sept, J=6.7 Hz, 0.77H, diastereomer B), 2.59˜1.61 (m, 8H), 1.12 (d, J=6.7 Hz, 6H).

### Example 85: 4-acetamido-N-(2-(difluoromethoxy)-6-methoxypyridin-3-yl)-1-(2-isopropylphenyl)cyclohexane-1-carboxamide

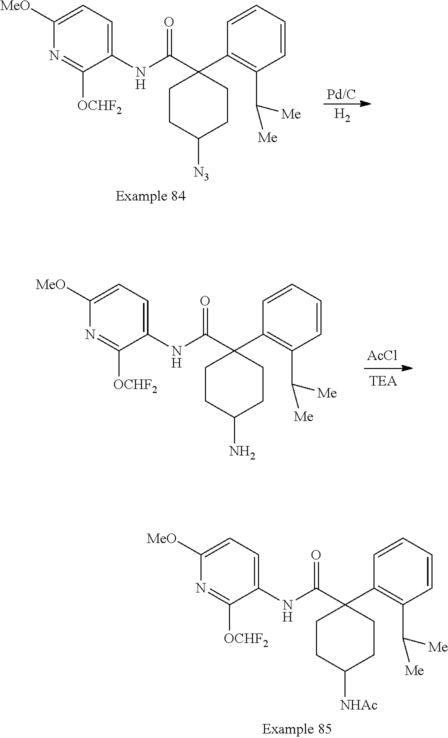

Step 1: In a dried, round-bottom flask equipped with a magnetic stirrer was dissolved Example 84 (1 equiv) in methanol (0.017 M). The resulting colorless solution was then deoxygenated via subsurface purging with nitrogen for 10 min before palladium (10% w/w over activated carbon, dry, 0.35 equiv, Sigma-Aldrich) was added in one rapid portion. The resulting black suspension was then subsurface purged with hydrogen for 10 min before it was stirred under a static hydrogen atmosphere (maintained with a balloon) at RT for 3 h. The reaction was subsequently quenched with dichloromethane and filtered through a bed of dichloromethane-wetted celite. The insolubles were washed further with dichloromethane and the filtrate was concentrated in vacuo to afford crude 4-amino-N-(2-(difluoromethoxy)-6-methoxypyridin-3-yl)-1-(2-isopropylphenyl)cyclohexane-1-carboxamide as a brown oil (>99% yield).

Step 2: In a dried, round-bottom flask equipped with a magnetic stirrer was combined 4-amino-N-(2-(difluoromethoxy)-6-methoxypyridin-3-yl)-1-(2-isopropylphenyl)cyclohexane-1-carboxamide (1 equiv) from the previous step, acetyl chloride (1.5 equiv, Sigma-Aldrich), and triethylamine (3 equiv, Sigma-Aldrich) in anhydrous dichloromethane (0.016 M). The resulting reaction mixture was stirred at RT for 1 h. The volatiles were then removed in vacuo and the residue thus obtained was subjected to reverse-phase column chromatography (C₁₈, gradient elution: 95:5 (v/v) H₂O: MeCN→5:95 (v/v) H₂O: MeCN) to afford the title compound as a 3.3:1 mixture of diastereomers (42% yield). LCMS: m/z=476.1 [M+H]⁺; ¹H NMR (CDCl₃): δ=8.48 (d, J=8.7 Hz, 0.77H, diastereomer A), 8.38 (d, J=8.7 Hz, 0.23H, diastereomer B), 7.55˜7.29 (m, 4H), 7.20 (t, J=72.7 Hz, 0.23H, diastereomer B), 7.17 (t, J=72.7 Hz, 0.77H, diastereomer A), 6.94 (s, 0.77H, diastereomer A), 6.83 (s, 0.23H, diastereomer B), 6.52 (d, J=8.7 Hz, 0.23H, diastereomer B), 6.51 (d, J=8.7 Hz, 0.77H, diastereomer A), 4.04˜3.88 (m, 1H), 3.81 (s, 0.69H, diastereomer B), 3.80 (s, 2.31H, diastereomer A), 3.29 (sept, J=6.7 Hz, 0.23H, diastereomer B), 3.05 (sept, J=6.7 Hz, 0.77H, diastereomer A), 2.51˜1.93 (m, 6H), 1.97 (s, 0.69H, diastereomer B), 1.92 (s, 2.31H, diastereomer A), 1.43˜1.31 (m, 2H), 1.12 (d, J=6.7 Hz, 1.38H, diastereomer B), 1.12 (d, J=6.7 Hz, 4.62H, diastereomer A).

### Example 86: N-(2-(difluoromethoxy)-6-methoxypyridin-3-yl)-8-(2-isopropylphenyl)-2,4-dioxo-1,3-diazaspiro[4.5]decane-8-carboxamide

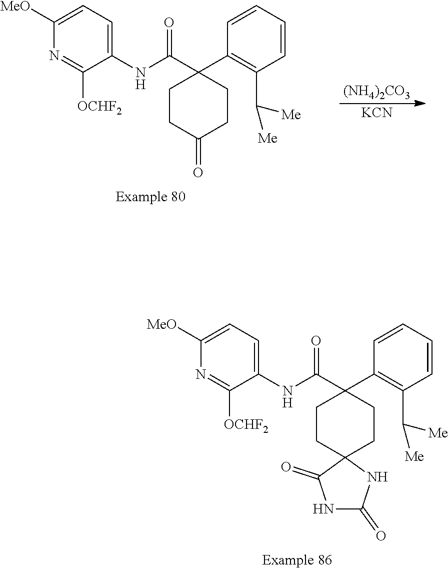

In a thick-walled glass reaction vessel equipped with a magnetic stirrer and a Teflon screwcap was combined Example 80 (1 equiv), ammonium carbonate (2.5 equiv, Combi-Blocks), and potassium cyanide (1 equiv, Combi-Blocks) in a 1:1 (v/v) solution of water and ethanol (0.1 M). The vessel was then tightly sealed, and the reaction mixture was heated at 55° C. for 120 h. The volatiles were then removed in vacuo and the residue thus obtained was subjected to reverse-phase column chromatography (C₁₈, gradient elution: 95:5 (v/v) H₂O: MeCN+0.1% formic acid→5:95 (v/v) H₂O: MeCN+0.1% formic acid) to afford the title compound as a 1:1 mixture of diastereomers (55% yield). LCMS: m/z=503.0 [M+H]⁺; ¹H NMR (acetone-d₆): δ=9.46 (s, 0.5H), 9.43 (s, 0.5H), 8.33 (d, J=8.6 Hz, 1H), 7.72˜7.24 (m, 6H), 7.51 (t, J=72.6 Hz, 1H), 6.65 (d, J=8.6 Hz, 0.5H), 6.62 (d, J=8.6 Hz, 0.5H), 3.83 (s, 3H), 3.46 (sept, J=6.6 Hz, 0.5H), 3.27 (sept, J=6.6 Hz, 0.5H), 2.67˜2.42 (m, 5H), 2.27˜2.01 (m, 2H), 1.76˜1.72 (m, 1H), 1.16 (d, J=6.6 Hz, 3H), 1.15 (d, J=6.6 Hz, 3H).

### Example 95: 4-cyano-N-(2-(difluoromethoxy)-6-methoxypyridin-3-yl)-1-(2-isopropylphenyl)cyclohexane-1-carboxamide

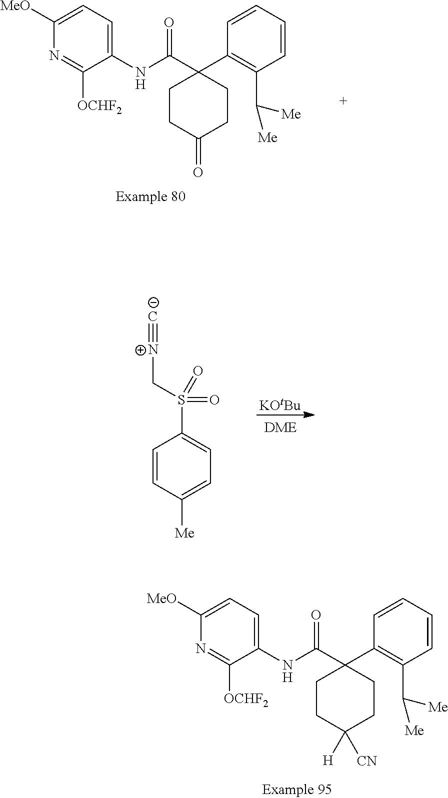 [SMILES: COc1ccc(NC(=O)C2(c3ccccc3C(C)C)CCC(=O)CC2)c(OC(F)F)n1 ; [C-]#[N+]CS(=O)(=O)c1ccc(C)cc1 ; COc1ccc(NC(=O)C2(c3ccccc3C(C)C)CCC(C#N)CC2)c(OC(F)F)n1 ; "KOtBu"]

In a round-bottom flask equipped with a magnetic stirrer was dissolved Example 80 (1 equiv) in 1,2-dimethoxyethane (0.09 M). To this solution was then added potassium tert-butoxide (1.2 equiv, Sigma-Aldrich) in one rapid portion, and the resulting reaction mixture was stirred at RT for 20 min. Finally, p-toluenesulfonylmethyl isocyanide (1.2 equiv, Sigma-Aldrich) was added at RT in one rapid portion and the resulting mixture was stirred at RT for an additional 1 h. The reaction was quenched with the addition of saturated aqueous NH₄C₁, and the volatiles were then removed in vacuo. The resulting residue was partitioned between EtOAc and water. The aqueous layer was separated and back extracted with EtOAc. The combined organic extracts were washed further with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN+0.1% trifluoroacetic acid→MeCN+0.1% trifluoroacetic acid) to afford the title compound as a 1:1 mixture of diastereomers (35% yield). LCMS: m/z=444.2 [M+H]⁺; ¹H NMR (methanol-d₄): δ=7.94 (d, J=8.7 Hz, 0.5H, diastereomer A or B), 7.93 (d, J=8.7 Hz, 0.5H, diastereomer A or B), 7.64˜7.54 (m, 1H), 7.47˜7.28 (m, 3H), 7.44 (t, J=72.6 Hz, 0.5H, diastereomer A or B), 7.42 (t, J=72.6 Hz, 0.5H, diastereomer A or B), 6.59 (d, J=8.7 Hz, 0.5H, diastereomer A or B), 6.58 (d, J=8.7 Hz, 0.5H, diastereomer A or B), 3.87 (s, 1.5H, diastereomer A or B), 3.86 (s, 1.5H, diastereomer A or B), 3.35˜3.28 (m, 1H), 3.04˜2.80 (m, 1H), 2.62˜2.52 (m, 1H), 2.47˜2.40 (m, 1H), 2.39˜2.27 (m, 3H), 2.06˜1.85 (m, 3H), 1.20 (d, J=6.6 Hz, 3H), 1.19 (d, J=6.6 Hz, 3H).

### Example 96: N¹-(2-(difluoromethoxy)-6-methoxypyridin-3-yl)-1-(2-isopropylphenyl)cyclohexane-1,4-dicarboxamide

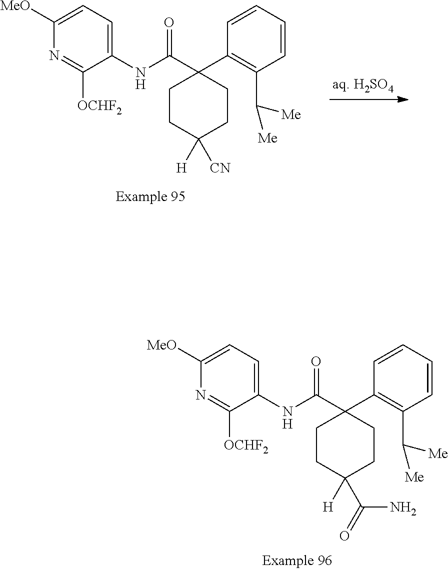 [SMILES: COc1ccc(NC(=O)C2(c3ccccc3C(C)C)CCC(C#N)CC2)c(OC(F)F)n1 ; COc1ccc(NC(=O)C2(c3ccccc3C(C)C)CCC(C(N)=O)CC2)c(OC(F)F)n1]

In a glass reaction vessel equipped with a magnetic stirrer was combined Example 95 (1 equiv) with wet sulfuric acid (500 equiv, Fisher Scientific). The resulting mixture was then stirred at RT for 4 h before it was carefully quenched with ice and solid sodium bicarbonate. The resulting suspension was then extracted with EtOAc. The combined organic extracts were washed further with water and brine, dried over Na₂SO₄, filtered, and the filtrate concentrated in vacuo. The crude product thus obtained was then subjected to reverse-phase column chromatography (C₁₈, gradient elution: 95:5 (v/v) H₂O: MeCN 4 MeCN) to afford the title compound as a 1.5:1 mixture of diastereomers (26% yield). LCMS: m/z=462.2 [M+H]⁺; ¹H NMR (methanol-d₄): δ=8.13˜7.15 (m, 6H), 6.68 (d, J=8.7 Hz, 0.4H, diastereomer A), 6.58 (d, J=8.7 Hz, 0.6H, diastereomer B), 3.93 (s, 1.2H, diastereomer A), 3.85 (s, 1.8H, diastereomer B), 3.42˜3.04 (m, 1H), 2.63˜2.34 (m, 2H), 2.27˜1.87 (m, 5H), 1.75˜1.60 (m, 2H), 1.18 (d, J=6.6 Hz, 6H).

### Example 97: N-(2-(difluoromethoxy)-6-methoxypyridin-3-yl)-1-(2-isopropylphenyl)-4-sulfamoylcyclohexane-1-carboxamide

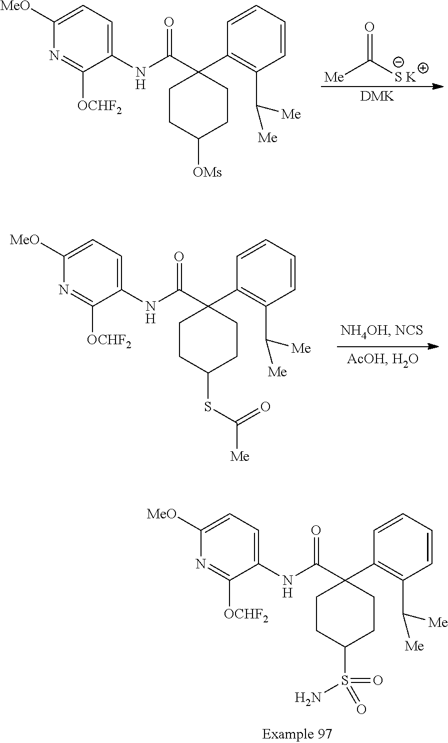

Step 1: In a dried, round-bottom flask equipped with a magnetic stirrer was combined 4-((2-(difluoromethoxy)-6-methoxypyridin-3-yl)carbamoyl)-4-(2-isopropylphenyl)cyclohexyl methanesulfonate (1 equiv, Example 84, Step 1) and potassium thioacetate (4 equiv, Sigma-Aldrich) in DMF (0.073 M). The resulting mixture was then heated at 75° C. for 16 h. Finally, the reaction mixture was cooled to RT and directly subjected to reverse-phase column chromatography (C₁₈, gradient elution: 95:5 (v/v) H₂O: MeCN 4 MeCN) to afford S-(4-((2-(difluoromethoxy)-6-methoxypyridin-3-yl)carbamoyl)-4-(2-isopropylphenyl)cyclohexyl) ethanethioate as a brown solid (71% yield).

Step 2: In a dried, round-bottom flask equipped with a magnetic stirrer was dissolved S-(4-((2-(difluoromethoxy)-6-methoxypyridin-3-yl)carbamoyl)-4-(2-isopropylphenyl)cyclohexyl) ethanethioate (1 equiv) from the previous step in a 5:1 (v/v) solution of glacial acetic acid and water (0.033 M). To this solution was then added, at 0° C., N-chlorosuccinimide (3.3 equiv, Sigma-Aldrich) portionwise over a period of 2 min, and the resulting mixture allowed to warm slowly to RT over 2 h. Finally, ammonium hydroxide (40 equiv, Supelco) was added at RT in one rapid portion and the resulting mixture was stirred at RT for an additional 16 h. The reaction mixture was then diluted further with water and extracted with dichloromethane. The combined organic extracts were washed further with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN→MeCN) to afford the title compound as a white solid (38% yield). LCMS: m/z=498.3 [M+H]⁺; ¹H NMR (methanol-d₄): δ=8.11 (d, J=8.6 Hz, 1H), 7.61˜7.14 (m, 5H), 6.58 (d, J=8.6 Hz, 1H), 3.83 (s, 3H), 3.13˜3.03 (m, 2H), 2.63˜2.58 (m, 2H), 2.31˜2.18 (m, 4H), 1.82˜1.74 (m, 2H), 1.18 (d, J=6.7 Hz, 6H).

### Example 98: N-(2-(difluoromethoxy)-6-methoxypyridin-3-yl)-4-(N,N-dimethylsulfamoyl)-1-(2-isopropylphenyl)cyclohexane-1-carboxamide

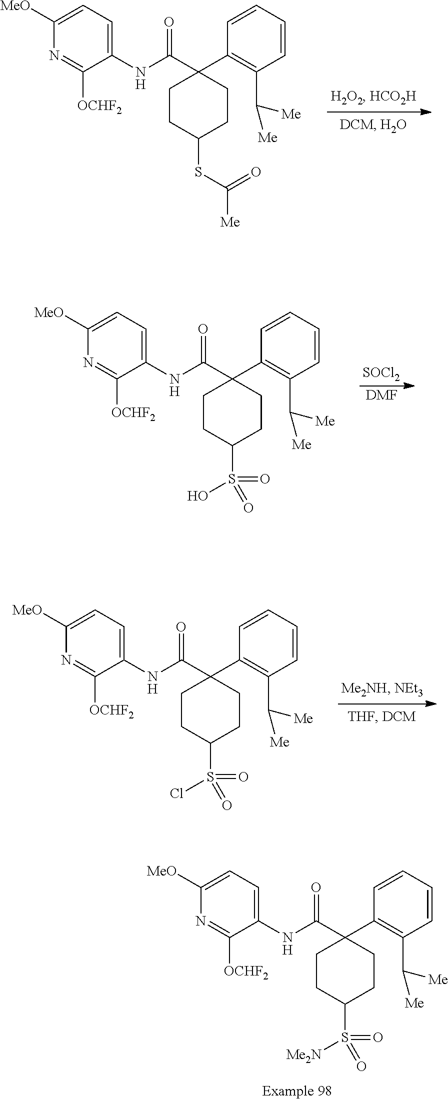

Step 1: In a round-bottom flask equipped with a magnetic stirrer was dissolved S-(4-((2-(difluoromethoxy)-6-methoxypyridin-3-yl)carbamoyl)-4-(2-isopropylphenyl)cyclohexyl) ethanethioate (1 equiv, Example 97, Step 1) in formic acid (0.029 M). To this solution was then added, at 0° C., hydrogen peroxide (30% w/w solution in water, 11 equiv) dropwise over a period of 3 min. The resulting mixture was allowed to warm slowly to RT and then stirred at RT for an additional 16 h. The reaction mixture was then quenched at 0° C. with the addition of sodium thiosulfate and filtered. The insolubles were rinsed further with DCM and the filtrate thus obtained was concentrated in vacuo. The resulting residue was directly subjected to column chromatography (SiO₂: 3:1 (v/v) DCM: MeOH) to afford 4-((2-(difluoromethoxy)-6-methoxypyridin-3-yl)carbamoyl)-4-(2-isopropylphenyl)cyclohexane-1-sulfonic acid as a yellow oil (86% yield).

Step 2: In a dried, round-bottom flask equipped with a magnetic stirrer was dissolved 4-((2-(difluoromethoxy)-6-methoxypyridin-3-yl)carbamoyl)-4-(2-isopropylphenyl)cyclohexane-1-sulfonic acid (1 equiv) from the previous step in chloroform (0.047 M). To this solution was then added sequentially at RT thionyl chloride (5 equiv, Sigma-Aldrich) and anhydrous DMF (0.2 equiv, Sigma-Aldrich), both neat and dropwise, over a period of 10 min. The resulting mixture was then heated at 75° C. for 12 h. After cooling to RT, removal of the volatiles in vacuo furnished crude 4-((2-(difluoromethoxy)-6-methoxypyridin-3-yl)carbamoyl)-4-(2-isopropylphenyl)cyclohexane-1-sulfonyl chloride as a yellow oil.

Step 3: In a dried, round-bottom flask equipped with a magnetic stirrer was combined 4-((2-(difluoromethoxy)-6-methoxypyridin-3-yl)carbamoyl)-4-(2-isopropylphenyl)cyclohexane-1-sulfonyl chloride (1 equiv) from the previous step and triethylamine (3 equiv, Sigma-Aldrich) in dichloromethane (0.1 M). To this solution was then added, at 0° C., dimethylamine (2 M solution in THF, 20 equiv, Thermo Scientific) neat and dropwise over a period of 2 min. The resulting mixture was allowed to warm slowly to RT and then stirred at RT for an additional 3 h. The volatiles were then removed in vacuo and the crude product thus obtained was directly subjected to reverse-phase column chromatography (C₁₈, gradient elution: 95:5 (v/v) H₂O: MeCN+0.1% trifluoroacetic acid→MeCN+0.1% trifluoroacetic acid) to afford the title compound as a 1:1 mixture of diastereomers (13% yield). LCMS: m/z=526.2 [M+H]⁺; ¹H NMR (methanol-d₄): δ=8.07 (d, J=8.8 Hz, 0.5H, diastereomer A or B), 7.92 (d, J=8.8 Hz, 0.5H, diastereomer A or B), 7.63˜7.23 (m, 5H), 6.57 (d, J=8.8 Hz, 0.5H, diastereomer A or B), 6.56 (d, J=8.8 Hz, 0.5H, diastereomer A or B), 3.84 (s, 1.5H, diastereomer A or B), 3.83 (s, 1.5H, diastereomer A or B), 3.42˜3.30 (m, 1H), 3.16˜3.04 (m, 1H), 2.93 (s, 3H), 2.88 (s, 3H), 2.69˜2.55 (m, 2H), 2.37˜2.27 (m, 2H), 2.10˜2.07 (m, 2H), 1.93˜1.62 (m, 2H), 1.35˜1.31 (m, 1H), 1.19 (d, J=6.6 Hz, 3H), 1.17 (d, J=6.6 Hz, 3H).

The following example was prepared in an analogous fashion to Example 98, but substituting dimethylamine in step 3 with the requisite amine.

| | | | LCMS |
|---|---|---|---|
| Example | Starting Amine | Structure | (m/z) |
| 99 | MeNH₂ (Sigma-Aldrich) | 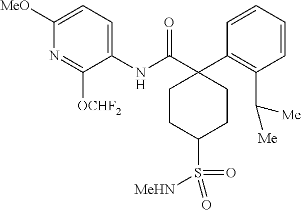 [SMILES: CNS(=O)(=O)C1CCC(C(=O)Nc2ccc(OC)nc2OC(F)F)(c2ccccc2C(C)C)CC1] N-(2-(difluoromethoxy)-6-methoxypyridin-3- yl)-1-(2-isopropylphenyl)-4-(N- methylsulfamoyl)cyclohexane-1-carboxamide | 512.2 [M+H]+ |

### Example 100: N-(2-(difluoromethoxy)-6-methoxypyridin-3-yl)-1-(2-isopropylphenyl)-4-(methylsulfonyl)cyclohexane-1-carboxamide

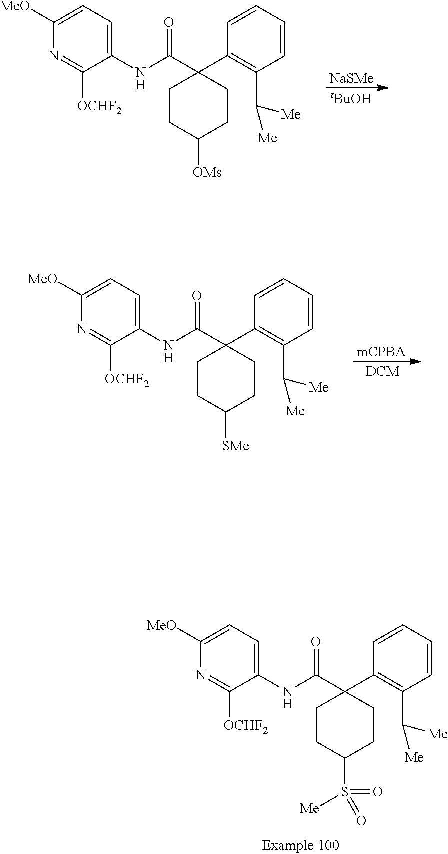

Step 1: In a dried, round-bottom flask equipped with a magnetic stirrer was combined 4-((2-(difluoromethoxy)-6-methoxypyridin-3-yl)carbamoyl)-4-(2-isopropylphenyl)cyclohexyl methanesulfonate (1 equiv, Example 84, Step 1) and sodium thiomethoxide (1.1 equiv, Sigma-Aldrich) in tert-butanol (0.067 M). The resulting mixture was heated at 70° C. for 16 h. After cooling to RT, the volatiles were removed in vacuo and the residue thus obtained was directly subjected to purification by way of column chromatography (SiO₂, gradient elution, Hex→EtOAc) to afford N-(2-(difluoromethoxy)-6-methoxypyridin-3-yl)-1-(2-isopropylphenyl)-4-(methylthio)cyclohexane-1-carboxamide as a white solid (66% yield).

Step 2: In a dried, round-bottom flask equipped with a magnetic stirrer was dissolved N-(2-(difluoromethoxy)-6-methoxypyridin-3-yl)-1-(2-isopropylphenyl)-4-(methylthio)cyclohexane-1-carboxamide (1 equiv) from the previous step in dichloromethane (0.055 M). To this was then added m-chloroperoxybenzoic acid (2 equiv, Sigma-Aldrich) and the resulting mixture was stirred at RT for 2 h. The reaction was then quenched with water and extracted with dichloromethane. The combined organic extracts were washed sequentially with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN+0.1% trifluoroacetic acid→5:95 (v/v) H₂O: MeCN+0.1% trifluoroacetic acid) to afford the title compound as a white solid (31% yield). LCMS: m/z=497.3 [M+H]⁺; ¹H NMR (methanol-d₄): δ=8.08 (d, J=8.6 Hz, 1H), 7.63˜7.14 (m, 5H), 6.58 (d, J=8.6 Hz, 1H), 3.83 (s, 3H), 3.29˜3.05 (m, 2H), 2.88 (s, 3H), 2.65˜2.60 (m, 2H), 2.35˜2.11 (m, 4H), 1.83˜1.71 (m, 2H), 1.19 (d, J=6.7 Hz, 6H).

### Example 101: Preparation of 3-(2-isopropylphenyl)-N-(2-methoxy-4-methylphenyl)-5-oxopyrrolidine-3-carboxamide

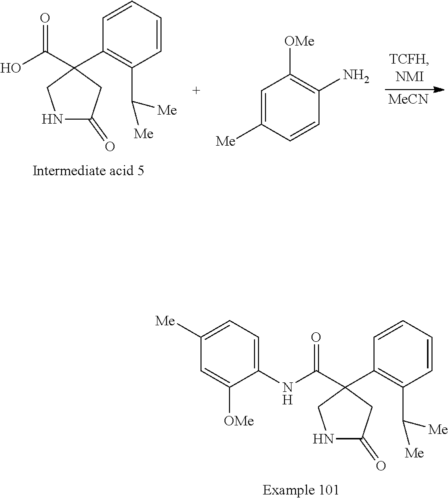

In a dried, thick-walled glass reaction vessel equipped with a magnetic stirrer and a Teflon screwcap was combined Intermediate acid 5 (1 equiv), 2-methoxy-4-methylaniline (2.5 equiv, Combi-Blocks), and 1-methylimidazole (8 equiv, Sigma-Aldrich) in anhydrous acetonitrile (0.04 M). To this was then added chloro-N,N,N′,N′-tetramethylformamidinium hexafluorophosphate (2.5 equiv, Chem-Impex) in one rapid portion, the glass vessel was tightly sealed, and the resulting orange solution was heated at 80° C. for 16 h. After cooling to RT, the volatiles were then removed in vacuo and the residue thus obtained was subjected first to reverse-phase column chromatography (C₁₈, gradient elution: 9:1 (v/v) H₂O: MeCN+0.1% formic acid 4 MeCN+0.1% formic acid), and then to regular phase column chromatography (SiO₂, gradient elution: 4:1 (v/v) Hex: EtOAc→EtOAc→10:1 (v/v) EtOAc: MeOH), to afford the title compound as a white solid (50% yield). LCMS: m/z=389.1 [M+Na]*; ¹H NMR (CDCl₃): δ=8.12 (d, J=8.2 Hz, 1H), 7.45 (s, 1H), 7.44˜7.38 (m, 3H), 7.32-7.28 (m, 1H), 6.75 (d, J=8.2 Hz, 1H), 6.56 (d, J=1.3 Hz, 1H), 5.70 (s, 1H), 4.29 (d, J=9.9 Hz, 1H), 3.78 (d, J=9.9 Hz, 1H), 3.53 (s, 3H), 3.34 (d, J=16.6 Hz, 1H), 2.86 (sept, J=6.6 Hz, 1H), 2.84 (d, J=16.6 Hz, 1H), 2.28 (s, 3H), 1.11 (d, J=6.6 Hz, 6H).

The following examples were prepared in an analogous fashion to Example 101, but substituting 2-methoxy-4-methyl aniline with the requisite amine. In Examples 102-isomer A, Intermediate acid 5 was also substituted with Intermediate acid 5-isomer A. In Examples 102-isomer B, Intermediate acid 5 was also substituted with Intermediate acid 5-isomer B.

| | | | LCMS |
|---|---|---|---|
| Example | Starting Amine | Structure | (m/z) |
| 102- isomer A | 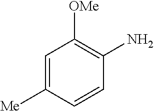 (Combi-Blocks) | 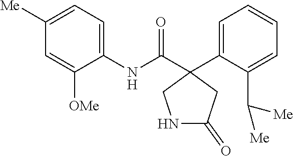 [SMILES: COc1cc(C)ccc1NC(=O)C1(c2ccccc2C(C)C)CNC(=O)C1] 3-(2-isopropylphenyl)-N-(2-methoxy-4- methylphenyl)-5-oxopyrrolidine-3- carboxamide | 365.2 [M + H]⁺ |
| 102- isomer B | 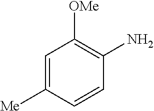 (Combi-Blocks) | 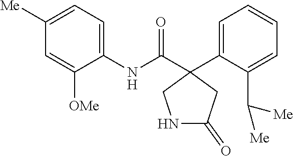 [SMILES: COc1cc(C)ccc1NC(=O)C1(c2ccccc2C(C)C)CNC(=O)C1] 3-(2-isopropylphenyl)-N-(2-methoxy-4- methylphenyl)-5-oxopyrrolidine-3- carboxamide | 365.2 [M + H]⁺ |
| 103 | 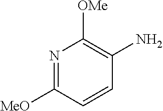 [SMILES: COc1ccc(N)c(OC)n1] (AmBeed) | 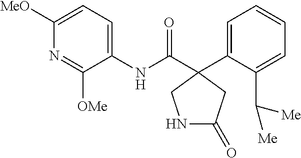 [SMILES: COc1ccc(NC(=O)C2(c3ccccc3C(C)C)CNC(=O)C2)c(OC)n1] N-(2,6-dimethoxypyridin-3-yl)-3-(2- isopropylphenyl)-5-oxopyrrolidine-3- carboxamide | 406.1 [M + Na]⁺ |
| 104 | 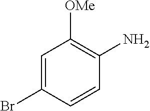 (Combi-Blocks) | 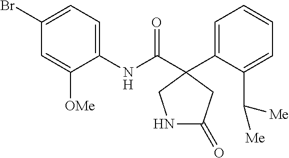 [SMILES: COc1cc(Br)ccc1NC(=O)C1(c2ccccc2C(C)C)CNC(=O)C1] N-(4-bromo-2-methoxyphenyl)-3-(2- isopropylphenyl)-5-oxopyrrolidine-3- carboxamide | 453.9, 455.8 [M + Na]⁺ |
| 105 | 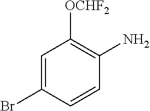 [SMILES: Nc1ccc(Br)cc1OC(F)F] (Combi-Blocks) | 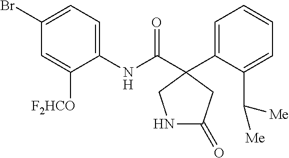 [SMILES: CC(C)c1ccccc1C1(C(=O)Nc2ccc(Br)cc2OC(F)F)CNC(=O)C1] N-(4-bromo-2- (difluoromethoxy)phenyl)-3-(2- isopropylphenyl)-5-oxopyrrolidine-3- carboxamide | 466.9 468.8 [M + H]⁺ |
| 106 | 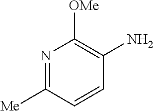 (Combi-Blocks) | 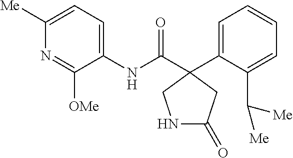 [SMILES: COc1nc(C)ccc1NC(=O)C1(c2ccccc2C(C)C)CNC(=O)C1] 3-(2-isopropylphenyl)-N-(2-methoxy-6- methylpyridin-3-yl)-5-oxopyrrolidine-3- carboxamide | 368.2 [M + H]⁺ |
| 107 | 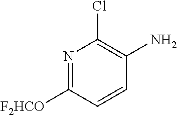 [SMILES: Nc1ccc(OC(F)F)nc1Cl] Intermediate amine 12 | 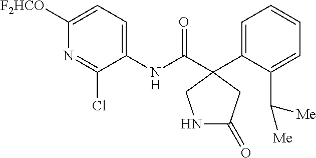 [SMILES: CC(C)c1ccccc1C1(C(=O)Nc2ccc(OC(F)F)nc2Cl)CNC(=O)C1] N-(2-chloro-6-(difluoromethoxy)pyridin- 3-yl)-3-(2-isopropylphenyl)-5- oxopyrrolidine-3-carboxamide | 424.0, 426.0 [M + H]⁺ |
| 108 | 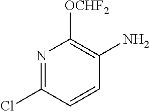 [SMILES: Nc1ccc(Cl)nc1OC(F)F] Intermediate amine 13 | 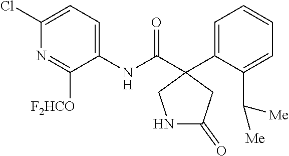 [SMILES: CC(C)c1ccccc1C1(C(=O)Nc2ccc(Cl)nc2OC(F)F)CNC(=O)C1] N-(6-chloro-2-(difluoromethoxy)pyridin- 3-yl)-3-(2-isopropylphenyl)-5- oxopyrrolidine-3-carboxamide | 424.0, 426.0 [M + H]⁺ |
| 114 | 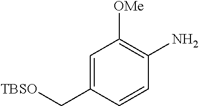 [SMILES: COc1cc(CO[Si](C)(C)C(C)(C)C)ccc1N] Intermediate amine 14 | 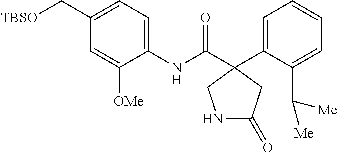 [SMILES: COc1cc(CO[Si](C)(C)C(C)(C)C)ccc1NC(=O)C1(c2ccccc2C(C)C)CNC(=O)C1] N-(4-(((tert- butyldimethylsilyl)oxy)methyl)-2- methoxyphenyl)-3-(2-isopropylphenyl)- 5-oxopyrrolidine-3-carboxamide | 497.9 [M + H]⁺ |

### Example 109: Preparation of N-(2-(difluoromethoxy)-4-methylphenyl)-3-(2-isopropylphenyl)-5-oxopyrrolidine-3-carboxamide

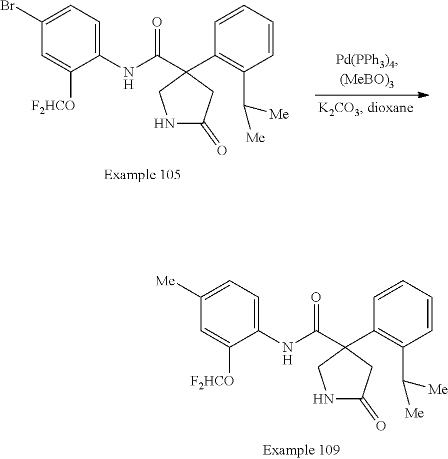 [SMILES: CC(C)c1ccccc1C1(C(=O)Nc2ccc(Br)cc2OC(F)F)CNC(=O)C1 ; Cc1ccc(NC(=O)C2(c3ccccc3C(C)C)CNC(=O)C2)c(OC(F)F)c1]

In a thick-walled, glass reaction vessel equipped with a magnetic stirrer and a Teflon screwcap was combined Example 105 (1 equiv), potassium carbonate (2 equiv, Sigma-Aldrich), and tetrakis(triphenylphosphine)palladium(0) (0.1 equiv, Sigma-Aldrich) in 1,4-dioxane (0.037 M). The resulting suspension was deoxygenated via subsurface purging with nitrogen for 10 min before trimethylboroxine (4 equiv, Sigma-Aldrich) was added neat and dropwise to the reaction suspension. The reaction vessel was then tightly sealed and heated at 100° C. for 16 h. After cooling to RT, the volatiles were then removed in vacuo and the residue thus obtained was subjected first to reverse-phase column chromatography (C₁₈, gradient elution: 9:1 (v/v) H₂O: MeCN+0.1% formic acid 4 MeCN+0.1% formic acid), and then to regular phase column chromatography (SiO₂, gradient elution: 4:1 (v/v) Hex: EtOAc→EtOAc→10:1 (v/v) EtOAc: MeOH), to afford the title compound as a colorless oil (50% yield). LCMS: m/z=403.1 [M+H]⁺; ¹H NMR (CDCl₃): δ=8.19 (d, J=8.4 Hz, 1H), 7.45˜7.39 (m, 3H), 7.33˜7.28 (m, 2H), 7.01 (dd, J=8.4, 1.3 Hz, 1H), 6.80 (s, 1H), 6.10 (t, J=73.4 Hz, 1H), 5.80 (s, 1H), 4.27 (d, J=9.9 Hz, 1H), 3.82 (d, J=9.9 Hz, 1H), 3.31 (d, J=16.3 Hz, 1H), 2.87 (sept, J=6.7 Hz, 1H), 2.86 (d, J=16.3 Hz, 1H), 2.28 (s, 3H), 1.13 (d, J=6.7 Hz, 6H).

The following example was prepared in an analogous fashion to Example 109, but substituting Example 105 with the requisite halide.

| | | | LCMS |
|---|---|---|---|
| Example | Starting Halide | Structure | (m/z) |
| 110 | 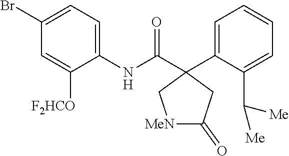 [SMILES: CC(C)c1ccccc1C1(C(=O)Nc2ccc(Br)cc2OC(F)F)CC(=O)N(C)C1] Example 87 | 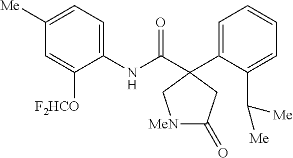 [SMILES: Cc1ccc(NC(=O)C2(c3ccccc3C(C)C)CC(=O)N(C)C2)c(OC(F)F)c1] N-(2-(difluoromethoxy)-4- methylphenyl)-3-(2- isopropylphenyl)-1-methyl-5- oxopyrrolidine-3-carboxamide | 417.2 [M + H]⁺ |

### Example 112: Preparation of 3-((1H-tetrazol-5-yl)methyl)-N-(2-(difluoromethoxy)-6-methylpyridin-3-yl)-1-(2-isopropylphenyl)cyclobutane-1-carboxamide

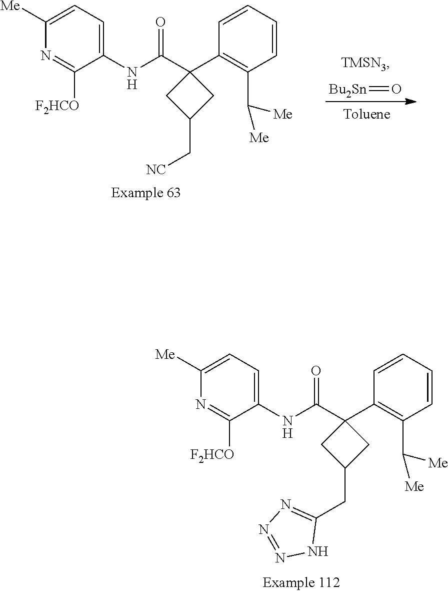

In a thick-walled, glass reaction vessel equipped with a magnetic stirrer and a Teflon screwcap was combined Example 63 (1 equiv), trimethylsilyl azide (2.2 equiv, Sigma-Aldrich), and dibutyltin(IV) oxide (0.2 equiv, TCI) in toluene (0.11 M). The resulting mixture was heated at 110° C. for 24 h. After cooling to RT, the reaction was quenched with methanol and the volatiles were then removed in vacuo. The resulting residue was suspended in water and extracted with EtOAc. The combined organic extracts were washed further with brine, dried over Na₂SO₄, filtered, and the filtrate concentrated in vacuo. The crude product thus obtained was subjected to reverse-phase column chromatography (C₁₈, gradient elution: 9:1 (v/v) H₂O: MeCN+0.1% formic acid→MeCN+0.1% formic acid) to afford the title compound as a 2.3:1 mixture of diastereomers (78% yield). LCMS: m/z=457.1 [M+H]⁺; ¹H NMR (CDCl₃): δ=8.44 (d, J=8.1 Hz, 0.7H, diastereomer A), 8.40 (d, J=8.1 Hz, 0.3H, diastereomer B), 7.49˜7.47 (m, 1H), 7.40˜7.29 (m, 4H), 7.24 (t, J=72.6 Hz, 0.7H, diastereomer A), 7.23 (t, J=72.6 Hz, 0.3H, diastereomer B), 7.18 (s, 0.3H, diastereomer B), 7.16 (s, 0.7H, diastereomer A), 6.90 (d, J=8.1 Hz, 0.7H, diastereomer A), 6.87 (d, J=8.1 Hz, 0.3H, diastereomer B), 3.38˜2.96 (m, 2H), 2.71 (sept, J=6.6 Hz, 0.3H, diastereomer B), 2.60 (sept, J=6.6 Hz, 0.7H, diastereomer A), 2.36 (s, 2.1H, diastereomer A), 2.34 (s, 0.9H, diastereomer B), 1.93˜1.26 (m, 4H), 1.08 (d, J=6.6 Hz, 6H), 0.90˜0.83 (m, 1H).

The following example was prepared in an analogous fashion to Example 112, but substituting Example 63 with the requisite nitrile.

| | | | LCMS |
|---|---|---|---|
| Example | Starting Nitrile | Structure | (m/z) |
| 113 | 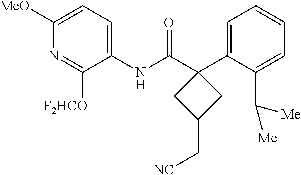 [SMILES: COc1ccc(NC(=O)C2(c3ccccc3C(C)C)CC(CC#N)C2)c(OC(F)F)n1] Example 111 | 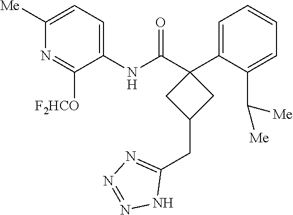 [SMILES: Cc1ccc(NC(=O)C2(c3ccccc3C(C)C)CC(Cc3nnn[nH]3)C2)c(OC(F)F)n1] 3-((1H-tetrazol-5-yl)methyl)-N- (2-(difluoromethoxy)-6- methoxypyridin-3-yl)-1-(2- isopropylphenyl)cyclobutane-1- carboxamide | 473.1 [M + H]⁺ |

### Example 115: N-(4-(fluoromethyl)-2-methoxyphenyl)-3-(2-isopropylphenyl)-5-oxopyrrolidine-3-carboxamide

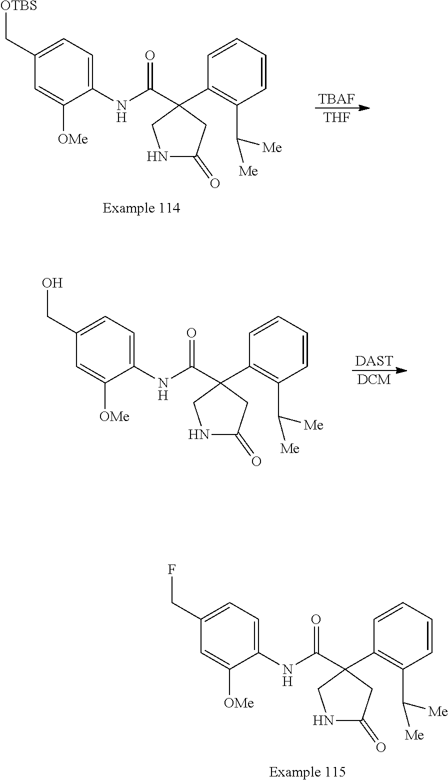

Step 1: In a dried, round-bottom flask equipped with a magnetic stirrer was dissolved Example 114 (1 equiv) in THE (0.037 M). To this colorless solution was then added, at 0° C., tetrabutylammonium fluoride (1 M solution in THF, 1 equiv, Sigma-Aldrich) dropwise over a period of 5 min. The resulting yellow solution was first stirred at 0° C. for 15 min and then at RT for 1 h. The reaction was then quenched with water and extracted with tert-butyl methyl ether. The combined organic extracts were washed further with brine, dried over Na₂SO₄, and filtered. Concentration of the filtrate in vacuo afforded crude N-(4-(hydroxymethyl)-2-methoxyphenyl)-3-(2-isopropylphenyl)-5-oxopyrrolidine-3-carboxamide as a white solid (>99% yield).

Step 2: In a Nalgene reaction vessel equipped with a magnetic stirrer was dissolved N-(4-(hydroxymethyl)-2-methoxyphenyl)-3-(2-isopropylphenyl)-5-oxopyrrolidine-3-carboxamide (1 equiv) from the previous step in dichloromethane (0.033 M). To this colorless solution was then added diethylaminosulfur trifluoride (0.5 M solution in dichloromethane, 1.1 equiv, Sigma-Aldrich) dropwise over a period of 5 min. After another 5 min of stirring at RT, the reaction was then carefully quenched with ice water and extracted with dichloromethane. The combined organic extracts were washed further with water and brine, dried over Na₂SO₄, filtered, and the filtrate concentrated in vacuo. The crude product thus obtained was subjected to reverse-phase column chromatography (C₁₈, gradient elution: 95:5 (v/v) H₂O: MeCN+0.1% formic acid→MeCN+0.1% formic acid) to afford the title compound as a white solid (44% yield). LCMS: m/z=407.1 [M+Na]*; ¹H NMR (CDCl₃): δ=8.30 (dd, J=8.2, 1.1 Hz, 1H), 7.58 (s, 1H), 7.43˜7.39 (m, 3H), 7.33˜7.28 (m, 1H), 6.94 (dt, J=8.2, 2.0 Hz, 1H), 6.80 (s, 1H), 5.89 (s, 1H), 5.29 (d, J=48.0 Hz, 1H), 4.30 (d, J=9.9 Hz, 1H), 3.80 (d, J=9.9 Hz, 1H), 3.58 (s, 3H), 3.33 (d, J=16.4 Hz, 1H), 2.87 (d, J=16.4 Hz, 1H), 2.84 (sept, J=6.7 Hz, 1H), 1.13 (d, J=6.6 Hz, 6H).

### Example 116: N-(2-(difluoromethoxy)-6-methoxypyridin-3-yl)-4-(2-isopropylphenyl)-2-oxopiperidine-4-carboxamide

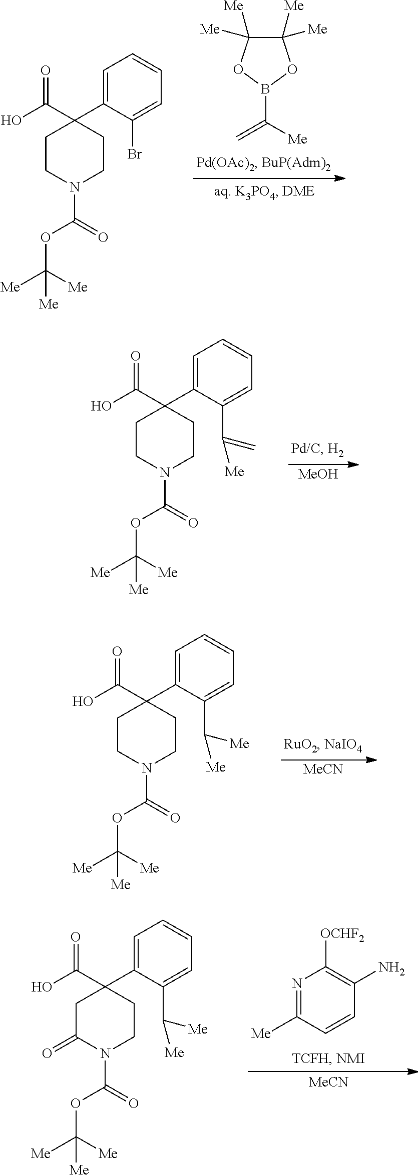

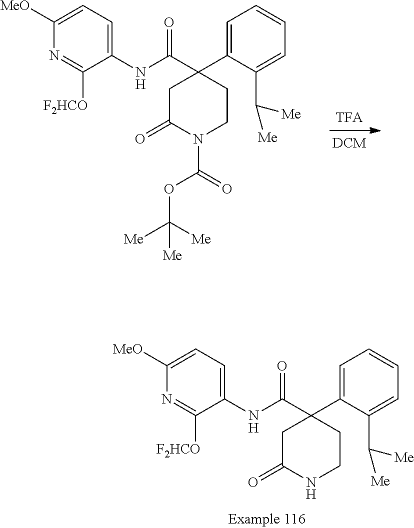 [SMILES: COc1ccc(NC(=O)C2(c3ccccc3C(C)C)CCN(C(=O)OC(C)(C)C)C(=O)C2)c(OC(F)F)n1 ; COc1ccc(NC(=O)C2(c3ccccc3C(C)C)CCNC(=O)C2)c(OC(F)F)n1]

Step 1: In a thick-walled glass reaction vessel equipped with a magnetic stirrer and a Teflon screwcap was combined 4-(2-bromophenyl)-1-(tert-butoxycarbonyl)piperidine-4-carboxylic acid (1 equiv, Amatek Chemical), 4,4,5,5-tetramethyl-2-(prop-1-en-2-yl)-1,3,2-dioxaborolane (1.5 equiv, Frontier Scientific), palladium(II) acetate (0.1 equiv, Sigma-Aldrich), di(1-adamantyl)-n-butylphosphine (0.2 equiv, Sigma-Aldrich), and potassium phosphate (5 equiv, Alfa Aesar) in a 2:1 (v/v) solution of 1,2-dimethoxyethane and water (0.034 M). The resulting mixture was deoxygenated via subsurface purging with nitrogen for 10 min before the reaction vessel was tightly sealed and heated at 100° C. for 48 h. The now dark brown suspension was cooled to RT, acidified to a pH of ˜4 with 1 M aqueous HCl, and extracted with EtOAc. The combined organic extracts were washed further with brine, dried over Na₂SO₄, filtered, and the filtrate concentrated in vacuo. Purification of the crude product thus obtained by way of column chromatography (SiO₂, gradient elution: Hex→EtOAc) afforded 1-(tert-butoxycarbonyl)-4-(2-(prop-1-en-2-yl)phenyl)piperidine-4-carboxylic acid as a yellow oil (83% yield).

Step 2: In a dried, round-bottom flask equipped with a magnetic stirrer was dissolved 1-(tert-butoxycarbonyl)-4-(2-(prop-1-en-2-yl)phenyl)piperidine-4-carboxylic acid (1 equiv) from the previous step in methanol (0.086 M). The resulting colorless solution was then deoxygenated via subsurface purging with nitrogen for 10 min before palladium (10% w/w over activated carbon, dry, 0.07 equiv, Sigma-Aldrich) was added in one rapid portion. The resulting black suspension was then subsurface purged with hydrogen for 10 min before it was stirred under a static hydrogen atmosphere at RT for 1 h. The reaction was subsequently quenched with dichloromethane and filtered through a bed of dichloromethane-wetted celite. Concentration of the filtrate in vacuo afforded the crude 1-(tert-butoxycarbonyl)-4-(2-isopropylphenyl)piperidine-4-carboxylic acid as a cloudy oil (93% yield).

Step 3: In a round-bottom flask equipped with a magnetic stirrer was dissolved 1-(tert-butoxycarbonyl)-4-(2-isopropylphenyl)piperidine-4-carboxylic acid (1 equiv) from the previous step in a 5:1 (v/v) solution of water and acetonitrile (0.058 M). To this solution was then added sodium periodate (10 equiv, Sigma-Aldrich) and ruthenium(IV) oxide (1 equiv, Sigma-Aldrich) in one rapid portion, and resulting black suspension was stirred at RT for 16 h. The reaction was subsequently quenched with dichloromethane and filtered through a bed of dichloromethane-wetted celite. The insolubles were rinsed further with dichloromethane and the filtrate thus obtained was then acidified to a pH of ˜4 with 1 M aqueous HCl. The organic layer was separated and concentrated in vacuo. Purification of the crude product thus obtained by way of column chromatography (SiO₂, gradient elution: DCM→1:1 (v/v) DCM: MeOH) afforded 1-(tert-butoxycarbonyl)-4-(2-isopropylphenyl)-2-oxopiperidine-4-carboxylic acid as a yellow oil (44% yield).

Step 4: In a dried, round-bottom flask equipped with a magnetic stirrer was combined 1-(tert-butoxycarbonyl)-4-(2-isopropylphenyl)-2-oxopiperidine-4-carboxylic acid (1 equiv) from the previous step, Intermediate amine 1 (2 equiv), and 1-methylimidazole (7 equiv, Sigma-Aldrich) in anhydrous acetonitrile (0.03 M). To this was then added chloro-N,N,N′,N′-tetramethylformamidinium hexafluorophosphate (5 equiv, Chem-Impex) in one rapid portion and the resulting mixture was stirred at 50° C. for 16 h. The volatiles were then removed in vacuo and the residue thus obtained was subjected to reverse-phase column chromatography (C₁₈, gradient elution: 9:1 (v/v) H₂O: MeCN 4 MeCN) to afford tert-butyl 4-((2-(difluoromethoxy)-6-methoxypyridin-3-yl)carbamoyl)-4-(2-isopropylphenyl)-2-oxopiperidine-1-carboxylate as a yellow oil (32% yield).

Step 5: In a round-bottom flask equipped with a magnetic stirrer was dissolved tert-butyl 4-((2-(difluoromethoxy)-6-methoxypyridin-3-yl)carbamoyl)-4-(2-isopropylphenyl)-2-oxopiperidine-1-carboxylate (1 equiv) from the previous step in dichloromethane (0.02 M). To this solution was then added trifluoroacetic acid (50 equiv, Sigma-Aldrich) and the resulting reaction mixture was stirred at RT for 30 min. The volatiles were then removed in vacuo via sequential azeotropic distillation with toluene and heptane. The crude product thus obtained was then subjected to reverse-phase column chromatography (C₁₈, gradient elution: 9:1 (v/v) H₂O: MeCN→MeCN) to afford the title compound as a colorless oil (25% yield). LCMS: m/z=456.2 [M+Na]*; ¹H NMR (methanol-d₄): δ=7.88 (t, J=8.4 Hz, 1H), 7.65˜7.23 (m, 5H), 6.57 (d, J=8.4 Hz, 1H), 3.85 (s, 3H), 3.40˜3.33 (m, 1H), 3.16 (sept, J=6.8 Hz, 1H), 3.04˜2.99 (m, 2H), 2.70˜2.43 (m, 3H), 1.24 (d, J=6.8 Hz, 3H), 1.20 (d, J=6.8 Hz, 3H).

### Example 117: Preparation of cis-3-(2-amino-2-oxoethyl)-N-(2-(difluoromethoxy)-6-methoxypyridin-3-yl)-1-(2-isopropylphenyl)cyclobutane-1-carboxamide

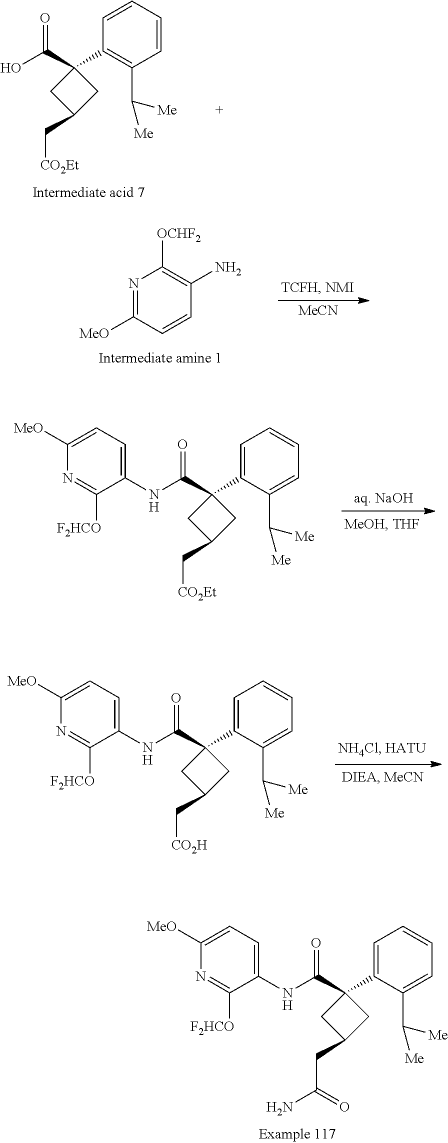 [SMILES: "MeCN" ; CCOC(=O)C[C@H]1C[C@](C(=O)O)(c2ccccc2C(C)C)C1 ; COc1ccc(N)c(OC(F)F)n1 ; CCOC(=O)C[C@H]1C[C@](C(=O)Nc2ccc(OC)nc2OC(F)F)(c2ccccc2C(C)C)C1 ; COc1ccc(NC(=O)[C@]2(c3ccccc3C(C)C)C[C@H](CC(=O)O)C2)c(OC(F)F)n1 ; COc1ccc(NC(=O)[C@]2(c3ccccc3C(C)C)C[C@H](CC(N)=O)C2)c(OC(F)F)n1]

Step 1: In a dried, thick-walled glass reaction vessel equipped with a magnetic stirrer and a Teflon screwcap was combined Intermediate acid 7 (1 equiv), Intermediate amine 1 (1 equiv), and 1-methylimidazole (4 equiv, Sigma-Aldrich) in anhydrous acetonitrile (0.041 M). To this was then added chloro-N,N,N′,N′-tetramethylformamidinium hexafluorophosphate (1.5 equiv, Chem-Impex) in one rapid portion and the glass vessel was tightly sealed. The resulting orange solution was heated at 60° C. for 6 h and then stirred at RT for 6 days. The volatiles were then removed in vacuo and the residue thus obtained was subjected first to reverse-phase column chromatography (C₁₈, gradient elution: 9:1 (v/v) H₂O: MeCN+0.1% formic acid 4 MeCN+0.1% formic acid), and then to regular phase column chromatography (SiO₂, gradient elution: 95:5 (v/v) Hex: EtOAc→45:55 (v/v) Hex: EtOAc), to afford ethyl 2-((1s,3s)-3-((2-(difluoromethoxy)-6-methoxypyridin-3-yl)carbamoyl)-3-(2-isopropylphenyl)cyclobutyl)acetate as a viscous oil (29% yield).

Step 2: In a round-bottom flask equipped with a magnetic stirrer was dissolved ethyl 2-((1s,3s)-3-((2-(difluoromethoxy)-6-methoxypyridin-3-yl)carbamoyl)-3-(2-isopropylphenyl)cyclobutyl)acetate (1 equiv) from the previous step in a 1:3 (v/v) solution of THF and methanol (0.17 M). To this colorless solution was then added sodium hydroxide (2 M solution in water, 3 equiv) and the resulting mixture was stirred at RT for 18 h. The reaction mixture was then carefully quenched with HCl (2 M solution in water, 3 equiv) and the volatiles were removed in vacuo. The resulting suspension was back extracted with EtOAc. The combined organic extracts were washed sequentially with water and brine, dried over MgSO₄, and filtered. Concentration of the filtrate in vacuo afforded 2-((1s,3s)-3-((2-(difluoromethoxy)-6-methoxypyridin-3-yl)carbamoyl)-3-(2-isopropylphenyl)cyclobutyl)acetic acid as a white foam (91% yield).

Step 3: In a glass reaction vessel equipped with a magnetic stirrer and a Teflon screwcap was combined 2-((1s,3s)-3-((2-(difluoromethoxy)-6-methoxypyridin-3-yl)carbamoyl)-3-(2-isopropylphenyl)cyclobutyl)acetic acid (1 equiv) from the previous step, ammonium chloride (4 equiv, Fisher Scientific), and N,N-diisopropylethylamine (5 equiv, Sigma-Aldrich) in a 2:1 (v/v) solution of acetonitrile and DMF (0.12 M). To this reaction mixture was then added 1-[bis(dimethylamino)methylene]-1H-1,2,3-triazolo[4,5-b]pyridinium 3-oxide hexafluorophosphate (1.5 equiv, Sigma-Aldrich) and the resulting solution was stirred at RT for 18 h. The reaction mixture was then quenched with the carefully addition of 1 M aq. HCl and extracted with EtOAc. The combined organic extracts were washed further with water and brine, dried over MgSO₄, filtered, and the filtrate concentrated in vacuo. Purification of the crude product thus obtained by way of column chromatography (SiO₂, gradient elution: 2:3 (v/v) Hex: EtOAc→EtOAc) afforded the title compound as a white solid (70% yield). LCMS: m/z=448.1 [M+H]⁺; ¹H NMR (CDCl₃): δ=8.42 (d, J=8.7 Hz, 1H), 7.49 (d, J=8.7 Hz, 1H), 7.39˜7.27 (m, 3H), 7.17 (t, J=72.6 Hz, 1H), 6.82 (s, 1H), 6.50 (d, J=8.7 Hz, 1H), 5.60 (br s, 1H), 5.41 (br s, 1H), 3.80 (s, 3H), 2.91˜2.55 (m, 8H), 1.09 (d, J=6.9 Hz, 6H).

The following example was prepared in an analogous fashion to Example 117, but substituting Intermediate acid 7 with the requisite starting acid.

| | | | LCMS |
|---|---|---|---|
| Example | Starting Acid | Structure | (m/z) |
| 118 | 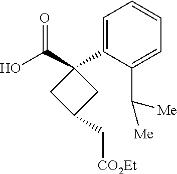 [SMILES: CCOC(=O)C[C@H]1C[C@@](C(=O)O)(c2ccccc2C(C)C)C1] Intermediate acid 8 | 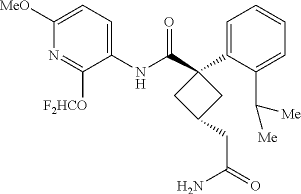 [SMILES: COc1ccc(NC(=O)[C@]2(c3ccccc3C(C)C)C[C@@H](CC(N)=O)C2)c(OC(F)F)n1] trans-3-(2-amino-2-oxoethyl)- N-(2-(difluoromethoxy)-6- methoxypyridin-3-yl)-1-(2- isopropylphenyl)cyclobutane-1- carboxamide | 448.1 [M + H]⁺ |

### Example 119: Preparation of cis-3-((1H-tetrazol-5-yl)methyl)-N-(2-(difluoromethoxy)-6-methoxypyridin-3-yl)-1-(2-isopropylphenyl)cyclobutane-1-carboxamide

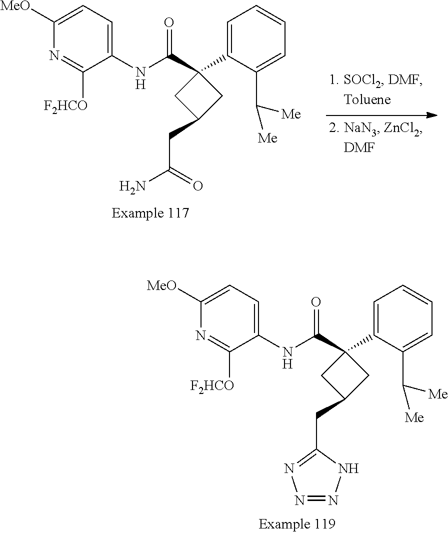 [SMILES: COc1ccc(NC(=O)[C@]2(c3ccccc3C(C)C)C[C@H](CC(N)=O)C2)c(OC(F)F)n1 ; COc1ccc(NC(=O)[C@]2(c3ccccc3C(C)C)C[C@H](Cc3nnn[nH]3)C2)c(OC(F)F)n1]

Step 1: In a glass reaction vessel equipped with a magnetic stirrer and a Teflon screwcap was combined Example 117 (1 equiv) and thionyl chloride (5 equiv, Sigma-Aldrich), in anhydrous toluene (0.15 M). To this was then added anhydrous DMF (0.5 equiv), neat and dropwise, at RT and the resulting mixture was stirred at RT for 16 h. The volatiles were then removed in vacuo and the residue thus obtained was partitioned between EtOAc and water. The organic layer was separated, washed further with brine, dried over MgSO₄, filtered, and the filtrate concentrated in vacuo. Purification of the crude product thus obtained by way of column chromatography (SiO₂, gradient elution: 95:5 (v/v) Hex: EtOAc→60:40 (v/v) Hex: EtOAc) afforded cis-3-(cyanomethyl)-N-(2-(difluoromethoxy)-6-methoxypyridin-3-yl)-1-(2-isopropylphenyl)cyclobutane-1-carboxamide as a viscous oil (68% yield).

Step 2: In a microwave reaction vessel equipped with a magnetic stirrer was combined cis-3-(cyanomethyl)-N-(2-(difluoromethoxy)-6-methoxypyridin-3-yl)-1-(2-isopropylphenyl)cyclobutane-1-carboxamide (1 equiv) from the previous step and sodium azide (4.5 equiv, Sigma-Aldrich) in DMF (0.09 M). To this colorless solution was then added zinc(II) chloride (1 M solution in 2-methyltetrahydrofuran, 4.2 equiv, Sigma-Aldrich) and the resulting mixture was heated via microwave radiation at 150° C. for 8 h. After cooling to RT, the volatiles were removed in vacuo and the residue thus obtained was subjected to reverse-phase column chromatography (C₁₈, gradient elution: 9:1 (v/v) H₂O: MeCN+0.1% formic acid 4 MeCN+0.1% formic acid) to afford the title compound as a white solid (70% yield). LCMS: m/z=473.0 [M+H]⁺; ¹H NMR (CDCl₃): δ=8.41 (d, J=8.7 Hz, 1H), 7.46 (d, J=7.5 Hz, 1H), 7.43˜7.27 (m, 3H), 7.18 (t, J=72.6 Hz, 1H), 6.98 (s, 1H), 6.53 (d, J=8.7 Hz, 1H), 3.81 (s, 3H), 3.36 (d, J=5.7 Hz, 2H), 3.15˜2.53 (m, 6H), 1.11 (d, J=6.3 Hz, 6H).

The following example was prepared in an analogous fashion to Example 119, but substituting Example 117 with the requisite starting amide.

| | | | LCMS |
|---|---|---|---|
| Example | Starting Amide | Structure | (m/z) |
| 120 | 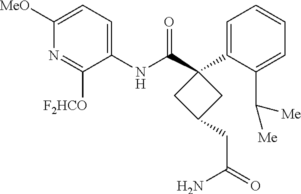 [SMILES: COc1ccc(NC(=O)[C@]2(c3ccccc3C(C)C)C[C@@H](CC(N)=O)C2)c(OC(F)F)n1] Example 118 | 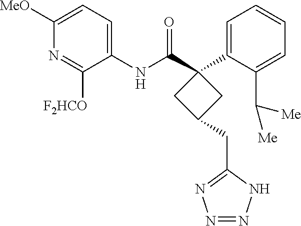 [SMILES: COc1ccc(NC(=O)[C@]2(c3ccccc3C(C)C)C[C@@H](Cc3nnn[nH]3)C2)c(OC(F)F)n1] trans-3-((1H-tetrazol-5- yl)methyl)-N-(2- (difluoromethoxy)-6- methoxypyridin-3-yl)-1-(2- isopropylphenyl)cyclobutane-1- carboxamide | 473.0 [M+H] |

### Additional Examples

| Example | Structure | Example | Structure |
|---|---|---|---|
| 121 | 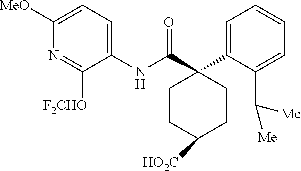 (1s,4s)-4-((2-(difluoromethoxy)- 6-methoxypyridin-3- yl)carbamoyl)-4-(2- isopropylphenyl)cyclohexane-1- carboxylic acid | 122 | 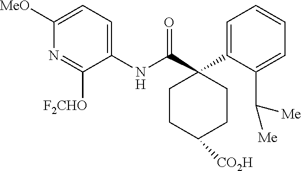 (1r,4r)-4-((2-(difluoromethoxy)- 6-methoxypyridin-3- yl)carbamoyl)-4-(2- isopropylphenyl)cyclohexane-1- carboxylic acid |
| 123 | 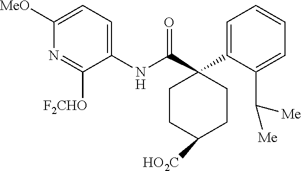 (1s,4s)-4-((2-(difluoromethoxy)- 6-methylpyridin-3- yl)carbamoyl)-4-(2- isopropylphenyl)cyclohexane-1- carboxylic acid | 124 | 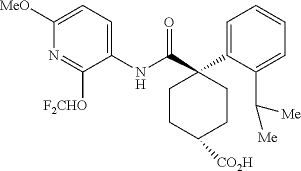 (1r,4r)-4-((2-(difluoromethoxy)- 6-methylpyridin-3- yl)carbamoyl)-4-(2- isopropylphenyl)cyclohexane-1- carboxylic acid |
| 125 | 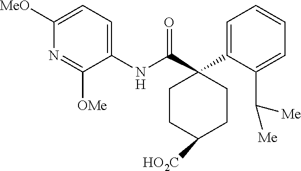 [SMILES: COc1ccc(NC(=O)[C@]2(c3ccccc3C(C)C)CC[C@H](C(=O)O)CC2)c(OC)n1] (1s,4s)-4-((2,6- dimethoxypyridin-3- yl)carbamoyl)-4-(2- isopropylphenyl)cyclohexane-1- carboxylic acid | 126 | 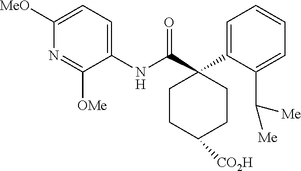 [SMILES: COc1ccc(NC(=O)[C@]2(c3ccccc3C(C)C)CC[C@@H](C(=O)O)CC2)c(OC)n1] (1r,4r)-4-((2,6- dimethoxypyridin-3- yl)carbamoyl)-4-(2- isopropylphenyl)cyclohexane-1- carboxylic acid |
| 127 | 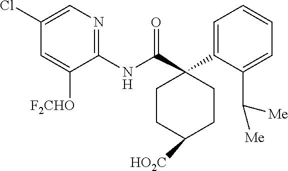 (1s,4s)-4-((5-chloro-3- (difluoromethoxy)pyridin-2- yl)carbamoyl)-4-(2- isopropylphenyl)cyclohexane-1- carboxylic acid | 128 | 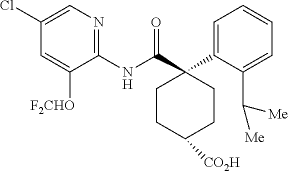 (1r,4r)-4-((5-chloro-3- (difluoromethoxy)pyridin-2- yl)carbamoyl)-4-(2- isopropylphenyl)cyclohexane-1- carboxylic acid |
| 129 | 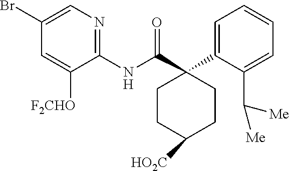 (1s,4s)-4-((5-bromo-3- (difluoromethoxy)pyridin-2- yl)carbamoyl)-4-(2- isopropylphenyl)cyclohexane-1- carboxylic acid | 130 | 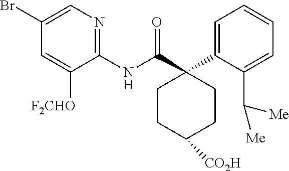 (1r,4r)-4-((5-bromo-3- (difluoromethoxy)pyridin-2- yl)carbamoyl)-4-(2- isopropylphenyl)cyclohexane-1- carboxylic acid |
| 131 | 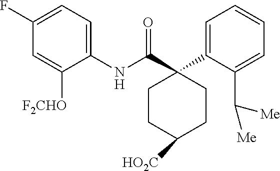 (1s,4s)-4-((2-(difluoromethoxy)- 4-fluorophenyl)carbamoyl)-4-(2- isopropylphenyl)cyclohexane-1- carboxylic acid | 132 | 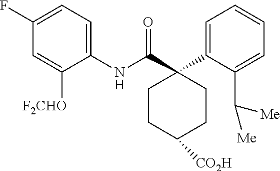 (1r,4r)-4-((2-(difluoromethoxy)- 4-fluorophenyl)carbamoyl)-4-(2- isopropylphenyl)cyclohexane-1- carboxylic acid |
| 133 | 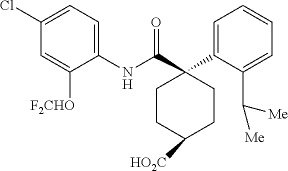 (1s,4s)-4-((4-chloro-2- (difluoromethoxy)phenyl) carbamoyl)-4-(2- isopropylphenyl)cyclohexane-1- carboxylic acid | 134 | 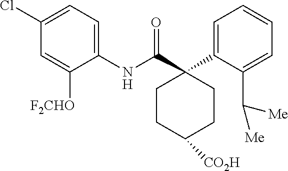 (1r,4r)-4-((4-chloro-2- (difluoromethoxy)phenyl) carbamoyl)-4-(2- isopropylphenyl)cyclohexane-1- carboxylic acid |
| 135 | 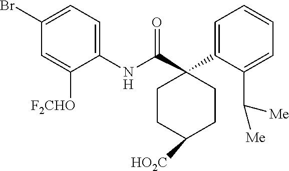 (1s,4s)-4-((4-bromo-2- (difluoromethoxy)phenyl) carbamoyl)-4-(2- isopropylphenyl)cyclohexane-1- carboxylic acid | 136 | 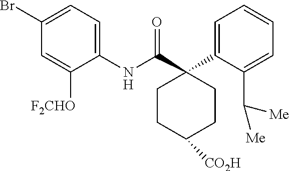 (1r,4r)-4-((4-bromo-2- (difluoromethoxy)phenyl) carbamoyl)-4-(2- isopropylphenyl)cyclohexane-1- carboxylic acid |
| 137 | 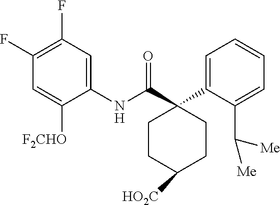 (1s,4s)-4-((2-(difluoromethoxy)- 4,5-difluorophenyl)carbamoyl)- 4-(2- isopropylphenyl)cyclohexane-1- carboxylic acid | 138 | 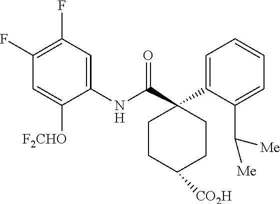 (1r,4r)-4-((2-(difluoromethoxy)- 4,5-difluorophenyl)carbamoyl)- 4-(2- isopropylphenyl)cyclohexane-1- carboxylic acid |
| 139 | 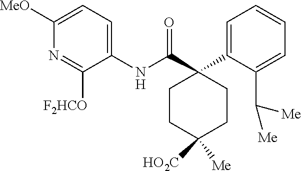 [SMILES: COc1ccc(NC(=O)[C@]2(c3ccccc3C(C)C)CC[C@](C)(C(=O)O)CC2)c(OC(F)F)n1] (1s,4s)-4-((2-(difluoromethoxy)- 6-methoxypyridin-3- yl)carbamoyl)-4-(2- isopropylphenyl)-1- methylcyclohexane-1-carboxylic acid | 140 | 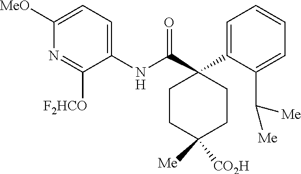 [SMILES: COc1ccc(NC(=O)[C@]2(c3ccccc3C(C)C)CC[C@@](C)(C(=O)O)CC2)c(OC(F)F)n1] (1r,4r)-4-((2-(difluoromethoxy)- 6-methoxypyridin-3- yl)carbamoyl)-4-(2- isopropylphenyl)-1- methylcyclohexane-1-carboxylic acid |
| 141 | 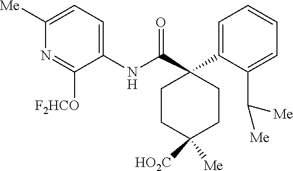 [SMILES: Cc1ccc(NC(=O)[C@]2(c3ccccc3C(C)C)CC[C@](C)(C(=O)O)CC2)c(OC(F)F)n1] (1s,4s)-4-((2-(difluoromethoxy)- 6-methylpyridin-3- yl)carbamoyl)-4-(2- isopropylphenyl)-1- methylcyclohexane-1-carboxylic acid | 142 | 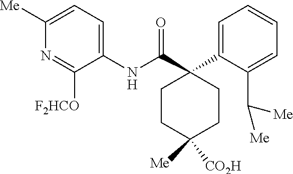 [SMILES: Cc1ccc(NC(=O)[C@]2(c3ccccc3C(C)C)CC[C@@](C)(C(=O)O)CC2)c(OC(F)F)n1] (1r,4r)-4-((2-(difluoromethoxy)- 6-methylpyridin-3- yl)carbamoyl)-4-(2- isopropylphenyl)-1- methylcyclohexane-1-carboxylic acid |
| 143 | 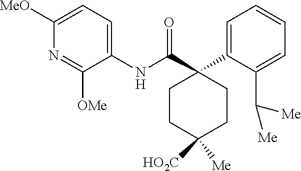 [SMILES: COc1ccc(NC(=O)[C@]2(c3ccccc3C(C)C)CC[C@](C)(C(=O)O)CC2)c(OC)n1] (1s,4s)-4-((2,6- dimethoxypyridin-3- yl)carbamoyl)-4-(2- isopropylphenyl)-1- methylcyclohexane-1-carboxylic acid | 144 | 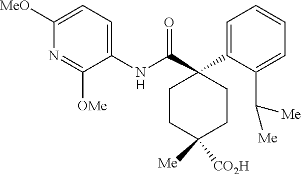 [SMILES: COc1ccc(NC(=O)[C@]2(c3ccccc3C(C)C)CC[C@@](C)(C(=O)O)CC2)c(OC)n1] (1r,4r)-4-((2,6- dimethoxypyridin-3- yl)carbamoyl)-4-(2- isopropylphenyl)-1- methylcyclohexane-1-carboxylic acid |
| 145 | 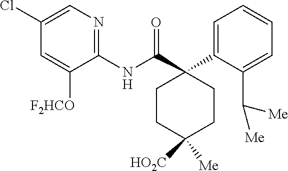 [SMILES: CC(C)c1ccccc1[C@]1(C(=O)Nc2ncc(Cl)cc2OC(F)F)CC[C@@](C)(C(=O)O)CC1] (1s,4s)-4-((5-chloro-3- (difluoromethoxy)pyridin-2- yl)carbamoyl)-4-(2- isopropylphenyl)-1- methylcyclohexane-1-carboxylic acid | 146 | 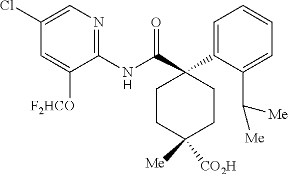 [SMILES: CC(C)c1ccccc1[C@]1(C(=O)Nc2ncc(Cl)cc2OC(F)F)CC[C@](C)(C(=O)O)CC1] (1r,4r)-4-((5-chloro-3- (difluoromethoxy)pyridin-2- yl)carbamoyl)-4-(2- isopropylphenyl)-1- methylcyclohexane-1-carboxylic acid |
| 147 | 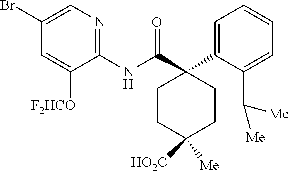 [SMILES: CC(C)c1ccccc1[C@]1(C(=O)Nc2ncc(Br)cc2OC(F)F)CC[C@@](C)(C(=O)O)CC1] (1s,4s)-4-((5-bromo-3- (difluoromethoxy)pyridin-2- yl)carbamoyl)-4-(2- isopropylphenyl)-1- methylcyclohexane-1-carboxylic acid | 148 | 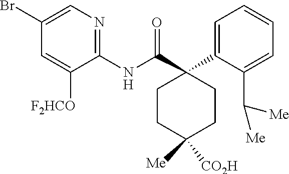 [SMILES: CC(C)c1ccccc1[C@]1(C(=O)Nc2ncc(Br)cc2OC(F)F)CC[C@](C)(C(=O)O)CC1] (1r,4r)-4-((5-bromo-3- (difluoromethoxy)pyridin-2- yl)carbamoyl)-4-(2- isopropylphenyl)-1- methylcyclohexane-1-carboxylic acid |
| 149 | 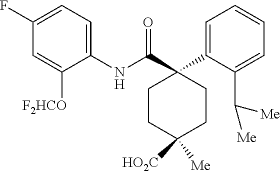 [SMILES: CC(C)c1ccccc1[C@]1(C(=O)Nc2ccc(F)cc2OC(F)F)CC[C@@](C)(C(=O)O)CC1] (1s,4s)-4-((2-(difluoromethoxy)- 4-fluorophenyl)carbamoyl)-4-(2- isopropylphenyl)-1- methylcyclohexane-1-carboxylic acid | 150 | 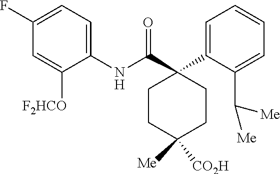 [SMILES: CC(C)c1ccccc1[C@]1(C(=O)Nc2ccc(F)cc2OC(F)F)CC[C@](C)(C(=O)O)CC1] (1r,4r)-4-((2-(difluoromethoxy)- 4-fluorophenyl)carbamoyl)-4-(2- isopropylphenyl)-1- methylcyclohexane-1-carboxylic acid |
| 151 | 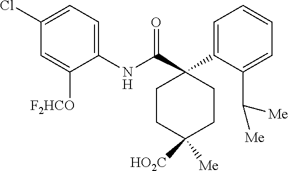 [SMILES: CC(C)c1ccccc1[C@]1(C(=O)Nc2ccc(Cl)cc2OC(F)F)CC[C@@](C)(C(=O)O)CC1] (1s,4s)-4-((4-chloro-2- (difluoromethoxy)phenyl) carbamoyl)-4-(2-isopropylphenyl)-1- methylcyclohexane-1-carboxylic acid | 152 | 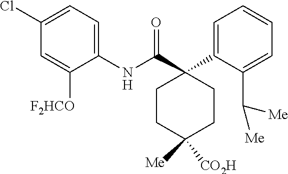 [SMILES: CC(C)c1ccccc1[C@]1(C(=O)Nc2ccc(Cl)cc2OC(F)F)CC[C@](C)(C(=O)O)CC1] (1r,4r)-4-((4-chloro-2- (difluoromethoxy)phenyl) carbamoyl)-4-(2-isopropylphenyl)-1- methylcyclohexane-1-carboxylic acid |
| 153 | 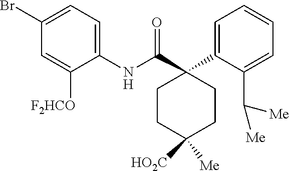 [SMILES: CC(C)c1ccccc1[C@]1(C(=O)Nc2ccc(Br)cc2OC(F)F)CC[C@@](C)(C(=O)O)CC1] (1s,4s)-4-((4-bromo-2- (difluoromethoxy)phenyl) carbamoyl)-4-(2-isopropylphenyl)-1- methylcyclohexane-1-carboxylic acid | 154 | 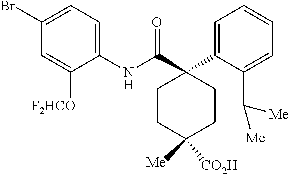 [SMILES: CC(C)c1ccccc1[C@]1(C(=O)Nc2ccc(Br)cc2OC(F)F)CC[C@](C)(C(=O)O)CC1] (1r,4r)-4-((4-bromo-2- (difluoromethoxy)phenyl) carbamoyl)-4-(2-isopropylphenyl)-1- methylcyclohexane-1-carboxylic acid |
| 155 | 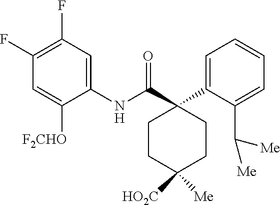 (1s,4s)-4-((2-(difluoromethoxy)- 4,5-difluorophenyl)carbamoyl)- 4-(2-isopropylphenyl)-1- methylcyclohexane-1-carboxylic acid | 156 | 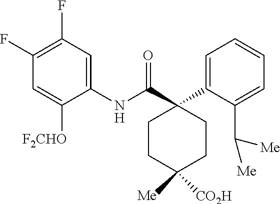 (1r,4r)-4-((2-(difluoromethoxy)- 4,5-difluorophenyl)carbamoyl)- 4-(2-isopropylphenyl)-1- methylcyclohexane-1-carboxylic acid |
| 157 | 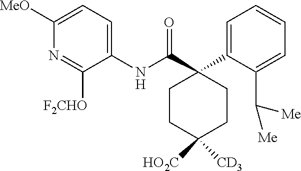 (1s,4s)-4-((2-(difluoromethoxy)- 6-methoxypyridin-3- yl)carbamoyl)-4-(2- isopropylphenyl)-1-(methyl- d₃)cyclohexane-1-carboxylic acid | 158 | 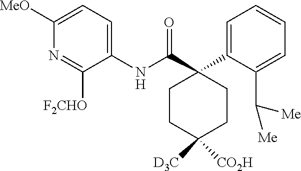 (1r,4r)- 4-((2-(difluoromethoxy)- 6-methoxypyridin-3- yl)carbamoyl)-4-(2- isopropylphenyl)-1-(methyl- d₃)cyclohexane-1-carboxylic acid |
| 159 | 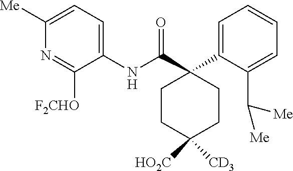 (1s,4s)-4-((2-(difluoromethoxy)- 6-methylpyridin-3- yl)carbamoyl)-4-(2- isopropylphenyl)-1-(methyl- d₃)cyclohexane-1-carboxylic acid | 160 | 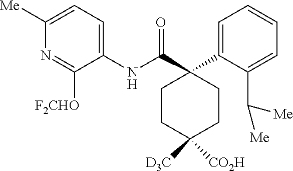 (1r,4r)-4-((2-(difluoromethoxy)- 6-methylpyridin-3- yl)carbamoyl)-4-(2- isopropylphenyl)-1-(methyl- d₃)cyclohexane-1-carboxylic acid |
| 161 | 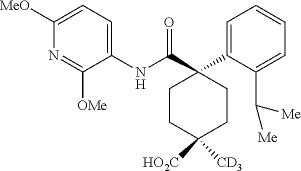 [SMILES: [2H]C([2H])([2H])[C@]1(C(=O)O)CC[C@](C(=O)Nc2ccc(OC)nc2OC)(c2ccccc2C(C)C)CC1] (1s,4s)-4-((2,6- dimethoxypyridin-3- yl)carbamoyl)-4-(2- isopropylphenyl)-1-(methyl- d₃)cyclohexane-1-carboxylic acid | 162 | 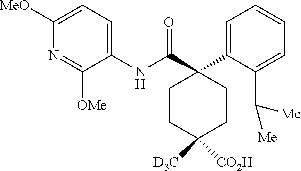 [SMILES: [2H]C([2H])([2H])[C@]1(C(=O)O)CC[C@@](C(=O)Nc2ccc(OC)nc2OC)(c2ccccc2C(C)C)CC1] (1r,4r)-4-((2,6- dimethoxypyridin-3- yl)carbamoyl)-4-(2- isopropylphenyl)-1-(methyl- d₃)cyclohexane-1-carboxylic acid |
| 163 | 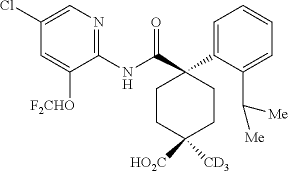 (1s,4s)-4-((5-chloro-3- (difluoromethoxy)pyridin-2- yl)carbamoyl)-4-(2- isopropylphenyl)-1-(methyl- d₃)cyclohexane-1-carboxylic acid | 164 | 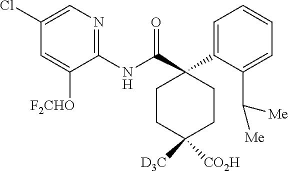 (1r,4r)-4-((5-chloro-3- (difluoromethoxy)pyridin-2- yl)carbamoyl)-4-(2- isopropylphenyl)-1-(methyl- d₃)cyclohexane-1-carboxylic acid |
| 165 | 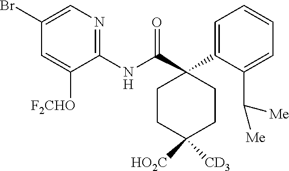 (1s,4s)-4-((5-bromo-3- (difluoromethoxy)pyridin-2- yl)carbamoyl)-4-(2- isopropylphenyl)-1-(methyl- d₃)cyclohexane-1-carboxylic acid | 166 | 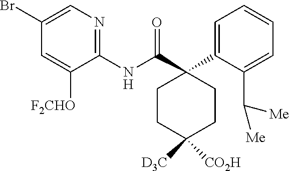 (1r,4r)-4-((5-bromo-3- (difluoromethoxy)pyridin-2- yl)carbamoyl)-4-(2- isopropylphenyl)-1-(methyl- d₃)cyclohexane-1-carboxylic acid |
| 167 | 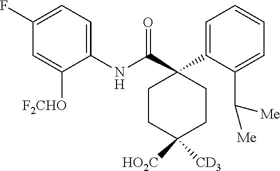 (1s,4s)-4-((2-(difluoromethoxy)- 4-fluorophenyl)carbamoyl)-4-(2- isopropylphenyl)-1-(methyl- d₃)cyclohexane-1-carboxylic acid | 168 | 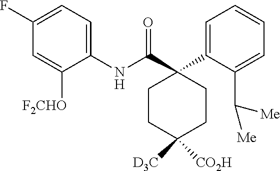 (1r,4r)-4-((2-(difluoromethoxy)- 4-fluorophenyl)carbamoyl)-4-(2- isopropylphenyl)-1-(methyl- d₃)cyclohexane-1-carboxylic acid |
| 169 | 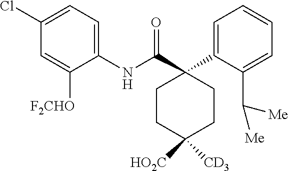 (1s,4s)-4-((4-chloro-2- (difluoromethoxy)phenyl) carbamoyl)-4-(2-isopropylphenyl)-1- (methyl-d₃)cyclohexane-1- carboxylic acid | 170 | 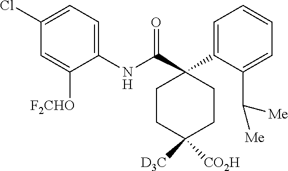 (1r,4r)-4-((4-chloro-2- (difluoromethoxy)phenyl) carbamoyl)-4-(2-isopropylphenyl)-1- (methyl-d₃)cyclohexane-1- carboxylic acid |
| 171 | 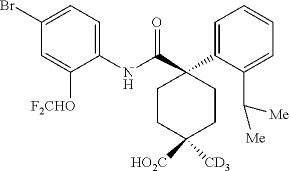 (1s,4s)-4-((4-bromo-2- (difluoromethoxy)phenyl) carbamoyl)-4-(2-isopropylphenyl)-1- (methyl-d₃)cyclohexane-1- carboxylic acid | 172 | 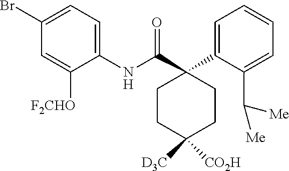 (1r,4r)-4-((4-bromo-2- (difluoromethoxy)phenyl) carbamoyl)-4-(2-isopropylphenyl)-1- (methyl-d₃)cyclohexane-1- carboxylic acid |
| 173 | 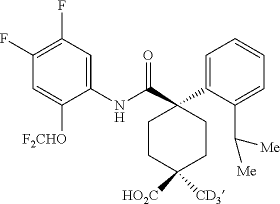 (1s,4s)-4-((2-(difluoromethoxy)- 4,5-difluorophenyl)carbamoyl)- 4-(2-isopropylphenyl)-1-(methyl- d₃)cyclohexane-1-carboxylic acid | 174 | 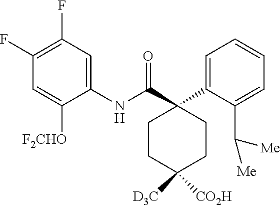 (1r,4r)-4-((2-(difluoromethoxy)- 4,5-difluorophenyl)carbamoyl)- 4-(2-isopropylphenyl)-1-(methyl- d₃)cyclohexane-1-carboxylic acid |

### 2. Biological Evaluations

In vitro functional assay of lysophosphatidic acid receptor 1 activity

Primary compound plates were prepared in 100% DMSO (Sigma-Aldrich), secondary compound plates were prepared at 10× concentration in DiVEM (Invitrogen), and tertiary compound plates were prepared at 3x concentration in assay buffer containing HBSS (no Ca⁺²/Mg⁺², Invitrogen) and 0.1% BSA (Sigma-Aldrich). Fluo-4 NW calcium assay dye (Invitrogen) was prepared as per manufacturer's recommendations in assay buffer. B103 cells stably expressing human LPA₁(J. Chun lab, UCSD) were grown to confluency in DMEM media (Invitrogen) containing 10% FBS (ATCC), 1000 Penicillin-Streptomycin (Sigma-Aldrich) and 50 μg Geneticin (Sigma-Aldrich), and detached with Accutase (Sigma-Aldrich) prior to assay. The freshly detached cells were resuspended in growth media and plated in black, clear-bottom 96-well plates (Costar) containing compound (i.e., secondary compound plate) at a density of 5×10⁴ cells/well. Once plated, cells were left at room temperature for 30 minutes and then transferred to a 37° C., 500 CO₂ incubator for 24 hours. After 24 hours, the growth media was then removed and freshly prepared Fluo-4 NW calcium assay dye was added to cells. Compounds (i.e. tertiary compound plates) were then added back to the dye/cells, returned to the incubator for 30 minutes, and then kept at room temperature for an additional 30 minutes. Finally, lysophosphatidic acid (18:1) at the EC₈₀ concentration was added and calcium flux measured using FlexStation 3 (Molecular Devices). Sigmoidal dose-response curves were generated by measuring luminescence over 45 sec and calculating the area under the curve. Dose response curves and IC₅₀ values were generated using Prism (GraphPad). Compounds were tested at a final concentration range of 100 pM to 10 μM in 0.10% DMSO. Results are shown in Table 1.

TABLE 1

| | LPA₁ | | LPA₁ | | LPA₁ | | LPA₁ |
|---|---|---|---|---|---|---|---|
| Example | IC₅₀ | Example | IC₅₀ | Example | IC₅₀ | Example | IC₅₀ |
| 1 | B | 2 | C | 3 | B | 4 | C |
| 5 | D | 6 | C | 7 | C | 8 | A |
| 9 | B | 10 | B | 11 | C | 12 | D |
| 13 | B | 14 | C | 15 | A | 16 | A |
| 17 | B | 18 | C | 19 | B | 20 | D |
| 21 | B | 22 | B | 23 | C | 24 | B |
| 25 | C | 26 | B | 27 | B | 28 | D |
| 29 | C | 30 | B | 31 | B | 32 | C |
| 33 | B | 34 | B | 35 | C | 36 | D |
| 37 | C | 38 | C | 39 | C | 40- | C |
| | | | | | | isomer A | |
| 40- | C | 41 | D | 42 | C | 43 | B |
| isomer B | | | | | | | |
| 44 | C | 45 | A | 46 | A | 47 | B |
| 48 | B | 49 | B | 50 | B | 51- | C |
| | | | | | | isomer A | |
| 51- | C | 52 | B | 53 | A | 54- | A |
| isomer B | | | | | | isomer A | |
| 54- | B | 55- | B | 55- | B | 56 | B |
| isomer B | | isomer A | | isomer B | | | |
| 57 | B | 58 | C | 59 | B | 60 | B |
| 61 | D | 62 | C | 63 | C | 64 | C |
| 65 | B | 66 | C | 67 | C | 68 | B |
| 69 | B | 70 | B | 71 | C | 72 | B |
| 73 | D | 74 | D | 75- | B | 75- | B |
| | | | | isomer A | | isomer B | |
| 76- | C | 76- | B | 77- | A | 77- | A |
| isomer A | | isomer B | | isomer A | | isomer B | |
| 78- | A | 78- | A | 79 | C | 80 | B |
| isomer A | | isomer B | | | | | |
| 81 | B | 82 | A | 83- | D | 83- | B |
| | | | | isomer A | | isomer B | |
| 84 | B | 85 | C | 86 | C | 87 | B |
| 88 | C | 89- | B | 89- | C | 90- | B |
| | | isomer A | | isomer B | | isomer A | |
| 90- | C | 92- | B | 92- | B | 93- | B |
| isomer B | | isomer A | | isomer B | | isomer A | |
| 93- | C | 94- | C | 94- | B | 95 | B |
| isomer B | | isomer A | | isomer B | | | |
| 96 | C | 97 | C | 98 | C | 99 | C |
| 100 | C | 101 | B | 102- | B | 102- | D |
| | | | | isomer A | | isomer B | |
| 103 | C | 104 | B | 105 | B | 106 | C |
| 107 | D | 108 | B | 109 | B | 110 | B |
| 111 | B | 112 | B | 113 | A | 115 | C |
| 116 | C | 117 | B | 118 | B | 119 | B |
| 120 | A | | | | | | |

A = IC₅₀ of less than 10 nM; B = IC₅₀ of less than 100 nM but greater or equal to 10 nM; C = IC₅₀ of less than 1,000 nM but greater or equal to 100 nM; D = IC₅₀ of greater or equal to 1,000 nM.

1. A compound, or a pharmaceutically acceptable salt or solvate thereof, having the formula: 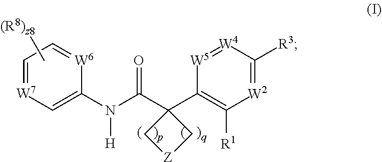
wherein
R¹ is unsubstituted C₂-C₅ alkyl;
W² is N or C(R²);
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁴ is N or C(R⁴);
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁵ is N or C(R⁵);
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted cycloalkyl, substituted or unsubstituted heterocycloalkyl, substituted or unsubstituted aryl, or substituted or unsubstituted heteroaryl;
R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl;
W⁶ is N or C(R⁶);
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR⁶CNR^{6A}R^{6B}, —NHC(O)NR^{6A}R^{6B}, —N(O)ₘ₆, —NR^{6A}R^{6B} C(O)R^{6C}, —C(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁷ is N, N⁺—O⁻, or C(R⁷);
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —NR^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX^{8.3}, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —N^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl;
Z is O, C(R¹¹)(R¹²), or C(O)N(R³);
R¹¹ is hydrogen, halogen, —CX¹¹₃, —CHX¹¹₂, —CH₂X¹¹, —OCX¹¹₃, —OCH₂X¹¹, —OCHX¹¹₂, —CN, —SOₙ₁₁R^{11D}, —SOᵥ₁₁NR^{11A}R^{11B}, —NR¹cNR^{11A}R^{11B}, —ONR^{11A}R^{11B}, —NHC(O)NR^{11C}NR^{11A}R^{11B}, —NHC(O)NR^{11A}R^{11B}, —N(O)ₘ₁, —NR^{11A}R^{11B}, —C(O)R^{11C}, —C(O)OR^{11C}, —C(O)NR^{11A}R^{11B}, —OR^{11D}, —SR^{11D}, —NR^{11A} SO₂R^{11D}, —NR^{11A} C(O)R^{11C}, —NR^{11A} C(O)OR^{11C}, —NR^{11A} OR^{11C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²³, —CHX¹²₂, —CH₂X¹², —OCX¹²₃, —OCH₂X¹², —OCHX¹²₂, —CN, —SOₙ₁₂R^{12D}, —SOᵥ₁₂NR^{12A}R^{12B}, —NR^{12C}NR^{12A}R^{12B}, —ONR^{12A}R^{12B}, —NHC(O)NR^{12C}NR^{12A}R^{12B}, —NHC(O)NR^{12A}R^{12B}, —N(O)ₘ₁₂, —NR^{12A}R^{12B}, —C(O)R^{12C}, —C(O)OR^{12C}, —C(O)NR^{12A}R^{12B}, —OR^{12D}, —SR^{12D}, —NR^{12A}SO₂R^{12D}, —NR^{12A}C(O)R^{12C}, —NR^{12A}C(O)OR^{12C}, —NR^{12A}OR^{12C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹¹ and R¹² substituents may optionally be joined to form an oxo, substituted or unsubstituted cycloalkyl, or substituted or unsubstituted heterocycloalkyl;
R¹³ is hydrogen, halogen, —CX¹³₃, —CHX¹³₂, —CH₂X¹³, —OCX¹³₃, —OCH₂X¹³, —OCHX¹³₂, —SOₙ₁₃R^{13D}, —SOᵥ₁₃NR^{13A}R^{13B}, —C(O)R^{13C}, —C(O)OR^{13C}, —C(O)NR^{13A}R^{13B}, —OR^{13D} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}R^{2C}R^{2D}R^{3A}R^{3B}R^{3C}R^{3D}R^{4A}R^{4B}R^{4C}R^{4D}R^{5A}R^{5B}R^{5C}, R^{5D}R^{6A}, R^{6B}, R^{6C}, R^{6D}, R^{7A}, R^{7B}, R^{7C}, R^{7D}, R^{8A}, R^{8B}, R^{8C}, R^{8D}, R^{11A}, R^{11B}, R^{11C}, R^{11D}, R^{12A}, R^{12B}R^{12C}, R^{12D}, R^{13A}, R^{13B}, R^{13C}, and R^{13D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B}, substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{12A} and R^{12B} substituents bonded to the same nitrogen atom may optionally be joined to form a substituted or unsubstituted heterocycloalkyl or substituted or unsubstituted heteroaryl; R^{13B} and R^{13B} substituents bonded to the same nitrogen atom may optionally be joined to form a substituted or unsubstituted heterocycloalkyl or substituted or unsubstituted heteroaryl;
X², X³, X⁴, X⁵, X⁶, X⁷, X⁸, X¹¹, X¹², and X¹³ are independently —F, —Cl, —Br, or —I;
n2, n3, n4, n5, n6, n7, n8, n11, n12, and n13 are independently an integer from 0 to 4;
m2, m3, m4, m5, m6, m7, m8, m11, m12, v2, v3, v4, v5, v6, v7, v8, v11, v12, and v13 are independently 1 or 2;
z8 is an integer from 0 to 3;
p is 1 or 2; and
q is 1 or 2.

2. The compound of claim 1, having the formula:

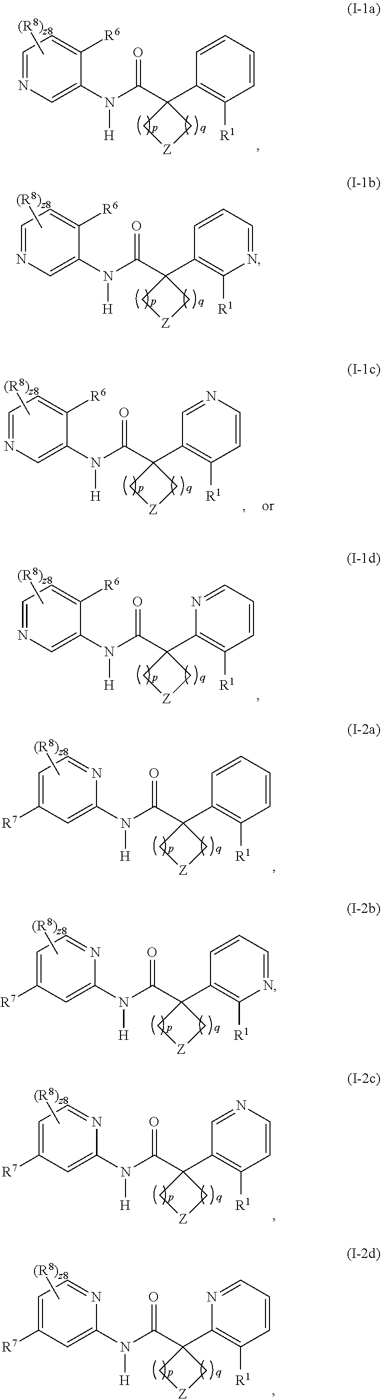

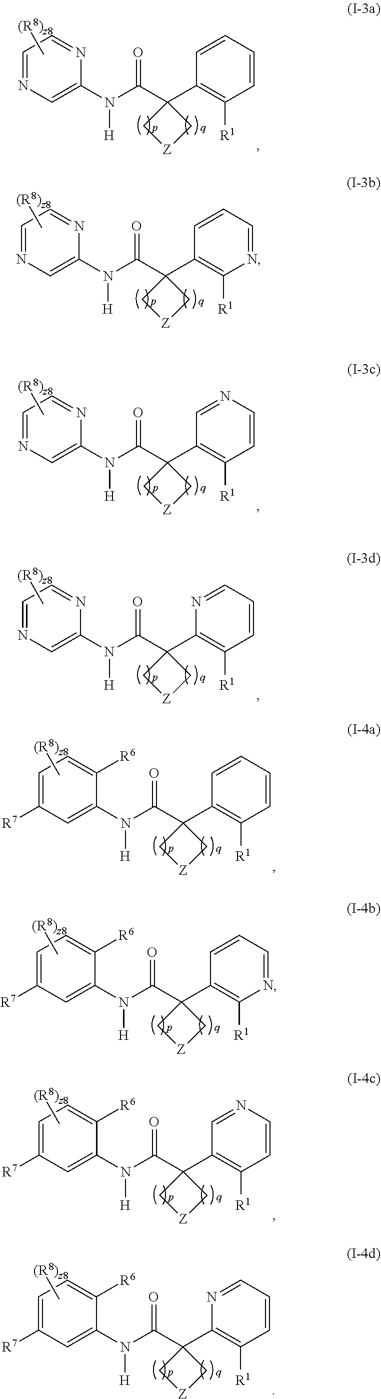

3.-6. (canceled)

7. The compound of claim 1, wherein R¹ is isopropyl.

8. A compound, or a pharmaceutically acceptable salt or solvate thereof, having the formula: 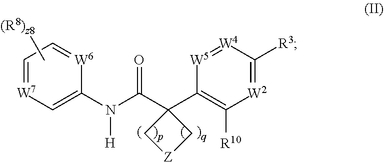
wherein
W² is N or C(R²);
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)OR^{3C}, —C(O)NR^{3A}R^{3B}, —OR^{3D}, —SR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁴ is N or C(R⁴);
R⁴ is hydrogen, halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁵ is N or C(R⁵);
R⁵ is hydrogen, halogen, —CX⁵₃, —CHX⁵₂, —CH₂X⁵, —OCX⁵₃, —OCH₂X⁵, —OCHX⁵₂, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NHC(O)NR^{5A}R^{5B}, —N(O)ₘ₅, —NR^{5A}R^{5B}, —C(O)R^{5C}, —C(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —NR^{5A}OR^{5C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ substituents may optionally be joined to form a substituted or unsubstituted cycloalkyl, substituted or unsubstituted heterocycloalkyl, substituted or unsubstituted aryl, or substituted or unsubstituted heteroaryl;
R³ and R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁴ and R⁵ substituents may optionally be joined to form a substituted or unsubstituted cycloalkyl, substituted or unsubstituted heterocycloalkyl, substituted or unsubstituted aryl, or substituted or unsubstituted heteroaryl;
W⁶ is N or C(R⁶);
R⁶ is hydrogen, halogen, —CX⁶₃, —CHX⁶₂, —CH₂X⁶, —OCX⁶₃, —OCH₂X⁶, —OCHX⁶₂, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR⁶CNR^{6A}R^{6B}, —NHC(O)NR^{6A}R^{6B}, —N(O)ₘ₆, —NR^{6A}R^{6B} C(O)R^{6C}, —C(O)OR^{5C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —NR^{6A}OR^{6C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
W⁷ is N, N⁺—O⁻, or C(R⁷);
R⁷ is hydrogen, halogen, —CX⁷₃, —CHX⁷₂, —CH₂X⁷, —OCX⁷₃, —OCH₂X⁷, —OCHX⁷₂, —CN, —SOₙ₇R^{7D}, —SOᵥ₇NR^{7A}R^{7B}, —NR^{7C}NR^{7A}R^{7B}, —ONR^{7A}R^{7B}, —NHC(O)NR^{7C}NR^{7A}R^{7B}, —NHC(O)NR^{7A}R^{7B}, —N(O)ₘ₇, —NR^{7A}R^{7B}, —C(O)R^{7C}, —C(O)OR^{7C}, —C(O)NR^{7A}R^{7B}, —OR^{7D}, —SR^{7D}, —NR^{7A}SO₂R^{7D}, —NR^{7A}C(O)R^{7C}, —NR^{7A}C(O)OR^{7C}, —NR^{7A}OR^{7C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently halogen, —CX^{8.3}, —CHX⁸₂, —CH₂X⁸, —OCX⁸₃, —OCH₂X⁸, —OCHX⁸₂, —CN, —SOₙ₈R^{8D}, —SOᵥ₈NR^{8A}R^{8B}, —NR^{8C}NR^{8A}R^{8B}, —ONR^{8A}R^{8B}, —NHC(O)NR^{8C}NR^{8A}R^{8B}, —NHC(O)NR^{8A}R^{8B}, —N(O)ₘ₈, —NR^{8A}R^{8B}, —C(O)R^{8C}, —C(O)OR^{8C}, —C(O)NR^{8A}R^{8B}, —OR^{8D}, —SR^{8D}, —N^{8A}SO₂R^{8D}, —NR^{8A}C(O)R^{8C}, —NR^{8A}C(O)OR^{8C}, —NR^{8A}OR^{8C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R⁸ substituents may optionally be joined to form a substituted or unsubstituted cycloalkyl, substituted or unsubstituted heterocycloalkyl, substituted or unsubstituted aryl, or substituted or unsubstituted heteroaryl;
R¹⁰ is hydrogen, halogen, —CX¹⁰ ₃, —CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{11B}, —ONR^{10A}R^{10B}, —NHC(O)NR^{10C}NR^{10A}R^{10B}, —NHC(O)NR^{10A}R^{11B}, —N(O)ₘ₁₀, —NR^{10A}R^{10B}, —C(O)R^{10C}, —C(O)OR^{10C}, —C(O)NR^{10A}R^{10B}, —OR^{10D}, —SR^{10D}, —NR^{10A}SO₂R^{10D}, —NR^{10A}C(O)R^{10C}, —NR^{10A}C(O)OR^{10C}, —NR^{10A} OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R² substituents may optionally be joined to form a substituted or unsubstituted cycloalkyl, substituted or unsubstituted heterocycloalkyl, substituted or unsubstituted aryl, or substituted or unsubstituted heteroaryl;
Z is O, C(R¹¹)(R¹²), or C(O)N(R¹³);
R¹¹ is hydrogen, halogen, —CX¹¹₃, —CHX¹¹₂, —CH₂X¹¹, —OCX¹¹₃, —OCH₂X¹¹, —OCHX¹¹₂, —CN, —SOₙ₁₁R^{11D}, —SOᵥ₁₁NR^{11A}R^{11B}, —NR^{11C}NR^{11A}R^{11B}, —ONR^{11A}R^{11B}, —NHC(O)NR¹¹CNR^{11A}R^{11B}, —NHC(O)NR^{11A}R^{11B}, —N(O)ₘ₁, —NR^{11A}R^{11B}, —C(O)R^{11C}, —C(O)OR^{11C}, —C(O)NR^{11A}R^{11B}, —OR^{11D}, —SR^{11D}, —NR^{11A} SO₂R^{11D}, —NR^{11A} C(O)R^{11C}, —NR^{11A} C(O)OR^{11C}, —NR^{11A} OR^{11C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²³, —CHX¹²₂, —CH₂X¹², —OCX¹²₃, —OCH₂X¹², —OCHX¹²₂, —CN, —SOₙ₁₂R^{12D}, —SOᵥ₁₂NR^{12A}R^{12B}, —NR^{12C}NR^{12A}R^{12B}, —ONR^{12A}R^{12B}, —NHC(O)NR^{12C}NR^{12A}R^{12B}, —NHC(O)NR^{12A}R^{12B}, —N(O)ₘ₁₂, —NR^{12A}R^{12B}, —C(O)R^{12C}, —C(O)OR^{12C}, —C(O)NR^{12A}R^{12B}, —OR^{12D}, —SR^{12D}, —NR^{12A}SO₂R^{12D}, —NR^{12A}C(O)R^{12C}, —NR^{12A}C(O)OR^{12C}, —NR^{12A}OR^{12C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹¹ and R¹² substituents may optionally be joined to form an oxo, substituted or unsubstituted cycloalkyl, or substituted or unsubstituted heterocycloalkyl;
R¹³ is hydrogen, halogen, —CX¹²₃, —CHX¹³₂, —CH₂X¹³, —OCX¹³₃, —OCH₂X¹³, —OCHX¹³₂, —SOₙ₁₃R^{13D}, —SOᵥ₁₃NR^{13A}R^{13B}, —C(O)R^{13C}, —C(O)OR^{13C}, —C(O)NR^{13A}R^{13B}, —OR^{13D} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}R^{6A}, R^{6B}, R^{6C}, R^{6D}, R^{7A}, R^{7B}, R^{7C}, R^{7D}, R^{8A}, R^{8B}, R^{8C}, R^{8D}, R^{10A}, R^{10B}, R^{10C}, R^{10D}, R^{11A}, R^{11B}R^{11C}, R^{11D}, R^{12A}, R^{12B}, R^{12C}, R^{12D}, R^{13A}, R^{13B}, R^{13C}, and R^{13D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B}, substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B}, substituents bonded to the same nitrogen atom may optionally be joined to form a substituted or unsubstituted heterocycloalkyl or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{12A} and R^{12B} substituents bonded to the same nitrogen atom may optionally be joined to form a substituted or unsubstituted heterocycloalkyl or substituted or unsubstituted heteroaryl; R^{13B} and R^{13B} substituents bonded to the same nitrogen atom may optionally be joined to form a substituted or unsubstituted heterocycloalkyl or substituted or unsubstituted heteroaryl;
X², X³, X⁴, X⁵, X⁶, X⁷, X⁸, X¹⁰, X¹¹, X¹², and X¹³ are independently —F, —Cl, —Br, or —I;
n2, n3, n4, n5, n6, n7, n8, n10, n11, n12, and n13 are independently an integer from 0 to 4;
m2, m3, m4, m5, m6, m7, m8, m10, m11, m12, v2, v3, v4, v5, v6, v7, v8, v10, v11, v12, and v13 are independently 1 or 2;
z8 is an integer from 0 to 3;
p is 1 or 2; and
q is 1 or 2;
wherein at least one of W⁶ or W⁷ is N;
wherein if W⁶ is N and W⁷ is C(R⁷), then R¹⁰ is not hydrogen, —F, —Cl, —CH₃, or —OCH₃;
wherein if W⁶ is C(R⁶) and W⁷ is N, then R¹⁰ is not hydrogen, —F, —Cl, —CH₃, or —OCH₃; and
wherein if W⁶ and W⁷ are N and Z is O or CH₂, then R¹⁰ is not hydrogen, —F, —Cl, —CH₃ or —OCH₃.

9. The compound of claim 8, having the formula:

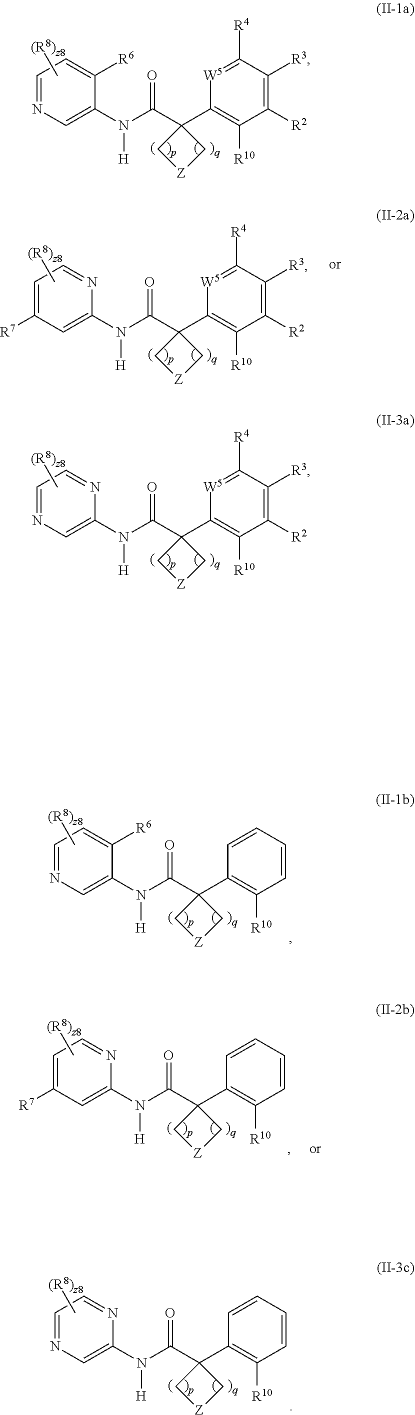

10. (canceled)

11. (canceled)

12. The compound of claim 8, wherein R¹⁰ is isopropyl.

13. (canceled)

14. The compound of claim 1, wherein R⁸ is independently halogen, —CF₃, —CHF₂, —OCHF₂, —OR^{8D}, unsubstituted C₁-C₆ alkyl, or unsubstituted 2 to 8 membered heteroalkyl.

15. (canceled)

16. (canceled)

17. The compound of claim 1, wherein 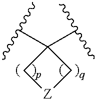
is

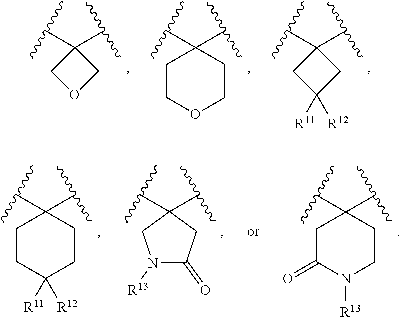

18. (canceled)

19. The compound of claim 17, wherein R¹¹ is hydrogen, halogen, —SOₙ₁₁R^{11D}, —SOᵥ₁₁NR^{11A}R^{11B}, —NR^{11A}R^{11B}, —C(O)OR^{11C}, —C(O)NR^{11A}R^{11B}, —OR^{11D}, —NR^{11A} SO₂R^{11D}—NR^{11A} C(O)R^{11C}, substituted or unsubstituted C₁-C₆ alkyl, substituted or unsubstituted 2 to 6 membered heteroalkyl, substituted or unsubstituted 3 to 8 membered heterocycloalkyl, or substituted or unsubstituted 5 to 6 membered heteroaryl.

20.-23. (canceled)

24. The compound of claim 17, wherein R¹² is hydrogen, halogen, —SOₙ₁₂R^{12D}, —Sᵥ₁₂NR^{12A}R^{12B}, —NR^{12A}R^{12B}, —C(O)OR^{12C}, —C(O)NR^{12A}R^{12B}, —OR^{12D}, —NR^{12A} SO₂R^{12D}, —NR^{12A}C(O)R^{12C}, substituted or unsubstituted C₁-C₆ alkyl, substituted or unsubstituted 2 to 6 membered heteroalkyl, substituted or unsubstituted 3 to 8 membered heterocycloalkyl, or substituted or unsubstituted 5 to 6 membered heteroaryl.

25.-30. (canceled)

31. The compound of claim 1, wherein 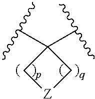
is

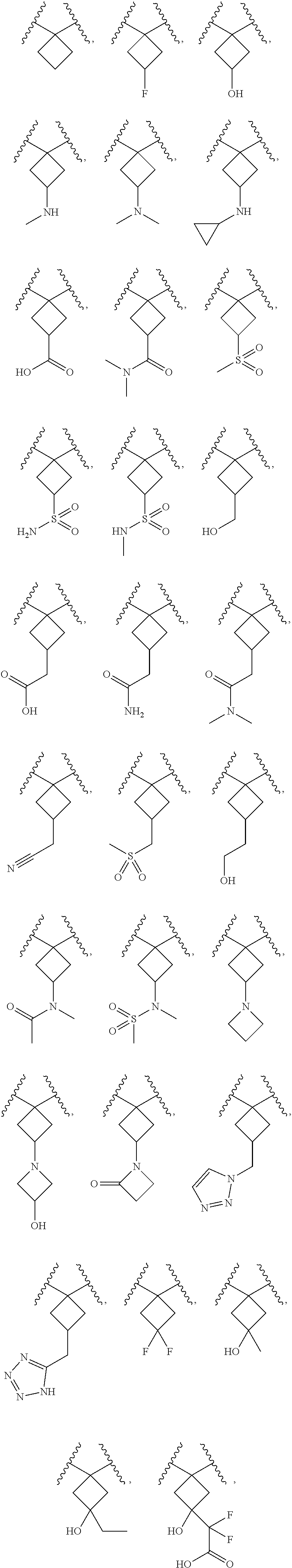

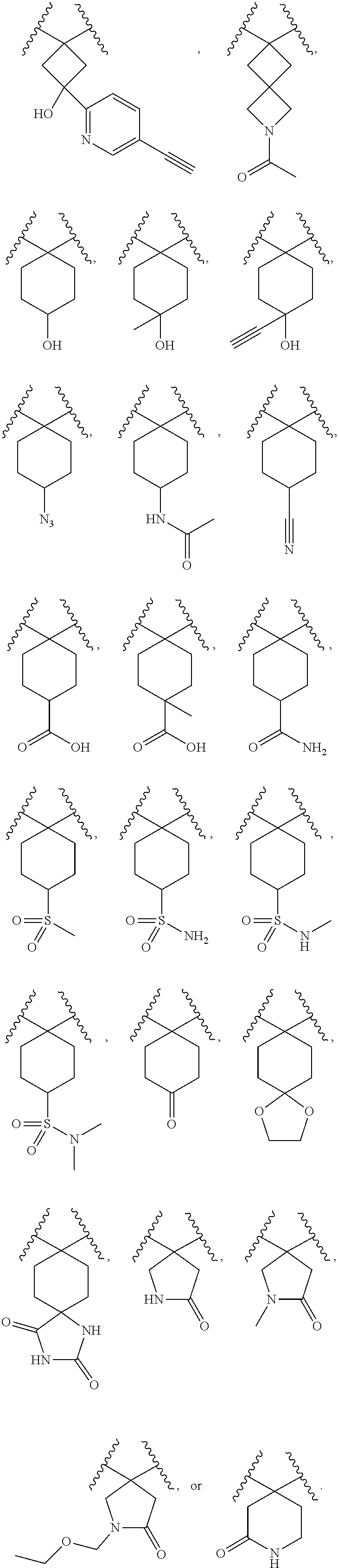

32. (canceled)

33. (canceled)

34. A compound, or a pharmaceutically acceptable salt or solvate thereof, having the formula:

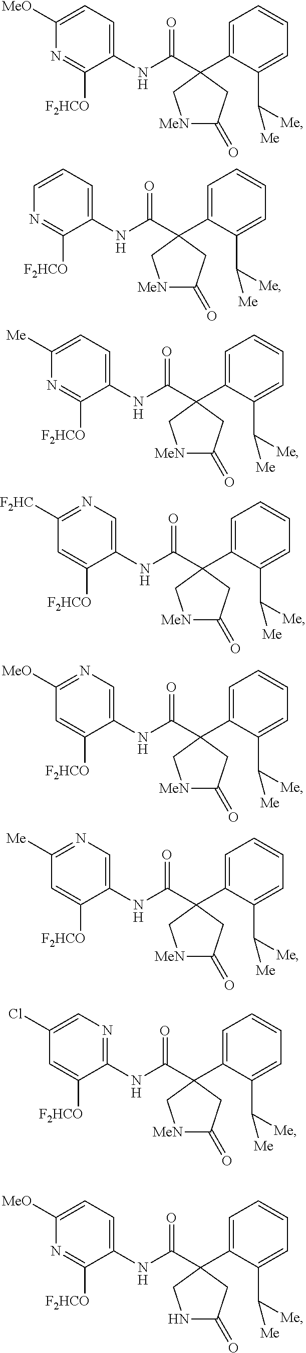

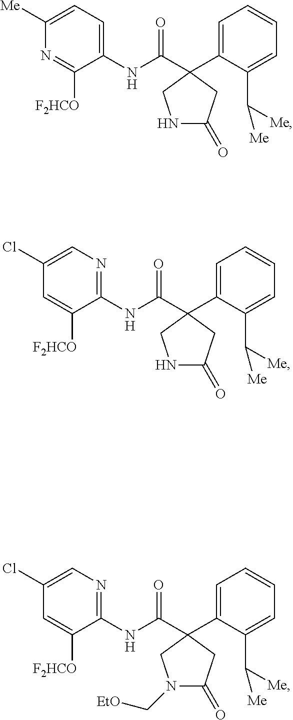 [SMILES: Cc1ccc(NC(=O)C2(c3ccccc3C(C)C)CNC(=O)C2)c(OC(F)F)n1 ; CC(C)c1ccccc1C1(C(=O)Nc2ncc(Cl)cc2OC(F)F)CNC(=O)C1 ; CCOCN1CC(C(=O)Nc2ncc(Cl)cc2OC(F)F)(c2ccccc2C(C)C)CC1=O]

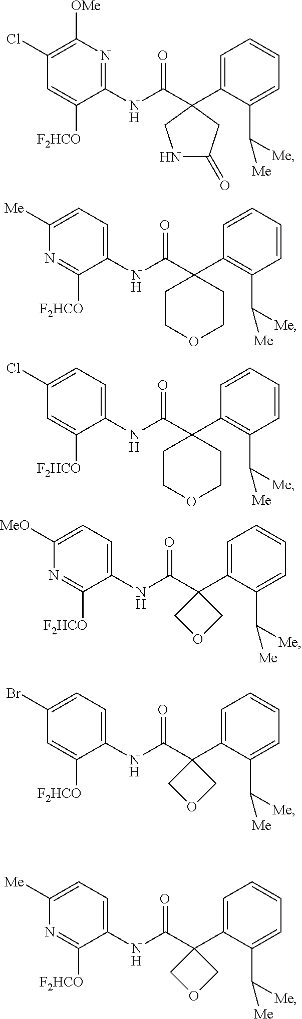

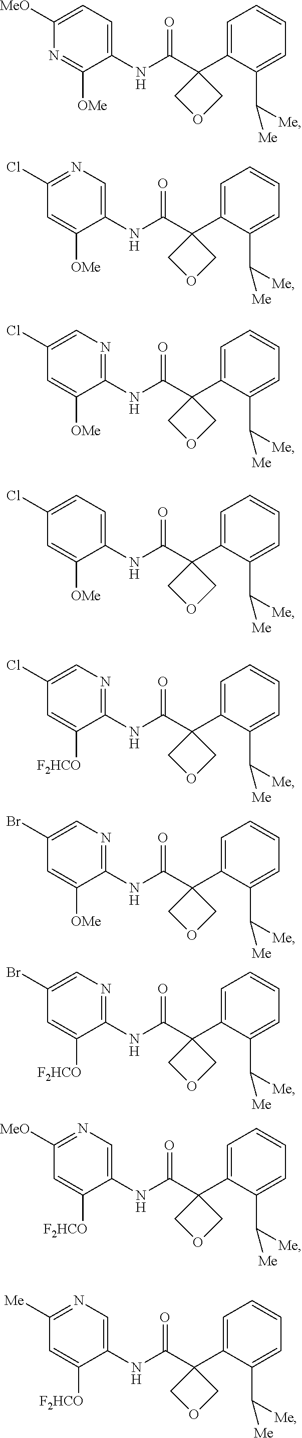

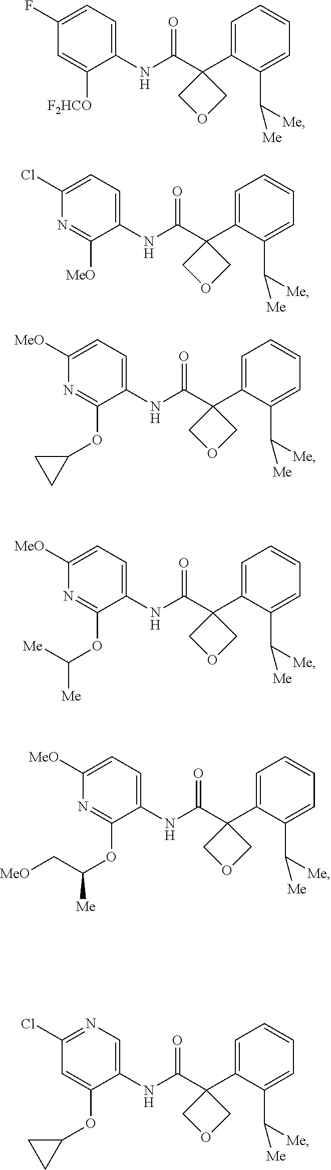

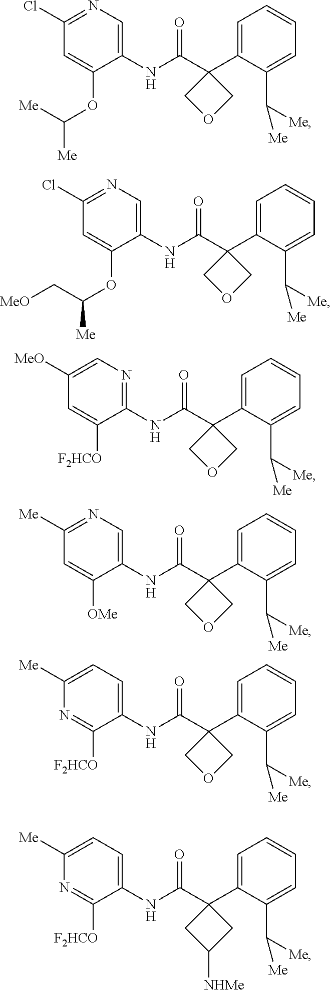

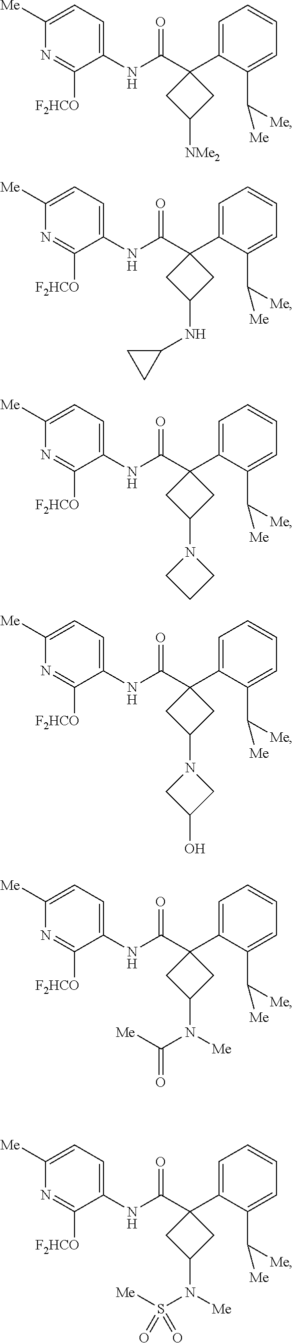

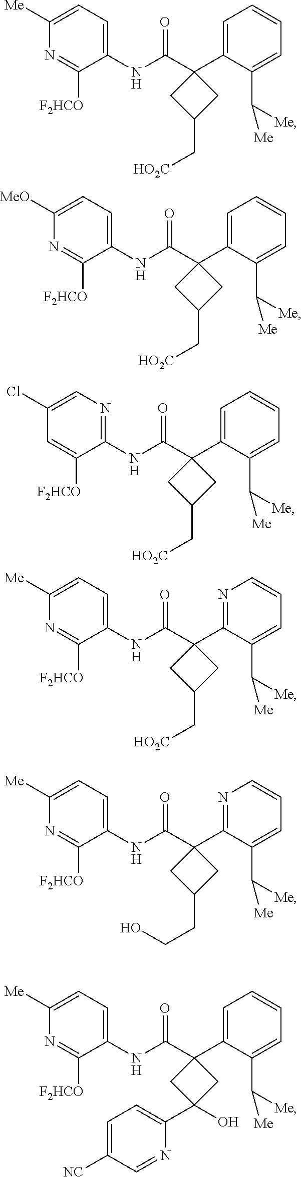

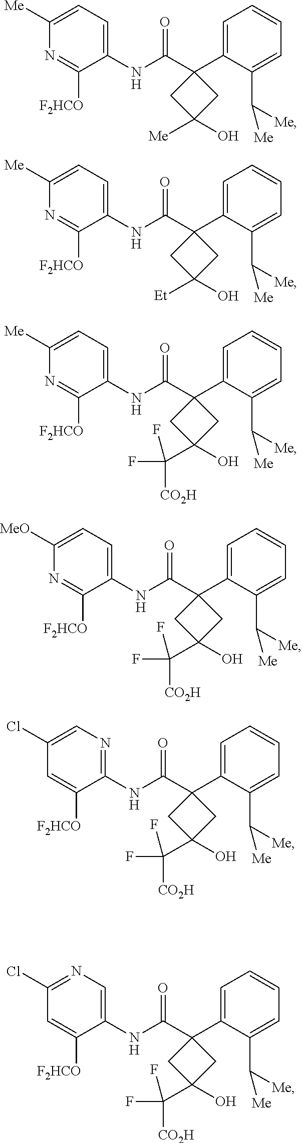

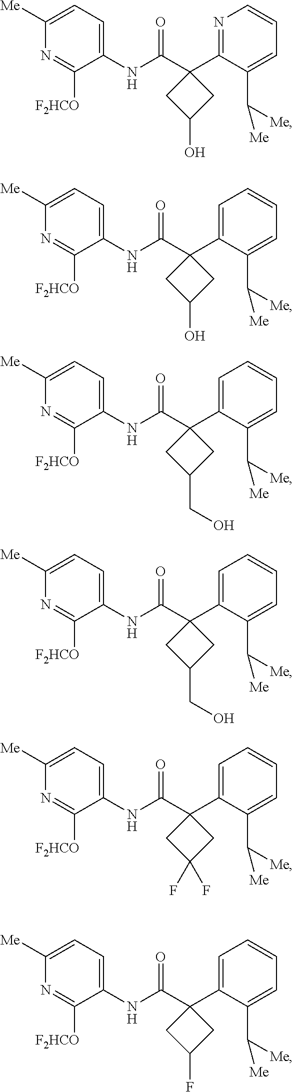

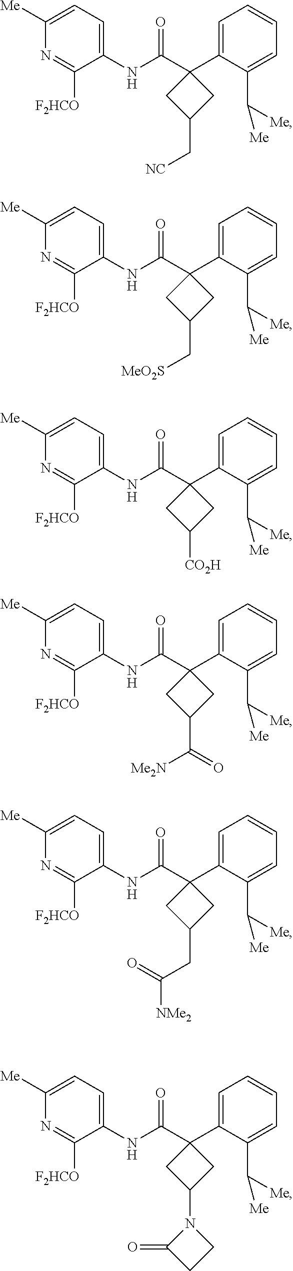 [SMILES: Cc1ccc(NC(=O)C2(c3ccccc3C(C)C)CC(CC#N)C2)c(OC(F)F)n1 ; Cc1ccc(NC(=O)C2(c3ccccc3C(C)C)CC(CS(C)(=O)=O)C2)c(OC(F)F)n1 ; Cc1ccc(NC(=O)C2(c3ccccc3C(C)C)CC(C(=O)O)C2)c(OC(F)F)n1 ; Cc1ccc(NC(=O)C2(c3ccccc3C(C)C)CC(C(=O)N(C)C)C2)c(OC(F)F)n1 ; Cc1ccc(NC(=O)C2(c3ccccc3C(C)C)CC(CC(=O)N(C)C)C2)c(OC(F)F)n1 ; Cc1ccc(NC(=O)C2(c3ccccc3C(C)C)CC(N3CCC3=O)C2)c(OC(F)F)n1]

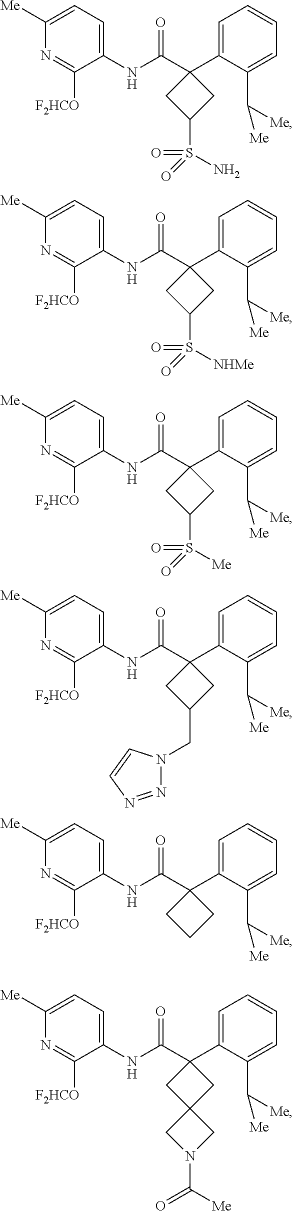

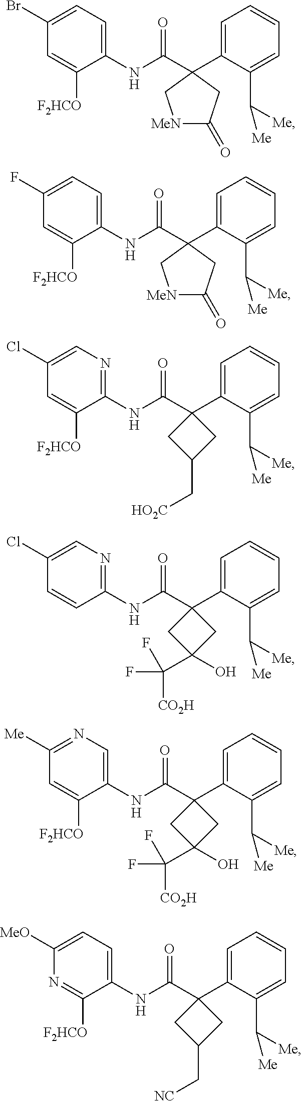

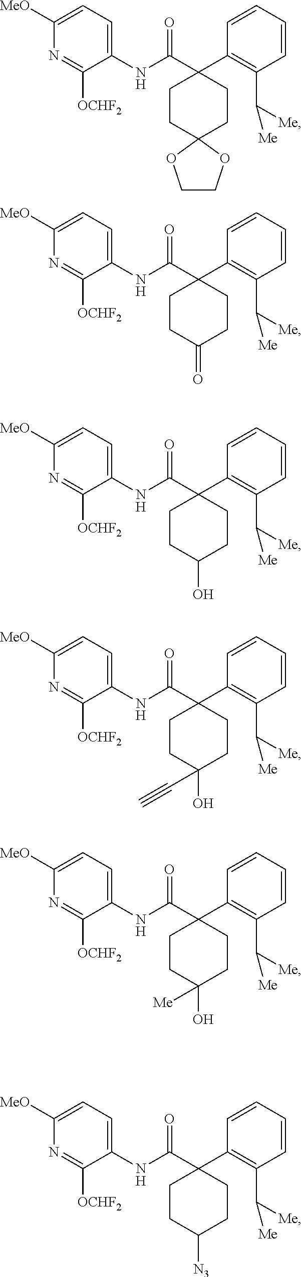

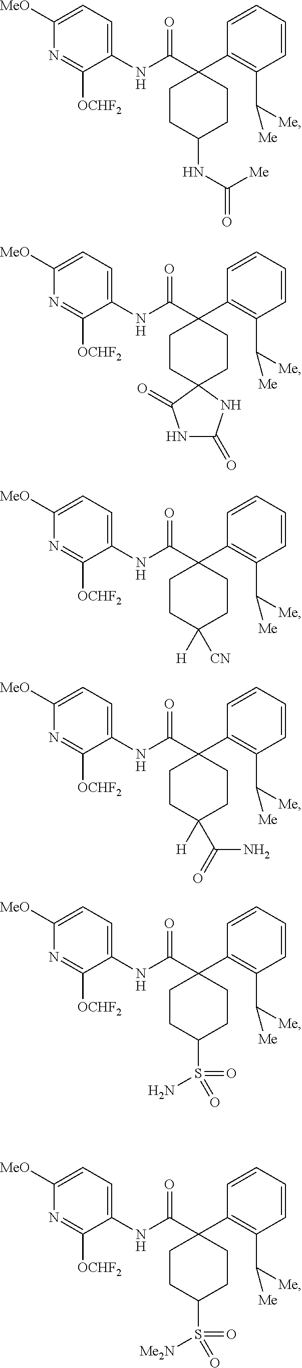

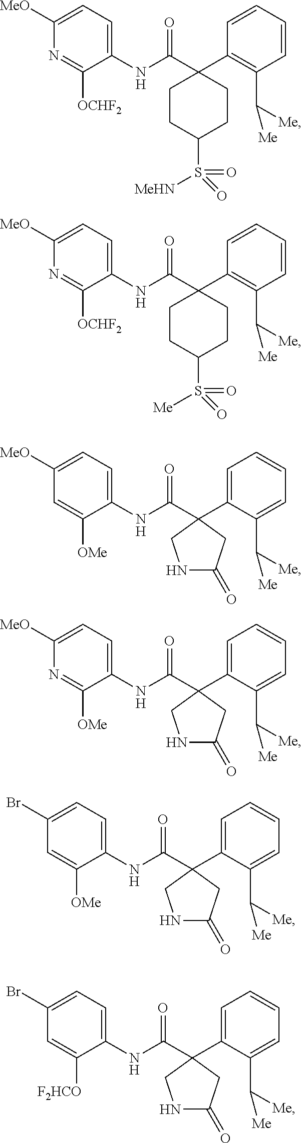

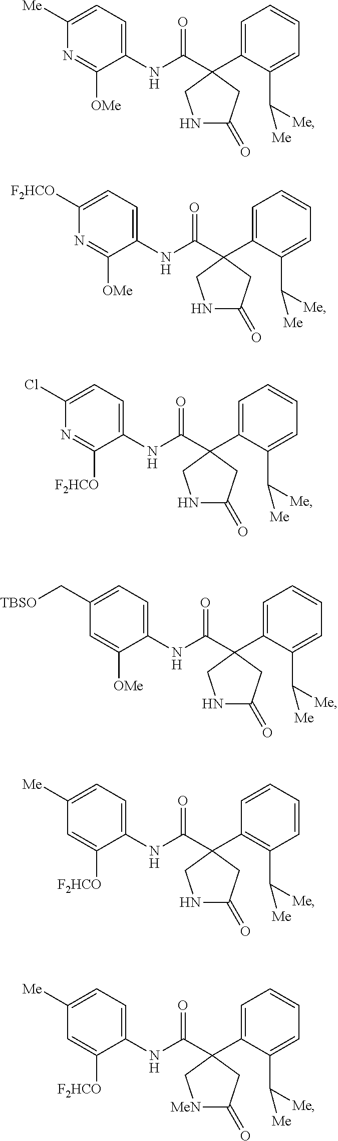 [SMILES: COc1nc(C)ccc1NC(=O)C1(c2ccccc2C(C)C)CNC(=O)C1 ; COc1nc(OC(F)F)ccc1NC(=O)C1(c2ccccc2C(C)C)CNC(=O)C1 ; CC(C)c1ccccc1C1(C(=O)Nc2ccc(Cl)nc2OC(F)F)CNC(=O)C1 ; COc1cc(CO[Si](C)(C)C(C)(C)C)ccc1NC(=O)C1(c2ccccc2C(C)C)CNC(=O)C1 ; Cc1ccc(NC(=O)C2(c3ccccc3C(C)C)CNC(=O)C2)c(OC(F)F)c1 ; Cc1ccc(NC(=O)C2(c3ccccc3C(C)C)CC(=O)N(C)C2)c(OC(F)F)c1]

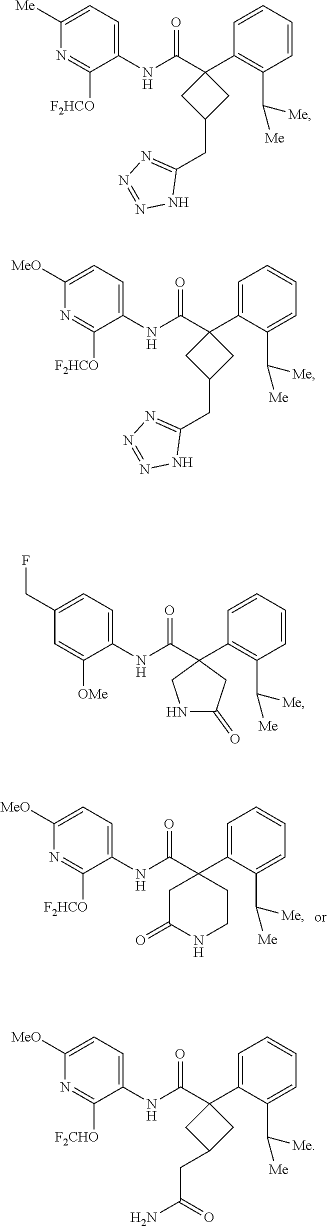

35. A compound, or a pharmaceutically acceptable salt or solvate thereof, having the formula:

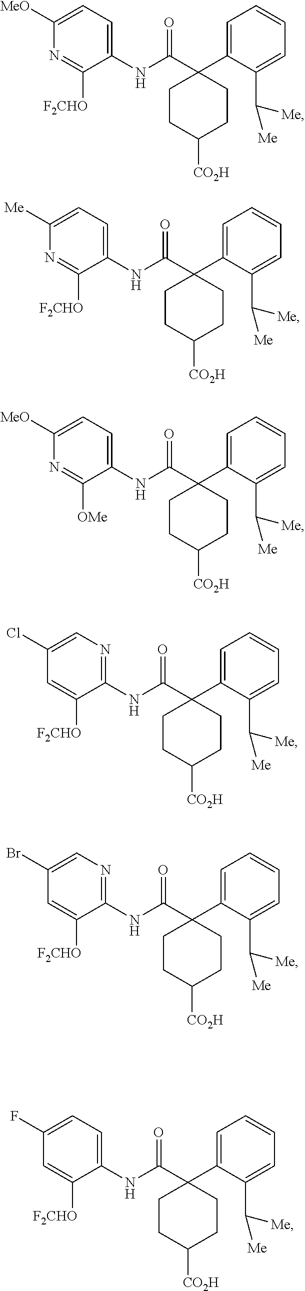

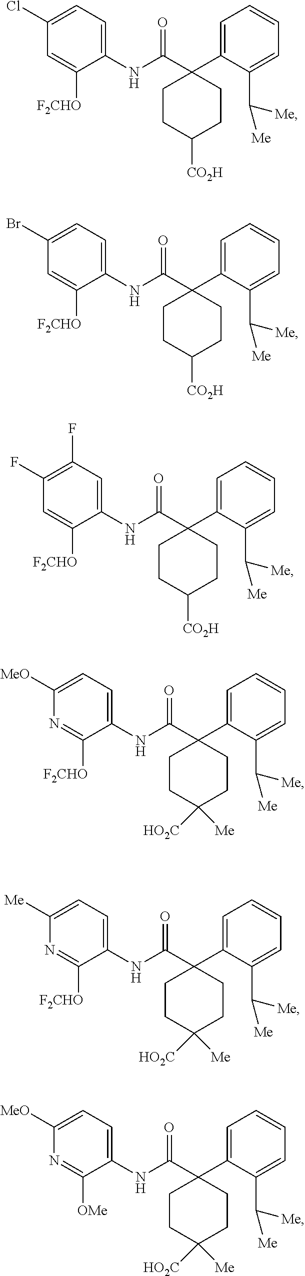

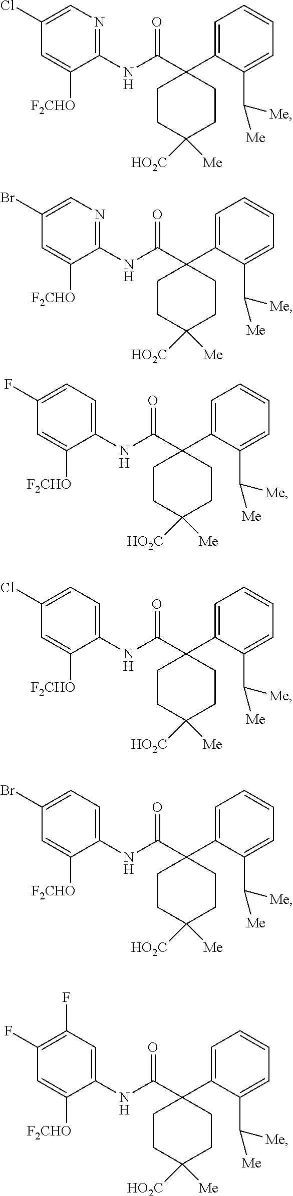

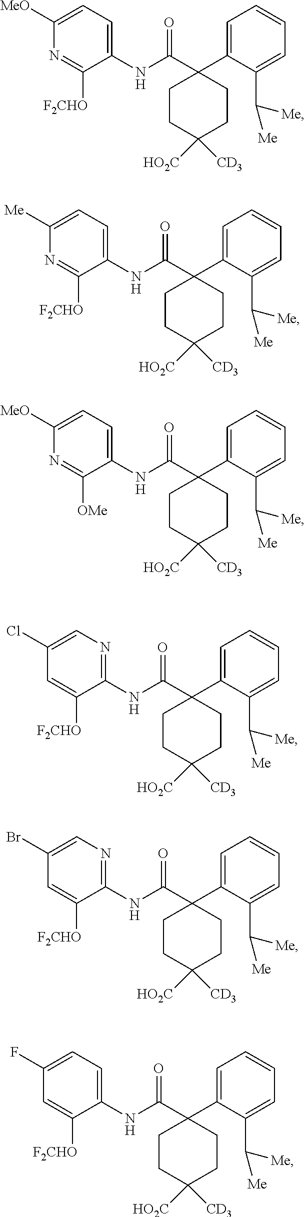

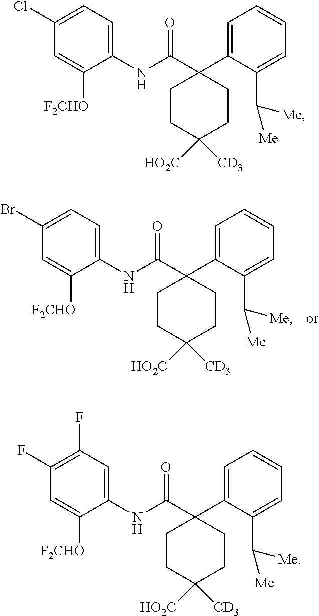

36. A pharmaceutical composition comprising the compound of claim 1, or a pharmaceutically acceptable salt or solvate thereof, and a pharmaceutically acceptable excipient.

37. A method of treating a neurodegenerative disorder in a subject in need thereof, said method comprising administering to the subject in need thereof a therapeutically effective amount of the compound of claim 1, or a pharmaceutically acceptable salt or solvate thereof.

38. A method of treating an inflammatory disease in a subject in need thereof, said method comprising administering to the subject in need thereof a therapeutically effective amount of the compound of claim 1, or a pharmaceutically acceptable salt or solvate thereof.

39. (canceled)

40. (canceled)

41. A method of treating a demyelinating disease in a subject in need thereof, said method comprising administering to the subject in need thereof a therapeutically effective amount of the compound of claim 1, or a pharmaceutically acceptable salt or solvate thereof.

42.-44. (canceled)

45. A method of treating a fibrotic disease in a subject in need thereof, said method comprising administering to the subject in need thereof a therapeutically effective amount of the compound of claim 1, or a pharmaceutically acceptable salt or solvate thereof.

46. (canceled)

47. (canceled)

48. A method of treating cancer in a subject in need thereof, said method comprising administering to the subject in need thereof a therapeutically effective amount of the compound of claim 1, or a pharmaceutically acceptable salt or solvate thereof.

49. (canceled)

50. (canceled)

51. A method of modulating LPAR1 activity in a subject, said method comprising administering to the subject the compound of claim 1, or a pharmaceutically acceptable salt or solvate thereof.
